(19) United States
(12) Patent Application Publication
(10) Pub. No.: US 2026/0347330 A1
(43) Pub. Date: Nov. 5, 2026

(54) N-METHYL-D-ASPARTATE RECEPTOR (NMDAR) ANTAGONISTS AND USES THEREOF

(71) Applicants: Bexson Biomedical, Inc., Santa Barbara, CA (US); Saint Joseph's University, Philadelphia, PA (US)

(72) Inventors: Jason WALLACH, Philadelphia, PA (US); Adeboye ADEJARE, Philadelphia, PA (US); James GAMRAT, Philadelphia, PA (US)

(21) Appl. No.: 19/079,348

(22) PCT Filed: Sep. 14, 2023
(86) PCT No.: PCT/US2023/074246

§ 371 Date: Mar. 13, 2025

Related U.S. Application Data
(60) Provisional application No. 63/407,052, filed on Sep. 15, 2022.

(51) Int. Cl. C07D 295/03; A61K 31/137; A61K 31/341; A61K 31/36; A61K 31/381; A61K 31/40; A61K 31/4402; A61K 31/4406; A61K 31/4409; A61K 31/4453; C07C 211/17; C07C 211/19; C07C 211/27; C07C 217/60; C07D 207/323; C07D 213/38; C07D 307/52; C07D 317/58; C07D 333/20; C07D 333/58; C07F 11/00

(52) U.S. Cl. CPC C07D 295/03; A61K 31/137; A61K 31/341; A61K 31/36; A61K 31/381; A61K 31/40; A61K 31/4402; A61K 31/4406; A61K 31/4409; A61K 31/4453; C07C 211/17; C07C 211/19; C07C 211/27; C07C 217/60; C07D 207/323; C07D 213/38; C07D 307/52; C07D 317/58; C07D 333/20; C07D 333/58; C07F 11/00

(57) ABSTRACT

A series of novel substituted (hetero)arylmethanamines is described which bind to the N-methyl-D-aspartate receptor. Such compounds have value in various therapeutic indications including, but not limited to, pain, depression, tinnitus, post-traumatic stress disorder, etc. Further disclosed a method for treating a physical, psychiatric, or neurological disorder comprising administering to a subject in need thereof a pharmaceutical composition comprising compounds of the invention.

### CROSS REFERENCE TO OTHER APPLICATIONS

This application claims the benefit of U.S. provisional application No. 63/407,052, filed Sep. 15, 2022, the entirety of which is incorporated by reference herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BACKGROUND

The N-methyl-D-aspartate receptor (“NMDAR”) is an ionotropic receptor that allows for the transfer of electrical signals between neurons in the brain and in the spinal cord. For electrical signals to pass, the NMDAR must be open. In order for the channel to open, glutamate and glycine must bind to the NMDAR and the voltage dependent Mg²⁺ block must be lifted. NMDARs in this state are called “activated.”

Chemicals that inhibit activation of the NMDAR are called antagonists. NMDAR antagonists fall into several categories: Competitive antagonists bind to and block the binding site of the neurotransmitter glutamate; glycine antagonists, which bind to and block the glycine site; noncompetitive antagonists inhibit NMDARs by binding to allosteric sites; and uncompetitive antagonists, which block the active channel. One example of NMDAR uncompetitive antagonists are compounds called channel blockers, like ketamine, that act by blocking the ion channel through binding to a site within NMDARs. These compounds show use dependent inhibition as they bind to “activated” NMDARs.

### SUMMARY

There remains a need for treatment of various physical, psychiatric, or neurological disorders associated with ionotropic receptor function, such as NMDAR, dopamine transporter (DAT), or serotonin receptor (SERT). In particular, compounds that selectively bind to NMDAR receptors over other ionotropic receptors may be valuable for the treatment of the various disorders, such as pain, depression, tinnitus, post-traumatic stress disorder, psychosis, schizophrenia, agitation, obsessive compulsive disorder, sexual dysfunction, anxiety, dementias, neurodegenerative diseases, pseudobulbar affect, headache, cluster headache, migraine, acute pain, post-operative pain, pain syndromes, neuropathic pain, chronic pain, complex regional pain syndrome, fibromyalgia, substance use disorders, drug addiction, alcoholism, hallucinations, delusions, insomnia, epilepsies, bipolar disorder, anorexia, or Parkinson's disease.

The compounds of the present disclosure exhibit varying degrees of selectivity for NMDARs over other CNS targets, such as DAT or SERT. NMDAR dysfunction has been implicated in various somatic and central nervous system diseases and disorders. Selectivity for NMDARs can reduce undesired off-target interactions which can improve drug efficacy and tolerability for certain therapeutic indications. However, in some instances, such as certain therapeutic indications including certain types of chronic and neuropathic pain, and mood disorders, polypharmacology for NMDAR and monoamine transporters including DAT, SERT and NET may enhance the efficacy and tolerability of NMDAR antagonists. While effective, existing NMDAR antagonists like ketamine, memantine and DXM have various pharmacological limitations there is thus a need for new NMDAR antagonists with improved clinical profiles.

Inhibiting NMDARs with uncompetitive antagonists which bind to the PCP site of active NMDARs (use-dependent channel blockers) is believed to underlie the therapeutic activity of ketamine as a general anesthetic and for its use in treatment resistant depression and memantine for Alzheimer's disease as a neuroprotective agent. Dextromethorphan, an uncompetitive NMDAR antagonist, is also used, in combination products to inhibit its metabolism, in pseudobulbar affect and in depression. NMDAR antagonism may lead to analgesic actions and can disrupt the processes of hyperalgesia and allodynia in pain disorders. However, these existing NMDAR antagonists have limitations related to their pharmacokinetic and pharmacodynamic properties. For example, DXM has potent off-target effects at SERT, which may contribute to its side effects, and has to be administered in combination with other compounds to inhibit its metabolism. Ketamine has poor oral bioavailability and a short half-life. Additionally, there are ambiguities as to the extent of dissociative effects as well as bladder issues that ketamine has been associated with. Thus, there remains a need for novel NMDAR antagonists with improved clinical profiles.

The present disclosure generally relates to substituted methanamine compounds, conjugates, or salts of Formula (I), (II), (III), (IV), (V), or (VI) and pharmaceutical compositions thereof. In certain aspects, the disclosure provides a compound represented by Formula (I):

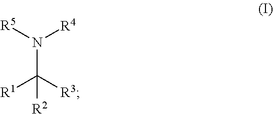

or an enantiomer, a mixture of enantiomers, or an isotopic variant thereof; or a pharmaceutically acceptable salt, solvate or hydrate thereof, wherein:
R¹ is C₃₋₁₀ carbocycle or 3- to 10-membered heterocycle; wherein the C₃₋₁₀ carbocycle and 3- to 10-membered heterocycle are each optionally substituted with one or more R⁶
R² is selected from
   hydrogen;
   halogen;
   C₁₋₈ alkyl, C₂₋₈ alkenyl, and C₂₋₈ alkynyl, each of which is optionally substituted with one or more R⁷; and
   C₃₋₁₀ carbocycle and 3- to 10-membered heterocycle; wherein the C₃₋₁₀ carbocycle and 3- to 10-membered heterocycle are each optionally substituted with one or more R⁷; R³ is selected from:
   C₁₋₆ alkyl, C₂₋₆ alkenyl, and C₂₋₆ alkynyl, each of which is optionally substituted with one or more R⁸; and
   C₃₋₁₀ carbocycle and 3- to 10-membered heterocycle; wherein the C₃₋₁₀ carbocycle and 3- to 10-membered heterocycle are each optionally substituted with one or more R⁸; R⁴, R⁵, R⁶, R⁷, and R⁸ are each independently selected from:
   hydrogen;
   C₁₋₁₀ alkyl, C₂₋₁₀ alkenyl, and C₂₋₁₀ alkynyl optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₁₋₆ cycloalkyl, and C₁₋₆ cycloalkenyl; and
   C₃₋₁₀ carbocycle or 3- to 10-membered heterocycle; wherein the C₃₋₁₀ carbocycle and 3- to 10-membered heterocycle are each optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), and C₁₋₈ alkyl;
   with the proviso that the compound of Formula I is not

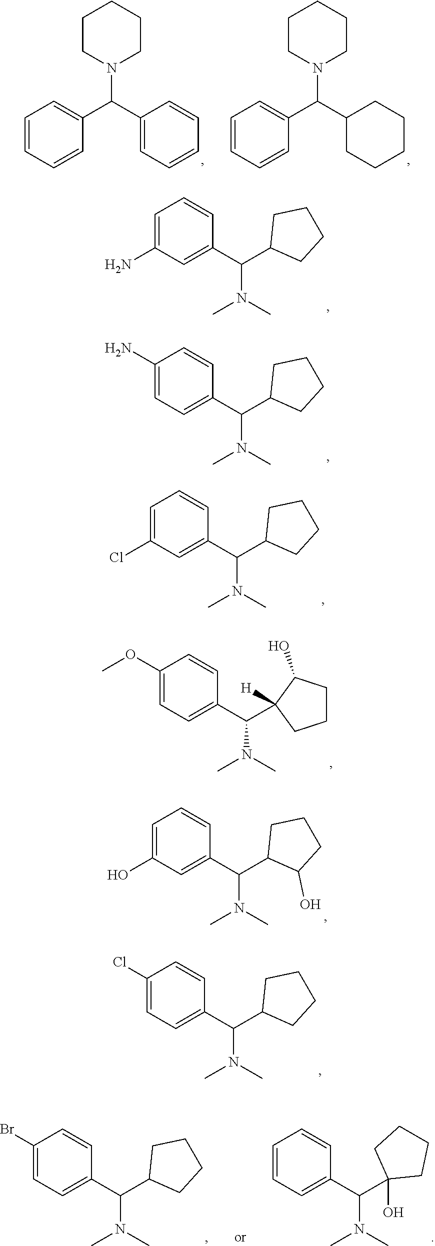

In certain aspects, the disclosure provides a compound represented by Formula (II):

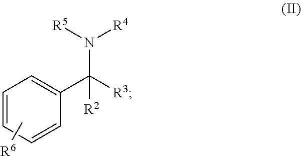

or an enantiomer, a mixture of enantiomers, or an isotopic variant thereof; or a pharmaceutically acceptable salt, solvate or hydrate thereof, wherein:
R² is selected from
   hydrogen;
   halogen;
   C₁₋₈ alkyl, C₂₋₈ alkenyl, and C₂₋₈ alkynyl, each of which is optionally substituted with one or more R⁷; and
   C₃₋₁₀ carbocycle and 3- to 10-membered heterocycle; wherein the C₃₋₁₀ carbocycle and 3- to 10-membered heterocycle are each optionally substituted with one or more R⁷; R³ is selected from:
   C₁₋₆ alkyl, C₂₋₆ alkenyl, and C₂₋₆ alkynyl, each of which is optionally substituted with one or more R⁸; and
   C₃₋₁₀ carbocycle and 3- to 10-membered heterocycle; wherein the C₃₋₁₀ carbocycle and 3- to 10-membered heterocycle are each optionally substituted with one or more R⁸ R⁴, R⁵, R⁶, R⁷, and R⁸ are each independently selected from:
   hydrogen;
   C₁₋₁₀ alkyl, C₂₋₁₀ alkenyl, and C₂₋₁₀ alkynyl optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₁₋₆ cycloalkyl, and C₁₋₆ cycloalkenyl; and
   C₃₋₁₀ carbocycle or 3- to 10-membered heterocycle; wherein the C₃₋₁₀ carbocycle and 3- to 10-membered heterocycle are each optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), and C₁₋₈ alkyl;
with the proviso that the compound of Formula II is not

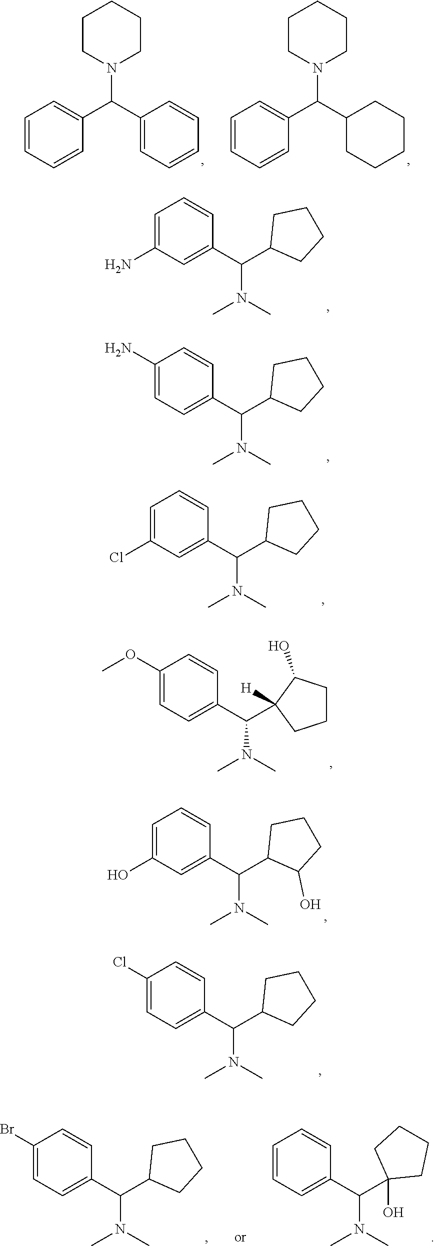

In certain aspects, the disclosure provides a compound represented by Formula (III):

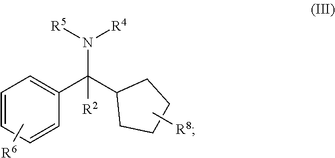

or an enantiomer, a mixture of enantiomers, or an isotopic variant thereof, or a pharmaceutically acceptable salt, solvate or hydrate thereof, wherein:
R² is selected from
   hydrogen;
   halogen;
   C₁₋₈ alkyl, C₂₋₈ alkenyl, and C₂₋₈ alkynyl, each of which is optionally substituted with one or more R⁷; and
   C₃₋₁₀ carbocycle and 3- to 10-membered heterocycle; wherein the C₃₋₁₀ carbocycle and 3- to 10-membered heterocycle are each optionally substituted with one or more R⁷; R⁴, R⁵, R⁶, R⁷, and R⁸ are each independently selected from:
   hydrogen;
   C₁₋₁₀ alkyl, C₂₋₁₀ alkenyl, and C₂₋₁₀ alkynyl optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₁₋₆ cycloalkyl, and C₁₋₆ cycloalkenyl; and
   C₃₋₁₀ carbocycle or 3- to 10-membered heterocycle; wherein the C₃₋₁₀ carbocycle and 3- to 10-membered heterocycle are each optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), and C₁₋₈ alkyl,
provided that the compound of Formula (III) is not

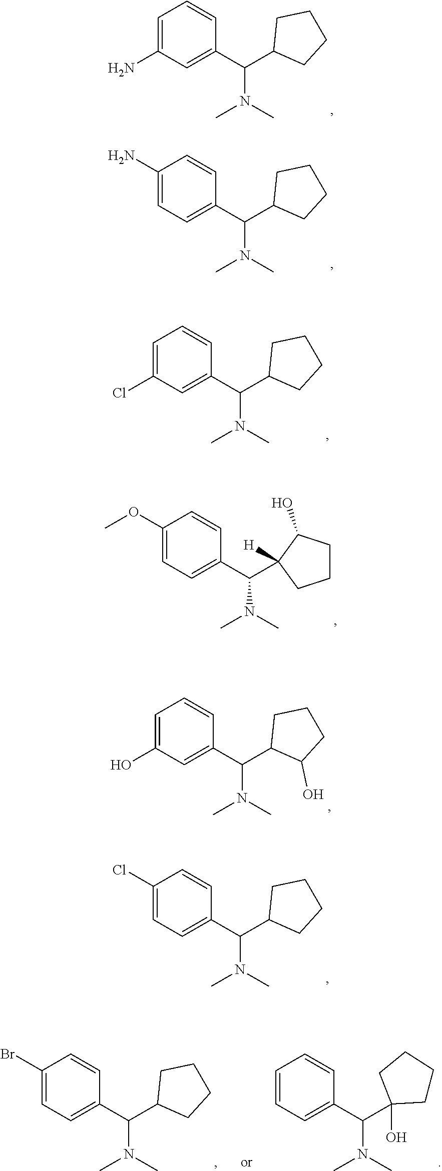

In certain aspects, the disclosure provides a compound represented by Formula (IV):

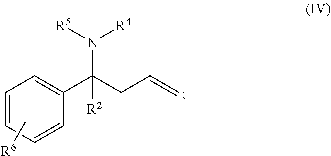

or an enantiomer, a mixture of enantiomers, or an isotopic variant thereof; or a pharmaceutically acceptable salt, solvate or hydrate thereof, wherein:
R² is selected from
   hydrogen;
   halogen;
   C₁₋₈ alkyl, C₂₋₈ alkenyl, and C₂₋₈ alkynyl, each of which is optionally substituted with one or more R⁷; and
   C₃₋₁₀ carbocycle and 3- to 10-membered heterocycle; wherein the C₃₋₁₀ carbocycle and 3- to 10-membered heterocycle are each optionally substituted with one or more R⁷; R⁴, R⁵, R⁶, R⁷, and R⁸ are each independently selected from:
   hydrogen;
   C₁₋₁₀ alkyl, C₂₋₁₀ alkenyl, and C₂₋₁₀ alkynyl optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₁₋₆ cycloalkyl, and C₁₋₆ cycloalkenyl; and
   C₃₋₁₀ carbocycle or 3- to 10-membered heterocycle; wherein the C₃₋₁₀ carbocycle and 3- to 10-membered heterocycle are each optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), and C₁₋₈ alkyl.

In certain aspects, the disclosure provides a compound represented by Formula (V):

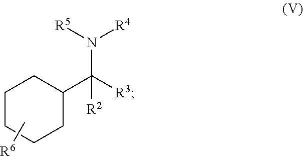

or an enantiomer, a mixture of enantiomers, or an isotopic variant thereof; or a pharmaceutically acceptable salt, solvate or hydrate thereof, wherein:
R² is selected from
   hydrogen;
   halogen;
   C₁₋₈ alkyl, C₂₋₈ alkenyl, and C₂₋₈ alkynyl, each of which is optionally substituted with one or more R⁷; and
   C₃₋₁₀ carbocycle and 3- to 10-membered heterocycle; wherein the C₃₋₁₀ carbocycle and 3- to 10-membered heterocycle are each optionally substituted with one or more R⁷; R³ is selected from:
   C₁₋₆ alkyl, C₂₋₆ alkenyl, and C₂₋₆ alkynyl, each of which is optionally substituted with one or more R⁸; and
   C₃₋₁₀ carbocycle and 3- to 10-membered heterocycle; wherein the C₃₋₁₀ carbocycle and 3- to 10-membered heterocycle are each optionally substituted with one or more R⁸; R⁴, R⁵, R⁶, R⁷, and R⁸ are each independently selected from:
   hydrogen;
   C₁₋₁₀ alkyl, C₂₋₁₀ alkenyl, and C₂₋₁₀ alkynyl optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₁₋₆ cycloalkyl, and C₁₋₆ cycloalkenyl; and
   C₃₋₁₀ carbocycle or 3- to 10-membered heterocycle; wherein the C₃₋₁₀ carbocycle and 3- to 10-membered heterocycle are each optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), and C₁₋₈ alkyl.

In certain aspects, the disclosure provides a compound represented by Formula (VI):

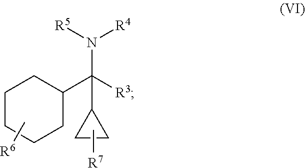

or an enantiomer, a mixture of enantiomers, or an isotopic variant thereof, or a pharmaceutically acceptable salt, solvate or hydrate thereof, wherein:
R³ is selected from:
   C₁₋₆ alkyl, C₂₋₆ alkenyl, and C₂₋₆ alkynyl, each of which is optionally substituted with one or more R⁸; and
   C₃₋₁₀ carbocycle and 3- to 10-membered heterocycle; wherein the C₃₋₁₀ carbocycle and 3- to 10-membered heterocycle are each optionally substituted with one or more R⁸; R⁴, R⁵, R⁶, R⁷, and R⁸ are each independently selected from:
   hydrogen;
   C₁₋₁₀ alkyl, C₂₋₁₀ alkenyl, and C₂₋₁₀ alkynyl optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₁₋₆ cycloalkyl, and C₁₋₆ cycloalkenyl; and
   C₃₋₁₀ carbocycle or 3- to 10-membered heterocycle; wherein the C₃₋₁₀ carbocycle and 3- to 10-membered heterocycle are each optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), and C₁₋₈ alkyl.

In certain aspects, the disclosure provides a pharmaceutical composition comprising a compound or salt of any one of Formulas (I), (II), (III), (IV), (V), or (VI), or a pharmaceutically acceptable excipient.

Furthermore, the present disclosure generally relates to methods for treating a physical, psychiatric, or neurological disorder comprising inhibiting an ionotropic receptor by contacting the ionotropic receptor with a compound represented by the structure of Formula (I), (II), (III), (IV), (V), or (VI) and pharmaceutical compositions thereof.

In another aspect, the present disclosure provides a method for treating a physical, psychiatric, or neurological disorder comprising inhibiting an inotropic receptor by contacting the receptor with a compound represented by the structure of Formula (I):

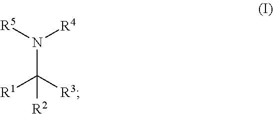

or an enantiomer, a mixture of enantiomers, or an isotopic variant thereof; or a pharmaceutically acceptable salt, solvate or hydrate thereof, wherein:
R¹ is C₃₋₁₀ carbocycle or 3- to 10-membered heterocycle; wherein the C₃₋₁₀ carbocycle and 3- to 10-membered heterocycle are each optionally substituted with one or more R⁶
R² is selected from
   hydrogen;
   halogen;
   C₁₋₈ alkyl, C₂₋₈ alkenyl, and C₂₋₈ alkynyl, each of which is optionally substituted with one or more R⁷; and
   C₃₋₁₀ carbocycle and 3- to 10-membered heterocycle; wherein the C₃₋₁₀ carbocycle and 3- to 10-membered heterocycle are each optionally substituted with one or more R⁷; R³ is selected from:
   C₁₋₆ alkyl, C₂₋₆ alkenyl, and C₂₋₆ alkynyl, each of which is optionally substituted with one or more R⁸; and
   C₃₋₁₀ carbocycle and 3- to 10-membered heterocycle; wherein the C₃₋₁₀ carbocycle and 3- to 10-membered heterocycle are each optionally substituted with one or more R⁸; R⁴, R⁵, R⁶, R⁷, and R⁸ are each independently selected from:
   hydrogen;
   C₁₋₁₀ alkyl, C₂₋₁₀ alkenyl, and C₂₋₁₀ alkynyl, each of which is optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₁₋₆ cycloalkyl, and C₁₋₆ cycloalkenyl; and
   C₃₋₁₀ carbocycle or 3- to 10-membered heterocycle; wherein the C₃₋₁₀ carbocycle and 3- to 10-membered heterocycle are each optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), and C₁₋₈ alkyl;

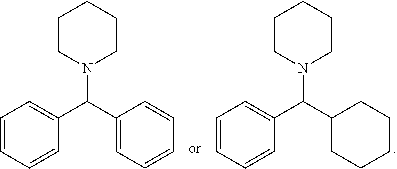

provided that the compound of Formula (I) is not or.

In some aspects, provided herein is a method for treating a physical, psychiatric, or neurological disorder comprising inhibiting an inotropic receptor by contacting the receptor with a pharmaceutical composition comprising a compound represented by the structure of Formula (II):

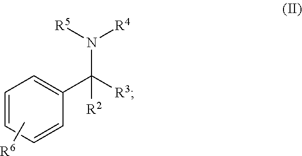

or an enantiomer, a mixture of enantiomers, or an isotopic variant thereof; or a pharmaceutically acceptable salt, solvate or hydrate thereof, wherein:
R² is selected from
   hydrogen;
   halogen;
   C₁₋₈ alkyl, C₂₋₈ alkenyl, and C₂₋₈ alkynyl, each of which is optionally substituted with one or more R⁷; and
   C₃₋₁₀ carbocycle and 3- to 10-membered heterocycle; wherein the C₃₋₁₀ carbocycle and 3- to 10-membered heterocycle are each optionally substituted with one or more R⁷; R³ is selected from:
   C₁₋₆ alkyl, C₂₋₆ alkenyl, and C₂₋₆ alkynyl, each of which is optionally substituted with one or more R⁸; and
   C₃₋₁₀ carbocycle and 3- to 10-membered heterocycle; wherein the C₃₋₁₀ carbocycle and 3- to 10-membered heterocycle are each optionally substituted with one or more R⁸; R⁴, R⁵, R⁶, R⁷, and R⁸ are each independently selected from:
   hydrogen;
   C₁₋₁₀ alkyl, C₂₋₁₀ alkenyl, and C₂₋₁₀ alkynyl, each of which is optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₁₋₆ cycloalkyl, and C₁₋₆ cycloalkenyl; and
   C₃₋₁₀ carbocycle or 3- to 10-membered heterocycle; wherein the C₃₋₁₀ carbocycle and 3- to 10-membered heterocycle are each optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), and C₁₋₈ alkyl;
provided that the compound of Formula (II) is not

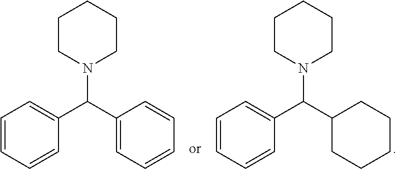

In another aspect, provided herein is a method for treating a physical, psychiatric, or neurological disorder comprising inhibiting an inotropic receptor by contacting the receptor with a pharmaceutical composition comprising a compound represented by the structure of Formula (III):

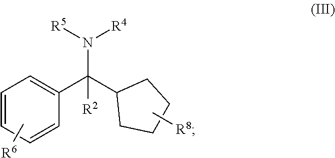

or an enantiomer, a mixture of enantiomers, or an isotopic variant thereof, or a pharmaceutically acceptable salt, solvate or hydrate thereof, wherein:
R² is selected from
   hydrogen;
   halogen;
   C₁₋₈ alkyl, C₂₋₈ alkenyl, and C₂₋₈ alkynyl, each of which is optionally substituted with one or more R⁷; and
   C₃₋₁₀ carbocycle and 3- to 10-membered heterocycle; wherein the C₃₋₁₀ carbocycle and 3- to 10-membered heterocycle are each optionally substituted with one or more R⁷; R⁴, R⁵, R⁶, R⁷, and R⁸ are each independently selected from:
   hydrogen;
   C₁₋₁₀ alkyl, C₂₋₁₀ alkenyl, and C₂₋₁₀ alkynyl, each of which is optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₁₋₆ cycloalkyl, and C₁₋₆ cycloalkenyl; and
   C₃₋₁₀ carbocycle or 3- to 10-membered heterocycle; wherein the C₃₋₁₀ carbocycle and 3- to 10-membered heterocycle are each optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), and C₁₋₈ alkyl.

In another aspect, provided herein is a method for treating a physical, psychiatric, or neurological disorder comprising inhibiting an inotropic receptor by contacting the receptor with a pharmaceutical composition comprising a compound represented by the structure of Formula (IV):

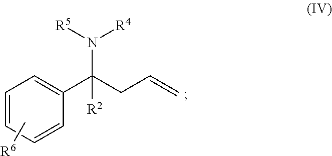

or an enantiomer, a mixture of enantiomers, or an isotopic variant thereof, or a pharmaceutically acceptable salt, solvate or hydrate thereof, wherein:
R² is selected from
   hydrogen;
   halogen;
   C₁₋₈ alkyl, C₂₋₈ alkenyl, and C₂₋₈ alkynyl, each of which is optionally substituted with one or more R⁷; and
   C₃₋₁₀ carbocycle and 3- to 10-membered heterocycle; wherein the C₃₋₁₀ carbocycle and 3- to 10-membered heterocycle are each optionally substituted with one or more R⁷; R⁴, R⁵, R⁶, R⁷, and R⁸ are each independently selected from:
   hydrogen;
   C₁₋₁₀ alkyl, C₂₋₁₀ alkenyl, and C₂₋₁₀ alkynyl, each of which is optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₁₋₆ cycloalkyl, and C₁₋₆ cycloalkenyl; and
   C₃₋₁₀ carbocycle or 3- to 10-membered heterocycle; wherein the C₃₋₁₀ carbocycle and 3- to 10-membered heterocycle are each optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), and C₁₋₈ alkyl.

In another aspect, provided herein is a method for treating a physical, psychiatric, or neurological disorder comprising inhibiting an inotropic receptor by contacting the receptor with a pharmaceutical composition comprising a compound represented by the structure of Formula (V):

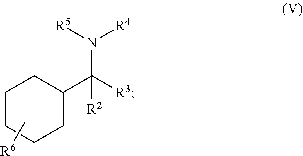

or an enantiomer, a mixture of enantiomers, or an isotopic variant thereof; or a pharmaceutically acceptable salt, solvate or hydrate thereof, wherein:
R² is selected from
   hydrogen;
   halogen;
   C₁₋₈ alkyl, C₂₋₈ alkenyl, and C₂₋₈ alkynyl, each of which is optionally substituted with one or more R⁷; and
   C₃₋₁₀ carbocycle and 3- to 10-membered heterocycle; wherein the C₃₋₁₀ carbocycle and 3- to 10-membered heterocycle are each optionally substituted with one or more R⁷;
R³ is selected from:
   C₁₋₆ alkyl, C₂₋₆ alkenyl, and C₂₋₆ alkynyl, each of which is optionally substituted with one or more R⁸; and
   C₃₋₁₀ carbocycle and 3- to 10-membered heterocycle; wherein the C₃₋₁₀ carbocycle and 3- to 10-membered heterocycle are each optionally substituted with one or more R⁸; R⁴, R⁵, R⁶, R⁷, and R⁸ are each independently selected from:
   hydrogen;
   C₁₋₁₀ alkyl, C₂₋₁₀ alkenyl, and C₂₋₁₀ alkynyl, each of which is optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₁₋₆ cycloalkyl, and C₁₋₆ cycloalkenyl; and
   C₃₋₁₀ carbocycle or 3- to 10-membered heterocycle; wherein the C₃₋₁₀ carbocycle and 3- to 10-membered heterocycle are each optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), and C₁₋₈ alkyl.

In another aspect, provided herein is a method for treating a physical, psychiatric, or neurological disorder comprising inhibiting an inotropic receptor by contacting the receptor with a pharmaceutical composition comprising a compound represented by the structure of Formula (VI):

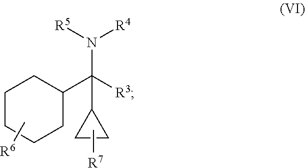

or an enantiomer, a mixture of enantiomers, or an isotopic variant thereof; or a pharmaceutically acceptable salt, solvate or hydrate thereof, wherein:
R³ is selected from:
   C₁₋₆ alkyl, C₂₋₆ alkenyl, and C₂₋₆ alkynyl, each of which is optionally substituted with one or more R⁸; and
   C₃₋₁₀ carbocycle and 3- to 10-membered heterocycle; wherein the C₃₋₁₀ carbocycle and 3- to 10-membered heterocycle are each optionally substituted with one or more R⁸; R⁴, R⁵, R⁶, R⁷, and R⁸ are each independently selected from:
   hydrogen;
   C₁₋₁₀ alkyl, C₂₋₁₀ alkenyl, and C₂₋₁₀ alkynyl, each of which is optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₁₋₆ cycloalkyl, and C₁₋₆ cycloalkenyl; and
   C₃₋₁₀ carbocycle or 3- to 10-membered heterocycle; wherein the C₃₋₁₀ carbocycle and 3- to 10-membered heterocycle are each optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), and C₁₋₈ alkyl.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure.

Accordingly, the drawings and description are to be regarded as illustrative in nature, and not as restrictive.

### DETAILED DESCRIPTION

While various embodiments of the disclosure have been shown and described herein, it will be obvious to those skilled in the art that such embodiments are provided by way of example only. Numerous variations, changes, and substitutions may occur to those skilled in the art without departing from the disclosure. It should be understood that various alternatives to the embodiments of the disclosure described herein may be employed.

Disclosed herein are a series of substituted methanamine compounds and pharmaceutical compositions for treating a physical, psychiatric, or neurological disorder. Examples of a physical, psychiatric, or neurological disorder include, but are not limited to, pain, depression, tinnitus, post-traumatic stress disorder, etc. In some embodiments, the compounds disclosed herein are N-methyl-D-aspartate receptor (NIDAR) antagonists. Further disclosed herein are methods for treating a physical, psychiatric, or neurological disorder comprising inhibiting an ionotropic receptor by contacting the ionotropic receptor with a compound disclosed herein and pharmaceutical compositions thereof.

### Compounds

The following is a discussion of compounds and salts thereof that may be used in the methods of this disclosure. In certain embodiments, the compounds and salts are described in Formulas (I), (II), (III), (IV), (V), or (VI).

In one aspect, disclosed herein is a compound represented by Formula (I):

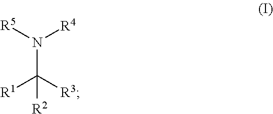

or an enantiomer, a mixture of enantiomers, or an isotopic variant thereof; or a pharmaceutically acceptable salt, solvate or hydrate thereof, wherein:
R¹ is C₃₋₁₀ carbocycle or 3- to 10-membered heterocycle; wherein the C₃₋₁₀ carbocycle and 3- to 10-membered heterocycle are each optionally substituted with one or more R⁶
R² is selected from
   hydrogen;
   halogen;
   C₁₋₈ alkyl, C₂₋₈ alkenyl, and C₂₋₈ alkynyl, each of which is optionally substituted with one or more R⁷; and
   C₃₋₁₀ carbocycle and 3- to 10-membered heterocycle; wherein the C₃₋₁₀ carbocycle and 3-to 10-membered heterocycle are each optionally substituted with one or more R⁷;
R³ is selected from:
   C₁₋₆ alkyl, C₂₋₆ alkenyl, and C₂₋₆ alkynyl, each of which is optionally substituted with one or more R⁸; and
   C₃₋₁₀ carbocycle and 3- to 10-membered heterocycle; wherein the C₃₋₁₀ carbocycle and 3-to 10-membered heterocycle are each optionally substituted with one or more R¹; R⁴, R⁵, R⁶, R⁷, and R⁸ are each independently selected from:
   hydrogen;
   C₁₋₁₀ alkyl, C₂₋₁₀ alkenyl, and C₂₋₁₀ alkynyl optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₁₋₆ cycloalkyl, and C₁₋₆ cycloalkenyl; and
   C₃₋₁₀ carbocycle or 3- to 10-membered heterocycle; wherein the C₃₋₁₀ carbocycle and 3- to 10-membered heterocycle are each optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), and C₁₋₈ alkyl;
      with the proviso that the compound of Formula (I) is not

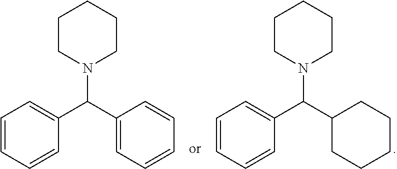

In some aspects, disclosed herein is a compound of Formula (I):

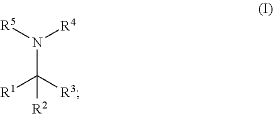

or an enantiomer, a mixture of enantiomers, or an isotopic variant thereof; or a pharmaceutically acceptable salt, solvate or hydrate thereof, wherein:
R¹ is C₃₋₁₀ carbocycle or 3- to 10-membered heterocycle; wherein the C₃₋₁₀ carbocycle and 3- to 10-membered heterocycle are each optionally substituted with one or more R⁶
R² is selected from
   hydrogen;
   halogen;
   C₁₋₈ alkyl, C₂₋₈ alkenyl, and C₂₋₈ alkynyl, each of which is optionally substituted with one or more R⁷; and
   C₃₋₁₀ carbocycle and 3- to 10-membered heterocycle; wherein the C₃₋₁₀ carbocycle and 3- to 10-membered heterocycle are each optionally substituted with one or more R⁷;
R³ is selected from: C₁₋₆ alkyl, C₂₋₆ alkenyl, and C₂₋₆ alkynyl, each of which is optionally substituted with one or more R⁸; and C₃₋₁₀ carbocycle and 3- to 10-membered heterocycle; wherein the C₃₋₁₀ carbocycle and 3- to 10-membered heterocycle are each optionally substituted with one or more R¹;
R⁴, R⁵, R⁶, R⁷, and R⁸ are each independently selected from: hydrogen; C₁₋₁₀ alkyl, C₂₋₁₀ alkenyl, C₂₋₁₀ alkynyl, and C₃₋₅ cycloalkyl, wherein C₁₋₁₀ alkyl, C₂₋₁₀ alkenyl, and C₂₋₁₀ alkynyl are optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₁₋₆ cycloalkyl, and C₁₋₆ cycloalkenyl; and C₃₋₁₀ carbocycle or 3- to 10-membered heterocycle; wherein the C₃₋₁₀ carbocycle and 3- to 10-membered heterocycle are each optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), and C₁₋₈ alkyl; or
R⁴ and R⁵ are combined with the nitrogen to which it is attached to form a 6-membered heterocycle;
with the proviso that the compound of Formula I is not

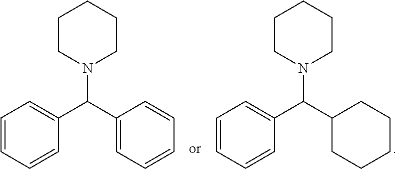

In some embodiments, for the compound or salt of Formula I, R¹ is selected from phenyl and 5- to 10-membered heteroaryl; wherein the phenyl and 5- to 10-membered heteroaryl are each optionally substituted with one or more R⁶
R² is selected from hydrogen, C₁₋₈ alkyl, C₂₋₈ alkenyl, C₂₋₈ alkynyl, and C₃₋₅ carbocycle, each of which is optionally substituted with one or more R⁷;
R³ is C₃₋₆ carbocycle, wherein the C₃₋₆ carbocycle is optionally substituted with one or more R⁸;
R⁴ and R⁵ are each independently selected from: hydrogen, C₁₋₁₀ alkyl, and C₃₋₅ cycloalkyl, wherein the C₁₋₁₀ alkyl is optionally substituted with C₃₋₅ cycloalkyl; or R⁴ and R⁵ are combined with the nitrogen to which it is attached to form a 6-membered heterocycle;
R⁶ is C₁₋₁₀ alkyl, wherein the C₁₋₁₀ alkyl is optionally substituted with halogen;
R⁷ is C₃₋₅ cycloalkyl; and
R⁸ is C₁₋₁₀ alkyl.

In some embodiments, R¹ is selected from phenyl, imidazolyl, thiophenyl, and selenophenyl; wherein the phenyl, imidazolyl, thiophenyl, and selenophenyl are each optionally substituted with one or more R⁶. In some embodiments, R² is selected from hydrogen, methyl, ethyl, propyl, butyl, ethenyl, propenyl, butenyl, ethynyl, propynyl, butynyl, cyclopropyl, cyclobutyl, and cyclopentyl, each of which is optionally substituted with one or more R⁷. In some embodiments, R³ is cyclobutyl, cyclopentyl, or cyclohexyl. In some embodiments, R⁴ and R⁵ are each independently selected from: hydrogen, methyl, ethyl, n-propyl, isopropyl, n-butyl, sec-butyl, iso-butyl, tert-butyl, methyl-cyclopropyl, methyl-cyclobutyl, methyl-cyclopentyl, cyclopropyl, cyclobutyl, or cyclopentyl; or R⁴ and R⁵ are combined with the nitrogen to which it is attached to form piperidinyl. In some embodiments, R⁶ is methyl or CF₃. In some embodiments, R⁷ is cyclopropyl or cyclobutyl.

In some embodiments, R¹ is selected from phenyl and selenophenyl; wherein the phenyl and selenophenyl are each optionally substituted with one or more R⁶. In some embodiments, R² is selected from hydrogen, methyl, butyl, ethenyl, propenyl, butenyl, ethynyl, cyclopropyl, and cyclobutyl, each of which is optionally substituted with one or more R⁷. In some embodiments, R³ is cyclopentyl. In some embodiments, R⁴ and R⁵ are each independently selected from: hydrogen, methyl, ethyl, isopropyl, sec-butyl, methyl-cyclopropyl, cyclobutyl; or R⁴ and R⁵ are combined with the nitrogen to which it is attached to form piperidinyl. In some embodiments, R⁶ is CF₃. In some embodiments, R⁷ is cyclopropyl or cyclobutyl. In some embodiments,

In some embodiments, R¹ is C₃₋₇ carbocycle or 3- to 7-membered heterocycle; wherein the C₃₋₇ carbocycle and 3- to 7-membered heterocycle are each optionally substituted with one or more R⁶.

In some embodiments, R¹ is phenyl, optionally substituted with one or more R⁶.

In some embodiments, R¹ is a cycloalkyl. In some embodiments, R¹ is a cycloalkyl selected from cyclooctane, cycloheptane, cyclohexane, cyclopentane, cyclobutane, and cyclopropane, any of which is optionally substituted with one or more R⁶.

In some embodiments, R¹ is a cycloalkenyl. In some embodiments, R¹ is a cycloalkenyl selected from cyclooctene, cycloheptene, cyclohexene, cyclopentene, cyclobutene, and cyclopropane, any of which is optionally substituted with one or more R⁶. In some embodiments, the cycloalkenyl can have 1, 2, 3, 4, or more degrees of unsaturation.

In some embodiments, R¹ is a 6-membered heteroaryl. In some embodiments, R¹ is a 6-membered heteroaryl selected from pyridine, pyrazine, and pyrimidine, any of which is optionally substituted with one or more R⁶. In some embodiments, the heteroatom of the heteroaryl is not bound to the methylene carbon of Formula (I).

In some embodiments, R¹ is a 5-membered heteroaryl. In some embodiments, R¹ is a 5-membered heteroaryl selected from thiophene, furan, pyrrole, pyrazole, selenophene, imidazole, thiazole, isothiazole, oxatriazole, oxadiazole, oxazole, and thiadiazole, any of which is optionally substituted with one or more R⁶. In some embodiments, the heteroatom of the heteroaryl is not bound to the methylene carbon of Formula (I).

In some embodiments, R¹ is a 6-membered heterocycloalkenyl. In some embodiments, R¹ is a 6-membered heterocycloalkenyl selected from dihydropyridine, tetrahydropyridine, dihydropyridazine, tetrahydropyridazine, dihydropyrimidine, tetrahydropyrimidine, dihydropyrazine, tetrahydropyridazine, dihydropyrimidine, tetrahydropyrimidine, dihydropyrazine, tetrahydropyrazine, pyran, dihydropyran, thiopyran, dihydrothiopyran, dioxine, dihydrodioxine, oxazine, dihydrooxazine, thiazine, and dihydrothiazine, any of which is optionally substituted with one or more R⁶. In some embodiments, the heteroatom of the heterocycloalkenyl is not bound to the methylene carbon of Formula (I).

In some embodiments, R¹ is a 5-membered heterocycloalkenyl. In some embodiments, R¹ is a 5-membered heterocycloalkenyl selected from pyrroline, pyrazoline, imidazoline, triazoline, dihydrofuran, dihydrothiophene, oxazoline, isooxazoline, thiazoline, isothiazoline, oxadiazoline, and thiadiazoline, any of which is optionally substituted with one or more R⁶. In some embodiments, the heteroatom of the heterocycloalkenyl is not bound to the methylene carbon of Formula (I).

In some embodiments, R¹ is a 6-membered heterocycloalkyl. In some embodiments, R¹ is a 6-membered heterocycloalkyl selected from piperidine, piperazine, tetrahydrothiopyran, tetrahydropyran, dioxane, morpholine, and thiomorpholine, any of which is optionally substituted with one or more R⁶. In some embodiments, the heteroatom of the heterocycloalkyl is not bound to the methylene carbon of Formula (I).

In some embodiments, R¹ is a 5-membered heterocycloalkyl. In some embodiments, R¹ is a 5-membered heterocycloalkyl selected from tetrahydrofuran, pyrrolidone, and tetrahydrothiophene, any of which is optionally substituted with one or more R⁶. In some embodiments, the heteroatom of the heterocycloalkyl is not bound to the methylene carbon of Formula (I).

In some embodiments, R¹ is a 4-membered heterocycloalkyl. In some embodiments, R¹ is a 4-membered heterocycloalkyl selected from azetidine, oxetane, and thietane, any of which is optionally substituted with one or more R⁶. In some embodiments, the heteroatom of the heterocycloalkyl is not bound to the methylene carbon of Formula (I).

In some embodiments, R¹ is a 6-6 fused ring system. In some embodiments, R¹ is a 6-6 fused ring system selected from naphthalene, quinoline, isoquinoline, pteridine, benzopyran, dihydroquinoline, dihydroisoquinoline, dihydronapthalene, and tetrahydronapthalene, any of which is optionally substituted with one or more R⁶.

In some embodiments, R¹ is a 5-6 fused ring system. In some embodiments, R¹ is a 5-6 fused ring system selected from benzimidazole, indole, azaindole, indazole, benzothiophene, benzofuran, benzoselenophene, benzimidazole, benzotriazole, benzothiazole, benzisothiazole, benzoxadiazole, benzoxazole, and benzothiadiazole, imidazo[4,5-b]-pyridine, imidazo[4,5-c]-pyridine, pyrazolo[3,4-b]pyridine, pyrazolo[3,4-c]pyridine, pyrazolo[4,3-b]pyridine, pyrazolo[4,3-b]pyridine, oxazolo[4,5-b]pyridine, oxazolo[4,5-c]pyridine, oxazolo[5,4-b]pyridine, oxazolo[5,4-c]pyridine, thiazolo[4,5-b]pyridine, thiazolo[4,5-c]pyridine, thiazolo[5,4-b]pyridine, thiazolo[5,4-c]pyridine, isooxazolo[4,5-b]pyridine, isooxazolo[4,5-c]pyridine, isooxazolo[3,4-b]pyridine, isooxazolo[3,4-c]pyridine, isooxazolo[4,3-b]pyridine, isooxazolo[4,3-c]pyridine, isooxazolo[5,4-b]pyridine, isooxazolo[5,4-c]pyridine, isothiazolo[4,5-b]pyridine, isothiazolo[4,5-c]pyridine, isothiazolo[5,4-b]pyridine, isothiazolo[5,4-c]pyridine, isothiazolo[3,4-b]pyridine, isothiazolo[3,4-c]pyridine, isothiazolo[4,3-b]pyridine, isothiazolo[4,3-c]pyridine and purine, any of which is optionally substituted with one or more R⁶.

In some embodiments, R¹ is a 5-5 fused ring system. In some embodiments, R¹ is a 5-5 fused ring system selected from pyrrolo[2,1-b]thiazole, pyrazolo[5,1-b]thiazole, imidazo[5,1-b]thiazole, imidazo[2,1-b]thiazole, thiazolo[3,2-b][1,2,4]triazole, thiazolo[3,2-c][1,2,4]triazole, imidazo[2,1-b]thiadiazole, imidazo[1,2-d][1,2,4]thiadiazole, thiazolo[3,2-d]tetrazole, thieno[3,2-b]thiophene, thieno[2,3-b]thiophene, thieno[3,4-b]thiophene, thieno[3,4-c]thiophene, any of which is optionally substituted with one or more R⁶.

In some embodiments, R¹ is a bridged ring system. In some embodiments, R¹ is a bridged ring system selected from norbornane, norbornene, bicyclo[1.1.0]butane, bicyclo[3.2.0]heptane, bicyclo[3.3.0]octane, bicyclo[4.3.0]nonane, bicyclo[1.1.1]pentane, bicyclo[2.2.1]hexane, bicyclo[3.3.1]nonane, bicyclo[2.2.2]octane, adamantane, cubane, housane, any of which is optionally substituted with one or more R⁶. In some embodiments, the bridged ring system can have 1, 2, 3, 4, or more degrees of unsaturation.

In some embodiments, R² is hydrogen, C₁₋₈ alkyl, C₂₋₈ alkenyl, or C₂₋₈ alkynyl, each of which is optionally substituted with one or more R⁷.

In some embodiments, R² is C₃₋₇ carbocycle or 3- to 7-membered heterocycle; wherein the C₃₋₇ carbocycle and 3- to 7-membered heterocycle are each optionally substituted with one or more R⁷.

In some embodiments, R² is phenyl, optionally substituted with one or more R⁷.

In some embodiments, R² is a cycloalkyl. In some embodiments, R² is a cycloalkyl selected from cyclooctane, cycloheptane, cyclohexane, cyclopentane, cyclobutane, and cyclopropane, any of which is optionally substituted with one or more R⁷.

In some embodiments, R² is a cycloalkenyl. In some embodiments, R² is a cycloalkenyl selected from cyclooctene, cycloheptene, cyclohexene, cyclopentene, cyclobutene, and cyclopropane, any of which is optionally substituted with one or more R⁷. In some embodiments, the cycloalkenyl can have 1, 2, 3, 4, or more degrees of unsaturation.

In some embodiments, R² is a 6-membered heteroaryl. In some embodiments, R² is a 6-membered heteroaryl selected from pyridine, pyrazine, and pyrimidine, any of which is optionally substituted with one or more R⁷.

In some embodiments, R² is a 5-membered heteroaryl. In some embodiments, R² is a 5-membered heteroaryl selected from thiophene, furan, pyrrole, pyrazole, selenophene, imidazole, thiazole, isothiazole, oxatriazole, oxadiazole, oxazole, and thiadiazole, any of which is optionally substituted with one or more R⁷. In some embodiments, the heteroatom of the heteroaryl is not bound to the methylene carbon of Formula (I).

In some embodiments, R² is a 6-membered heterocycloalkenyl. In some embodiments, R² is a 6-membered heterocycloalkenyl selected from dihydropyridine, tetrahydropyridine, dihydropyridazine, tetrahydropyridazine, dihydropyrimidine, tetrahydropyrimidine, dihydropyrazine, tetrahydropyridazine, dihydropyrimidine, tetrahydropyrimidine, dihydropyrazine, tetrahydropyrazine, pyran, dihydropyran, thiopyran, dihydrothiopyran, dioxine, dihydrodioxine, oxazine, dihydrooxazine, thiazine, and dihydrothiazine, any of which is optionally substituted with one or more R⁷. In some embodiments, the heteroatom of the heterocycloalkenyl is not bound to the methylene carbon of Formula (I).

In some embodiments, R² is a 5-membered heterocycloalkenyl. In some embodiments, R² is a 5-membered heterocycloalkenyl selected from pyrroline, pyrazoline, imidazoline, triazoline, dihydrofuran, dihydrothiophene, oxazoline, isooxazoline, thiazoline, isothiazoline, oxadiazoline, and thiadiazoline, any of which is optionally substituted with one or more R⁷. In some embodiments, the heteroatom of the heterocycloalkenyl is not bound to the methylene carbon of Formula (I).

In some embodiments, R² is a 6-membered heterocycloalkyl. In some embodiments, R² is a 6-membered heterocycloalkyl selected from piperidine, piperazine, tetrahydrothiopyran, tetrahydropyran, dioxane, morpholine, and thiomorpholine, any of which is optionally substituted with one or more R⁷. In some embodiments, the heteroatom of the heterocycloalkyl is not bound to the methylene carbon of Formula (I).

In some embodiments, R² is a 5-membered heterocycloalkyl. In some embodiments, R² is a 5-membered heterocycloalkyl selected from tetrahydrofuran, pyrrolidone, and tetrahydrothiophene, any of which is optionally substituted with one or more R⁷. In some embodiments, the heteroatom of the heterocycloalkyl is not bound to the methylene carbon of Formula (I).

In some embodiments, R² is a 4-membered heterocycloalkyl. In some embodiments, R² is a 4-membered heterocycloalkyl selected from azetidine, oxetane, and thietane, any of which is optionally substituted with one or more R⁷. In some embodiments, the heteroatom of the heterocycloalkyl is not bound to the methylene carbon of Formula (I).

In some embodiments, R² is a 6-6 fused ring system. In some embodiments, R² is a 6-6 fused ring system selected from naphthalene, quinoline, isoquinoline, pteridine, benzopyran, dihydroquinoline, dihydroisoquinoline, dihydronapthalene, and tetrahydronapthalene, any of which is optionally substituted with one or more R⁷.

In some embodiments, R² is a 5-6 fused ring system. In some embodiments, R² is a 5-6 fused ring system selected from benzimidazole, indole, azaindole, indazole, benzothiophene, benzofuran, benzoselenophene, benzimidazole, benzotriazole, benzothiazole, benzisothiazole, benzoxadiazole, benzoxazole, and benzothiadiazole, imidazo[4,5-b]-pyridine, imidazo[4,5-c]-pyridine, pyrazolo[3,4-b]pyridine, pyrazolo[3,4-c]pyridine, pyrazolo[4,3-b]pyridine, pyrazolo[4,3-b]pyridine, oxazolo[4,5-b]pyridine, oxazolo[4,5-c]pyridine, oxazolo[5,4-b]pyridine, oxazolo[5,4-c]pyridine, thiazolo[4,5-b]pyridine, thiazolo[4,5-c]pyridine, thiazolo[5,4-b]pyridine, thiazolo[5,4-c]pyridine, isooxazolo[4,5-b]pyridine, isooxazolo[4,5-c]pyridine, isooxazolo[3,4-b]pyridine, isooxazolo[3,4-c]pyridine, isooxazolo[4,3-b]pyridine, isooxazolo[4,3-c]pyridine, isooxazolo[5,4-b]pyridine, isooxazolo[5,4-c]pyridine, isothiazolo[4,5-b]pyridine, isothiazolo[4,5-c]pyridine, isothiazolo[5,4-b]pyridine, isothiazolo[5,4-c]pyridine, isothiazolo[3,4-b]pyridine, isothiazolo[3,4-c]pyridine, isothiazolo[4,3-b]pyridine, isothiazolo[4,3-c]pyridine and purine, any of which is optionally substituted with one or more R⁷.

In some embodiments, R² is a 5-5 fused ring system. In some embodiments, R² is a 5-5 fused ring system selected from pyrrolo[2,1-b]thiazole, pyrazolo[5,1-b]thiazole, imidazo[5,1-b]thiazole, imidazo[2,1-b]thiazole, thiazolo[3,2-b][1,2,4]triazole, thiazolo[3,2-c][1,2,4]triazole, imidazo[2,1-b]thiadiazole, imidazo[1,2-d][1,2,4]thiadiazole, thiazolo[3,2-d]tetrazole, thieno[3,2-b]thiophene, thieno[2,3-b]thiophene, thieno[3,4-b]thiophene, thieno[3,4-c]thiophene, any of which is optionally substituted with one or more R⁷.

In some embodiments, R² is a bridged ring system. In some embodiments, R² is a bridged ring system selected from norbornane, norbornene, bicyclo[1.1.0]butane, bicyclo[3.2.0]heptane, bicyclo[3.3.0]octane, bicyclo[4.3.0]nonane, bicyclo[1.1.1]pentane, bicyclo[2.2.1]hexane, bicyclo[3.3.1]nonane, bicyclo[2.2.2]octane, adamantane, cubane, housane, any of which is optionally substituted with one or more R⁷. In some embodiments, the bridged ring system can have 1, 2, 3, 4, or more degrees of unsaturation.

In some embodiments, R³ is hydrogen, C₁₋₆ alkyl, C₂₋₆ alkenyl, or C₂₋₆ alkynyl, each of which is optionally substituted with one or more R⁸.

In some embodiments, R³ is C₃₋₇ carbocycle or 3- to 7-membered heterocycle; wherein the C₃₋₇ carbocycle and 3- to 7-membered heterocycle are each optionally substituted with one or more R⁸.

In some embodiments, R³ is phenyl, optionally substituted with one or more R⁸.

In some embodiments, R³ is a cycloalkyl. In some embodiments, R³ is a cycloalkyl selected from cyclooctane, cycloheptane, cyclohexane, cyclopentane, cyclobutane, and cyclopropane, any of which is optionally substituted with one or more R⁸.

In some embodiments, R³ is a cycloalkenyl. In some embodiments, R³ is a cycloalkenyl selected from cyclooctene, cycloheptene, cyclohexene, cyclopentene, cyclobutene, and cyclopropane, any of which is optionally substituted with one or more R⁸. In some embodiments, the cycloalkenyl can have 1, 2, 3, 4, or more degrees of unsaturation.

In some embodiments, R³ is a 6-membered heteroaryl. In some embodiments, R³ is a 6-membered heteroaryl selected from pyridine, pyrazine, and pyrimidine, any of which is optionally substituted with one or more R⁸. In some embodiments, the heteroatom of the heteroaryl is not bound to the methylene carbon of Formula (I).

In some embodiments, R³ is a 5-membered heteroaryl. In some embodiments, R³ is a 5-membered heteroaryl selected from thiophene, furan, pyrrole, pyrazole, selenophene, imidazole, thiazole, isothiazole, oxatriazole, oxadiazole, oxazole, and thiadiazole, any of which is optionally substituted with one or more R⁸. In some embodiments, the heteroatom of the heteroaryl is not bound to the methylene carbon of Formula (I).

In some embodiments, R³ is a 6-membered heterocycloalkenyl. In some embodiments, R³ is a 6-membered heterocycloalkenyl selected from dihydropyridine, tetrahydropyridine, dihydropyridazine, tetrahydropyridazine, dihydropyrimidine, tetrahydropyrimidine, dihydropyrazine, tetrahydropyridazine, dihydropyrimidine, tetrahydropyrimidine, dihydropyrazine, tetrahydropyrazine, pyran, dihydropyran, thiopyran, dihydrothiopyran, dioxine, dihydrodioxine, oxazine, dihydrooxazine, thiazine, and dihydrothiazine, any of which is optionally substituted with one or more R⁸. In some embodiments, the heteroatom of the heterocycloalkenyl is not bound to the methylene carbon of Formula (I).

In some embodiments, R³ is a 5-membered heterocycloalkenyl. In some embodiments, R³ is a 5-membered heterocycloalkenyl selected from pyrroline, pyrazoline, imidazoline, triazoline, dihydrofuran, dihydrothiophene, oxazoline, isooxazoline, thiazoline, isothiazoline, oxadiazoline, and thiadiazoline, any of which is optionally substituted with one or more R⁸. In some embodiments, the heteroatom of the heterocycloalkenyl is not bound to the methylene carbon of Formula (I).

In some embodiments, R³ is a 6-membered heterocycloalkyl. In some embodiments, R³ is a 6-membered heterocycloalkyl selected from piperidine, piperazine, tetrahydrothiopyran, tetrahydropyran, dioxane, morpholine, and thiomorpholine, any of which is optionally substituted with one or more R⁸. In some embodiments, the heteroatom of the heterocycloalkyl is not bound to the methylene carbon of Formula (I).

In some embodiments, R³ is a 5-membered heterocycloalkyl. In some embodiments, R³ is a 5-membered heterocycloalkyl selected from tetrahydrofuran, pyrrolidone, and tetrahydrothiophene, any of which is optionally substituted with one or more R⁸. In some embodiments, the heteroatom of the heterocycloalkyl is not bound to the methylene carbon of Formula (I).

In some embodiments, R³ is a 4-membered heterocycloalkyl. In some embodiments, R³ is a 4-membered heterocycloalkyl selected from azetidine, oxetane, and thietane, any of which is optionally substituted with one or more R⁸. In some embodiments, the heteroatom of the heterocycloalkyl is not bound to the methylene carbon of Formula (I).

In some embodiments, R³ is a 6-6 fused ring system. In some embodiments, R³ is a 6-6 fused ring system selected from naphthalene, quinoline, isoquinoline, pteridine, benzopyran, dihydroquinoline, dihydroisoquinoline, dihydronapthalene, and tetrahydronapthalene, any of which is optionally substituted with one or more R⁸.

In some embodiments, R³ is a 5-6 fused ring system. In some embodiments, R³ is a 5-6 fused ring system selected from benzimidazole, indole, azaindole, indazole, benzothiophene, benzofuran, benzoselenophene, benzimidazole, benzotriazole, benzothiazole, benzisothiazole, benzoxadiazole, benzoxazole, and benzothiadiazole, imidazo[4,5-b]-pyridine, imidazo[4,5-c]-pyridine, pyrazolo[3,4-b]pyridine, pyrazolo[3,4-c]pyridine, pyrazolo[4,3-b]pyridine, pyrazolo[4,3-b]pyridine, oxazolo[4,5-b]pyridine, oxazolo[4,5-c]pyridine, oxazolo[5,4-b]pyridine, oxazolo[5,4-c]pyridine, thiazolo[4,5-b]pyridine, thiazolo[4,5-c]pyridine, thiazolo[5,4-b]pyridine, thiazolo[5,4-c]pyridine, isooxazolo[4,5-b]pyridine, isooxazolo[4,5-c]pyridine, isooxazolo[3,4-b]pyridine, isooxazolo[3,4-c]pyridine, isooxazolo[4,3-b]pyridine, isooxazolo[4,3-c]pyridine, isooxazolo[5,4-b]pyridine, isooxazolo[5,4-c]pyridine, isothiazolo[4,5-b]pyridine, isothiazolo[4,5-c]pyridine, isothiazolo[5,4-b]pyridine, isothiazolo[5,4-c]pyridine, isothiazolo[3,4-b]pyridine, isothiazolo[3,4-c]pyridine, isothiazolo[4,3-b]pyridine, isothiazolo[4,3-c]pyridine and purine, any of which is optionally substituted with one or more R⁸.

In some embodiments, R³ is a 5-5 fused ring system. In some embodiments, R³ is a 5-5 fused ring system selected from pyrrolo[2,1-b]thiazole, pyrazolo[5,1-b]thiazole, imidazo[5,1-b]thiazole, imidazo[2,1-b]thiazole, thiazolo[3,2-b][1,2,4]triazole, thiazolo[3,2-c][1,2,4]triazole, imidazo[2,1-b]thiadiazole, imidazo[1,2-d][1,2,4]thiadiazole, thiazolo[3,2-d]tetrazole, thieno[3,2-b]thiophene, thieno[2,3-b]thiophene, thieno[3,4-b]thiophene, thieno[3,4-c]thiophene, any of which is optionally substituted with one or more R⁸.

In some embodiments, R³ is a bridged ring system. In some embodiments, R³ is a bridged ring system selected from norbornane, norbornene, bicyclo[1.1.0]butane, bicyclo[3.2.0]heptane, bicyclo[3.3.0]octane, bicyclo[4.3.0]nonane, bicyclo[1.1.1]pentane, bicyclo[2.2.1]hexane, bicyclo[3.3.1]nonane, bicyclo[2.2.2]octane, adamantane, cubane, housane, any of which is optionally substituted with one or more R⁸. In some embodiments, the bridged ring system can have 1, 2, 3, 4, or more degrees of unsaturation.

In some embodiments, each of R⁴ or R⁵ may independently be hydrogen, C₁₋₁₀ alkyl, C₂₋₁₀ alkenyl, or C₂₋₁₀ alkynyl, each of which is optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₁₋₆ cycloalkyl, and C₁₋₆ cycloalkenyl.

In some embodiments, each of R⁴ or R⁵ may independently be 3- to 10-membered heterocycle. In some embodiments, each of R⁴ or R⁵ may independently be 3- to 10-membered heterocycle selected from azacyclopropane, pyrrolidine, piperidine, azepane, azocane, azonane, morpholine, thiomorpholine, 1,4-piperazine, 1,4-oxazepane, and 1,4-diazepane.

In some embodiments, the compound has the structure:

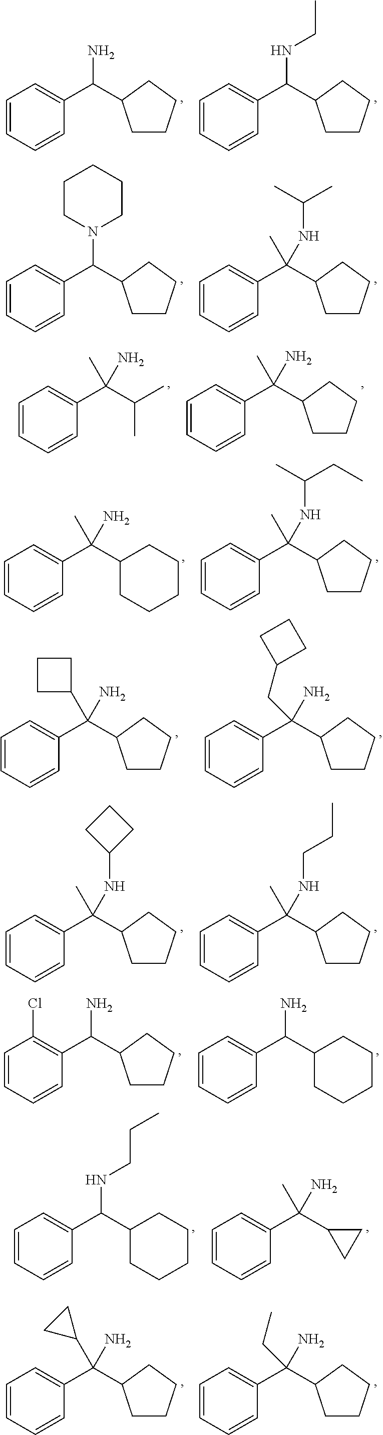

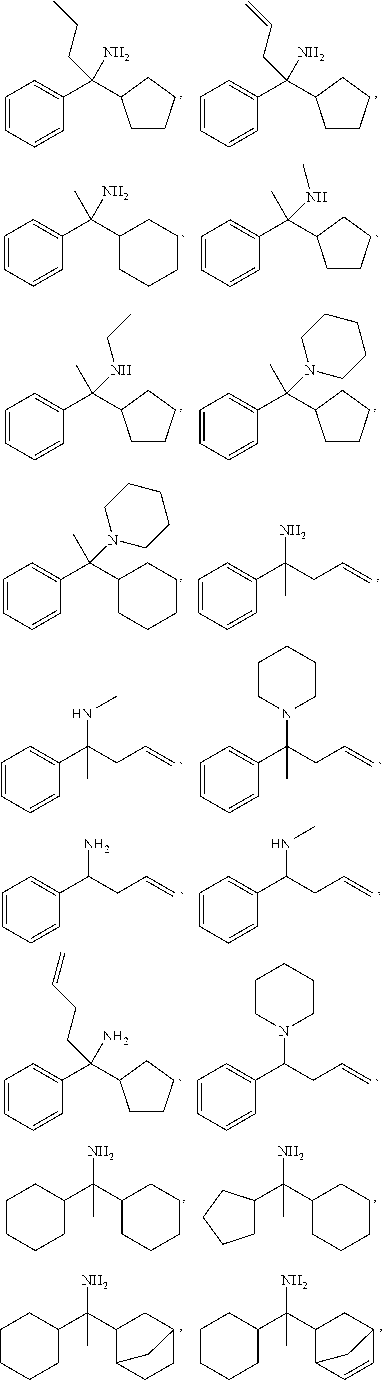

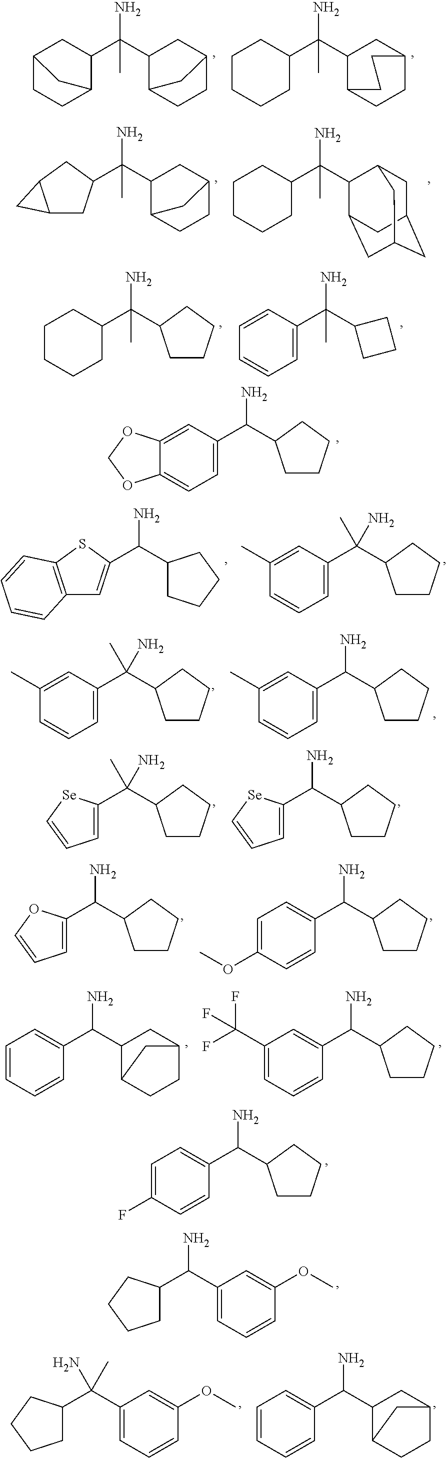

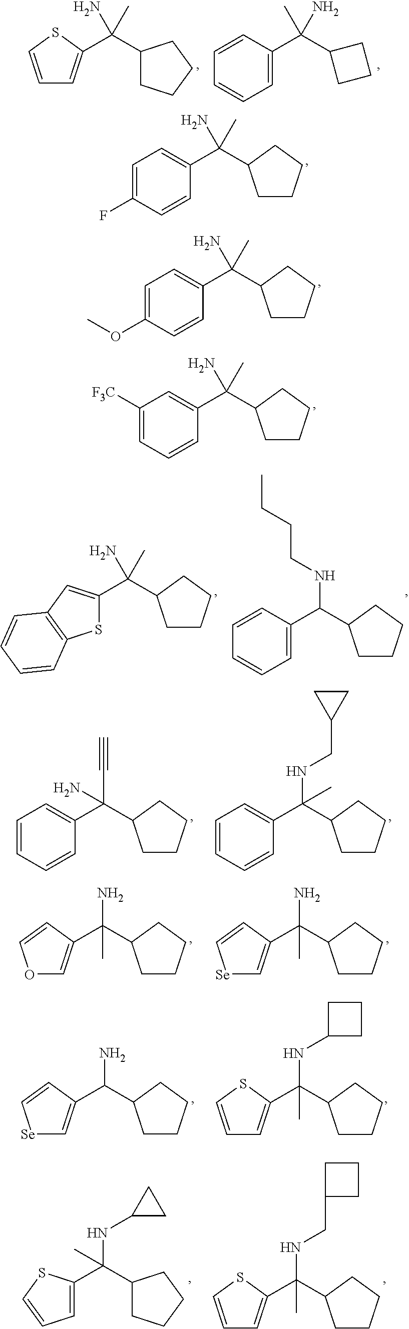

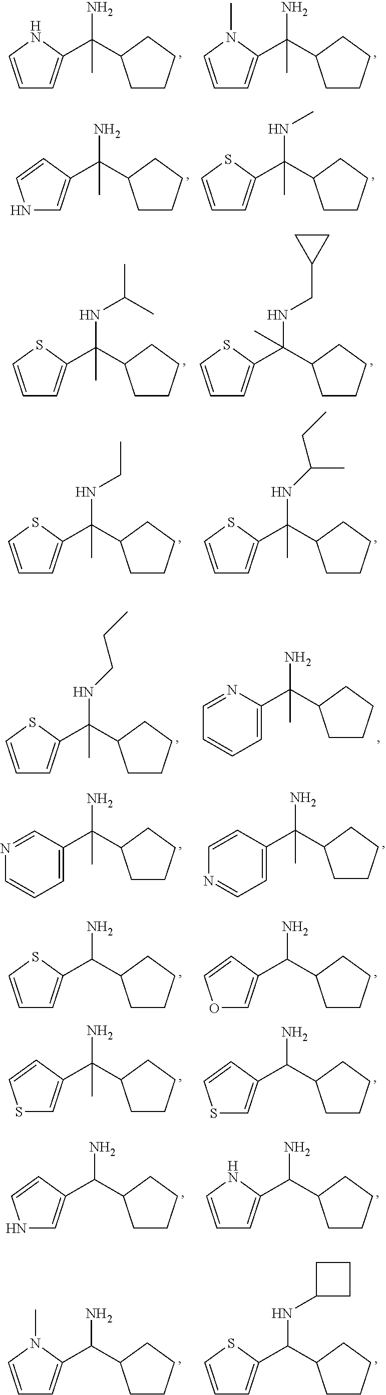

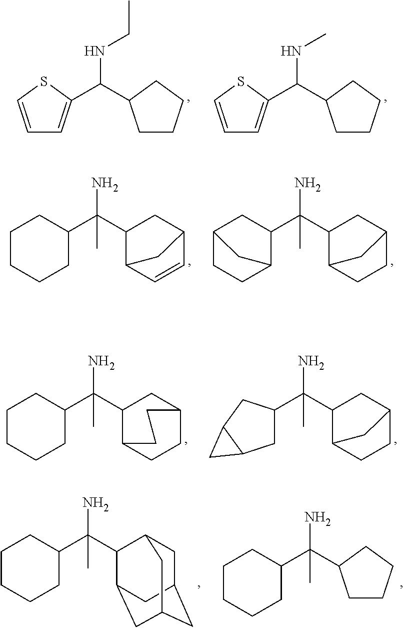

or a pharmaceutically acceptable salt of the compound.

In another aspect, disclosed herein is a compound represented by Formula (II):

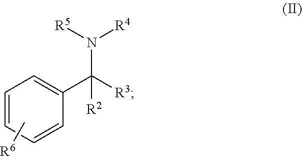

or an enantiomer, a mixture of enantiomers, or an isotopic variant thereof; or a pharmaceutically acceptable salt, solvate or hydrate thereof, wherein:
R² is selected from
   hydrogen;
   halogen;
   C₁₋₈ alkyl, C₂₋₈ alkenyl, and C₂₋₈ alkynyl, each of which is optionally substituted with one or more R⁷; and
   C₃₋₁₀ carbocycle and 3- to 10-membered heterocycle; wherein the C₃₋₁₀ carbocycle and 3-to 10-membered heterocycle are each optionally substituted with one or more R⁷; R³ is selected from:
   C₁₋₆ alkyl, C₂₋₆ alkenyl, and C₂₋₆ alkynyl, each of which is optionally substituted with one or more R⁸; and
   C₃₋₁₀ carbocycle and 3- to 10-membered heterocycle; wherein the C₃₋₁₀ carbocycle and 3-to 10-membered heterocycle are each optionally substituted with one or more R¹; R⁴, R⁵, R⁶, R⁷, and R⁸ are each independently selected from:
   hydrogen;
   C₁₋₁₀ alkyl, C₂₋₁₀ alkenyl, and C₂₋₁₀ alkynyl optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₃₋₆ cycloalkyl, and C₃₋₆ cycloalkenyl; and
   C₃₋₁₀ carbocycle or 3- to 10-membered heterocycle; wherein the C₃₋₁₀ carbocycle and 3- to 10-membered heterocycle are each optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), and C₁₋₈ alkyl; with the proviso that the compound of Formula I is not

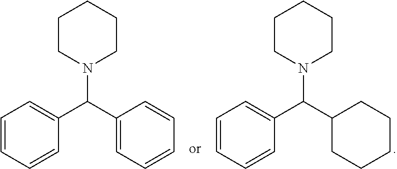

In some embodiments, R² is hydrogen, C₁₋₈ alkyl, C₂₋₈ alkenyl, or C₂₋₈ alkynyl, each of which is optionally substituted with one or more R⁷.

In some embodiments, R² is C₃₋₇ carbocycle or 3- to 7-membered heterocycle; wherein the C₃₋₇ carbocycle and 3- to 7-membered heterocycle are each optionally substituted with one or more R⁷.

In some embodiments, R² is phenyl, optionally substituted with one or more R⁷.

In some embodiments, R² is a cycloalkyl. In some embodiments, R² is a cycloalkyl selected from cyclooctane, cycloheptane, cyclohexane, cyclopentane, cyclobutane, and cyclopropane, any of which is optionally substituted with one or more R⁷.

In some embodiments, R² is a cycloalkenyl. In some embodiments, R² is a cycloalkenyl selected from cyclooctene, cycloheptene, cyclohexene, cyclopentene, cyclobutene, and cyclopropane, any of which is optionally substituted with one or more R⁷. In some embodiments, the cycloalkenyl can have 1, 2, 3, 4, or more degrees of unsaturation.

In some embodiments, R² is a 6-membered heteroaryl. In some embodiments, R² is a 6-membered heteroaryl selected from pyridine, pyrazine, and pyrimidine, any of which is optionally substituted with one or more R⁷. In some embodiments, the heteroatom of the heteroaryl is not bound to the methylene carbon of Formula (II).

In some embodiments, R² is a 5-membered heteroaryl. In some embodiments, R² is a 5-membered heteroaryl selected from thiophene, furan, pyrrole, pyrazole, selenophene, imidazole, thiazole, isothiazole, oxatriazole, oxadiazole, oxazole, and thiadiazole, any of which is optionally substituted with one or more R⁷. In some embodiments, the heteroatom of the heteroaryl is not bound to the methylene carbon of Formula (II).

In some embodiments, R² is a 6-membered heterocycloalkenyl. In some embodiments, R² is a 6-membered heterocycloalkenyl selected from dihydropyridine, tetrahydropyridine, dihydropyridazine, tetrahydropyridazine, dihydropyrimidine, tetrahydropyrimidine, dihydropyrazine, tetrahydropyridazine, dihydropyrimidine, tetrahydropyrimidine, dihydropyrazine, tetrahydropyrazine, pyran, dihydropyran, thiopyran, dihydrothiopyran, dioxine, dihydrodioxine, oxazine, dihydrooxazine, thiazine, and dihydrothiazine, any of which is optionally substituted with one or more R⁷. In some embodiments, the heteroatom of the heterocycloalkenyl is not bound to the methylene carbon of Formula (II).

In some embodiments, R² is a 5-membered heterocycloalkenyl. In some embodiments, R² is a 5-membered heterocycloalkenyl selected from pyrroline, pyrazoline, imidazoline, triazoline, dihydrofuran, dihydrothiophene, oxazoline, isooxazoline, thiazoline, isothiazoline, oxadiazoline, and thiadiazoline, any of which is optionally substituted with one or more R⁷. In some embodiments, the heteroatom of the heterocycloalkenyl is not bound to the methylene carbon of Formula (II).

In some embodiments, R² is a 6-membered heterocycloalkyl. In some embodiments, R² is a 6-membered heterocycloalkyl selected from piperidine, piperazine, tetrahydrothiopyran, tetrahydropyran, dioxane, morpholine, and thiomorpholine, any of which is optionally substituted with one or more R⁷. In some embodiments, the heteroatom of the heterocycloalkyl is not bound to the methylene carbon of Formula (II).

In some embodiments, R² is a 5-membered heterocycloalkyl. In some embodiments, R² is a 5-membered heterocycloalkyl selected from tetrahydrofuran, pyrrolidone, and tetrahydrothiophene, any of which is optionally substituted with one or more R⁷. In some embodiments, the heteroatom of the heterocycloalkyl is not bound to the methylene carbon of Formula (II).

In some embodiments, R² is a 4-membered heterocycloalkyl. In some embodiments, R² is a 4-membered heterocycloalkyl selected from azetidine, oxetane, and thietane, any of which is optionally substituted with one or more R⁷. In some embodiments, the heteroatom of the heterocycloalkyl is not bound to the methylene carbon of Formula (II).

In some embodiments, R² is a 6-6 fused ring system. In some embodiments, R² is a 6-6 fused ring system selected from naphthalene, quinoline, isoquinoline, pteridine, benzopyran, dihydroquinoline, dihydroisoquinoline, dihydronapthalene, and tetrahydronapthalene, any of which is optionally substituted with one or more R⁷.

In some embodiments, R² is a 5-6 fused ring system. In some embodiments, R² is a 5-6 fused ring system selected from benzimidazole, indole, azaindole, indazole, benzothiophene, benzofuran, benzoselenophene, benzimidazole, benzotriazole, benzothiazole, benzisothiazole, benzoxadiazole, benzoxazole, and benzothiadiazole, imidazo[4,5-b]-pyridine, imidazo[4,5-c]-pyridine, pyrazolo[3,4-b]pyridine, pyrazolo[3,4-c]pyridine, pyrazolo[4,3-b]pyridine, pyrazolo[4,3-b]pyridine, oxazolo[4,5-b]pyridine, oxazolo[4,5-c]pyridine, oxazolo[5,4-b]pyridine, oxazolo[5,4-c]pyridine, thiazolo[4,5-b]pyridine, thiazolo[4,5-c]pyridine, thiazolo[5,4-b]pyridine, thiazolo[5,4-c]pyridine, isooxazolo[4,5-b]pyridine, isooxazolo[4,5-c]pyridine, isooxazolo[3,4-b]pyridine, isooxazolo[3,4-c]pyridine, isooxazolo[4,3-b]pyridine, isooxazolo[4,3-c]pyridine, isooxazolo[5,4-b]pyridine, isooxazolo[5,4-c]pyridine, isothiazolo[4,5-b]pyridine, isothiazolo[4,5-c]pyridine, isothiazolo[5,4-b]pyridine, isothiazolo[5,4-c]pyridine, isothiazolo[3,4-b]pyridine, isothiazolo[3,4-c]pyridine, isothiazolo[4,3-b]pyridine, isothiazolo[4,3-c]pyridine and purine, any of which is optionally substituted with one or more R⁷.

In some embodiments, R² is a 5-5 fused ring system. In some embodiments, R² is a 5-5 fused ring system selected from pyrrolo[2,1-b]thiazole, pyrazolo[5,1-b]thiazole, imidazo[5,1-b]thiazole, imidazo[2,1-b]thiazole, thiazolo[3,2-b][1,2,4]triazole, thiazolo[3,2-c][1,2,4]triazole, imidazo[2,1-b]thiadiazole, imidazo[1,2-d][1,2,4]thiadiazole, thiazolo[3,2-d]tetrazole, thieno[3,2-b]thiophene, thieno[2,3-b]thiophene, thieno[3,4-b]thiophene, thieno[3,4-c]thiophene, any of which is optionally substituted with one or more R⁷.

In some embodiments, R² is a bridged ring system. In some embodiments, R² is a bridged ring system selected from norbornane, norbornene, bicyclo[1.1.0]butane, bicyclo[3.2.0]heptane, bicyclo[3.3.0]octane, bicyclo[4.3.0]nonane, bicyclo[1.1.1]pentane, bicyclo[2.2.1]hexane, bicyclo[3.3.1]nonane, bicyclo[2.2.2]octane, adamantane, cubane, housane, any of which is optionally substituted with one or more R⁷. In some embodiments, the bridged ring system can have 1, 2, 3, 4, or more degrees of unsaturation.

In some embodiments, R³ is hydrogen, C₁₋₆ alkyl, C₂₋₆ alkenyl, or C₂₋₆ alkynyl, each of which is optionally substituted with one or more R⁸.

In some embodiments, R³ is C₃₋₇ carbocycle or 3- to 7-membered heterocycle; wherein the C₃₋₇ carbocycle and 3- to 7-membered heterocycle are each optionally substituted with one or more R⁸.

In some embodiments, R³ is phenyl, optionally substituted with one or more R⁸.

In some embodiments, R³ is a cycloalkyl. In some embodiments, R³ is a cycloalkyl selected from cyclooctane, cycloheptane, cyclohexane, cyclopentane, cyclobutane, and cyclopropane, any of which is optionally substituted with one or more R⁸.

In some embodiments, R³ is a cycloalkenyl. In some embodiments, R³ is a cycloalkenyl selected from cyclooctene, cycloheptene, cyclohexene, cyclopentene, cyclobutene, and cyclopropane, any of which is optionally substituted with one or more R⁸. In some embodiments, the cycloalkenyl can have 1, 2, 3, 4, or more degrees of unsaturation.

In some embodiments, R³ is a 6-membered heteroaryl. In some embodiments, R³ is a 6-membered heteroaryl selected from pyridine, pyrazine, and pyrimidine, any of which is optionally substituted with one or more R⁸. In some embodiments, the heteroatom of the heteroaryl is not bound to the methylene carbon of Formula (II).

In some embodiments, R³ is a 5-membered heteroaryl. In some embodiments, R³ is a 5-membered heteroaryl selected from thiophene, furan, pyrrole, pyrazole, selenophene, imidazole, thiazole, isothiazole, oxatriazole, oxadiazole, oxazole, and thiadiazole, any of which is optionally substituted with one or more R⁸. In some embodiments, the heteroatom of the heteroaryl is not bound to the methylene carbon of Formula (II).

In some embodiments, R³ is a 6-membered heterocycloalkenyl. In some embodiments, R³ is a 6-membered heterocycloalkenyl selected from dihydropyridine, tetrahydropyridine, dihydropyridazine, tetrahydropyridazine, dihydropyrimidine, tetrahydropyrimidine, dihydropyrazine, tetrahydropyridazine, dihydropyrimidine, tetrahydropyrimidine, dihydropyrazine, tetrahydropyrazine, pyran, dihydropyran, thiopyran, dihydrothiopyran, dioxine, dihydrodioxine, oxazine, dihydrooxazine, thiazine, and dihydrothiazine, any of which is optionally substituted with one or more R⁸. In some embodiments, the heteroatom of the heterocycloalkenyl is not bound to the methylene carbon of Formula (II).

In some embodiments, R³ is a 5-membered heterocycloalkenyl. In some embodiments, R³ is a 5-membered heterocycloalkenyl selected from pyrroline, pyrazoline, imidazoline, triazoline, dihydrofuran, dihydrothiophene, oxazoline, isooxazoline, thiazoline, isothiazoline, oxadiazoline, and thiadiazoline, any of which is optionally substituted with one or more R⁸. In some embodiments, the heteroatom of the heterocycloalkenyl is not bound to the methylene carbon of Formula (II).

In some embodiments, R³ is a 6-membered heterocycloalkyl. In some embodiments, R³ is a 6-membered heterocycloalkyl selected from piperidine, piperazine, tetrahydrothiopyran, tetrahydropyran, dioxane, morpholine, and thiomorpholine, any of which is optionally substituted with one or more R⁸. In some embodiments, the heteroatom of the heterocycloalkyl is not bound to the methylene carbon of Formula (II).

In some embodiments, R³ is a 5-membered heterocycloalkyl. In some embodiments, R³ is a 5-membered heterocycloalkyl selected from tetrahydrofuran, pyrrolidone, and tetrahydrothiophene, any of which is optionally substituted with one or more R⁸. In some embodiments, the heteroatom of the heterocycloalkyl is not bound to the methylene carbon of Formula (II).

In some embodiments, R³ is a 4-membered heterocycloalkyl. In some embodiments, R³ is a 4-membered heterocycloalkyl selected from azetidine, oxetane, and thietane, any of which is optionally substituted with one or more R⁸. In some embodiments, the heteroatom of the heterocycloalkyl is not bound to the methylene carbon of Formula (II).

In some embodiments, R³ is a 6-6 fused ring system. In some embodiments, R³ is a 6-6 fused ring system selected from naphthalene, quinoline, isoquinoline, pteridine, benzopyran, dihydroquinoline, dihydroisoquinoline, dihydronapthalene, and tetrahydronapthalene, any of which is optionally substituted with one or more R⁸.

In some embodiments, R³ is a 5-6 fused ring system. In some embodiments, R³ is a 5-6 fused ring system selected from benzimidazole, indole, azaindole, indazole, benzothiophene, benzofuran, benzoselenophene, benzimidazole, benzotriazole, benzothiazole, benzisothiazole, benzoxadiazole, benzoxazole, and benzothiadiazole, imidazo[4,5-b]-pyridine, imidazo[4,5-c]-pyridine, pyrazolo[3,4-b]pyridine, pyrazolo[3,4-c]pyridine, pyrazolo[4,3-b]pyridine, pyrazolo[4,3-b]pyridine, oxazolo[4,5-b]pyridine, oxazolo[4,5-c]pyridine, oxazolo[5,4-b]pyridine, oxazolo[5,4-c]pyridine, thiazolo[4,5-b]pyridine, thiazolo[4,5-c]pyridine, thiazolo[5,4-b]pyridine, thiazolo[5,4-c]pyridine, isooxazolo[4,5-b]pyridine, isooxazolo[4,5-c]pyridine, isooxazolo[3,4-b]pyridine, isooxazolo[3,4-c]pyridine, isooxazolo[4,3-b]pyridine, isooxazolo[4,3-c]pyridine, isooxazolo[5,4-b]pyridine, isooxazolo[5,4-c]pyridine, isothiazolo[4,5-b]pyridine, isothiazolo[4,5-c]pyridine, isothiazolo[5,4-b]pyridine, isothiazolo[5,4-c]pyridine, isothiazolo[3,4-b]pyridine, isothiazolo[3,4-c]pyridine, isothiazolo[4,3-b]pyridine, isothiazolo[4,3-c]pyridine and purine, any of which is optionally substituted with one or more R⁸.

In some embodiments, R³ is a 5-5 fused ring system. In some embodiments, R³ is a 5-5 fused ring system selected from pyrrolo[2,1-b]thiazole, pyrazolo[5,1-b]thiazole, imidazo[5,1-b]thiazole, imidazo[2,1-b]thiazole, thiazolo[3,2-b][1,2,4]triazole, thiazolo[3,2-c][1,2,4]triazole, imidazo[2,1-b]thiadiazole, imidazo[1,2-d][1,2,4]thiadiazole, thiazolo[3,2-d]tetrazole, thieno[3,2-b]thiophene, thieno[2,3-b]thiophene, thieno[3,4-b]thiophene, thieno[3,4-c]thiophene, any of which is optionally substituted with one or more R⁸.

In some embodiments, R³ is a bridged ring system. In some embodiments, R³ is a bridged ring system selected from norbornane, norbornene, bicyclo[1.1.0]butane, bicyclo[3.2.0]heptane, bicyclo[3.3.0]octane, bicyclo[4.3.0]nonane, bicyclo[1.1.1]pentane, bicyclo[2.2.1]hexane, bicyclo[3.3.1]nonane, bicyclo[2.2.2]octane, adamantane, cubane, housane, any of which is optionally substituted with one or more R⁸. In some embodiments, the bridged ring system can have 1, 2, 3, 4, or more degrees of unsaturation.

In some embodiments, each of R⁴ or R⁵ may independently be hydrogen, C₁₋₁₀ alkyl, C₂₋₁₀ alkenyl, or C₂₋₁₀ alkynyl, each of which is optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₁₋₆ cycloalkyl, and C₁₋₆ cycloalkenyl.

In some embodiments, each of R⁴ or R⁵ may independently be a 3- to 10-membered heterocycle. In some embodiments, each of R⁴ or R⁵ may independently be a 3- to 10-membered heterocycle selected from azacyclopropane, pyrrolidine, piperidine, azepane, azocane, azonane, morpholine, thiomorpholine, 1,4-piperazine, 1,4-oxazepane, and 1,4-diazepane.

In some embodiments, compound of Formula (II) is selected from:

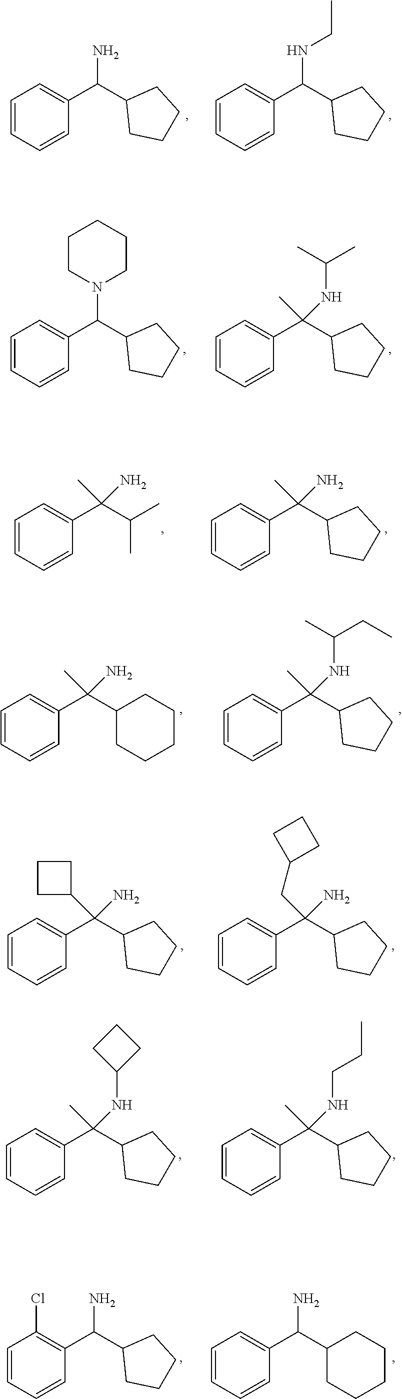

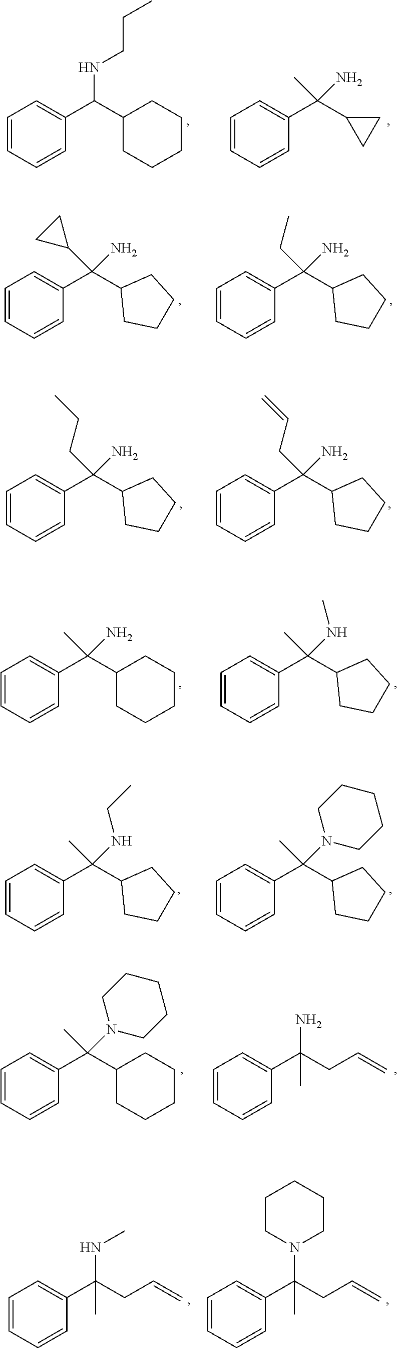

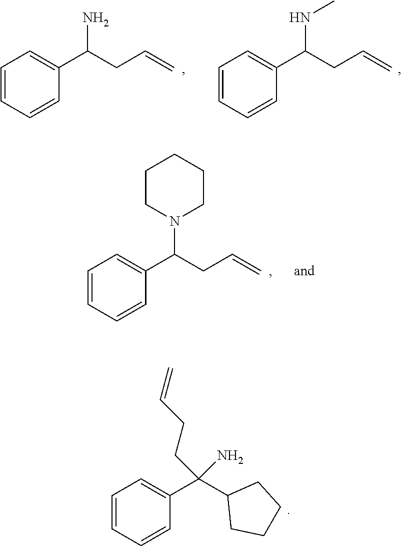

In another aspect, disclosed herein is a compound represented by Formula (III):

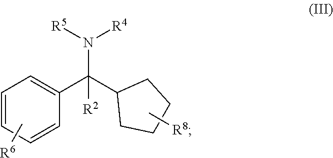

or an enantiomer, a mixture of enantiomers, or an isotopic variant thereof; or a pharmaceutically acceptable salt, solvate or hydrate thereof, wherein:
R² is selected from
   hydrogen;
   halogen;
   C₁₋₈ alkyl, C₂₋₈ alkenyl, and C₂₋₈ alkynyl, each of which is optionally substituted with one or more R⁷; and
   C₃₋₁₀ carbocycle and 3- to 10-membered heterocycle; wherein the C₃₋₁₀ carbocycle and 3-to 10-membered heterocycle are each optionally substituted with one or more R⁷; R⁴, R⁵, R⁶, R⁷, and R⁸ are each independently selected from:
   hydrogen;
   C₁₋₁₀ alkyl, C₂₋₁₀ alkenyl, and C₂₋₁₀ alkynyl optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₁₋₆ cycloalkyl, and C₁₋₆ cycloalkenyl; and
   C₃₋₁₀ carbocycle or 3- to 10-membered heterocycle; wherein the C₃₋₁₀ carbocycle and 3- to 10-membered heterocycle are each optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), and C₁₋₈ alkyl.

In some embodiments, R² is hydrogen, C₁₋₈ alkyl, C₂₋₈ alkenyl, or C₂₋₈ alkynyl, each of which is optionally substituted with one or more R⁷.

In some embodiments, R² is C₃₋₇ carbocycle or 3- to 7-membered heterocycle; wherein the C₃₋₇ carbocycle and 3- to 7-membered heterocycle are each optionally substituted with one or more R⁷.

In some embodiments, R² is phenyl, optionally substituted with one or more R⁷.

In some embodiments, R² is a cycloalkyl. In some embodiments, R² is a cycloalkyl selected from cyclooctane, cycloheptane, cyclohexane, cyclopentane, cyclobutane, and cyclopropane, any of which is optionally substituted with one or more R⁷.

In some embodiments, R² is a cycloalkenyl. In some embodiments, R² is a cycloalkenyl selected from cyclooctene, cycloheptene, cyclohexene, cyclopentene, cyclobutene, and cyclopropane, any of which is optionally substituted with one or more R⁷. In some embodiments, the cycloalkenyl can have 1, 2, 3, 4, or more degrees of unsaturation.

In some embodiments, R² is a 6-membered heteroaryl. In some embodiments, R² is a 6-membered heteroaryl selected from pyridine, pyrazine, and pyrimidine, any of which is optionally substituted with one or more R⁷. In some embodiments, the heteroatom of the heteroaryl is not bound to the methylene carbon of Formula (III).

In some embodiments, R² is a 5-membered heteroaryl. In some embodiments, R² is a 5-membered heteroaryl selected from thiophene, furan, pyrrole, pyrazole, selenophene, imidazole, thiazole, isothiazole, oxatriazole, oxadiazole, oxazole, and thiadiazole, any of which is optionally substituted with one or more R⁷. In some embodiments, the heteroatom of the heteroaryl is not bound to the methylene carbon of Formula (III).

In some embodiments, R² is a 6-membered heterocycloalkenyl. In some embodiments, R² is a 6-membered heterocycloalkenyl selected from dihydropyridine, tetrahydropyridine, dihydropyridazine, tetrahydropyridazine, dihydropyrimidine, tetrahydropyrimidine, dihydropyrazine, tetrahydropyridazine, dihydropyrimidine, tetrahydropyrimidine, dihydropyrazine, tetrahydropyrazine, pyran, dihydropyran, thiopyran, dihydrothiopyran, dioxine, dihydrodioxine, oxazine, dihydrooxazine, thiazine, and dihydrothiazine, any of which is optionally substituted with one or more R⁷. In some embodiments, the heteroatom of the heterocycloalkenyl is not bound to the methylene carbon of Formula (III).

In some embodiments, R² is a 5-membered heterocycloalkenyl. In some embodiments, R² is a 5-membered heterocycloalkenyl selected from pyrroline, pyrazoline, imidazoline, triazoline, dihydrofuran, dihydrothiophene, oxazoline, isooxazoline, thiazoline, isothiazoline, oxadiazoline, and thiadiazoline, any of which is optionally substituted with one or more R⁷. In some embodiments, the heteroatom of the heterocycloalkenyl is not bound to the methylene carbon of Formula (III).

In some embodiments, R² is a 6-membered heterocycloalkyl. In some embodiments, R² is a 6-membered heterocycloalkyl selected from piperidine, piperazine, tetrahydrothiopyran, tetrahydropyran, dioxane, morpholine, and thiomorpholine, any of which is optionally substituted with one or more R⁷. In some embodiments, the heteroatom of the heterocycloalkyl is not bound to the methylene carbon of Formula (III).

In some embodiments, R² is a 5-membered heterocycloalkyl. In some embodiments, R² is a 5-membered heterocycloalkyl selected from tetrahydrofuran, pyrrolidone, and tetrahydrothiophene, any of which is optionally substituted with one or more R⁷. In some embodiments, the heteroatom of the heterocycloalkyl is not bound to the methylene carbon of Formula (III).

In some embodiments, R² is a 4-membered heterocycloalkyl. In some embodiments, R² is a 4-membered heterocycloalkyl selected from azetidine, oxetane, and thietane, any of which is optionally substituted with one or more R⁷. In some embodiments, the heteroatom of the heterocycloalkyl is not bound to the methylene carbon of Formula (III).

In some embodiments, R² is a 6-6 fused ring system. In some embodiments, R² is a 6-6 fused ring system selected from naphthalene, quinoline, isoquinoline, pteridine, benzopyran, dihydroquinoline, dihydroisoquinoline, dihydronapthalene, and tetrahydronapthalene, any of which is optionally substituted with one or more R⁷.

In some embodiments, R² is a 5-6 fused ring system. In some embodiments, R² is a 5-6 fused ring system selected from benzimidazole, indole, azaindole, indazole, benzothiophene, benzofuran, benzoselenophene, benzimidazole, benzotriazole, benzothiazole, benzisothiazole, benzoxadiazole, benzoxazole, and benzothiadiazole, imidazo[4,5-b]-pyridine, imidazo[4,5-c]-pyridine, pyrazolo[3,4-b]pyridine, pyrazolo[3,4-c]pyridine, pyrazolo[4,3-b]pyridine, pyrazolo[4,3-b]pyridine, oxazolo[4,5-b]pyridine, oxazolo[4,5-c]pyridine, oxazolo[5,4-b]pyridine, oxazolo[5,4-c]pyridine, thiazolo[4,5-b]pyridine, thiazolo[4,5-c]pyridine, thiazolo[5,4-b]pyridine, thiazolo[5,4-c]pyridine, isooxazolo[4,5-b]pyridine, isooxazolo[4,5-c]pyridine, isooxazolo[3,4-b]pyridine, isooxazolo[3,4-c]pyridine, isooxazolo[4,3-b]pyridine, isooxazolo[4,3-c]pyridine, isooxazolo[5,4-b]pyridine, isooxazolo[5,4-c]pyridine, isothiazolo[4,5-b]pyridine, isothiazolo[4,5-c]pyridine, isothiazolo[5,4-b]pyridine, isothiazolo[5,4-c]pyridine, isothiazolo[3,4-b]pyridine, isothiazolo[3,4-c]pyridine, isothiazolo[4,3-b]pyridine, isothiazolo[4,3-c]pyridine and purine, any of which is optionally substituted with one or more R⁷.

In some embodiments, R² is a 5-5 fused ring system selected. In some embodiments, R² is a 5-5 fused ring system selected from pyrrolo[2,1-b]thiazole, pyrazolo[5,1-b]thiazole, imidazo[5,1-b]thiazole, imidazo[2,1-b]thiazole, thiazolo[3,2-b][1,2,4]triazole, thiazolo[3,2-c][1,2,4]triazole, imidazo[2,1-b]thiadiazole, imidazo[1,2-d][1,2,4]thiadiazole, thiazolo[3,2-d]tetrazole, thieno[3,2-b]thiophene, thieno[2,3-b]thiophene, thieno[3,4-b]thiophene, thieno[3,4-c]thiophene, any of which is optionally substituted with one or more R⁷.

In some embodiments, R² is a bridged ring system. In some embodiments, R² is a bridged ring system selected from norbornane, norbornene, bicyclo[1.1.0]butane, bicyclo[3.2.0]heptane, bicyclo[3.3.0]octane, bicyclo[4.3.0]nonane, bicyclo[1.1.1]pentane, bicyclo[2.2.1]hexane, bicyclo[3.3.1]nonane, bicyclo[2.2.2]octane, adamantane, cubane, housane, any of which is optionally substituted with one or more R⁷. In some embodiments, the bridged ring system can have 1, 2, 3, 4, or more degrees of unsaturation.

In some embodiments, each of R⁴ or R⁵ may independently be hydrogen, C₁₋₁₀ alkyl, C₂₋₁₀ alkenyl, or C₂₋₁₀ alkynyl, each of which is optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₁₋₆ cycloalkyl, and C₁₋₆ cycloalkenyl.

In some embodiments, each of R⁴ or R⁵ may independently be 3- to 10-membered heterocycle. In some embodiments, each of R⁴ or R⁵ may independently be 3- to 10-membered heterocycle selected from azacyclopropane, pyrrolidine, piperidine, azepane, azocane, azonane, morpholine, thiomorpholine, 1,4-piperazine, 1,4-oxazepane, and 1,4-diazepane.

In some embodiments, the compound of Formula (III) is selected from:

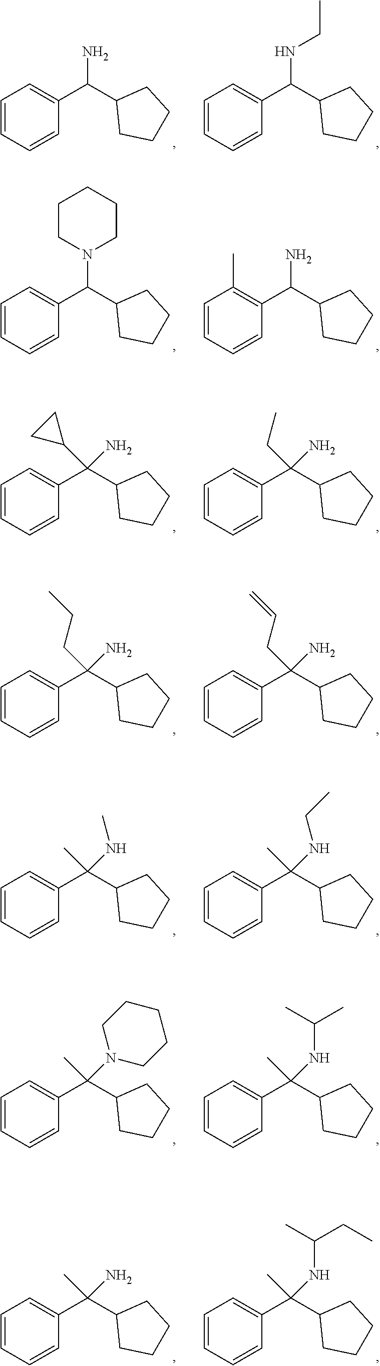

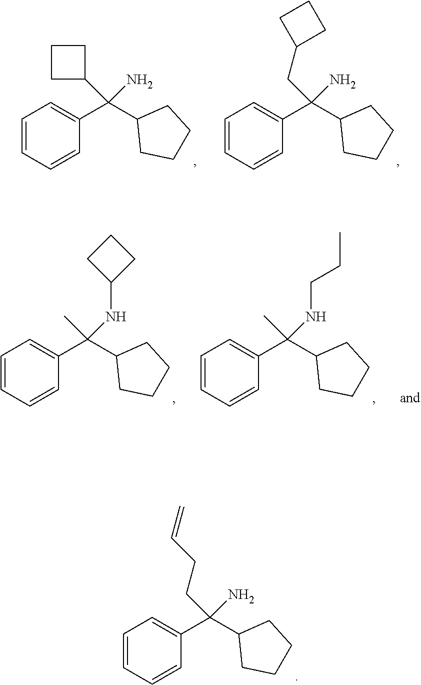

In some embodiments, the compound of Formula (III) is selected from:

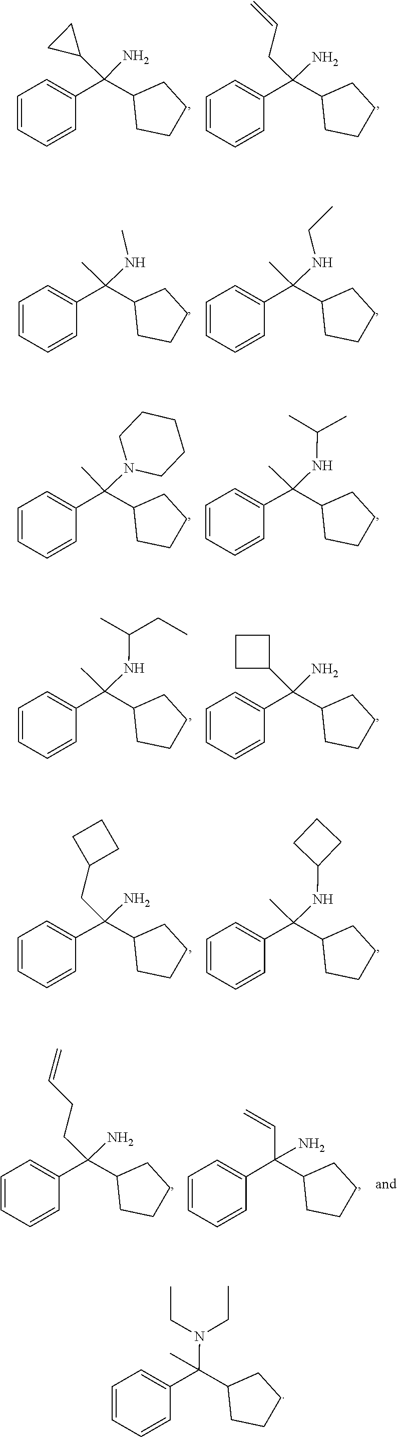

In another aspect, disclosed herein is a compound represented by Formula (IV):

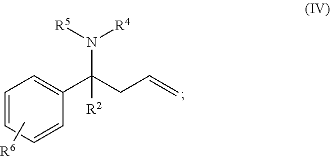

or an enantiomer, a mixture of enantiomers, or an isotopic variant thereof; or a pharmaceutically acceptable salt, solvate or hydrate thereof, wherein:
R² is selected from
   hydrogen;
   halogen;
   C₁₋₈ alkyl, C₂₋₈ alkenyl, and C₂₋₈ alkynyl, each of which is optionally substituted with one or more R⁷; and
   C₃₋₁₀ carbocycle and 3- to 10-membered heterocycle; wherein the C₃₋₁₀ carbocycle and 3-to 10-membered heterocycle are each optionally substituted with one or more R⁷;
R⁴, R⁵, R⁶, R⁷, and R⁸ are each independently selected from:
   hydrogen;
   C₁₋₁₀ alkyl, C₂₋₁₀ alkenyl, and C₂₋₁₀ alkynyl optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₁₋₆ cycloalkyl, and C₁₋₆ cycloalkenyl; and
   C₃₋₁₀ carbocycle or 3- to 10-membered heterocycle; wherein the C₃₋₁₀ carbocycle and 3- to 10-membered heterocycle are each optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), and C₁₋₈ alkyl.

In some embodiments, R² is hydrogen, C₁₋₈ alkyl, C₂₋₈ alkenyl, or C₂₋₈ alkynyl, each of which is optionally substituted with one or more R⁷.

In some embodiments, R² is C₃₋₇ carbocycle or 3- to 7-membered heterocycle; wherein the C₃₋₇ carbocycle and 3- to 7-membered heterocycle are each optionally substituted with one or more R⁷.

In some embodiments, R² is phenyl, optionally substituted with one or more R⁷.

In some embodiments, R² is a cycloalkyl. In some embodiments, R² is a cycloalkyl selected from cyclooctane, cycloheptane, cyclohexane, cyclopentane, cyclobutane, and cyclopropane, any of which is optionally substituted with one or more R⁷.

In some embodiments, R² is a cycloalkenyl. In some embodiments, R² is a cycloalkenyl selected from cyclooctene, cycloheptene, cyclohexene, cyclopentene, cyclobutene, and cyclopropane, any of which is optionally substituted with one or more R⁷. In some embodiments, the cycloalkenyl can have 1, 2, 3, 4, or more degrees of unsaturation.

In some embodiments, R² is a 6-membered heteroaryl. In some embodiments, R² is a 6-membered heteroaryl selected from pyridine, pyrazine, and pyrimidine, any of which is optionally substituted with one or more R⁷. In some embodiments, the heteroatom of the heteroaryl is not bound to the methylene carbon of Formula (IV).

In some embodiments, R² is a 5-membered heteroaryl. In some embodiments, R² is a 5-membered heteroaryl selected from thiophene, furan, pyrrole, pyrazole, selenophene, imidazole, thiazole, isothiazole, oxatriazole, oxadiazole, oxazole, and thiadiazole, any of which is optionally substituted with one or more R⁷. In some embodiments, the heteroatom of the heteroaryl is not bound to the methylene carbon of Formula (IV).

In some embodiments, R² is a 6-membered heterocycloalkenyl. In some embodiments, R² is a 6-membered heterocycloalkenyl selected from dihydropyridine, tetrahydropyridine, dihydropyridazine, tetrahydropyridazine, dihydropyrimidine, tetrahydropyrimidine, dihydropyrazine, tetrahydropyridazine, dihydropyrimidine, tetrahydropyrimidine, dihydropyrazine, tetrahydropyrazine, pyran, dihydropyran, thiopyran, dihydrothiopyran, dioxine, dihydrodioxine, oxazine, dihydrooxazine, thiazine, and dihydrothiazine, any of which is optionally substituted with one or more R⁷. In some embodiments, the heteroatom of the heterocycloalkenyl is not bound to the methylene carbon of Formula (IV).

In some embodiments, R² is a 5-membered heterocycloalkenyl. In some embodiments, R² is a 5-membered heterocycloalkenyl selected from pyrroline, pyrazoline, imidazoline, triazoline, dihydrofuran, dihydrothiophene, oxazoline, isooxazoline, thiazoline, isothiazoline, oxadiazoline, and thiadiazoline, any of which is optionally substituted with one or more R⁷. In some embodiments, the heteroatom of the heterocycloalkenyl is not bound to the methylene carbon of Formula (IV).

In some embodiments, R² is a 6-membered heterocycloalkyl. In some embodiments, R² is a 6-membered heterocycloalkyl selected from piperidine, piperazine, tetrahydrothiopyran, tetrahydropyran, dioxane, morpholine, and thiomorpholine, any of which is optionally substituted with one or more R⁷. In some embodiments, the heteroatom of the heterocycloalkyl is not bound to the methylene carbon of Formula (IV).

In some embodiments, R² is a 5-membered heterocycloalkyl. In some embodiments, R² is a 5-membered heterocycloalkyl selected from tetrahydrofuran, pyrrolidone, and tetrahydrothiophene, any of which is optionally substituted with one or more R⁷. In some embodiments, the heteroatom of the heterocycloalkyl is not bound to the methylene carbon of Formula (IV).

In some embodiments, R² is a 4-membered heterocycloalkyl. In some embodiments, R² is a 4-membered heterocycloalkyl selected from azetidine, oxetane, and thietane, any of which is optionally substituted with one or more R⁷. In some embodiments, the heteroatom of the heterocycloalkyl is not bound to the methylene carbon of Formula (IV).

In some embodiments, R² is a 6-6 fused ring system. In some embodiments, R² is a 6-6 fused ring system selected from naphthalene, quinoline, isoquinoline, pteridine, benzopyran, dihydroquinoline, dihydroisoquinoline, dihydronapthalene, and tetrahydronapthalene, any of which is optionally substituted with one or more R⁷.

In some embodiments, R² is a 5-6 fused ring system. In some embodiments, R² is a 5-6 fused ring system selected from benzimidazole, indole, azaindole, indazole, benzothiophene, benzofuran, benzoselenophene, benzimidazole, benzotriazole, benzothiazole, benzisothiazole, benzoxadiazole, benzoxazole, and benzothiadiazole, imidazo[4,5-b]-pyridine, imidazo[4,5-c]-pyridine, pyrazolo[3,4-b]pyridine, pyrazolo[3,4-c]pyridine, pyrazolo[4,3-b]pyridine, pyrazolo[4,3-b]pyridine, oxazolo[4,5-b]pyridine, oxazolo[4,5-c]pyridine, oxazolo[5,4-b]pyridine, oxazolo[5,4-c]pyridine, thiazolo[4,5-b]pyridine, thiazolo[4,5-c]pyridine, thiazolo[5,4-b]pyridine, thiazolo[5,4-c]pyridine, isooxazolo[4,5-b]pyridine, isooxazolo[4,5-c]pyridine, isooxazolo[3,4-b]pyridine, isooxazolo[3,4-c]pyridine, isooxazolo[4,3-b]pyridine, isooxazolo[4,3-c]pyridine, isooxazolo[5,4-b]pyridine, isooxazolo[5,4-c]pyridine, isothiazolo[4,5-b]pyridine, isothiazolo[4,5-c]pyridine, isothiazolo[5,4-b]pyridine, isothiazolo[5,4-c]pyridine, isothiazolo[3,4-b]pyridine, isothiazolo[3,4-c]pyridine, isothiazolo[4,3-b]pyridine, isothiazolo[4,3-c]pyridine and purine, any of which is optionally substituted with one or more R⁷.

In some embodiments, R² is a 5-5 fused ring system. In some embodiments, R² is a 5-5 fused ring system selected from pyrrolo[2,1-b]thiazole, pyrazolo[5,1-b]thiazole, imidazo[5,1-b]thiazole, imidazo[2,1-b]thiazole, thiazolo[3,2-b][1,2,4]triazole, thiazolo[3,2-c][1,2,4]triazole, imidazo[2,1-b]thiadiazole, imidazo[1,2-d][1,2,4]thiadiazole, thiazolo[3,2-d]tetrazole, thieno[3,2-b]thiophene, thieno[2,3-b]thiophene, thieno[3,4-b]thiophene, thieno[3,4-c]thiophene, any of which is optionally substituted with one or more R⁷.

In some embodiments, R² is a bridged ring system. In some embodiments, R² is a bridged ring system selected from norbornane, norbornene, bicyclo[1.1.0]butane, bicyclo[3.2.0]heptane, bicyclo[3.3.0]octane, bicyclo[4.3.0]nonane, bicyclo[1.1.1]pentane, bicyclo[2.2.1]hexane, bicyclo[3.3.1]nonane, bicyclo[2.2.2]octane, adamantane, cubane, housane, any of which is optionally substituted with one or more R⁷. In some embodiments, the bridged ring system can have 1, 2, 3, 4, or more degrees of unsaturation.

In some embodiments, each of R⁴ or R⁵ may independently be hydrogen, C₁₋₁₀ alkyl, C₂₋₁₀ alkenyl, or C₂₋₁₀ alkynyl, each of which is optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₁₋₆ cycloalkyl, and C₁₋₆ cycloalkenyl.

In some embodiments, each of R⁴ or R⁵ may independently be 3- to 10-membered heterocycle. In some embodiments, each of R⁴ or R⁵ may independently be 3- to 10-membered heterocycle selected from azacyclopropane, pyrrolidine, piperidine, azepane, azocane, azonane, morpholine, thiomorpholine, 1,4-piperazine, 1,4-oxazepane, and 1,4-diazepane.

In some embodiments, the compound of Formula (IV) is selected from:

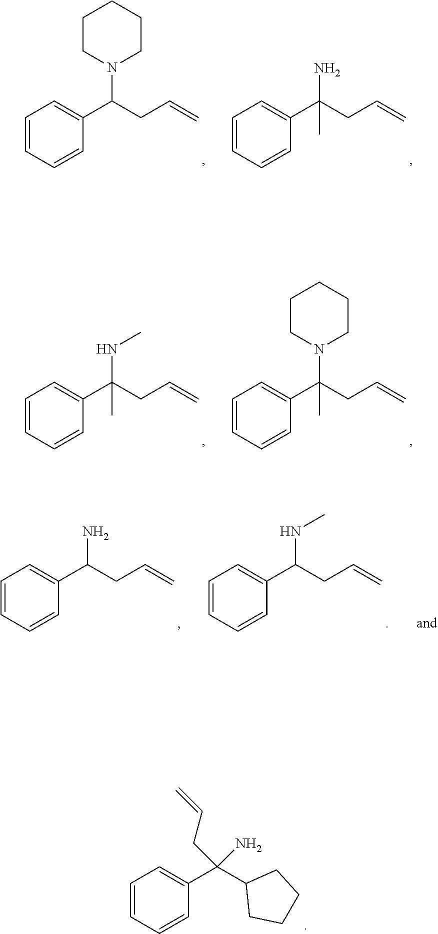

In another aspect, disclosed herein is a compound represented by Formula (V):

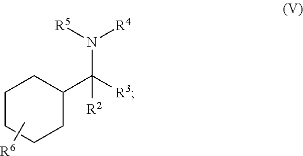

or an enantiomer, a mixture of enantiomers, or an isotopic variant thereof; or a pharmaceutically acceptable salt, solvate or hydrate thereof, wherein:
R² is selected from
   hydrogen;
   halogen;
   C₁₋₈ alkyl, C₂₋₈ alkenyl, and C₂₋₈ alkynyl, each of which is optionally substituted with one or more R⁷; and
   C₃₋₁₀ carbocycle and 3- to 10-membered heterocycle; wherein the C₃₋₁₀ carbocycle and 3-to 10-membered heterocycle are each optionally substituted with one or more R⁷;
R³ is selected from:
   C₁₋₆ alkyl, C₂₋₆ alkenyl, and C₂₋₆ alkynyl, each of which is optionally substituted with one or more R⁸; and
   C₃₋₁₀ carbocycle and 3- to 10-membered heterocycle; wherein the C₃₋₁₀ carbocycle and 3-to 10-membered heterocycle are each optionally substituted with one or more R¹; R⁴, R⁵, R⁶, R⁷, and R⁸ are each independently selected from:
   hydrogen;
   C₁₋₁₀ alkyl, C₂₋₁₀ alkenyl, and C₂₋₁₀ alkynyl optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₁₋₆ cycloalkyl, and C₁₋₆ cycloalkenyl; and
   C₃₋₁₀ carbocycle or 3- to 10-membered heterocycle; wherein the C₃₋₁₀ carbocycle and 3- to 10-membered heterocycle are each optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), and C₁₋₈ alkyl.

In some embodiments, R² is hydrogen, C₁₋₈ alkyl, C₂₋₈ alkenyl, or C₂₋₈ alkynyl, each of which is optionally substituted with one or more R⁷.

In some embodiments, R² is C₃₋₇ carbocycle or 3- to 7-membered heterocycle; wherein the C₃₋₇ carbocycle and 3- to 7-membered heterocycle are each optionally substituted with one or more R⁷.

In some embodiments, R² is phenyl, optionally substituted with one or more R⁷.

In some embodiments, R² is a cycloalkyl. In some embodiments, R² is a cycloalkyl selected from cyclooctane, cycloheptane, cyclohexane, cyclopentane, cyclobutane, and cyclopropane, any of which is optionally substituted with one or more R⁷.

In some embodiments, R² is a cycloalkenyl. In some embodiments, R² is a cycloalkenyl selected from cyclooctene, cycloheptene, cyclohexene, cyclopentene, cyclobutene, and cyclopropane, any of which is optionally substituted with one or more R⁷. In some embodiments, the cycloalkenyl can have 1, 2, 3, 4, or more degrees of unsaturation.

In some embodiments, R² is a 6-membered heteroaryl. In some embodiments, R² is a 6-membered heteroaryl selected from pyridine, pyrazine, and pyrimidine, any of which is optionally substituted with one or more R⁷. In some embodiments, the heteroatom of the heteroaryl is not bound to the methylene carbon of Formula (V).

In some embodiments, R² is a 5-membered heteroaryl. In some embodiments, R² is a 5-membered heteroaryl selected from thiophene, furan, pyrrole, pyrazole, selenophene, imidazole, thiazole, isothiazole, oxatriazole, oxadiazole, oxazole, and thiadiazole, any of which is optionally substituted with one or more R⁷. In some embodiments, the heteroatom of the heteroaryl is not bound to the methylene carbon of Formula (V).

In some embodiments, R² is a 6-membered heterocycloalkenyl. In some embodiments, R² is a 6-membered heterocycloalkenyl selected from dihydropyridine, tetrahydropyridine, dihydropyridazine, tetrahydropyridazine, dihydropyrimidine, tetrahydropyrimidine, dihydropyrazine, tetrahydropyridazine, dihydropyrimidine, tetrahydropyrimidine, dihydropyrazine, tetrahydropyrazine, pyran, dihydropyran, thiopyran, dihydrothiopyran, dioxine, dihydrodioxine, oxazine, dihydrooxazine, thiazine, and dihydrothiazine, any of which is optionally substituted with one or more R⁷. In some embodiments, the heteroatom of the heterocycloalkenyl is not bound to the methylene carbon of Formula (V).

In some embodiments, R² is a 5-membered heterocycloalkenyl. In some embodiments, R² is a 5-membered heterocycloalkenyl selected from pyrroline, pyrazoline, imidazoline, triazoline, dihydrofuran, dihydrothiophene, oxazoline, isooxazoline, thiazoline, isothiazoline, oxadiazoline, and thiadiazoline, any of which is optionally substituted with one or more R⁷. In some embodiments, the heteroatom of the heterocycloalkenyl is not bound to the methylene carbon of Formula (V).

In some embodiments, R² is a 6-membered heterocycloalkyl. In some embodiments, R² is a 6-membered heterocycloalkyl selected from piperidine, piperazine, tetrahydrothiopyran, tetrahydropyran, dioxane, morpholine, and thiomorpholine, any of which is optionally substituted with one or more R⁷. In some embodiments, the heteroatom of the heterocycloalkyl is not bound to the methylene carbon of Formula (V).

In some embodiments, R² is a 5-membered heterocycloalkyl. In some embodiments, R² is a 5-membered heterocycloalkyl selected from tetrahydrofuran, pyrrolidone, and tetrahydrothiophene, any of which is optionally substituted with one or more R⁷. In some embodiments, the heteroatom of the heterocycloalkyl is not bound to the methylene carbon of Formula (V).

In some embodiments, R² is a 4-membered heterocycloalkyl. In some embodiments, R² is a 4-membered heterocycloalkyl selected from azetidine, oxetane, and thietane, any of which is optionally substituted with one or more R⁷. In some embodiments, the heteroatom of the heterocycloalkyl is not bound to the methylene carbon of Formula (V).

In some embodiments, R² is a 6-6 fused ring system. In some embodiments, R² is a 6-6 fused ring system selected from naphthalene, quinoline, isoquinoline, pteridine, benzopyran, dihydroquinoline, dihydroisoquinoline, dihydronapthalene, and tetrahydronapthalene, any of which is optionally substituted with one or more R⁷.

In some embodiments, R² is a 5-6 fused ring system. In some embodiments, R² is a 5-6 fused ring system selected from benzimidazole, indole, azaindole, indazole, benzothiophene, benzofuran, benzoselenophene, benzimidazole, benzotriazole, benzothiazole, benzisothiazole, benzoxadiazole, benzoxazole, and benzothiadiazole, imidazo[4,5-b]-pyridine, imidazo[4,5-c]-pyridine, pyrazolo[3,4-b]pyridine, pyrazolo[3,4-c]pyridine, pyrazolo[4,3-b]pyridine, pyrazolo[4,3-b]pyridine, oxazolo[4,5-b]pyridine, oxazolo[4,5-c]pyridine, oxazolo[5,4-b]pyridine, oxazolo[5,4-c]pyridine, thiazolo[4,5-b]pyridine, thiazolo[4,5-c]pyridine, thiazolo[5,4-b]pyridine, thiazolo[5,4-c]pyridine, isooxazolo[4,5-b]pyridine, isooxazolo[4,5-c]pyridine, isooxazolo[3,4-b]pyridine, isooxazolo[3,4-c]pyridine, isooxazolo[4,3-b]pyridine, isooxazolo[4,3-c]pyridine, isooxazolo[5,4-b]pyridine, isooxazolo[5,4-c]pyridine, isothiazolo[4,5-b]pyridine, isothiazolo[4,5-c]pyridine, isothiazolo[5,4-b]pyridine, isothiazolo[5,4-c]pyridine, isothiazolo[3,4-b]pyridine, isothiazolo[3,4-c]pyridine, isothiazolo[4,3-b]pyridine, isothiazolo[4,3-c]pyridine and purine, any of which is optionally substituted with one or more R⁷.

In some embodiments, R² is a 5-5 fused ring system. In some embodiments, R² is a 5-5 fused ring system selected from pyrrolo[2,1-b]thiazole, pyrazolo[5,1-b]thiazole, imidazo[5,1-b]thiazole, imidazo[2,1-b]thiazole, thiazolo[3,2-b][1,2,4]triazole, thiazolo[3,2-c][1,2,4]triazole, imidazo[2,1-b]thiadiazole, imidazo[1,2-d][1,2,4]thiadiazole, thiazolo[3,2-d]tetrazole, thieno[3,2-b]thiophene, thieno[2,3-b]thiophene, thieno[3,4-b]thiophene, thieno[3,4-c]thiophene, any of which is optionally substituted with one or more R⁷.

In some embodiments, R² is a bridged ring system. In some embodiments, R² is a bridged ring system selected from norbornane, bicyclo[1.1.0]butane, bicyclo[3.2.0]heptane, bicyclo[3.3.0]octane, bicyclo[4.3.0]nonane, bicyclo[1.1.1]pentane, bicyclo[2.2.1]hexane, bicyclo[3.3.1]nonane, bicyclo[2.2.2]octane, adamantane, cubane, housane, any of which is optionally substituted with one or more R⁷. In some embodiments, the bridged ring system can have 1, 2, 3, 4, or more degrees of unsaturation.

In some embodiments, R³ is hydrogen, C₁₋₆ alkyl, C₂₋₆ alkenyl, or C₂₋₆ alkynyl, each of which is optionally substituted with one or more R⁸.

In some embodiments, R³ is C₃₋₇ carbocycle or 3- to 7-membered heterocycle; wherein the C₃₋₇ carbocycle and 3- to 7-membered heterocycle are each optionally substituted with one or more R⁸.

In some embodiments, R³ is phenyl, optionally substituted with one or more R⁸.

In some embodiments, R³ is a cycloalkyl. In some embodiments, R³ is a cycloalkyl selected from cyclooctane, cycloheptane, cyclohexane, cyclopentane, cyclobutane, and cyclopropane, any of which is optionally substituted with one or more R⁸.

In some embodiments, R³ is a cycloalkenyl. In some embodiments, R³ is a cycloalkenyl selected from cyclooctene, cycloheptene, cyclohexene, cyclopentene, cyclobutene, and cyclopropane, any of which is optionally substituted with one or more R⁸. In some embodiments, the cycloalkenyl can have 1, 2, 3, 4, or more degrees of unsaturation.

In some embodiments, R³ is a 6-membered heteroaryl. In some embodiments, R³ is a 6-membered heteroaryl selected from pyridine, pyrazine, and pyrimidine, any of which is optionally substituted with one or more R⁸. In some embodiments, the heteroatom of the heteroaryl is not bound to the methylene carbon of Formula (V).

In some embodiments, R³ is a 5-membered heteroaryl. In some embodiments, R³ is a 5-membered heteroaryl selected from thiophene, furan, pyrrole, pyrazole, selenophene, imidazole, thiazole, isothiazole, oxatriazole, oxadiazole, oxazole, and thiadiazole, any of which is optionally substituted with one or more R⁸. In some embodiments, the heteroatom of the heteroaryl is not bound to the methylene carbon of Formula (V).

In some embodiments, R³ is a 6-membered heterocycloalkenyl. In some embodiments, R³ is a 6-membered heterocycloalkenyl selected from dihydropyridine, tetrahydropyridine, dihydropyridazine, tetrahydropyridazine, dihydropyrimidine, tetrahydropyrimidine, dihydropyrazine, tetrahydropyridazine, dihydropyrimidine, tetrahydropyrimidine, dihydropyrazine, tetrahydropyrazine, pyran, dihydropyran, thiopyran, dihydrothiopyran, dioxine, dihydrodioxine, oxazine, dihydrooxazine, thiazine, and dihydrothiazine, any of which is optionally substituted with one or more R⁸. In some embodiments, the heteroatom of the heterocycloalkenyl is not bound to the methylene carbon of Formula (V).

In some embodiments, R³ is a 5-membered heterocycloalkenyl. In some embodiments, R³ is a 5-membered heterocycloalkenyl selected from pyrroline, pyrazoline, imidazoline, triazoline, dihydrofuran, dihydrothiophene, oxazoline, isooxazoline, thiazoline, isothiazoline, oxadiazoline, and thiadiazoline, any of which is optionally substituted with one or more R⁸. In some embodiments, the heteroatom of the heterocycloalkenyl is not bound to the methylene carbon of Formula (V).

In some embodiments, R³ is a 6-membered heterocycloalkyl. In some embodiments, R³ is a 6-membered heterocycloalkyl selected from piperidine, piperazine, tetrahydrothiopyran, tetrahydropyran, dioxane, morpholine, and thiomorpholine, any of which is optionally substituted with one or more R⁸. In some embodiments, the heteroatom of the heterocycloalkyl is not bound to the methylene carbon of Formula (V).

In some embodiments, R³ is a 5-membered heterocycloalkyl. In some embodiments, R³ is a 5-membered heterocycloalkyl selected from tetrahydrofuran, pyrrolidone, and tetrahydrothiophene, any of which is optionally substituted with one or more R⁸. In some embodiments, the heteroatom of the heterocycloalkyl is not bound to the methylene carbon of Formula (V).

In some embodiments, R³ is a 4-membered heterocycloalkyl. In some embodiments, R³ is a 4-membered heterocycloalkyl selected from azetidine, oxetane, and thietane, any of which is optionally substituted with one or more R⁸. In some embodiments, the heteroatom of the heterocycloalkyl is not bound to the methylene carbon of Formula (V).

In some embodiments, R³ is a 6-6 fused ring system. In some embodiments, R³ is a 6-6 fused ring system selected from naphthalene, quinoline, isoquinoline, pteridine, benzopyran, dihydroquinoline, dihydroisoquinoline, dihydronapthalene, and tetrahydronapthalene, any of which is optionally substituted with one or more R⁸.

In some embodiments, R³ is a 5-6 fused ring system. In some embodiments, R³ is a 5-6 fused ring system selected from benzimidazole, indole, azaindole, indazole, benzothiophene, benzofuran, benzoselenophene, benzimidazole, benzotriazole, benzothiazole, benzisothiazole, benzoxadiazole, benzoxazole, and benzothiadiazole, imidazo[4,5-b]-pyridine, imidazo[4,5-c]-pyridine, pyrazolo[3,4-b]pyridine, pyrazolo[3,4-c]pyridine, pyrazolo[4,3-b]pyridine, pyrazolo[4,3-b]pyridine, oxazolo[4,5-b]pyridine, oxazolo[4,5-c]pyridine, oxazolo[5,4-b]pyridine, oxazolo[5,4-c]pyridine, thiazolo[4,5-b]pyridine, thiazolo[4,5-c]pyridine, thiazolo[5,4-b]pyridine, thiazolo[5,4-c]pyridine, isooxazolo[4,5-b]pyridine, isooxazolo[4,5-c]pyridine, isooxazolo[3,4-b]pyridine, isooxazolo[3,4-c]pyridine, isooxazolo[4,3-b]pyridine, isooxazolo[4,3-c]pyridine, isooxazolo[5,4-b]pyridine, isooxazolo[5,4-c]pyridine, isothiazolo[4,5-b]pyridine, isothiazolo[4,5-c]pyridine, isothiazolo[5,4-b]pyridine, isothiazolo[5,4-c]pyridine, isothiazolo[3,4-b]pyridine, isothiazolo[3,4-c]pyridine, isothiazolo[4,3-b]pyridine, isothiazolo[4,3-c]pyridine and purine, any of which is optionally substituted with one or more R⁸.

In some embodiments, R³ is a 5-5 fused ring system. In some embodiments, R³ is a 5-5 fused ring system selected from pyrrolo[2,1-b]thiazole, pyrazolo[5,1-b]thiazole, imidazo[5,1-b]thiazole, imidazo[2,1-b]thiazole, thiazolo[3,2-b][1,2,4]triazole, thiazolo[3,2-c][1,2,4]triazole, imidazo[2,1-b]thiadiazole, imidazo[1,2-d][1,2,4]thiadiazole, thiazolo[3,2-d]tetrazole, thieno[3,2-b]thiophene, thieno[2,3-b]thiophene, thieno[3,4-b]thiophene, thieno[3,4-c]thiophene, any of which is optionally substituted with one or more R⁸.

In some embodiments, R³ is a bridged ring system. In some embodiments, R³ is a bridged ring system selected from norbornane, norbornene, bicyclo[1.1.0]butane, bicyclo[3.2.0]heptane, bicyclo[3.3.0]octane, bicyclo[4.3.0]nonane, bicyclo[1.1.1]pentane, bicyclo[2.2.1]hexane, bicyclo[3.3.1]nonane, bicyclo[2.2.2]octane, adamantane, cubane, housane, any of which is optionally substituted with one or more R⁸. In some embodiments, the bridged ring system can have 1, 2, 3, 4, or more degrees of unsaturation.

In some embodiments, each of R⁴ or R⁵ may independently be hydrogen, C₁₋₁₀ alkyl, C₂₋₁₀ alkenyl, or C₂₋₁₀ alkynyl, each of which is optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₁₋₆ cycloalkyl, and C₁₋₆ cycloalkenyl.

In some embodiments, each of R⁴ or R⁵ may independently be 3- to 10-membered heterocycle. In some embodiments, each of R⁴ or R⁵ may independently be 3- to 10-membered heterocycle selected from azacyclopropane, pyrrolidine, piperidine, azepane, azocane, azonane, morpholine, thiomorpholine, 1,4-piperazine, 1,4-oxazepane, and 1,4-diazepane.

In some embodiments, compound of Formula (V) is selected from:

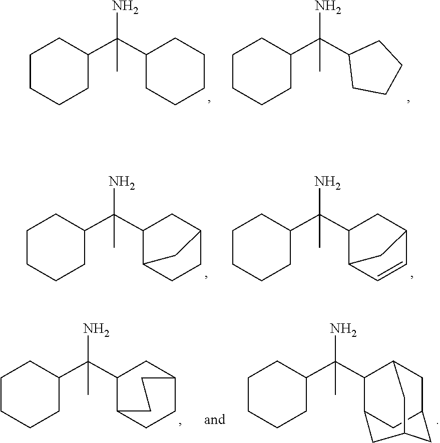

In another aspect, disclosed herein is a compound represented by Formula (VI):

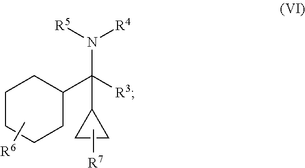

or an enantiomer, a mixture of enantiomers, or an isotopic variant thereof; or a pharmaceutically acceptable salt, solvate or hydrate thereof, wherein:
R³ is selected from:
   C₁₋₁₀ alkyl, C₂₋₁₀ alkenyl, and C₂₋₁₀ alkynyl, each of which is optionally substituted with one or more R⁸; and
   C₃₋₁₀ carbocycle and 3- to 10-membered heterocycle; wherein the C₃₋₁₀ carbocycle and 3-to 10-membered heterocycle are each optionally substituted with one or more R¹;
R⁴, R⁵, R⁶, R⁷, and R⁸ are each independently selected from:
   hydrogen;
   C₁₋₆ alkyl, C₂₋₆ alkenyl, and C₂₋₆ alkynyl optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₁₋₆ cycloalkyl, and C₁₋₆ cycloalkenyl; and
   C₃₋₁₀ carbocycle or 3- to 10-membered heterocycle; wherein the C₃₋₁₀ carbocycle and 3- to 10-membered heterocycle are each optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), and C₁₋₈ alkyl.

In some embodiments, R³ is hydrogen, C₁₋₆ alkyl, C₂₋₆ alkenyl, or C₂₋₆ alkynyl, each of which is optionally substituted with one or more R⁸.

In some embodiments, R³ is C₃₋₇ carbocycle or 3- to 7-membered heterocycle; wherein the C₃₋₇ carbocycle and 3- to 7-membered heterocycle are each optionally substituted with one or more R⁸.

In some embodiments, R³ is phenyl, optionally substituted with one or more R⁸.

In some embodiments, R³ is a cycloalkyl. In some embodiments, R³ is a cycloalkyl selected from cyclooctane, cycloheptane, cyclohexane, cyclopentane, cyclobutane, and cyclopropane, any of which is optionally substituted with one or more R⁸.

In some embodiments, R³ is a cycloalkenyl. In some embodiments, R³ is a cycloalkenyl selected from cyclooctene, cycloheptene, cyclohexene, cyclopentene, cyclobutene, and cyclopropane, any of which is optionally substituted with one or more R⁸. In some embodiments, the cycloalkenyl can have 1, 2, 3, 4, or more degrees of unsaturation.

In some embodiments, R³ is a 6-membered heteroaryl. In some embodiments, R³ is a 6-membered heteroaryl selected from pyridine, pyrazine, and pyrimidine, any of which is optionally substituted with one or more R⁸. In some embodiments, the heteroatom of the heteroaryl is not bound to the methylene carbon of Formula (VI).

In some embodiments, R³ is a 5-membered heteroaryl. In some embodiments, R³ is a 5-membered heteroaryl selected from thiophene, furan, pyrrole, pyrazole, selenophene, imidazole, thiazole, isothiazole, oxatriazole, oxadiazole, oxazole, and thiadiazole, any of which is optionally substituted with one or more R⁸. In some embodiments, the heteroatom of the heteroaryl is not bound to the methylene carbon of Formula (VI).

In some embodiments, R³ is a 6-membered heterocycloalkenyl. In some embodiments, R³ is a 6-membered heterocycloalkenyl selected from dihydropyridine, tetrahydropyridine, dihydropyridazine, tetrahydropyridazine, dihydropyrimidine, tetrahydropyrimidine, dihydropyrazine, tetrahydropyridazine, dihydropyrimidine, tetrahydropyrimidine, dihydropyrazine, tetrahydropyrazine, pyran, dihydropyran, thiopyran, dihydrothiopyran, dioxine, dihydrodioxine, oxazine, dihydrooxazine, thiazine, and dihydrothiazine, any of which is optionally substituted with one or more R⁸. In some embodiments, the heteroatom of the heterocycloalkenyl is not bound to the methylene carbon of Formula (VI).

In some embodiments, R³ is a 5-membered heterocycloalkenyl. In some embodiments, R³ is a 5-membered heterocycloalkenyl selected from pyrroline, pyrazoline, imidazoline, triazoline, dihydrofuran, dihydrothiophene, oxazoline, isooxazoline, thiazoline, isothiazoline, oxadiazoline, and thiadiazoline, any of which is optionally substituted with one or more R⁸. In some embodiments, the heteroatom of the heterocycloalkenyl is not bound to the methylene carbon of Formula (VI).

In some embodiments, R³ is a 6-membered heterocycloalkyl. In some embodiments, R³ is a 6-membered heterocycloalkyl selected from piperidine, piperazine, tetrahydrothiopyran, tetrahydropyran, dioxane, morpholine, and thiomorpholine, any of which is optionally substituted with one or more R⁸. In some embodiments, the heteroatom of the heterocycloalkyl is not bound to the methylene carbon of Formula (VI).

In some embodiments, R³ is a 5-membered heterocycloalkyl. In some embodiments, R³ is a 5-membered heterocycloalkyl selected from tetrahydrofuran, pyrrolidone, and tetrahydrothiophene, any of which is optionally substituted with one or more R⁸. In some embodiments, the heteroatom of the heterocycloalkyl is not bound to the methylene carbon of Formula (VI).

In some embodiments, R³ is a 4-membered heterocycloalkyl. In some embodiments, R³ is a 4-membered heterocycloalkyl selected from azetidine, oxetane, and thietane, any of which is optionally substituted with one or more R⁸. In some embodiments, the heteroatom of the heterocycloalkyl is not bound to the methylene carbon of Formula (VI).

In some embodiments, R³ is a 6-6 fused ring system. In some embodiments, R³ is a 6-6 fused ring system selected from naphthalene, quinoline, isoquinoline, pteridine, benzopyran, dihydroquinoline, dihydroisoquinoline, dihydronapthalene, and tetrahydronapthalene, any of which is optionally substituted with one or more R⁸.

In some embodiments, R³ is a 5-6 fused ring system. In some embodiments, R³ is a 5-6 fused ring system selected from benzimidazole, indole, azaindole, indazole, benzothiophene, benzofuran, benzoselenophene, benzimidazole, benzotriazole, benzothiazole, benzisothiazole, benzoxadiazole, benzoxazole, and benzothiadiazole, imidazo[4,5-b]-pyridine, imidazo[4,5-c]-pyridine, pyrazolo[3,4-b]pyridine, pyrazolo[3,4-c]pyridine, pyrazolo[4,3-b]pyridine, pyrazolo[4,3-b]pyridine, oxazolo[4,5-b]pyridine, oxazolo[4,5-c]pyridine, oxazolo[5,4-b]pyridine, oxazolo[5,4-c]pyridine, thiazolo[4,5-b]pyridine, thiazolo[4,5-c]pyridine, thiazolo[5,4-b]pyridine, thiazolo[5,4-c]pyridine, isooxazolo[4,5-b]pyridine, isooxazolo[4,5-c]pyridine, isooxazolo[3,4-b]pyridine, isooxazolo[3,4-c]pyridine, isooxazolo[4,3-b]pyridine, isooxazolo[4,3-c]pyridine, isooxazolo[5,4-b]pyridine, isooxazolo[5,4-c]pyridine, isothiazolo[4,5-b]pyridine, isothiazolo[4,5-c]pyridine, isothiazolo[5,4-b]pyridine, isothiazolo[5,4-c]pyridine, isothiazolo[3,4-b]pyridine, isothiazolo[3,4-c]pyridine, isothiazolo[4,3-b]pyridine, isothiazolo[4,3-c]pyridine and purine, any of which is optionally substituted with one or more R⁸.

In some embodiments, R³ is a 5-5 fused ring system. In some embodiments, R³ is a 5-5 fused ring system selected from pyrrolo[2,1-b]thiazole, pyrazolo[5,1-b]thiazole, imidazo[5,1-b]thiazole, imidazo[2,1-b]thiazole, thiazolo[3,2-b][1,2,4]triazole, thiazolo[3,2-c][1,2,4]triazole, imidazo[2,1-b]thiadiazole, imidazo[1,2-d][1,2,4]thiadiazole, thiazolo[3,2-d]tetrazole, thieno[3,2-b]thiophene, thieno[2,3-b]thiophene, thieno[3,4-b]thiophene, thieno[3,4-c]thiophene, any of which is optionally substituted with one or more R⁸.

In some embodiments, R³ is a bridged ring system. In some embodiments, R³ is a bridged ring system selected from norbornane, norbornene, bicyclo[1.1.0]butane, bicyclo[3.2.0]heptane, bicyclo[3.3.0]octane, bicyclo[4.3.0]nonane, bicyclo[1.1.1]pentane, bicyclo[2.2.1]hexane, bicyclo[3.3.1]nonane, bicyclo[2.2.2]octane, adamantane, cubane, housane, any of which is optionally substituted with one or more R⁸. In some embodiments, the bridged ring system can have 1, 2, 3, 4, or more degrees of unsaturation.

In some embodiments, each of R⁴ or R⁵ may independently be hydrogen, C₁₋₁₀ alkyl, C₂₋₁₀ alkenyl, or C₂₋₁₀ alkynyl, each of which is optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₁₋₆ cycloalkyl, and C₁₋₆ cycloalkenyl.

In some embodiments, each of R⁴ or R⁵ may independently be 3- to 10-membered heterocycle. In some embodiments, each of R⁴ or R⁵ may independently be 3- to 10-membered heterocycle selected from azacyclopropane, pyrrolidine, piperidine, azepane, azocane, azonane, morpholine, thiomorpholine, 1,4-piperazine, 1,4-oxazepane, and 1,4-diazepane.

In some embodiments, compound of Formula (VI) is selected from:

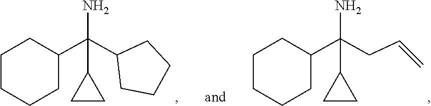 [SMILES: NC(C1CCCCC1)(C1CCCC1)C1CC1 ; C=CCC(N)(C1CCCCC1)C1CC1]

In some embodiments, a compound of Formula (I), (II), (III), (IV), (V), or (VI) is selected from Table 1 or Table 1a.

TABLE 1
Compounds.

| Compound No. | Structure | Name |
|---|---|---|
| 2 | 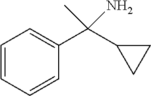 | 1-cyclopropyl-1-phenylethan-1-amine |
| 3 | 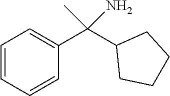 | 1-cyclopentyl-1-phenylethan-1-amine |
| 4 | 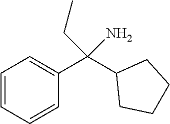 | 1-cyclopentyl-1-phenylpropan-1-amine |
| 5 | 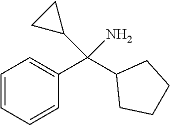 [SMILES: NC(c1ccccc1)(C1CCCC1)C1CC1] | cyclopentyl(cyclopropyl)- (phenyl)methanamine |
| 6 | 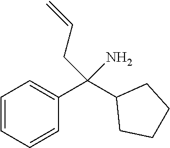 [SMILES: C=CCC(N)(c1ccccc1)C1CCCC1] | 1-cyclopentyl-1-phenylbut-3-en-1-amine |
| 7 | 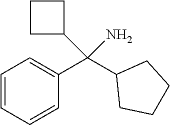 [SMILES: NC(c1ccccc1)(C1CCCC1)C1CCC1] | cyclobutyl(cyclopentyl)-(phenyl)methanamine |
| 8 | 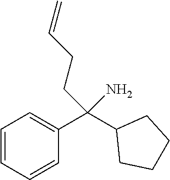 [SMILES: C=CCCC(N)(c1ccccc1)C1CCCC1] | 1-cyclopentyl-1-phenylpent-4-en-1-amine |
| 9 | 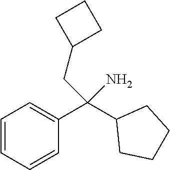 [SMILES: NC(CC1CCC1)(c1ccccc1)C1CCCC1] | 2-cyclobutyl-1-cyclopentyl-1-phenylethan-1- amine |
| 10 | 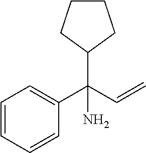 | 1-cyclopentyl-1-phenylprop-2-en-1-amine |
| 11 | 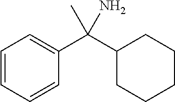 | 1-cyclohexyl-1-phenylethan-1-amine |
| 12 | 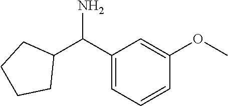 | cyclopentyl(3-methoxyphenyl)methanamine |
| 13 | 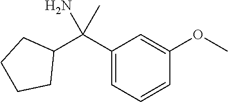 [SMILES: COc1cccc(C(C)(N)C2CCCC2)c1] | 1-cyclopentyl-1-(3-methoxyphenyl)ethan-1- amine |
| 14 | 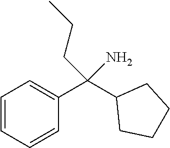 [SMILES: CCCC(N)(c1ccccc1)C1CCCC1] | 1-cyclopentyl-1-phenylbutan-1-amine |
| 15 | 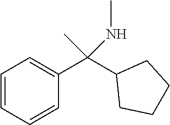 | 1-cyclopentyl-N-methyl-1-phenylethan-1- amine |
| 16 | 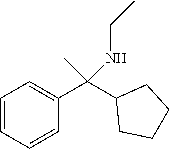 [SMILES: CCNC(C)(c1ccccc1)C1CCCC1] | 1-cyclopentyl-N-ethyl-1-phenylethan-1-amine |
| 19 | 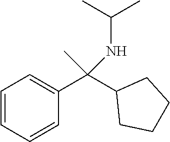 [SMILES: CC(C)NC(C)(c1ccccc1)C1CCCC1] | N-(1-cyclopentyl-1-phenylethyl)propan-2- amine |
| 20 | 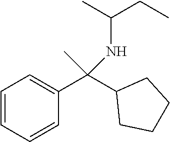 [SMILES: CCC(C)NC(C)(c1ccccc1)C1CCCC1] | N-(1-cyclopentyl-1-phenylethyl)butan-2- amine |
| 21 | 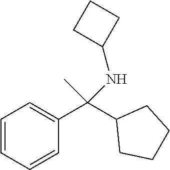 [SMILES: CC(NC1CCC1)(c1ccccc1)C1CCCC1] | N-(1-cyclopentyl-1- phenylethyl)cyclobutanamine |
| 22 | 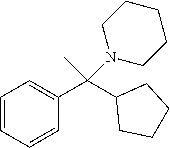 [SMILES: CC(c1ccccc1)(C1CCCC1)N1CCCCC1] | 1-(1-cyclopentyl-1-phenylethyl)piperidine |
| 23 | 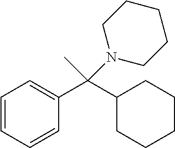 [SMILES: CC(c1ccccc1)(C1CCCCC1)N1CCCCC1] | 1-(1-cyclohexyl-1-phenylethyl)piperidine |
| 24 | 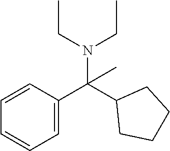 [SMILES: CCN(CC)C(C)(c1ccccc1)C1CCCC1] | 1-cyclopentyl-N,N-diethyl-1-phenylethan-1- amine |
| 25 | 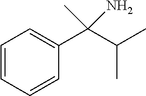 | 3-methyl-2-phenylbutan-2-amine |
| 26 | 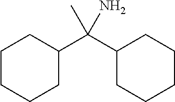 | 1,1-dicyclohexylethan-1-amine |
| 27 | 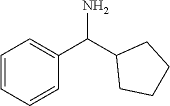 [SMILES: NC(c1ccccc1)C1CCCC1] | cyclopentyl(phenyl)methanamine |
| 28 | 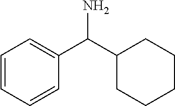 [SMILES: NC(c1ccccc1)C1CCCCC1] | cyclohexyl(phenyl)methanamine |
| 29 | 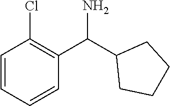 [SMILES: NC(c1ccccc1Cl)C1CCCC1] | (2-chlorophenyl)(cyclopentyl)methanamine |
| 30 | 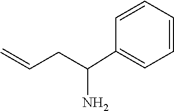 | 1-phenylbut-3-en-1-amine |
| 31 | 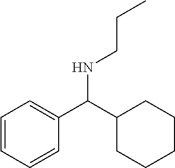 [SMILES: CCCNC(c1ccccc1)C1CCCCC1] | N-(cyclohexyl(phenyl)methyl)propan-1-amine |
| 32 | 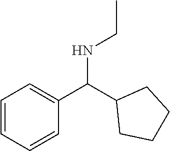 | N-(cyclopentyl(phenyl)methyl)ethanamine |
| 33 | 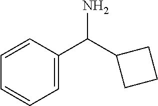 [SMILES: NC(c1ccccc1)C1CCC1] | cyclobutyl(phenyl)methanamine |
| 34 | 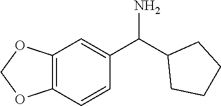 [SMILES: NC(c1ccc2c(c1)OCO2)C1CCCC1] | benzo[d][1,3]dioxol-5- yl(cyclopentyl)methanamine |
| 35 | 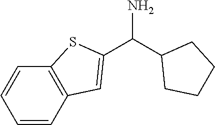 [SMILES: NC(c1cc2ccccc2s1)C1CCCC1] | benzo[b]thiophen-2- yl(cyclopentyl)methanamine |
| 36 | 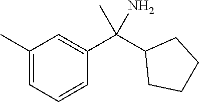 | 1-cyclopentyl-1-(m-tolyl)ethan-1-amine |
| 37 | 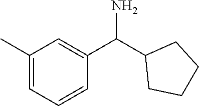 | cyclopentyl(m-tolyl)methanamine |
| 38 | 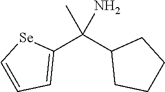 | 1-cyclopentyl-1-(selenophen-2-yl)ethan-1- amine |
| 39 | 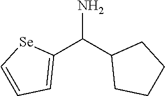 [SMILES: NC(c1ccc[se]1)C1CCCC1] | cyclopentyl(selenophen-2-yl)methanamine |
| 40 | 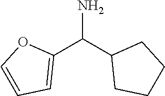 [SMILES: NC(c1ccco1)C1CCCC1] | cyclopentyl(furan-2-yl)methanamine |
| 41 | 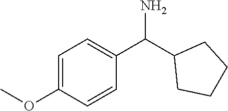 | cyclopentyl(4-methoxyphenyl)methanamine |
| 42 | 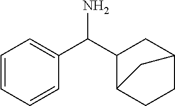 [SMILES: NC(c1ccccc1)C1CC2CCC1C2] | bicyclo[2.2.1]heptan-2- yl(phenyl)methanamine |
| 43 | 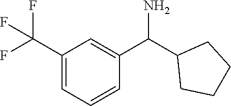 [SMILES: NC(c1cccc(C(F)(F)F)c1)C1CCCC1] | cyclopentyl(3- (trifluoromethyl)phenyl)methanamine |
| 44 | 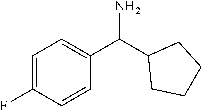 [SMILES: NC(c1ccc(F)cc1)C1CCCC1] | cyclopentyl(4-fluorophenyl)methanamine |
| 45 | 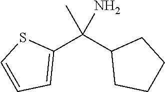 | 1-cyclopentyl-1-(thiophen-2-yl)ethan-1-amine |
| 46 | 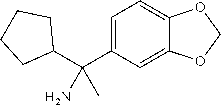 [SMILES: CC(N)(c1ccc2c(c1)OCO2)C1CCCC1] | 1-(benzo[d][1,3]dioxol-5-yl)-1- cyclopentylethan-1-amine |
| 47 | 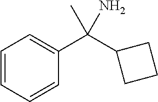 | 1-cyclobutyl-1-phenylethan-1-amine |
| 48 | 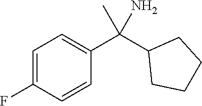 | 1-cyclopentyl-1-(4-fluorophenyl)ethan-1- amine |
| 49 | 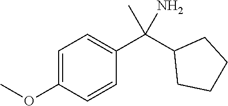 [SMILES: COc1ccc(C(C)(N)C2CCCC2)cc1] | 1-cyclopentyl-1-(4-methoxyphenyl)ethan-1- amine |
| 50 | 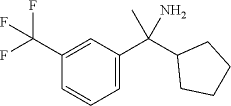 [SMILES: CC(N)(c1cccc(C(F)(F)F)c1)C1CCCC1] | 1-cyclopentyl-1-(3- (trifluoromethyl)phenyl)ethan-1-amine |
| 51 | 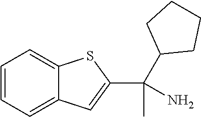 [SMILES: CC(N)(c1cc2ccccc2s1)C1CCCC1] | 1-(benzo[b]thiophen-2-yl)-1- cyclopentylethan-1-amine |
| 52 | 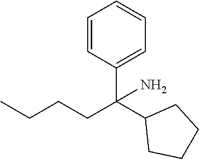 [SMILES: CCCCC(N)(c1ccccc1)C1CCCC1] | 1-cyclopentyl-1-phenylpentan-1-amine |
| 53 | 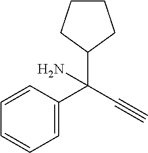 | 1-cyclopentyl-1-phenylprop-2-yn-1-amine |
| 54 | 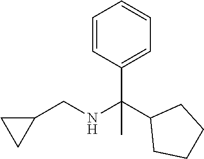 [SMILES: CC(NCC1CC1)(c1ccccc1)C1CCCC1] | 1-cyclopentyl-N-(cyclopropylmethyl)-1- phenylethan-1-amine |
| 55 | 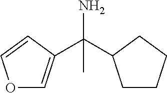 | 1-cyclopentyl-1-(furan-3-yl)ethan-1-amine |
| 56 | 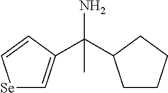 | 1-cyclopentyl-1-(selenophen-3-yl)ethan-1- amine |
| 57 | 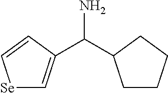 [SMILES: NC(c1cc[se]c1)C1CCCC1] | cyclopentyl(selenophen-3-yl)methanamine |
| 58 | 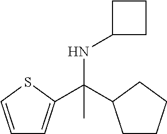 [SMILES: CC(NC1CCC1)(c1cccs1)C1CCCC1] | N-(1-cyclopentyl-1-(thiophen-2- yl)ethyl)cyclobutanamine |
| 59 | 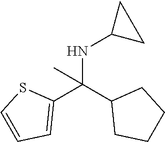 [SMILES: CC(NC1CC1)(c1cccs1)C1CCCC1] | N-(1-cyclopentyl-1-(thiophen-2- yl)ethyl)cyclopropanamine |
| 60 | 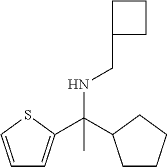 [SMILES: CC(NCC1CCC1)(c1cccs1)C1CCCC1] | N-(cyclobutylmethyl)-1-cyclopentyl-1- (thiophen-2-yl)ethan-1-amine |
| 61 | 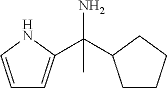 | 1-cyclopentyl-1-(1H-pyrrol-2-yl)ethan-1- amine |
| 62 | 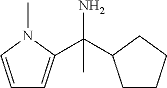 | 1-cyclopentyl-1-(1-methyl-1H-pyrrol-2- yl)ethan-1-amine |
| 63 | 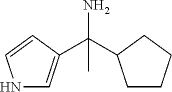 | 1-cyclopentyl-1-(1H-pyrrol-3-yl)ethan-1- amine |
| 64 | 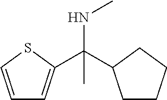 | 1-cyclopentyl-N-methyl-1-(thiophen-2- yl)ethan-1-amine |
| 65 | 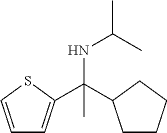 [SMILES: CC(C)NC(C)(c1cccs1)C1CCCC1] | N-(1-cyclopentyl-1-(thiophen-2- yl)ethyl)propan-2-amine |
| 66 | 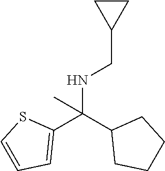 [SMILES: CC(NCC1CC1)(c1cccs1)C1CCCC1] | 1-cyclopentyl-N-(cyclopropylmethyl)-1- (thiophen-2-yl)ethan-1-amine |
| 67 | 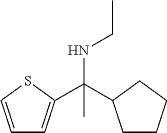 | 1-cyclopentyl-N-ethyl-1-(thiophen-2-yl)ethan- 1-amine |
| 68 | 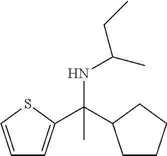 [SMILES: CCC(C)NC(C)(c1cccs1)C1CCCC1] | N-(1-cyclopentyl-1-(thiophen-2- yl)ethyl)butan-2-amine |
| 70 | 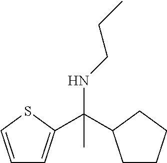 [SMILES: CCCNC(C)(c1cccs1)C1CCCC1] | N-(1-cyclopentyl-1-(thiophen-2- yl)ethyl)propan-1-amine |
| 71 | 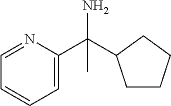 | 1-cyclopentyl-1-(pyridin-2-yl)ethan-1-amine |
| 72 | 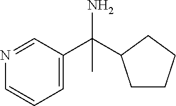 | 1-cyclopentyl-1-(pyridin-3-yl)ethan-1-amine |
| 73 | 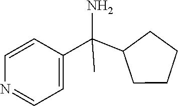 | 1-cyclopentyl-1-(pyridin-4-yl)ethan-1-amine |
| 74 | 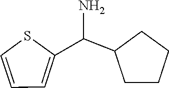 [SMILES: NC(c1cccs1)C1CCCC1] | cyclopentyl(thiophen-2-yl)methanamine |
| 75 | 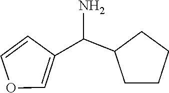 [SMILES: NC(c1ccoc1)C1CCCC1] | cyclopentyl(furan-3-yl)methanamine |
| 76 | 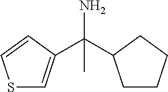 | 1-cyclopentyl-1-(thiophen-3-yl)ethan-1-amine |
| 77 | 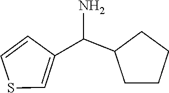 [SMILES: NC(c1ccsc1)C1CCCC1] | cyclopentyl(thiophen-3-yl)methanamine |
| 78 | 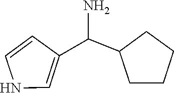 [SMILES: NC(c1cc[nH]c1)C1CCCC1] | cyclopentyl(1H-pyrrol-3-yl)methanamine |
| 79 | 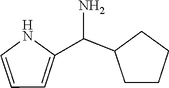 [SMILES: NC(c1ccc[nH]1)C1CCCC1] | cyclopentyl(1H-pyrrol-2-yl)methanamine |
| 80 | 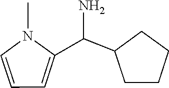 | cyclopentyl(1-methyl-1H-pyrrol-2- yl)methanamine |
| 81 | 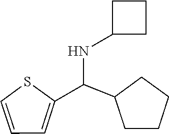 [SMILES: c1csc(C(NC2CCC2)C2CCCC2)c1] | N-(cyclopentyl(thiophen-2- yl)methyl)cyclobutanamine |
| 82 | 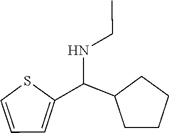 | N-(cyclopentyl(thiophen-2- yl)methyl)ethanamine |
| 83 | 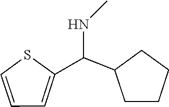 | 1-cyclopentyl-N-methyl-1-(thiophen-2- yl)methanamine |
| 84 | 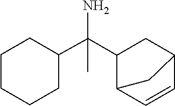 [SMILES: CC(N)(C1CCCCC1)C1CC2C=CC1C2] | 1-(bicyclo[2.2.1]hept-5-en-2-yl)-1- cyclohexylethan-1-amine |
| 85 | 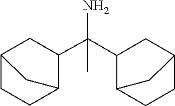 [SMILES: CC(N)(C1CC2CCC1C2)C1CC2CCC1C2] | 1,1-di(bicyclo[2.2.1]heptan-2-yl)ethan-1- amine |
| 86 | 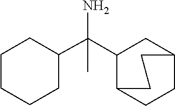 [SMILES: CC(N)(C1CCCCC1)C1CC2CCC1CC2] | 1-(bicyclo[2.2.2]octan-2-yl)-1- cyclohexylethan-1-amine |
| 87 | 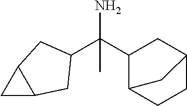 [SMILES: CC(N)(C1CC2CC2C1)C1CC2CCC1C2] | 1-(bicyclo[2.2.1]heptan-2-yl)-1- (bicyclo[3.1.0]hexan-3-yl)ethan-1-amine |
| 88 | 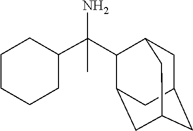 [SMILES: CC(N)(C1CCCCC1)C1C2CC3CC(C2)CC1C3] | 1-((1r,3r,5r,7r)-adamantan-2-yl)-1- cyclohexylethan-1-amine |
| 89 | 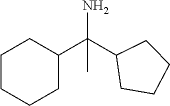 | 1-cyclohexyl-1-cyclopentylethan-1-amine |

TABLE 1a
Compounds.

| Compound No. | Structure | Name |
|---|---|---|
| 5 | 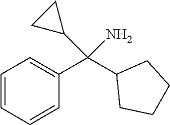 [SMILES: NC(c1ccccc1)(C1CCCC1)C1CC1] | cyclopentyl(cyclopropyl)- (phenyl)methanamine |
| 6 | 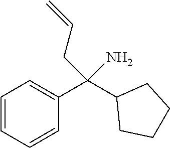 [SMILES: C=CCC(N)(c1ccccc1)C1CCCC1] | 1-cyclopentyl-1-phenylbut-3-en-1-amine |
| 7 | 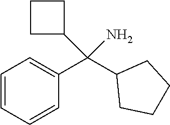 [SMILES: NC(c1ccccc1)(C1CCCC1)C1CCC1] | cyclobutyl(cyclopentyl)- (phenyl)methanamine |
| 8 | 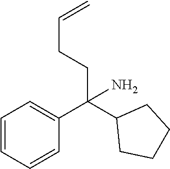 [SMILES: C=CCCC(N)(c1ccccc1)C1CCCC1] | 1-cyclopentyl-1-phenylpent-4-en-1-amine |
| 9 | 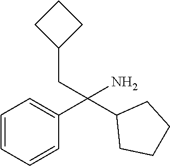 [SMILES: NC(CC1CCC1)(c1ccccc1)C1CCCC1] | 2-cyclobutyl-1-cyclopentyl-1-phenylethan- 1-amine |
| 10 | 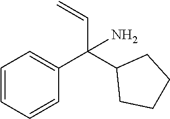 | 1-cyclopentyl-1-phenyl- prop-2-en-1-amine |
| 15 | 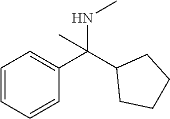 | 1-cyclopentyl-N-methyl-1-phenylethan-1- amine |
| 16 | 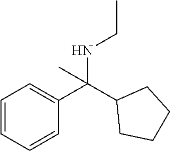 [SMILES: CCNC(C)(c1ccccc1)C1CCCC1] | 1-cyclopentyl-N-ethyl-1-phenylethan-1- amine |
| 19 | 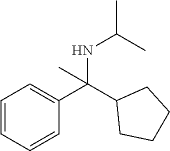 [SMILES: CC(C)NC(C)(c1ccccc1)C1CCCC1] | N-(1-cyclopentyl-1-phenylethyl)propan-2- amine |
| 20 | 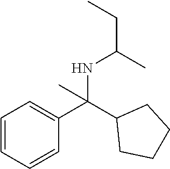 [SMILES: CCC(C)NC(C)(c1ccccc1)C1CCCC1] | N-(1-cyclopentyl-1-phenylethyl)butan-2- amine |
| 21 | 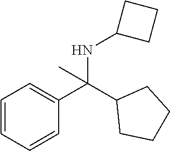 [SMILES: CC(NC1CCC1)(c1ccccc1)C1CCCC1] | N-(1-cyclopentyl-1- phenylethyl)cyclobutanamine |
| 22 | 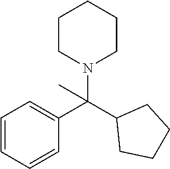 [SMILES: CC(c1ccccc1)(C1CCCC1)N1CCCCC1] | 1-(1-cyclopentyl-1-phenylethyl)piperidine |
| 23 | 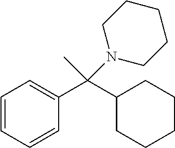 [SMILES: CC(c1ccccc1)(C1CCCCC1)N1CCCCC1] | 1-(1-cyclohexyl-1-phenylethyl)piperidine |
| 24 | 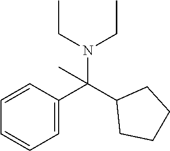 [SMILES: CCN(CC)C(C)(c1ccccc1)C1CCCC1] | 1-cyclopentyl-N,N-diethyl-1-phenylethan- 1-amine |
| 38 | 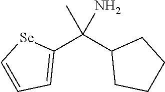 | 1-cyclopentyl-1-(selenophen-2-yl)ethan-1- amine |
| 39 | 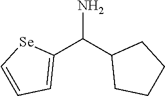 [SMILES: NC(c1ccc[se]1)C1CCCC1] | cyclopentyl(selenophen-2-yl)methanamine |
| 46 | 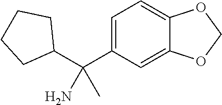 [SMILES: CC(N)(c1ccc2c(c1)OCO2)C1CCCC1] | 1-(benzo[d][1,3]dioxol-5-yl)-1- cyclopentylethan-1-amine |
| 50 | 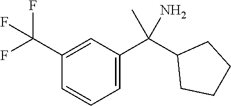 [SMILES: CC(N)(c1cccc(C(F)(F)F)c1)C1CCCC1] | 1-cyclopentyl-1-(3- (trifluoromethyl)phenyl)ethan-1-amine |
| 51 | 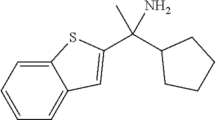 [SMILES: CC(N)(c1cc2ccccc2s1)C1CCCC1] | 1-(benzo[b]thiophen-2-yl)-1- cyclopentylethan-1-amine |
| 52 | 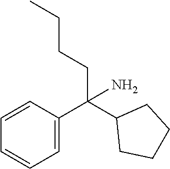 [SMILES: CCCCC(N)(c1ccccc1)C1CCCC1] | 1-cyclopentyl-1-phenylpentan-1-amine |
| 53 | 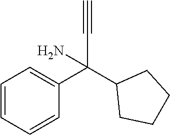 | 1-cyclopentyl-1-phenylprop-2-yn-1-amine |
| 54 | 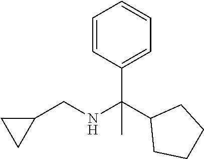 [SMILES: CC(NCC1CC1)(c1ccccc1)C1CCCC1] | 1-cyclopentyl-N-(cyclopropylmethyl)-1- phenylethan-1-amine |
| 55 | 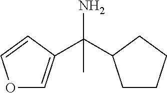 | 1-cyclopenty1-1-(furan-3-yl)ethan-1-amine |
| 56 | 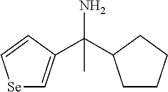 | 1-cyclopentyl-1-(selenophen-3-yl)ethan-1- amine |
| 57 | 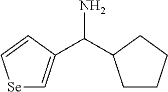 [SMILES: NC(c1cc[se]c1)C1CCCC1] | cyclopentyl(selenophen-3-yl)methanamine |
| 58 | 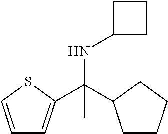 [SMILES: CC(NC1CCC1)(c1cccs1)C1CCCC1] | N-(1-cyclopentyl-1-(thiophen-2- yl)ethyl)cyclobutanamine |
| 59 | 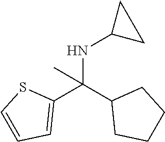 [SMILES: CC(NC1CC1)(c1cccs1)C1CCCC1] | N-(1-cyclopentyl-1-(thiophen-2- yl)ethyl)cyclopropanamine |
| 60 | 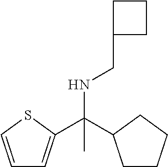 [SMILES: CC(NCC1CCC1)(c1cccs1)C1CCCC1] | N-(cyclobutylmethyl)-1-cyclopentyl-1- (thiophen-2-yl)ethan-1-amine |
| 61 | 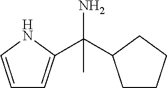 | 1-cyclopentyl-1-(1H-pyrrol-2-yl)ethan-1- amine |
| 62 | 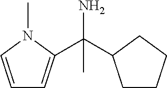 | 1-cyclopentyl-1-(1-methyl-1H-pyrrol-2- yl)ethan-1-amine |
| 63 | 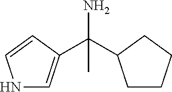 | 1-cyclopentyl-1-(1H-pyrrol-3-yl)ethan-1- amine |
| 64 | 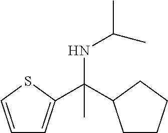 [SMILES: CC(C)NC(C)(c1cccs1)C1CCCC1] | 1-cyclopentyl-N-methyl-1-(thiophen-2- yl)ethan-1-amine |
| 65 | 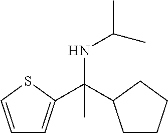 [SMILES: CC(C)NC(C)(c1cccs1)C1CCCC1] | N-(1-cyclopentyl-1-(thiophen-2- yl)ethyl)propan-2-amine |
| 66 | 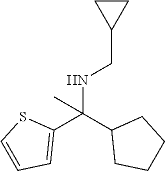 [SMILES: CC(NCC1CC1)(c1cccs1)C1CCCC1] | 1-cyclopentyl-N-(cyclopropylmethyl)-1- (thiophen-2-yl)ethan-1-amine |
| 67 | 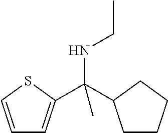 | 1-cyclopentyl-N-ethyl-1-(thiophen-2- yl)ethan-1-amine |
| 68 | 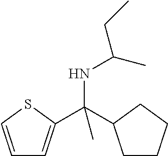 [SMILES: CCC(C)NC(C)(c1cccs1)C1CCCC1] | N-(1-cyclopentyl-1-(thiophen-2- yl)ethyl)butan-2-amine |
| 70 | 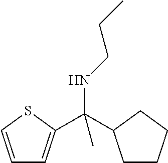 [SMILES: CCCNC(C)(c1cccs1)C1CCCC1] | N-(1-cyclopentyl-1-(thiophen-2- yl)ethyl)propan-1-amine |
| 84 | 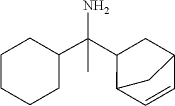 [SMILES: CC(N)(C1CCCCC1)C1CC2C=CC1C2] | 1-(bicyclo[2.2.1]hept-5-en-2-yl)-1- cyclohexylethan-1-amine |
| 85 | 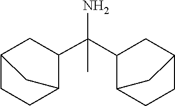 [SMILES: CC(N)(C1CC2CCC1C2)C1CC2CCC1C2] | 1,1-di(bicyclo[2.2.1]heptan-2-yl)ethan-1- amine |
| 86 | 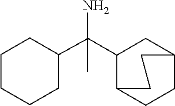 [SMILES: CC(N)(C1CCCCC1)C1CC2CCC1CC2] | 1-(bicyclo[2.2.2]octan-2-yl)-1- cyclohexylethan-1-amine |
| 87 | 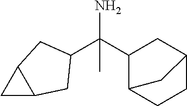 [SMILES: CC(N)(C1CC2CC2C1)C1CC2CCC1C2] | 1-(bicyclo[2.2.1]heptan-2-yl)-1- (bicyclo[3.1.0]hexan-3-yl)ethan-1-amine |
| 88 | 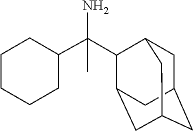 [SMILES: CC(N)(C1CCCCC1)C1C2CC3CC(C2)CC1C3] | 1-((1r,3r,5r,7r)-adamantan-2-yl)-1- cyclohexylethan-1-amine |

Methods of administration of a compound or salt of Formula (I), (II), (III), (IV), (V), or (VI) as discussed herein may be used for the treatment of a physical, psychiatric, or neurological disorder. In some embodiments, the compounds disclosed herein are n-methyl-d-aspartate receptor (NMDAR) antagonists. In some embodiments, the compounds disclosed herein are ionotropic receptor inhibitors.

### Pharmaceutical Formulations

The compositions and methods described herein may be considered useful as pharmaceutical compositions for administration to a subject in need thereof. Pharmaceutical compositions may comprise at least a compound or salt of Formula (I), (II), (III), (IV), (V), or (VI) described herein and one or more pharmaceutically acceptable carriers, diluents, excipients, stabilizers, dispersing agents, suspending agents, and/or thickening agents.

Pharmaceutical compositions comprising a compound or salt of Formula (I), (II), (III), (IV), (V), or (VI) may be formulated using one or more physiologically-acceptable carriers comprising excipients and auxiliaries. Formulation may be modified depending upon the route of administration chosen. Pharmaceutical compositions comprising a compound, salt or conjugate may be manufactured, for example, by lyophilizing the compound, salt or conjugate, mixing, dissolving, emulsifying, encapsulating or entrapping the conjugate. The pharmaceutical compositions may also include the compounds, salts or conjugates in a free-base form or pharmaceutically-acceptable salt form.

Methods for formulation of a compound or salt of Formula (I), (II), (III), (IV), (V), or (VI) may include formulating any of the compounds, salts or conjugates with one or more inert, pharmaceutically-acceptable excipients or carriers to form a solid, semi-solid, or liquid composition. Solid compositions may include, for example, powders, tablets, dispersible granules and capsules, and in some aspects, the solid compositions further contain nontoxic, auxiliary substances, for example wetting or emulsifying agents, pH buffering agents, and other pharmaceutically-acceptable additives. Alternatively, the compounds, salts or conjugates may be lyophilized or in powder form for re-constitution with a suitable vehicle, e.g., sterile pyrogen-free water, before use.

Pharmaceutical compositions comprising a compound or salt of Formula (I), (II), (III), (IV), (V), or (VI) may comprise at least one active ingredient (e.g., a compound, salt or conjugate and other agents). The active ingredients may be entrapped in microcapsules prepared, for example, by coacervation techniques or by interfacial polymerization (e.g., hydroxymethylcellulose or gelatin microcapsules and poly-(methylmethacylate) microcapsules, respectively), in colloidal drug-delivery systems (e.g., liposomes, albumin microspheres, microemulsions, nano-particles and nanocapsules) or in macroemulsions.

The compositions and formulations may be sterilized. Sterilization may be accomplished by filtration through sterile filtration, heat, light, radiation, chemicals or other forms.

The compositions described herein are administered to a subject by appropriate administration routes, including but not limited to, oral, parenteral (e.g., intravenous, subcutaneous, intramuscular), intranasal, buccal, topical, rectal, or transdermal administration routes. The compositions described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

The compositions comprising a compound or salt of Formula (I), (II), (III), (IV), (V), or (VI) may be formulated for administration as an injection. Non-limiting examples of formulations for injection may include a sterile suspension, solution or emulsion in oily or aqueous vehicles. Suitable oily vehicles may include, but are not limited to, lipophilic solvents or vehicles such as fatty oils or synthetic fatty acid esters, or liposomes. Aqueous injection suspensions may contain substances which increase the viscosity or tonicity of the solution or suspension. The solution or suspension may also contain suitable stabilizers. Injections may be formulated for bolus injection or continuous infusion. Alternatively, the compositions may be lyophilized or in powder form for reconstitution with a suitable vehicle, e.g., sterile pyrogen-free water, before use.

For parenteral administration, a compound or salt of Formula (I), (II), (III), (IV), (V), or (VI) may be formulated in a unit dosage injectable form (e.g., solution, suspension, emulsion) in association with a pharmaceutically acceptable parenteral vehicle. Such vehicles may be inherently non-toxic, and non-therapeutic. Vehicles may be water, saline, Ringer's solution, dextrose solution, organic solvents (e.g., ethanol, DMF, DMSO), human serum albumin, and combinations thereof. Non-aqueous vehicles such as fixed oils and ethyl oleate may also be used.

Liposomes may be used as carriers. The vehicle may contain minor amounts of additives such as substances that enhance isotonicity and chemical stability (e.g., buffers and preservatives).

In one embodiment the disclosure relates to methods and compositions of Formula (I), (II), (III), (IV), (V), or (VI) formulated for formulated into a pharmaceutical composition suitable for injection into the body including intramuscular, subcutaneous, intravenous, intratympanic, intraocular, epidural injection. In one aspect, formulations suitable for injection include physiologically acceptable sterile aqueous or non-aqueous solutions, dispersions, suspensions or emulsions, and sterile powders for reconstitution into sterile injectable solutions or dispersions.

Examples of suitable aqueous and non-aqueous carriers, diluents, solvents, complexing agents (e.g., cyclodextrins) or vehicles include water, ethanol, polyols (propyleneglycol, polyethylene-glycol, glycerol, cremophor and the like), vegetable oils and organic esters, such as ethyl oleate.

In some aspects of the disclosure, formulations suitable for subcutaneous injection contain additives such as preserving, wetting, emulsifying, and dispensing agents. Prolonged absorption of the injectable pharmaceutical form can be brought about by the use of agents delaying absorption, such as aluminum monostearate and gelatin.

For intravenous injections, compounds described herein are formulated in aqueous solutions, in physiologically compatible buffers such as Hank's solution, Ringer's solution, or physiological saline buffer.

Parenteral injections may involve bolus injection or continuous infusion. Formulations for injection may be presented in unit dosage form, e.g., in ampoules or in multi-dose containers, with an added preservative. The pharmaceutical composition described herein may be in a form suitable for parenteral injection as a sterile suspensions, solutions or emulsions in oily or aqueous vehicles, and may contain formulatory agents such as suspending, stabilizing and/or dispersing agents. In one aspect, the active ingredient is in powder form for constitution with a suitable vehicle, e.g., sterile pyrogen-free water, before use.

In certain aspects of the disclosure, delivery systems for pharmaceutical compounds may be employed, such as, for example, liposomes and emulsions. In certain aspects of the disclosure, compositions provided herein can also include an mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

In one embodiment the disclosure relates to methods and compositions of Formula (I), (II), (III), (IV), (V), or (VI) formulated for oral delivery to a subject in need. In one embodiment a composition is formulated so as to deliver one or more pharmaceutically active agents to a subject through a mucosa layer in the mouth or esophagus. In another embodiment the composition is formulated to deliver one or more pharmaceutically active agents to a subject through a mucosa layer in the stomach and/or intestines.

In one embodiment compositions of Formula (I), (II), (III), (IV), (V), or (VI) are provided in modified release dosage forms. Suitable modified release dosage vehicles include, but are not limited to, hydrophilic or hydrophobic matrix devices, water-soluble separating layer coatings, enteric coatings, osmotic devices, multi-particulate devices, and combinations thereof. The compositions may also comprise non-release controlling excipients.

In another embodiment compositions of Formula (I), (II), (III), (IV), (V), or (VI) are provided in enteric coated dosage forms. These enteric coated dosage forms can also comprise non-release controlling excipients. In one embodiment the compositions are in the form of enteric-coated granules, as controlled-release capsules for oral administration. The compositions can further comprise cellulose, cyclodextrins, disodium hydrogen phosphate, hydroxypropyl cellulose, pyridazine, lactose, mannitol, or sodium lauryl sulfate. In another embodiment the compositions are in the form of enteric-coated pellets, as controlled-release capsules for oral administration. The compositions can further comprise cyclodextrins, glycerol monostearate 40-50, hydroxypropyl cellulose, pyridazine, magnesium stearate, methacrylic acid copolymer type C, polysorbate 80, sugar spheres, talc, or triethyl citrate.

In another embodiment the compositions of Formula (I), (II), (III), (IV), (V), or (VI) are enteric-coated controlled-release tablets for oral administration. The compositions can further comprise carnauba wax, crospovidone, cyclodextrins, diacetylated monoglycerides, ethylcellulose, hydroxypropyl cellulose, pyridazine phthalate, magnesium stearate, mannitol, sodium hydroxide, sodium stearyl fumarate, talc, titanium dioxide, or yellow ferric oxide.

Sustained-release preparations comprising a compound or salt of Formula (I), (II), (III), (IV), (V), or (VI) may also be prepared. Examples of sustained-release preparations may include semipermeable matrices of solid hydrophobic polymers that may contain the compound, salt or conjugate, and these matrices may be in the form of shaped articles (e.g., films or microcapsules). Examples of sustained-release matrices may include polyesters, hydrogels (e.g., poly(2-hydroxyethyl-methacrylate), or poly(vinyl alcohol)), polylactides, copolymers of L-glutamic acid and y ethyl-L-glutamate, non-degradable ethylene-vinyl acetate, degradable lactic acid-glycolic acid copolymers such as the LUPRON DEPO™ (i.e., injectable microspheres composed of lactic acid-glycolic acid copolymer and leuprolide acetate), and poly-D-(−)-3-hydroxybutyric acid.

Pharmaceutical formulations comprising a compound or salt of Formula (I), (II), (III), (IV), (V), or (VI) may be prepared for storage by mixing a compound, salt or conjugate with a pharmaceutically acceptable carrier, excipient, and/or a stabilizer. This formulation may be a lyophilized formulation or an aqueous solution. Acceptable carriers, excipients, and/or stabilizers may be nontoxic to recipients at the dosages and concentrations used. Acceptable carriers, excipients, and/or stabilizers may include buffers such as phosphate, citrate, and other organic acids; antioxidants including ascorbic acid and methionine; preservatives, polypeptides; proteins, such as serum albumin or gelatin; hydrophilic polymers; amino acids; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes; and/or non-ionic surfactants or polyethylene glycol.

In another embodiment the compositions of Formula (I), (II), (III), (IV), (V), or (VI) can further comprise calcium stearate, crospovidone, cyclodextrins, hydroxypropyl methylcellulose, iron oxide, mannitol, methacrylic acid copolymer, polysorbate 80, povidone, propylene glycol, sodium carbonate, sodium lauryl sulfate, titanium dioxide, and triethyl citrate.

In another embodiment compositions of Formula (I), (II), (III), (IV), (V), or (VI) are provided in effervescent dosage forms. These effervescent dosage forms can also comprise non-release controlling excipients.

In another embodiment compositions of Formula (I), (II), (III), (IV), (V), or (VI) can be provided in a dosage form that has at least one component that can facilitate the immediate release of an active agent, and at least one component that can facilitate the controlled release of an active agent. In a further embodiment the dosage form can be capable of giving a discontinuous release of the compound in the form of at least two consecutive pulses separated in time from 0.1 up to 24 hours. The compositions can comprise one or more release controlling and non-release controlling excipients, such as those excipients suitable for a disruptable semi-permeable membrane and as swellable substances.

In another embodiment compositions Formula (I), (II), (III), (IV), (V), or (VI) are provided in a dosage form for oral administration to a subject, which comprise one or more pharmaceutically acceptable excipients or carriers, enclosed in an intermediate reactive layer comprising a gastric juice-resistant polymeric layered material partially neutralized with alkali and having cation exchange capacity and a gastric juice-resistant outer layer.

In some embodiments, the compositions of Formula (I), (II), (III), (IV), (V), or (VI) provided herein can be in unit-dosage forms or multiple-dosage forms. Unit-dosage forms, as used herein, refer to physically discrete units suitable for administration to human or non-human animal subjects and packaged individually. Each unit-dose can contain a predetermined quantity of an active ingredient(s) sufficient to produce the desired therapeutic effect, in association with the required pharmaceutical carriers or excipients. Examples of unit-dosage forms include, but are not limited to, ampoules, syringes, and individually packaged tablets and capsules. In some embodiments, unit-dosage forms may be administered in fractions or multiples thereof. A multiple-dosage form is a plurality of identical unit-dosage forms packaged in a single container, which can be administered in segregated unit-dosage form. Examples of multiple-dosage forms include, but are not limited to, vials, bottles of tablets or capsules, or bottles of pints or gallons.

In another embodiment the multiple dosage forms comprise different pharmaceutically active agents.

In some embodiments, the compositions of Formula (I), (II), (III), (IV), (V), or (VI) may also be formulated as a modified release dosage form, including immediate-, delayed-, extended-, prolonged-, sustained-, pulsatile-, controlled-, extended, accelerated- and fast-, targeted-, programmed-release, and gastric retention dosage forms. These dosage forms can be prepared according to known methods and techniques (see, Remington: The Science and Practice of Pharmacy, supra; Modified-Release Drug Delivery Technology, Rathbone et al., Eds., Drugs and the Pharmaceutical Science, Marcel Dekker, Inc.: New York, N.Y., 2002; Vol. 126, which are herein incorporated by reference in their entirety).

### Therapeutic Applications

The disclosure provides methanamines and substituted methanamine compounds and salts thereof of Formula (I), (II), (III), (IV), (V), or (VI) for treating physical, psychiatric, or neurological disorders. In some embodiments, treating physical, psychiatric, or neurological disorders comprises inhibiting an ionotropic receptor. In some embodiments, the ionotropic receptor comprises NMDAR or DAT. In some embodiments, treating physical, psychiatric, or neurological disorders comprises inhibiting NMDAR. In some embodiments, treating physical, psychiatric, or neurological disorders comprises inhibiting DAT. In some embodiments, inhibiting an ionotropic receptor comprises administering a compound or a salt thereof of Formula (I), (II), (III), (IV), (V), or (VI). In some embodiments, the physical, psychiatric, or neurological disorder comprises pain, depression, tinnitus, post-traumatic stress disorder, psychosis, schizophrenia, agitation, obsessive compulsive disorder, sexual dysfunction, anxiety, dementias, neurodegenerative diseases, pseudobulbar affect, headache, cluster headache, migraine, acute pain, post-operative pain, pain syndromes, neuropathic pain, chronic pain, complex regional pain syndrome, fibromyalgia, substance use disorders, drug addiction, alcoholism, hallucinations, delusions, insomnia, epilepsies, bipolar disorder, anorexia, or Parkinson's disease.

In some aspects, provided herein is a method for treating a physical, psychiatric, or neurological disorder comprising inhibiting an ionotropic receptor by contacting the ionotropic receptor with a compound represented by the structure of Formula (I):

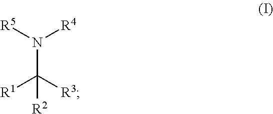

or an enantiomer, a mixture of enantiomers, or an isotopic variant thereof; or a pharmaceutically acceptable salt, solvate or hydrate thereof, wherein:
R¹ is C₃₋₁₀ carbocycle or 3- to 10-membered heterocycle; wherein the C₃₋₁₀ carbocycle and 3- to 10-membered heterocycle are each optionally substituted with one or more R⁶
R² is selected from
   hydrogen;
   halogen;
   C₁₋₈ alkyl, C₂₋₈ alkenyl, and C₂₋₈ alkynyl, each of which is optionally substituted with one or more R⁷; and
   C₃₋₁₀ carbocycle and 3- to 10-membered heterocycle; wherein the C₃₋₁₀ carbocycle and 3- to 10-membered heterocycle are each optionally substituted with one or more R⁷;
R³ is selected from:
   C₁₋₆ alkyl, C₂₋₆ alkenyl, and C₂₋₆ alkynyl, each of which is optionally substituted with one or more R⁸; and
   C₃₋₁₀ carbocycle and 3- to 10-membered heterocycle; wherein the C₃₋₁₀ carbocycle and 3- to 10-membered heterocycle are each optionally substituted with one or more R⁸
R⁴, R⁵, R⁶, R⁷, and R⁸ are each independently selected from:
   hydrogen;
   C₁₋₁₀ alkyl, C₂₋₁₀ alkenyl, and C₂₋₁₀ alkynyl, each of which is optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₁₋₆ cycloalkyl, and C₁₋₆ cycloalkenyl; and
   C₃₋₁₀ carbocycle or 3- to 10-membered heterocycle; wherein the C₃₋₁₀ carbocycle and 3- to 10-membered heterocycle are each optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), and C₁₋₈ alkyl;
provided that the compound of Formula (I) is not

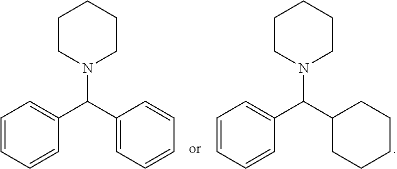

In some embodiments, R¹ is C₃₋₇ carbocycle or 3- to 7-membered heterocycle; wherein the C₃₋₇ carbocycle and 3- to 7-membered heterocycle are each optionally substituted with one or more R⁶.

In some embodiments, R¹ is phenyl, optionally substituted with one or more R⁶.

In some embodiments, R¹ is a cycloalkyl. In some embodiments, R¹ is a cycloalkyl selected from cyclooctane, cycloheptane, cyclohexane, cyclopentane, cyclobutane, and cyclopropane, any of which is optionally substituted with one or more R⁶.

In some embodiments, R¹ is a cycloalkenyl. In some embodiments, R¹ is a cycloalkenyl selected from cyclooctene, cycloheptene, cyclohexene, cyclopentene, cyclobutene, and cyclopropane, any of which is optionally substituted with one or more R⁶. In some embodiments, the cycloalkenyl can have 1, 2, 3, 4, or more degrees of unsaturation.

In some embodiments, R¹ is a 6-membered heteroaryl. In some embodiments, R¹ is a 6-membered heteroaryl selected from pyridine, pyrazine, and pyrimidine, any of which is optionally substituted with one or more R⁶. In some embodiments, the heteroatom of the heteroaryl is not bound to the methylene carbon of Formula (I).

In some embodiments, R¹ is a 5-membered heteroaryl. In some embodiments, R¹ is a 5-membered heteroaryl selected from thiophene, furan, pyrrole, pyrazole, selenophene, imidazole, thiazole, isothiazole, oxatriazole, oxadiazole, oxazole, and thiadiazole, any of which is optionally substituted with one or more R⁶. In some embodiments, the heteroatom of the heteroaryl is not bound to the methylene carbon of Formula (I).

In some embodiments, R¹ is a 6-membered heterocycloalkenyl. In some embodiments, R¹ is a 6-membered heterocycloalkenyl selected from dihydropyridine, tetrahydropyridine, dihydropyridazine, tetrahydropyridazine, dihydropyrimidine, tetrahydropyrimidine, dihydropyrazine, tetrahydropyridazine, dihydropyrimidine, tetrahydropyrimidine, dihydropyrazine, tetrahydropyrazine, pyran, dihydropyran, thiopyran, dihydrothiopyran, dioxine, dihydrodioxine, oxazine, dihydrooxazine, thiazine, and dihydrothiazine, any of which is optionally substituted with one or more R⁶. In some embodiments, the heteroatom of the heterocycloalkenyl is not bound to the methylene carbon of Formula (I).

In some embodiments, R¹ is a 5-membered heterocycloalkenyl. In some embodiments, R¹ is a 5-membered heterocycloalkenyl selected from pyrroline, pyrazoline, imidazoline, triazoline, dihydrofuran, dihydrothiophene, oxazoline, isooxazoline, thiazoline, isothiazoline, oxadiazoline, and thiadiazoline, any of which is optionally substituted with one or more R⁶. In some embodiments, the heteroatom of the heterocycloalkenyl is not bound to the methylene carbon of Formula (I).

In some embodiments, R¹ is a 6-membered heterocycloalkyl. In some embodiments, R¹ is a 6-membered heterocycloalkyl selected from piperidine, piperazine, tetrahydrothiopyran, tetrahydropyran, dioxane, morpholine, and thiomorpholine, any of which is optionally substituted with one or more R⁶. In some embodiments, the heteroatom of the heterocycloalkyl is not bound to the methylene carbon of Formula (I).

In some embodiments, R¹ is a 5-membered heterocycloalkyl. In some embodiments, R¹ is a 5-membered heterocycloalkyl selected from tetrahydrofuran, pyrrolidone, and tetrahydrothiophene, any of which is optionally substituted with one or more R⁶. In some embodiments, the heteroatom of the heterocycloalkyl is not bound to the methylene carbon of Formula (I).

In some embodiments, R¹ is a 4-membered heterocycloalkyl. In some embodiments, R¹ is a 4-membered heterocycloalkyl selected from azetidine, oxetane, and thietane, any of which is optionally substituted with one or more R⁶. In some embodiments, the heteroatom of the heterocycloalkyl is not bound to the methylene carbon of Formula (I).

In some embodiments, R¹ is a 6-6 fused ring system. In some embodiments, R¹ is a 6-6 fused ring system selected from naphthalene, quinoline, isoquinoline, pteridine, benzopyran, dihydroquinoline, dihydroisoquinoline, dihydronapthalene, and tetrahydronapthalene, any of which is optionally substituted with one or more R⁶.

In some embodiments, R¹ is a 5-6 fused ring system. In some embodiments, R¹ is a 5-6 fused ring system selected from benzimidazole, indole, azaindole, indazole, benzothiophene, benzofuran, benzoselenophene, benzimidazole, benzotriazole, benzothiazole, benzisothiazole, benzoxadiazole, benzoxazole, and benzothiadiazole, imidazo[4,5-b]-pyridine, imidazo[4,5-c]-pyridine, pyrazolo[3,4-b]pyridine, pyrazolo[3,4-c]pyridine, pyrazolo[4,3-b]pyridine, pyrazolo[4,3-b]pyridine, oxazolo[4,5-b]pyridine, oxazolo[4,5-c]pyridine, oxazolo[5,4-b]pyridine, oxazolo[5,4-c]pyridine, thiazolo[4,5-b]pyridine, thiazolo[4,5-c]pyridine, thiazolo[5,4-b]pyridine, thiazolo[5,4-c]pyridine, isooxazolo[4,5-b]pyridine, isooxazolo[4,5-c]pyridine, isooxazolo[3,4-b]pyridine, isooxazolo[3,4-c]pyridine, isooxazolo[4,3-b]pyridine, isooxazolo[4,3-c]pyridine, isooxazolo[5,4-b]pyridine, isooxazolo[5,4-c]pyridine, isothiazolo[4,5-b]pyridine, isothiazolo[4,5-c]pyridine, isothiazolo[5,4-b]pyridine, isothiazolo[5,4-c]pyridine, isothiazolo[3,4-b]pyridine, isothiazolo[3,4-c]pyridine, isothiazolo[4,3-b]pyridine, isothiazolo[4,3-c]pyridine and purine, any of which is optionally substituted with one or more R⁶.

In some embodiments, R¹ is a 5-5 fused ring system. In some embodiments, R¹ is a 5-5 fused ring system selected from pyrrolo[2,1-b]thiazole, pyrazolo[5,1-b]thiazole, imidazo[5,1-b]thiazole, imidazo[2,1-b]thiazole, thiazolo[3,2-b][1,2,4]triazole, thiazolo[3,2-c][1,2,4]triazole, imidazo[2,1-b]thiadiazole, imidazo[1,2-d][1,2,4]thiadiazole, thiazolo[3,2-d]tetrazole, thieno[3,2-b]thiophene, thieno[2,3-b]thiophene, thieno[3,4-b]thiophene, thieno[3,4-c]thiophene, any of which is optionally substituted with one or more R⁶.

In some embodiments, R¹ is a bridged ring system. In some embodiments, R¹ is a bridged ring system selected from norbornane, norbornene, bicyclo[1.1.0]butane, bicyclo[3.2.0]heptane, bicyclo[3.3.0]octane, bicyclo[4.3.0]nonane, bicyclo[1.1.1]pentane, bicyclo[2.2.1]hexane, bicyclo[3.3.1]nonane, bicyclo[2.2.2]octane, adamantane, cubane, housane, any of which is optionally substituted with one or more R⁶. In some embodiments, the bridged ring system can have 1, 2, 3, 4, or more degrees of unsaturation.

In some embodiments, R² is hydrogen, C₁₋₈ alkyl, C₂₋₈ alkenyl, or C₂₋₈ alkynyl, each of which is optionally substituted with one or more R⁷.

In some embodiments, R² is C₃₋₇ carbocycle or 3- to 7-membered heterocycle; wherein the C₃₋₇ carbocycle and 3- to 7-membered heterocycle are each optionally substituted with one or more R⁷.

In some embodiments, R² is phenyl, optionally substituted with one or more R⁷.

In some embodiments, R² is a cycloalkyl. In some embodiments, R² is a cycloalkyl selected from cyclooctane, cycloheptane, cyclohexane, cyclopentane, cyclobutane, and cyclopropane, any of which is optionally substituted with one or more R⁷.

In some embodiments, R² is a cycloalkenyl. In some embodiments, R² is a cycloalkenyl selected from cyclooctene, cycloheptene, cyclohexene, cyclopentene, cyclobutene, and cyclopropane, any of which is optionally substituted with one or more R⁷. In some embodiments, the cycloalkenyl can have 1, 2, 3, 4, or more degrees of unsaturation.

In some embodiments, R² is a 6-membered heteroaryl. In some embodiments, R² is a 6-membered heteroaryl selected from pyridine, pyrazine, and pyrimidine, any of which is optionally substituted with one or more R⁷.

In some embodiments, R² is a 5-membered heteroaryl. In some embodiments, R² is a 5-membered heteroaryl selected from thiophene, furan, pyrrole, pyrazole, selenophene, imidazole, thiazole, isothiazole, oxatriazole, oxadiazole, oxazole, and thiadiazole, any of which is optionally substituted with one or more R⁷. In some embodiments, the heteroatom of the heteroaryl is not bound to the methylene carbon of Formula (I).

In some embodiments, R² is a 6-membered heterocycloalkenyl. In some embodiments, R² is a 6-membered heterocycloalkenyl selected from dihydropyridine, tetrahydropyridine, dihydropyridazine, tetrahydropyridazine, dihydropyrimidine, tetrahydropyrimidine, dihydropyrazine, tetrahydropyridazine, dihydropyrimidine, tetrahydropyrimidine, dihydropyrazine, tetrahydropyrazine, pyran, dihydropyran, thiopyran, dihydrothiopyran, dioxine, dihydrodioxine, oxazine, dihydrooxazine, thiazine, and dihydrothiazine, any of which is optionally substituted with one or more R⁷. In some embodiments, the heteroatom of the heterocycloalkenyl is not bound to the methylene carbon of Formula (I).

In some embodiments, R² is a 5-membered heterocycloalkenyl. In some embodiments, R² is a 5-membered heterocycloalkenyl selected from pyrroline, pyrazoline, imidazoline, triazoline, dihydrofuran, dihydrothiophene, oxazoline, isooxazoline, thiazoline, isothiazoline, oxadiazoline, and thiadiazoline, any of which is optionally substituted with one or more R⁷. In some embodiments, the heteroatom of the heterocycloalkenyl is not bound to the methylene carbon of Formula (I).

In some embodiments, R² is a 6-membered heterocycloalkyl. In some embodiments, R² is a 6-membered heterocycloalkyl selected from piperidine, piperazine, tetrahydrothiopyran, tetrahydropyran, dioxane, morpholine, and thiomorpholine, any of which is optionally substituted with one or more R⁷. In some embodiments, the heteroatom of the heterocycloalkyl is not bound to the methylene carbon of Formula (I).

In some embodiments, R² is a 5-membered heterocycloalkyl. In some embodiments, R² is a 5-membered heterocycloalkyl selected from tetrahydrofuran, pyrrolidone, and tetrahydrothiophene, any of which is optionally substituted with one or more R⁷. In some embodiments, the heteroatom of the heterocycloalkyl is not bound to the methylene carbon of Formula (I).

In some embodiments, R² is a 4-membered heterocycloalkyl. In some embodiments, R² is a 4-membered heterocycloalkyl selected from azetidine, oxetane, and thietane, any of which is optionally substituted with one or more R⁷. In some embodiments, the heteroatom of the heterocycloalkyl is not bound to the methylene carbon of Formula (I).

In some embodiments, R² is a 6-6 fused ring system. In some embodiments, R² is a 6-6 fused ring system selected from naphthalene, quinoline, isoquinoline, pteridine, benzopyran, dihydroquinoline, dihydroisoquinoline, dihydronapthalene, and tetrahydronapthalene, any of which is optionally substituted with one or more R⁷.

In some embodiments, R² is a 5-6 fused ring system. In some embodiments, R² is a 5-6 fused ring system selected from benzimidazole, indole, azaindole, indazole, benzothiophene, benzofuran, benzoselenophene, benzimidazole, benzotriazole, benzothiazole, benzisothiazole, benzoxadiazole, benzoxazole, and benzothiadiazole, imidazo[4,5-b]-pyridine, imidazo[4,5-c]-pyridine, pyrazolo[3,4-b]pyridine, pyrazolo[3,4-c]pyridine, pyrazolo[4,3-b]pyridine, pyrazolo[4,3-b]pyridine, oxazolo[4,5-b]pyridine, oxazolo[4,5-c]pyridine, oxazolo[5,4-b]pyridine, oxazolo[5,4-c]pyridine, thiazolo[4,5-b]pyridine, thiazolo[4,5-c]pyridine, thiazolo[5,4-b]pyridine, thiazolo[5,4-c]pyridine, isooxazolo[4,5-b]pyridine, isooxazolo[4,5-c]pyridine, isooxazolo[3,4-b]pyridine, isooxazolo[3,4-c]pyridine, isooxazolo[4,3-b]pyridine, isooxazolo[4,3-c]pyridine, isooxazolo[5,4-b]pyridine, isooxazolo[5,4-c]pyridine, isothiazolo[4,5-b]pyridine, isothiazolo[4,5-c]pyridine, isothiazolo[5,4-b]pyridine, isothiazolo[5,4-c]pyridine, isothiazolo[3,4-b]pyridine, isothiazolo[3,4-c]pyridine, isothiazolo[4,3-b]pyridine, isothiazolo[4,3-c]pyridine and purine, any of which is optionally substituted with one or more R⁷.

In some embodiments, R² is a 5-5 fused ring system. In some embodiments, R² is a 5-5 fused ring system selected from pyrrolo[2,1-b]thiazole, pyrazolo[5,1-b]thiazole, imidazo[5,1-b]thiazole, imidazo[2,1-b]thiazole, thiazolo[3,2-b][1,2,4]triazole, thiazolo[3,2-c][1,2,4]triazole, imidazo[2,1-b]thiadiazole, imidazo[1,2-d][1,2,4]thiadiazole, thiazolo[3,2-d]tetrazole, thieno[3,2-b]thiophene, thieno[2,3-b]thiophene, thieno[3,4-b]thiophene, thieno[3,4-c]thiophene, any of which is optionally substituted with one or more R⁷.

In some embodiments, R² is a bridged ring system. In some embodiments, R² is a bridged ring system selected from norbornane, norbornene, bicyclo[1.1.0]butane, bicyclo[3.2.0]heptane, bicyclo[3.3.0]octane, bicyclo[4.3.0]nonane, bicyclo[1.1.1]pentane, bicyclo[2.2.1]hexane, bicyclo[3.3.1]nonane, bicyclo[2.2.2]octane, adamantane, cubane, housane, any of which is optionally substituted with one or more R⁷. In some embodiments, the bridged ring system can have 1, 2, 3, 4, or more degrees of unsaturation.

In some embodiments, R³ is hydrogen, C₁₋₆ alkyl, C₂₋₆ alkenyl, or C₂₋₆ alkynyl, each of which is optionally substituted with one or more R⁸.

In some embodiments, R³ is C₃₋₇ carbocycle or 3- to 7-membered heterocycle; wherein the C₃₋₇ carbocycle and 3- to 7-membered heterocycle are each optionally substituted with one or more R⁸.

In some embodiments, R³ is phenyl, optionally substituted with one or more R⁸.

In some embodiments, R³ is a cycloalkyl. In some embodiments, R³ is a cycloalkyl selected from cyclooctane, cycloheptane, cyclohexane, cyclopentane, cyclobutane, and cyclopropane, any of which is optionally substituted with one or more R⁸.

In some embodiments, R³ is a cycloalkenyl. In some embodiments, R³ is a cycloalkenyl selected from cyclooctene, cycloheptene, cyclohexene, cyclopentene, cyclobutene, and cyclopropane, any of which is optionally substituted with one or more R⁸. In some embodiments, the cycloalkenyl can have 1, 2, 3, 4, or more degrees of unsaturation.

In some embodiments, R³ is a 6-membered heteroaryl. In some embodiments, R³ is a 6-membered heteroaryl selected from pyridine, pyrazine, and pyrimidine, any of which is optionally substituted with one or more R⁸. In some embodiments, the heteroatom of the heteroaryl is not bound to the methylene carbon of Formula (I).

In some embodiments, R³ is a 5-membered heteroaryl. In some embodiments, R³ is a 5-membered heteroaryl selected from thiophene, furan, pyrrole, pyrazole, selenophene, imidazole, thiazole, isothiazole, oxatriazole, oxadiazole, oxazole, and thiadiazole, any of which is optionally substituted with one or more R⁸. In some embodiments, the heteroatom of the heteroaryl is not bound to the methylene carbon of Formula (I).

In some embodiments, R³ is a 6-membered heterocycloalkenyl. In some embodiments, R³ is a 6-membered heterocycloalkenyl selected from dihydropyridine, tetrahydropyridine, dihydropyridazine, tetrahydropyridazine, dihydropyrimidine, tetrahydropyrimidine, dihydropyrazine, tetrahydropyridazine, dihydropyrimidine, tetrahydropyrimidine, dihydropyrazine, tetrahydropyrazine, pyran, dihydropyran, thiopyran, dihydrothiopyran, dioxine, dihydrodioxine, oxazine, dihydrooxazine, thiazine, and dihydrothiazine, any of which is optionally substituted with one or more R⁸. In some embodiments, the heteroatom of the heterocycloalkenyl is not bound to the methylene carbon of Formula (I).

In some embodiments, R³ is a 5-membered heterocycloalkenyl. In some embodiments, R³ is a 5-membered heterocycloalkenyl selected from pyrroline, pyrazoline, imidazoline, triazoline, dihydrofuran, dihydrothiophene, oxazoline, isooxazoline, thiazoline, isothiazoline, oxadiazoline, and thiadiazoline, any of which is optionally substituted with one or more R⁸. In some embodiments, the heteroatom of the heterocycloalkenyl is not bound to the methylene carbon of Formula (I).

In some embodiments, R³ is a 6-membered heterocycloalkyl. In some embodiments, R³ is a 6-membered heterocycloalkyl selected from piperidine, piperazine, tetrahydrothiopyran, tetrahydropyran, dioxane, morpholine, and thiomorpholine, any of which is optionally substituted with one or more R⁸. In some embodiments, the heteroatom of the heterocycloalkyl is not bound to the methylene carbon of Formula (I).

In some embodiments, R³ is a 5-membered heterocycloalkyl. In some embodiments, R³ is a 5-membered heterocycloalkyl selected from tetrahydrofuran, pyrrolidone, and tetrahydrothiophene, any of which is optionally substituted with one or more R⁸. In some embodiments, the heteroatom of the heterocycloalkyl is not bound to the methylene carbon of Formula (I).

In some embodiments, R³ is a 4-membered heterocycloalkyl. In some embodiments, R³ is a 4-membered heterocycloalkyl selected from azetidine, oxetane, and thietane, any of which is optionally substituted with one or more R⁸. In some embodiments, the heteroatom of the heterocycloalkyl is not bound to the methylene carbon of Formula (I).

In some embodiments, R³ is a 6-6 fused ring system. In some embodiments, R³ is a 6-6 fused ring system selected from naphthalene, quinoline, isoquinoline, pteridine, benzopyran, dihydroquinoline, dihydroisoquinoline, dihydronapthalene, and tetrahydronapthalene, any of which is optionally substituted with one or more R⁸.

In some embodiments, R³ is a 5-6 fused ring system. In some embodiments, R³ is a 5-6 fused ring system selected from benzimidazole, indole, azaindole, indazole, benzothiophene, benzofuran, benzoselenophene, benzimidazole, benzotriazole, benzothiazole, benzisothiazole, benzoxadiazole, benzoxazole, and benzothiadiazole, imidazo[4,5-b]-pyridine, imidazo[4,5-c]-pyridine, pyrazolo[3,4-b]pyridine, pyrazolo[3,4-c]pyridine, pyrazolo[4,3-b]pyridine, pyrazolo[4,3-b]pyridine, oxazolo[4,5-b]pyridine, oxazolo[4,5-c]pyridine, oxazolo[5,4-b]pyridine, oxazolo[5,4-c]pyridine, thiazolo[4,5-b]pyridine, thiazolo[4,5-c]pyridine, thiazolo[5,4-b]pyridine, thiazolo[5,4-c]pyridine, isooxazolo[4,5-b]pyridine, isooxazolo[4,5-c]pyridine, isooxazolo[3,4-b]pyridine, isooxazolo[3,4-c]pyridine, isooxazolo[4,3-b]pyridine, isooxazolo[4,3-c]pyridine, isooxazolo[5,4-b]pyridine, isooxazolo[5,4-c]pyridine, isothiazolo[4,5-b]pyridine, isothiazolo[4,5-c]pyridine, isothiazolo[5,4-b]pyridine, isothiazolo[5,4-c]pyridine, isothiazolo[3,4-b]pyridine, isothiazolo[3,4-c]pyridine, isothiazolo[4,3-b]pyridine, isothiazolo[4,3-c]pyridine and purine, any of which is optionally substituted with one or more R⁸.

In some embodiments, R³ is a 5-5 fused ring system. In some embodiments, R³ is a 5-5 fused ring system selected from pyrrolo[2,1-b]thiazole, pyrazolo[5,1-b]thiazole, imidazo[5,1-b]thiazole, imidazo[2,1-b]thiazole, thiazolo[3,2-b][1,2,4]triazole, thiazolo[3,2-c][1,2,4]triazole, imidazo[2,1-b]thiadiazole, imidazo[1,2-d][1,2,4]thiadiazole, thiazolo[3,2-d]tetrazole, thieno[3,2-b]thiophene, thieno[2,3-b]thiophene, thieno[3,4-b]thiophene, thieno[3,4-c]thiophene, any of which is optionally substituted with one or more R⁸.

In some embodiments, R³ is a bridged ring system. In some embodiments, R³ is a bridged ring system selected from norbornane, norbornene, bicyclo[1.1.0]butane, bicyclo[3.2.0]heptane, bicyclo[3.3.0]octane, bicyclo[4.3.0]nonane, bicyclo[1.1.1]pentane, bicyclo[2.2.1]hexane, bicyclo[3.3.1]nonane, bicyclo[2.2.2]octane, adamantane, cubane, housane, any of which is optionally substituted with one or more R⁸. In some embodiments, the bridged ring system can have 1, 2, 3, 4, or more degrees of unsaturation.

In some embodiments, each of R⁴ or R⁵ may independently be hydrogen, C₁₋₁₀ alkyl, C₂₋₁₀ alkenyl, or C₂₋₁₀ alkynyl, each of which is optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₁₋₆ cycloalkyl, and C₁₋₆ cycloalkenyl.

In some embodiments, each of R⁴ or R⁵ may independently be 3- to 10-membered heterocycle. In some embodiments, each of R⁴ or R⁵ may independently be 3- to 10-membered heterocycle selected from azacyclopropane, pyrrolidine, piperidine, azepane, azocane, azonane, morpholine, thiomorpholine, 1,4-piperazine, 1,4-oxazepane, and 1,4-diazepane.

In some embodiments, the compound of Formula (I) is selected from:

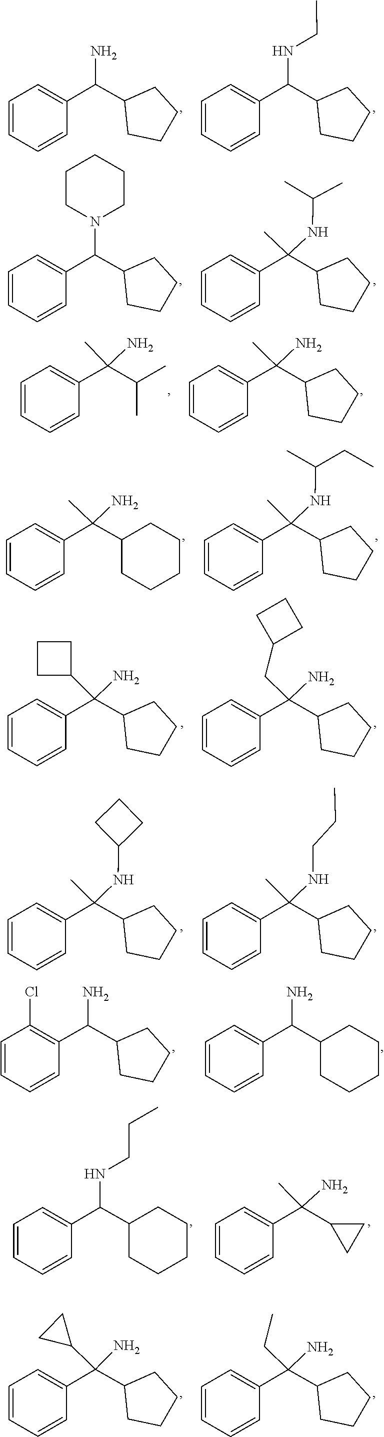

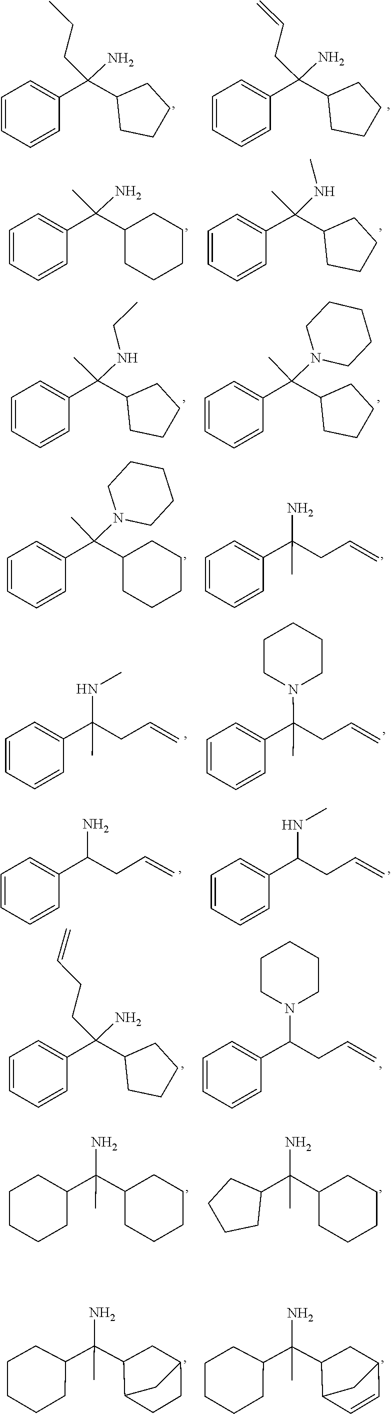

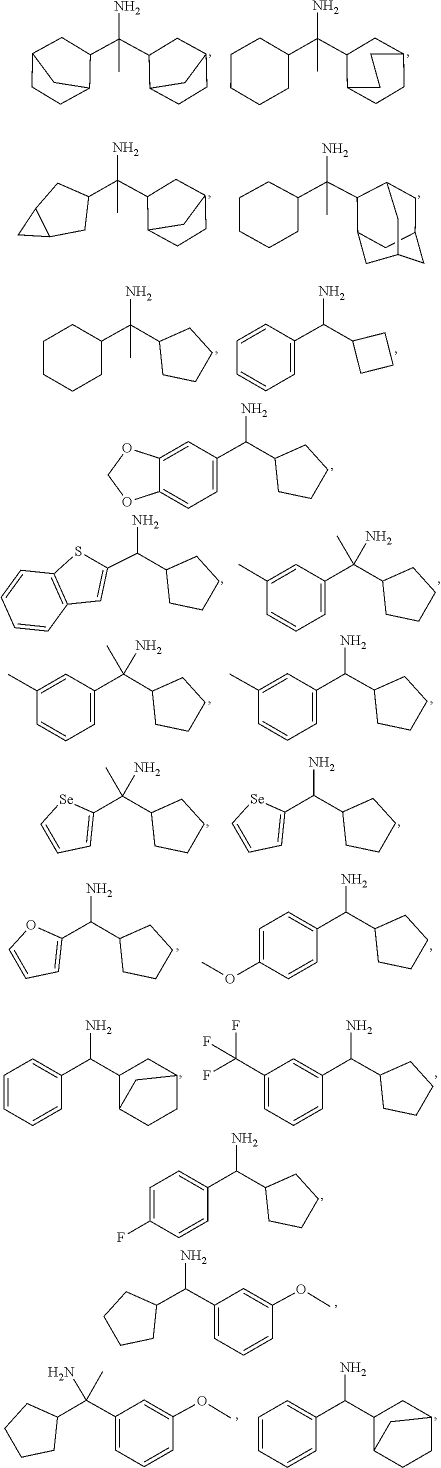

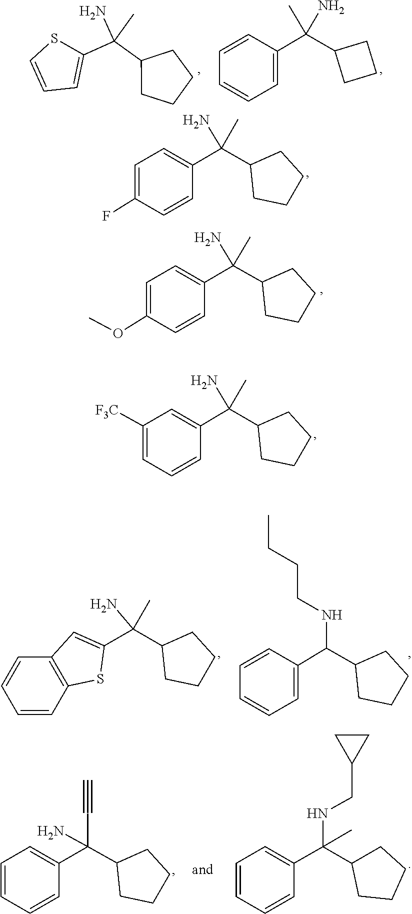

In another aspect, disclosed herein is a method for treating a physical, psychiatric, or neurological disorder comprising inhibiting an inotropic receptor by contacting the receptor with a pharmaceutical composition comprising a compound represented by the structure of Formula (II):

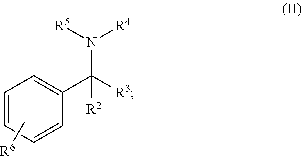

or an enantiomer, a mixture of enantiomers, or an isotopic variant thereof; or a pharmaceutically acceptable salt, solvate or hydrate thereof, wherein:
R² is selected from
   hydrogen;
   halogen;
   C₁₋₈ alkyl, C₂₋₈ alkenyl, and C₂₋₈ alkynyl, each of which is optionally substituted with one or more R⁷; and
   C₃₋₁₀ carbocycle and 3- to 10-membered heterocycle; wherein the C₃₋₁₀ carbocycle and 3-to 10-membered heterocycle are each optionally substituted with one or more R⁷;
R³ is selected from:
   C₁₋₆ alkyl, C₂₋₆ alkenyl, and C₂₋₆ alkynyl, each of which is optionally substituted with one or more R⁸; and
   C₃₋₁₀ carbocycle and 3- to 10-membered heterocycle; wherein the C₃₋₁₀ carbocycle and 3-to 10-membered heterocycle are each optionally substituted with one or more R¹;
R⁴, R⁵, R⁶, R⁷, and R⁸ are each independently selected from:
   hydrogen;
   C₁₋₁₀ alkyl, C₂₋₁₀ alkenyl, and C₂₋₁₀ alkynyl optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₃₋₆ cycloalkyl, and C₃₋₆ cycloalkenyl; and
   C₃₋₁₀ carbocycle or 3- to 10-membered heterocycle; wherein the C₃₋₁₀ carbocycle and 3- to 10-membered heterocycle are each optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), and C₁₋₈ alkyl;
provided that the compound of Formula II is not

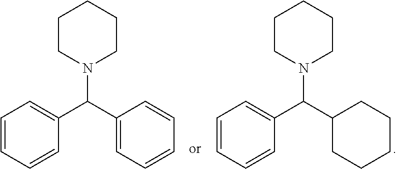

In some embodiments, R² is hydrogen, C₁₋₈ alkyl, C₂₋₈ alkenyl, or C₂₋₈ alkynyl, each of which is optionally substituted with one or more R⁷.

In some embodiments, R² is C₃₋₇ carbocycle or 3- to 7-membered heterocycle; wherein the C₃₋₇ carbocycle and 3- to 7-membered heterocycle are each optionally substituted with one or more R⁷.

In some embodiments, R² is phenyl, optionally substituted with one or more R⁷.

In some embodiments, R² is a cycloalkyl. In some embodiments, R² is a cycloalkyl selected from cyclooctane, cycloheptane, cyclohexane, cyclopentane, cyclobutane, and cyclopropane, any of which is optionally substituted with one or more R⁷.

In some embodiments, R² is a cycloalkenyl. In some embodiments, R² is a cycloalkenyl selected from cyclooctene, cycloheptene, cyclohexene, cyclopentene, cyclobutene, and cyclopropane, any of which is optionally substituted with one or more R⁷. In some embodiments, the cycloalkenyl can have 1, 2, 3, 4, or more degrees of unsaturation.

In some embodiments, R² is a 6-membered heteroaryl. In some embodiments, R² is a 6-membered heteroaryl selected from pyridine, pyrazine, and pyrimidine, any of which is optionally substituted with one or more R⁷. In some embodiments, the heteroatom of the heteroaryl is not bound to the methylene carbon of Formula (II).

In some embodiments, R² is a 5-membered heteroaryl. In some embodiments, R² is a 5-membered heteroaryl selected from thiophene, furan, pyrrole, pyrazole, selenophene, imidazole, thiazole, isothiazole, oxatriazole, oxadiazole, oxazole, and thiadiazole, any of which is optionally substituted with one or more R⁷. In some embodiments, the heteroatom of the heteroaryl is not bound to the methylene carbon of Formula (II).

In some embodiments, R² is a 6-membered heterocycloalkenyl. In some embodiments, R² is a 6-membered heterocycloalkenyl selected from dihydropyridine, tetrahydropyridine, dihydropyridazine, tetrahydropyridazine, dihydropyrimidine, tetrahydropyrimidine, dihydropyrazine, tetrahydropyridazine, dihydropyrimidine, tetrahydropyrimidine, dihydropyrazine, tetrahydropyrazine, pyran, dihydropyran, thiopyran, dihydrothiopyran, dioxine, dihydrodioxine, oxazine, dihydrooxazine, thiazine, and dihydrothiazine, any of which is optionally substituted with one or more R⁷. In some embodiments, the heteroatom of the heterocycloalkenyl is not bound to the methylene carbon of Formula (II).

In some embodiments, R² is a 5-membered heterocycloalkenyl. In some embodiments, R² is a 5-membered heterocycloalkenyl selected from pyrroline, pyrazoline, imidazoline, triazoline, dihydrofuran, dihydrothiophene, oxazoline, isooxazoline, thiazoline, isothiazoline, oxadiazoline, and thiadiazoline, any of which is optionally substituted with one or more R⁷. In some embodiments, the heteroatom of the heterocycloalkenyl is not bound to the methylene carbon of Formula (II).

In some embodiments, R² is a 6-membered heterocycloalkyl. In some embodiments, R² is a 6-membered heterocycloalkyl selected from piperidine, piperazine, tetrahydrothiopyran, tetrahydropyran, dioxane, morpholine, and thiomorpholine, any of which is optionally substituted with one or more R⁷. In some embodiments, the heteroatom of the heterocycloalkyl is not bound to the methylene carbon of Formula (II).

In some embodiments, R² is a 5-membered heterocycloalkyl. In some embodiments, R² is a 5-membered heterocycloalkyl selected from tetrahydrofuran, pyrrolidone, and tetrahydrothiophene, any of which is optionally substituted with one or more R⁷. In some embodiments, the heteroatom of the heterocycloalkyl is not bound to the methylene carbon of Formula (II).

In some embodiments, R² is a 4-membered heterocycloalkyl. In some embodiments, R² is a 4-membered heterocycloalkyl selected from azetidine, oxetane, and thietane, any of which is optionally substituted with one or more R⁷. In some embodiments, the heteroatom of the heterocycloalkyl is not bound to the methylene carbon of Formula (II).

In some embodiments, R² is a 6-6 fused ring system. In some embodiments, R² is a 6-6 fused ring system selected from naphthalene, quinoline, isoquinoline, pteridine, benzopyran, dihydroquinoline, dihydroisoquinoline, dihydronapthalene, and tetrahydronapthalene, any of which is optionally substituted with one or more R⁷.

In some embodiments, R² is a 5-6 fused ring system. In some embodiments, R² is a 5-6 fused ring system selected from benzimidazole, indole, azaindole, indazole, benzothiophene, benzofuran, benzoselenophene, benzimidazole, benzotriazole, benzothiazole, benzisothiazole, benzoxadiazole, benzoxazole, and benzothiadiazole, imidazo[4,5-b]-pyridine, imidazo[4,5-c]-pyridine, pyrazolo[3,4-b]pyridine, pyrazolo[3,4-c]pyridine, pyrazolo[4,3-b]pyridine, pyrazolo[4,3-b]pyridine, oxazolo[4,5-b]pyridine, oxazolo[4,5-c]pyridine, oxazolo[5,4-b]pyridine, oxazolo[5,4-c]pyridine, thiazolo[4,5-b]pyridine, thiazolo[4,5-c]pyridine, thiazolo[5,4-b]pyridine, thiazolo[5,4-c]pyridine, isooxazolo[4,5-b]pyridine, isooxazolo[4,5-c]pyridine, isooxazolo[3,4-b]pyridine, isooxazolo[3,4-c]pyridine, isooxazolo[4,3-b]pyridine, isooxazolo[4,3-c]pyridine, isooxazolo[5,4-b]pyridine, isooxazolo[5,4-c]pyridine, isothiazolo[4,5-b]pyridine, isothiazolo[4,5-c]pyridine, isothiazolo[5,4-b]pyridine, isothiazolo[5,4-c]pyridine, isothiazolo[3,4-b]pyridine, isothiazolo[3,4-c]pyridine, isothiazolo[4,3-b]pyridine, isothiazolo[4,3-c]pyridine and purine, any of which is optionally substituted with one or more R⁷.

In some embodiments, R² is a 5-5 fused ring system. In some embodiments, R² is a 5-5 fused ring system selected from pyrrolo[2,1-b]thiazole, pyrazolo[5,1-b]thiazole, imidazo[5,1-b]thiazole, imidazo[2,1-b]thiazole, thiazolo[3,2-b][1,2,4]triazole, thiazolo[3,2-c][1,2,4]triazole, imidazo[2,1-b]thiadiazole, imidazo[1,2-d][1,2,4]thiadiazole, thiazolo[3,2-d]tetrazole, thieno[3,2-b]thiophene, thieno[2,3-b]thiophene, thieno[3,4-b]thiophene, thieno[3,4-c]thiophene, any of which is optionally substituted with one or more R⁷.

In some embodiments, R² is a bridged ring system. In some embodiments, R² is a bridged ring system selected from norbornane, norbornene, bicyclo[1.1.0]butane, bicyclo[3.2.0]heptane, bicyclo[3.3.0]octane, bicyclo[4.3.0]nonane, bicyclo[1.1.1]pentane, bicyclo[2.2.1]hexane, bicyclo[3.3.1]nonane, bicyclo[2.2.2]octane, adamantane, cubane, housane, any of which is optionally substituted with one or more R⁷. In some embodiments, the bridged ring system can have 1, 2, 3, 4, or more degrees of unsaturation.

In some embodiments, R³ is hydrogen, C₁₋₆ alkyl, C₂₋₆ alkenyl, or C₂₋₆ alkynyl, each of which is optionally substituted with one or more R⁸.

In some embodiments, R³ is C₃₋₇ carbocycle or 3- to 7-membered heterocycle; wherein the C₃₋₇ carbocycle and 3- to 7-membered heterocycle are each optionally substituted with one or more R⁸.

In some embodiments, R³ is phenyl, optionally substituted with one or more R⁸.

In some embodiments, R³ is a cycloalkyl. In some embodiments, R³ is a cycloalkyl selected from cyclooctane, cycloheptane, cyclohexane, cyclopentane, cyclobutane, and cyclopropane, any of which is optionally substituted with one or more R⁸.

In some embodiments, R³ is a cycloalkenyl. In some embodiments, R³ is a cycloalkenyl selected from cyclooctene, cycloheptene, cyclohexene, cyclopentene, cyclobutene, and cyclopropane, any of which is optionally substituted with one or more R⁸. In some embodiments, the cycloalkenyl can have 1, 2, 3, 4, or more degrees of unsaturation.

In some embodiments, R³ is a 6-membered heteroaryl. In some embodiments, R³ is a 6-membered heteroaryl selected from pyridine, pyrazine, and pyrimidine, any of which is optionally substituted with one or more R⁸. In some embodiments, the heteroatom of the heteroaryl is not bound to the methylene carbon of Formula (II).

In some embodiments, R³ is a 5-membered heteroaryl. In some embodiments, R³ is a 5-membered heteroaryl selected from thiophene, furan, pyrrole, pyrazole, selenophene, imidazole, thiazole, isothiazole, oxatriazole, oxadiazole, oxazole, and thiadiazole, any of which is optionally substituted with one or more R⁸. In some embodiments, the heteroatom of the heteroaryl is not bound to the methylene carbon of Formula (II).

In some embodiments, R³ is a 6-membered heterocycloalkenyl. In some embodiments, R³ is a 6-membered heterocycloalkenyl selected from dihydropyridine, tetrahydropyridine, dihydropyridazine, tetrahydropyridazine, dihydropyrimidine, tetrahydropyrimidine, dihydropyrazine, tetrahydropyridazine, dihydropyrimidine, tetrahydropyrimidine, dihydropyrazine, tetrahydropyrazine, pyran, dihydropyran, thiopyran, dihydrothiopyran, dioxine, dihydrodioxine, oxazine, dihydrooxazine, thiazine, and dihydrothiazine, any of which is optionally substituted with one or more R⁸. In some embodiments, the heteroatom of the heterocycloalkenyl is not bound to the methylene carbon of Formula (II).

In some embodiments, R³ is a 5-membered heterocycloalkenyl. In some embodiments, R³ is a 5-membered heterocycloalkenyl selected from pyrroline, pyrazoline, imidazoline, triazoline, dihydrofuran, dihydrothiophene, oxazoline, isooxazoline, thiazoline, isothiazoline, oxadiazoline, and thiadiazoline, any of which is optionally substituted with one or more R⁸. In some embodiments, the heteroatom of the heterocycloalkenyl is not bound to the methylene carbon of Formula (II).

In some embodiments, R³ is a 6-membered heterocycloalkyl. In some embodiments, R³ is a 6-membered heterocycloalkyl selected from piperidine, piperazine, tetrahydrothiopyran, tetrahydropyran, dioxane, morpholine, and thiomorpholine, any of which is optionally substituted with one or more R⁸. In some embodiments, the heteroatom of the heterocycloalkyl is not bound to the methylene carbon of Formula (II).

In some embodiments, R³ is a 5-membered heterocycloalkyl. In some embodiments, R³ is a 5-membered heterocycloalkyl selected from tetrahydrofuran, pyrrolidone, and tetrahydrothiophene, any of which is optionally substituted with one or more R⁸. In some embodiments, the heteroatom of the heterocycloalkyl is not bound to the methylene carbon of Formula (II).

In some embodiments, R³ is a 4-membered heterocycloalkyl. In some embodiments, R³ is a 4-membered heterocycloalkyl selected from azetidine, oxetane, and thietane, any of which is optionally substituted with one or more R⁸. In some embodiments, the heteroatom of the heterocycloalkyl is not bound to the methylene carbon of Formula (II).

In some embodiments, R³ is a 6-6 fused ring system. In some embodiments, R³ is a 6-6 fused ring system selected from naphthalene, quinoline, isoquinoline, pteridine, benzopyran, dihydroquinoline, dihydroisoquinoline, dihydronapthalene, and tetrahydronapthalene, any of which is optionally substituted with one or more R⁸.

In some embodiments, R³ is a 5-6 fused ring system. In some embodiments, R³ is a 5-6 fused ring system selected from benzimidazole, indole, azaindole, indazole, benzothiophene, benzofuran, benzoselenophene, benzimidazole, benzotriazole, benzothiazole, benzisothiazole, benzoxadiazole, benzoxazole, and benzothiadiazole, imidazo[4,5-b]-pyridine, imidazo[4,5-c]-pyridine, pyrazolo[3,4-b]pyridine, pyrazolo[3,4-c]pyridine, pyrazolo[4,3-b]pyridine, pyrazolo[4,3-b]pyridine, oxazolo[4,5-b]pyridine, oxazolo[4,5-c]pyridine, oxazolo[5,4-b]pyridine, oxazolo[5,4-c]pyridine, thiazolo[4,5-b]pyridine, thiazolo[4,5-c]pyridine, thiazolo[5,4-b]pyridine, thiazolo[5,4-c]pyridine, isooxazolo[4,5-b]pyridine, isooxazolo[4,5-c]pyridine, isooxazolo[3,4-b]pyridine, isooxazolo[3,4-c]pyridine, isooxazolo[4,3-b]pyridine, isooxazolo[4,3-c]pyridine, isooxazolo[5,4-b]pyridine, isooxazolo[5,4-c]pyridine, isothiazolo[4,5-b]pyridine, isothiazolo[4,5-c]pyridine, isothiazolo[5,4-b]pyridine, isothiazolo[5,4-c]pyridine, isothiazolo[3,4-b]pyridine, isothiazolo[3,4-c]pyridine, isothiazolo[4,3-b]pyridine, isothiazolo[4,3-c]pyridine and purine, any of which is optionally substituted with one or more R⁸.

In some embodiments, R³ is a 5-5 fused ring system. In some embodiments, R³ is a 5-5 fused ring system selected from pyrrolo[2,1-b]thiazole, pyrazolo[5,1-b]thiazole, imidazo[5,1-b]thiazole, imidazo[2,1-b]thiazole, thiazolo[3,2-b][1,2,4]triazole, thiazolo[3,2-c][1,2,4]triazole, imidazo[2,1-b]thiadiazole, imidazo[1,2-d][1,2,4]thiadiazole, thiazolo[3,2-d]tetrazole, thieno[3,2-b]thiophene, thieno[2,3-b]thiophene, thieno[3,4-b]thiophene, thieno[3,4-c]thiophene, any of which is optionally substituted with one or more R⁸.

In some embodiments, R³ is a bridged ring system. In some embodiments, R³ is a bridged ring system selected from norbornane, norbornene, bicyclo[1.1.0]butane, bicyclo[3.2.0]heptane, bicyclo[3.3.0]octane, bicyclo[4.3.0]nonane, bicyclo[1.1.1]pentane, bicyclo[2.2.1]hexane, bicyclo[3.3.1]nonane, bicyclo[2.2.2]octane, adamantane, cubane, housane, any of which is optionally substituted with one or more R⁸. In some embodiments, the bridged ring system can have 1, 2, 3, 4, or more degrees of unsaturation.

In some embodiments, each of R⁴ or R⁵ may independently be hydrogen, C₁₋₁₀ alkyl, C₂₋₁₀ alkenyl, or C₂₋₁₀ alkynyl, each of which is optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₁₋₆ cycloalkyl, and C₁₋₆ cycloalkenyl.

In some embodiments, each of R⁴ or R⁵ may independently be 3- to 10-membered heterocycle. In some embodiments, each of R⁴ or R⁵ may independently be 3- to 10-membered heterocycle selected from azacyclopropane, pyrrolidine, piperidine, azepane, azocane, azonane, morpholine, thiomorpholine, 1,4-piperazine, 1,4-oxazepane, and 1,4-diazepane.

In some embodiments, compound of Formula (II) is selected from:

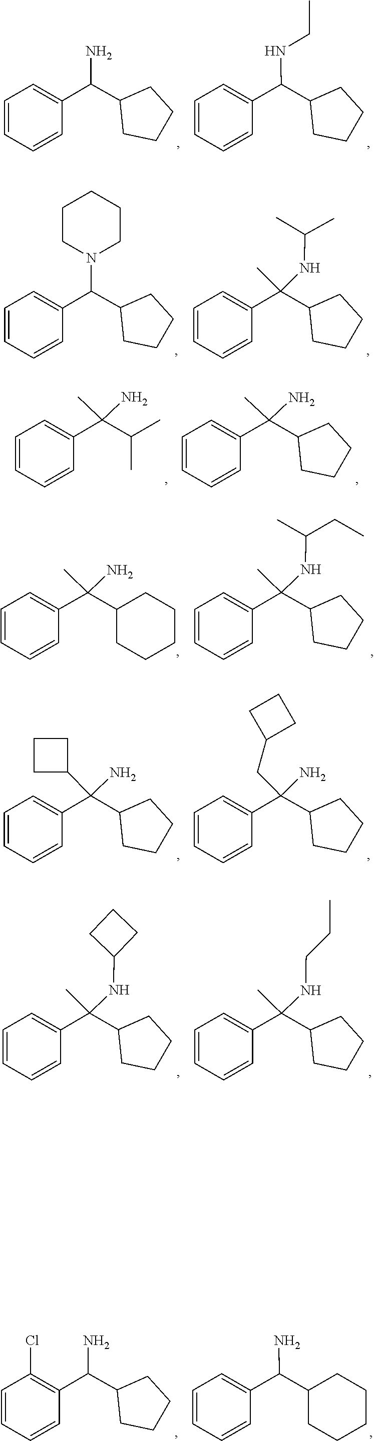

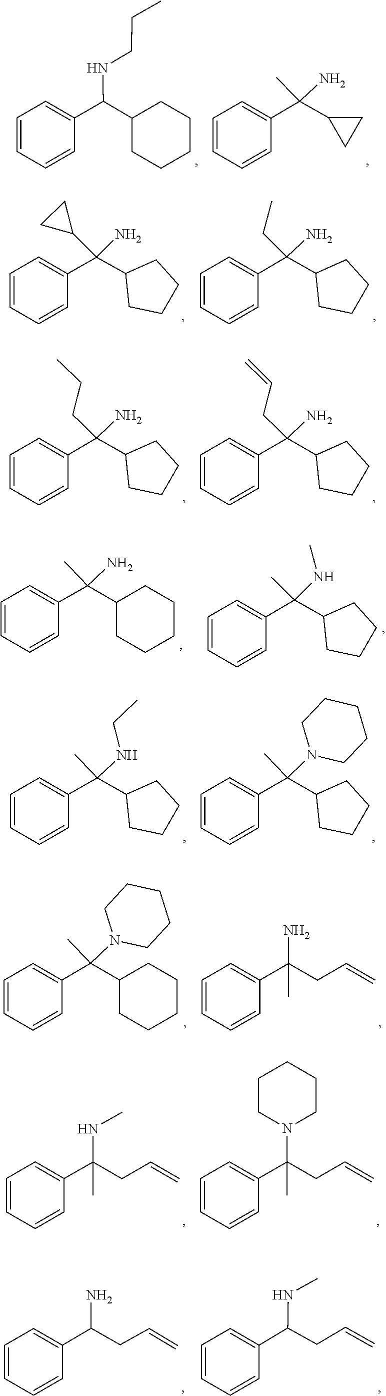

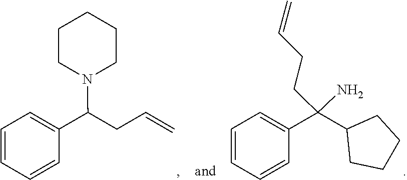 [SMILES: C=CCC(c1ccccc1)N1CCCCC1 ; C=CCCC(N)(c1ccccc1)C1CCCC1]

In another aspect, disclosed herein is a method for treating a physical, psychiatric, or neurological disorder comprising inhibiting an inotropic receptor by contacting the receptor with a pharmaceutical composition comprising a compound represented by the structure of Formula

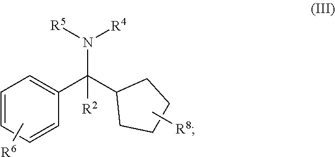

or an enantiomer, a mixture of enantiomers, or an isotopic variant thereof, or a pharmaceutically acceptable salt, solvate or hydrate thereof, wherein:
R² is selected from
   hydrogen;
   halogen;
   C₁₋₈ alkyl, C₂₋₈ alkenyl, and C₂₋₈ alkynyl, each of which is optionally substituted with one or more R⁷; and
   C₃₋₁₀ carbocycle and 3- to 10-membered heterocycle; wherein the C₃₋₁₀ carbocycle and 3-to 10-membered heterocycle are each optionally substituted with one or more R⁷;
R⁴, R⁵, R⁶, R⁷, and R⁸ are each independently selected from:
   hydrogen;
   C₁₋₁₀ alkyl, C₂₋₁₀ alkenyl, and C₂₋₁₀ alkynyl optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₁₋₆ cycloalkyl, and C₁₋₆ cycloalkenyl; and
   C₃₋₁₀ carbocycle or 3- to 10-membered heterocycle; wherein the C₃₋₁₀ carbocycle and 3- to 10-membered heterocycle are each optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), and C₁₋₈ alkyl.

In some embodiments, R² is hydrogen, C₁₋₈ alkyl, C₂₋₈ alkenyl, or C₂₋₈ alkynyl, each of which is optionally substituted with one or more R⁷.

In some embodiments, R² is C₃₋₇ carbocycle or 3- to 7-membered heterocycle; wherein the C₃₋₇ carbocycle and 3- to 7-membered heterocycle are each optionally substituted with one or more R⁷.

In some embodiments, R² is phenyl, optionally substituted with one or more R⁷.

In some embodiments, R² is a cycloalkyl. In some embodiments, R² is a cycloalkyl selected from cyclooctane, cycloheptane, cyclohexane, cyclopentane, cyclobutane, and cyclopropane, any of which is optionally substituted with one or more R⁷.

In some embodiments, R² is a cycloalkenyl. In some embodiments, R² is a cycloalkenyl selected from cyclooctene, cycloheptene, cyclohexene, cyclopentene, cyclobutene, and cyclopropane, any of which is optionally substituted with one or more R⁷. In some embodiments, the cycloalkenyl can have 1, 2, 3, 4, or more degrees of unsaturation.

In some embodiments, R² is a 6-membered heteroaryl. In some embodiments, R² is a 6-membered heteroaryl selected from pyridine, pyrazine, and pyrimidine, any of which is optionally substituted with one or more R⁷. In some embodiments, the heteroatom of the heteroaryl is not bound to the methylene carbon of Formula (III).

In some embodiments, R² is a 5-membered heteroaryl. In some embodiments, R² is a 5-membered heteroaryl selected from thiophene, furan, pyrrole, pyrazole, selenophene, imidazole, thiazole, isothiazole, oxatriazole, oxadiazole, oxazole, and thiadiazole, any of which is optionally substituted with one or more R⁷. In some embodiments, the heteroatom of the heteroaryl is not bound to the methylene carbon of Formula (III).

In some embodiments, R² is a 6-membered heterocycloalkenyl. In some embodiments, R² is a 6-membered heterocycloalkenyl selected from dihydropyridine, tetrahydropyridine, dihydropyridazine, tetrahydropyridazine, dihydropyrimidine, tetrahydropyrimidine, dihydropyrazine, tetrahydropyridazine, dihydropyrimidine, tetrahydropyrimidine, dihydropyrazine, tetrahydropyrazine, pyran, dihydropyran, thiopyran, dihydrothiopyran, dioxine, dihydrodioxine, oxazine, dihydrooxazine, thiazine, and dihydrothiazine, any of which is optionally substituted with one or more R⁷. In some embodiments, the heteroatom of the heterocycloalkenyl is not bound to the methylene carbon of Formula (III).

In some embodiments, R² is a 5-membered heterocycloalkenyl. In some embodiments, R² is a 5-membered heterocycloalkenyl selected from pyrroline, pyrazoline, imidazoline, triazoline, dihydrofuran, dihydrothiophene, oxazoline, isooxazoline, thiazoline, isothiazoline, oxadiazoline, and thiadiazoline, any of which is optionally substituted with one or more R⁷. In some embodiments, the heteroatom of the heterocycloalkenyl is not bound to the methylene carbon of Formula (III).

In some embodiments, R² is a 6-membered heterocycloalkyl. In some embodiments, R² is a 6-membered heterocycloalkyl selected from piperidine, piperazine, tetrahydrothiopyran, tetrahydropyran, dioxane, morpholine, and thiomorpholine, any of which is optionally substituted with one or more R⁷. In some embodiments, the heteroatom of the heterocycloalkyl is not bound to the methylene carbon of Formula (III).

In some embodiments, R² is a 5-membered heterocycloalkyl. In some embodiments, R² is a 5-membered heterocycloalkyl selected from tetrahydrofuran, pyrrolidone, and tetrahydrothiophene, any of which is optionally substituted with one or more R⁷. In some embodiments, the heteroatom of the heterocycloalkyl is not bound to the methylene carbon of Formula (III).

In some embodiments, R² is a 4-membered heterocycloalkyl. In some embodiments, R² is a 4-membered heterocycloalkyl selected from azetidine, oxetane, and thietane, any of which is optionally substituted with one or more R⁷. In some embodiments, the heteroatom of the heterocycloalkyl is not bound to the methylene carbon of Formula (III).

In some embodiments, R² is a 6-6 fused ring system. In some embodiments, R² is a 6-6 fused ring system selected from naphthalene, quinoline, isoquinoline, pteridine, benzopyran, dihydroquinoline, dihydroisoquinoline, dihydronapthalene, and tetrahydronapthalene, any of which is optionally substituted with one or more R⁷.

In some embodiments, R² is a 5-6 fused ring system. In some embodiments, R² is a 5-6 fused ring system selected from benzimidazole, indole, azaindole, indazole, benzothiophene, benzofuran, benzoselenophene, benzimidazole, benzotriazole, benzothiazole, benzisothiazole, benzoxadiazole, benzoxazole, and benzothiadiazole, imidazo[4,5-b]-pyridine, imidazo[4,5-c]-pyridine, pyrazolo[3,4-b]pyridine, pyrazolo[3,4-c]pyridine, pyrazolo[4,3-b]pyridine, pyrazolo[4,3-b]pyridine, oxazolo[4,5-b]pyridine, oxazolo[4,5-c]pyridine, oxazolo[5,4-b]pyridine, oxazolo[5,4-c]pyridine, thiazolo[4,5-b]pyridine, thiazolo[4,5-c]pyridine, thiazolo[5,4-b]pyridine, thiazolo[5,4-c]pyridine, isooxazolo[4,5-b]pyridine, isooxazolo[4,5-c]pyridine, isooxazolo[3,4-b]pyridine, isooxazolo[3,4-c]pyridine, isooxazolo[4,3-b]pyridine, isooxazolo[4,3-c]pyridine, isooxazolo[5,4-b]pyridine, isooxazolo[5,4-c]pyridine, isothiazolo[4,5-b]pyridine, isothiazolo[4,5-c]pyridine, isothiazolo[5,4-b]pyridine, isothiazolo[5,4-c]pyridine, isothiazolo[3,4-b]pyridine, isothiazolo[3,4-c]pyridine, isothiazolo[4,3-b]pyridine, isothiazolo[4,3-c]pyridine and purine, any of which is optionally substituted with one or more R⁷.

In some embodiments, R² is a 5-5 fused ring system selected. In some embodiments, R² is a 5-5 fused ring system selected from pyrrolo[2,1-b]thiazole, pyrazolo[5,1-b]thiazole, imidazo[5,1-b]thiazole, imidazo[2,1-b]thiazole, thiazolo[3,2-b][1,2,4]triazole, thiazolo[3,2-c][1,2,4]triazole, imidazo[2,1-b]thiadiazole, imidazo[1,2-d][1,2,4]thiadiazole, thiazolo[3,2-d]tetrazole, thieno[3,2-b]thiophene, thieno[2,3-b]thiophene, thieno[3,4-b]thiophene, thieno[3,4-c]thiophene, any of which is optionally substituted with one or more R⁷.

In some embodiments, R² is a bridged ring system. In some embodiments, R² is a bridged ring system selected from norbornane, norbornene, bicyclo[1.1.0]butane, bicyclo[3.2.0]heptane, bicyclo[3.3.0]octane, bicyclo[4.3.0]nonane, bicyclo[1.1.1]pentane, bicyclo[2.2.1]hexane, bicyclo[3.3.1]nonane, bicyclo[2.2.2]octane, adamantane, cubane, housane, any of which is optionally substituted with one or more R⁷. In some embodiments, the bridged ring system can have 1, 2, 3, 4, or more degrees of unsaturation.

In some embodiments, each of R⁴ or R⁵ may independently be hydrogen, C₁₋₁₀ alkyl, C₂₋₁₀ alkenyl, or C₂₋₁₀ alkynyl, each of which is optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₁₋₆ cycloalkyl, and C₁₋₆ cycloalkenyl.

In some embodiments, each of R⁴ or R⁵ may independently be 3- to 10-membered heterocycle. In some embodiments, each of R⁴ or R⁵ may independently be 3- to 10-membered heterocycle selected from azacyclopropane, pyrrolidine, piperidine, azepane, azocane, azonane, morpholine, thiomorpholine, 1,4-piperazine, 1,4-oxazepane, and 1,4-diazepane.

In some embodiments, the compound of Formula (III) is selected from:

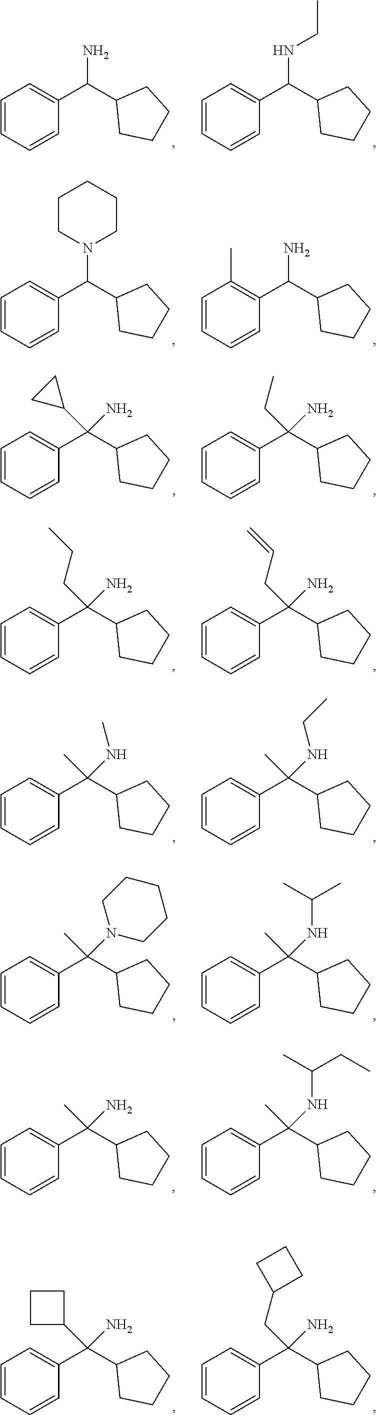

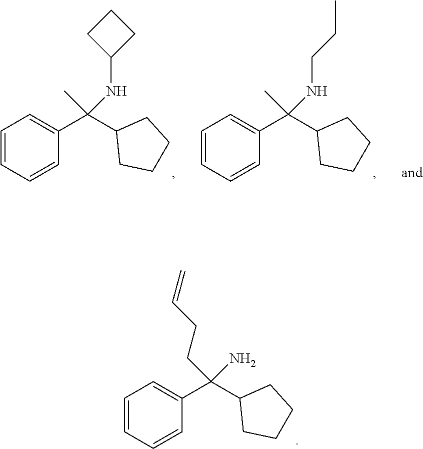

In another aspect, disclosed herein is a method for treating a physical, psychiatric, or neurological disorder comprising inhibiting an inotropic receptor by contacting the receptor with a pharmaceutical composition comprising a compound represented by the structure of Formula (IV):

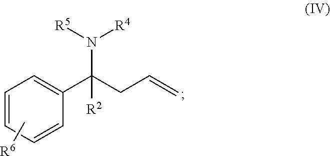

or an enantiomer, a mixture of enantiomers, or an isotopic variant thereof; or a pharmaceutically acceptable salt, solvate or hydrate thereof, wherein:
R² is selected from
   hydrogen;
   halogen;
   C₁₋₈ alkyl, C₂₋₈ alkenyl, and C₂₋₈ alkynyl, each of which is optionally substituted with one or more R⁷; and
   C₃₋₁₀ carbocycle and 3- to 10-membered heterocycle; wherein the C₃₋₁₀ carbocycle and 3-to 10-membered heterocycle are each optionally substituted with one or more R⁷;
R⁴, R⁵, R⁶, R⁷, and R⁸ are each independently selected from:
   hydrogen;
   C₁₋₁₀ alkyl, C₂₋₁₀ alkenyl, and C₂₋₁₀ alkynyl optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₁₋₆ cycloalkyl, and C₁₋₆ cycloalkenyl; and
   C₃₋₁₀ carbocycle or 3- to 10-membered heterocycle; wherein the C₃₋₁₀ carbocycle and 3- to 10-membered heterocycle are each optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), and C₁₋₈ alkyl.

In some embodiments, R² is hydrogen, C₁₋₈ alkyl, C₂₋₈ alkenyl, or C₂₋₈ alkynyl, each of which is optionally substituted with one or more R⁷.

In some embodiments, R² is C₃₋₇ carbocycle or 3- to 7-membered heterocycle; wherein the C₃₋₇ carbocycle and 3- to 7-membered heterocycle are each optionally substituted with one or more R⁷.

In some embodiments, R² is phenyl, optionally substituted with one or more R⁷.

In some embodiments, R² is a cycloalkyl. In some embodiments, R² is a cycloalkyl selected from cyclooctane, cycloheptane, cyclohexane, cyclopentane, cyclobutane, and cyclopropane, any of which is optionally substituted with one or more R⁷.

In some embodiments, R² is a cycloalkenyl. In some embodiments, R² is a cycloalkenyl selected from cyclooctene, cycloheptene, cyclohexene, cyclopentene, cyclobutene, and cyclopropane, any of which is optionally substituted with one or more R⁷. In some embodiments, the cycloalkenyl can have 1, 2, 3, 4, or more degrees of unsaturation.

In some embodiments, R² is a 6-membered heteroaryl. In some embodiments, R² is a 6-membered heteroaryl selected from pyridine, pyrazine, and pyrimidine, any of which is optionally substituted with one or more R⁷. In some embodiments, the heteroatom of the heteroaryl is not bound to the methylene carbon of Formula (IV).

In some embodiments, R² is a 5-membered heteroaryl. In some embodiments, R² is a 5-membered heteroaryl selected from thiophene, furan, pyrrole, pyrazole, selenophene, imidazole, thiazole, isothiazole, oxatriazole, oxadiazole, oxazole, and thiadiazole, any of which is optionally substituted with one or more R⁷. In some embodiments, the heteroatom of the heteroaryl is not bound to the methylene carbon of Formula (IV).

In some embodiments, R² is a 6-membered heterocycloalkenyl. In some embodiments, R² is a 6-membered heterocycloalkenyl selected from dihydropyridine, tetrahydropyridine, dihydropyridazine, tetrahydropyridazine, dihydropyrimidine, tetrahydropyrimidine, dihydropyrazine, tetrahydropyridazine, dihydropyrimidine, tetrahydropyrimidine, dihydropyrazine, tetrahydropyrazine, pyran, dihydropyran, thiopyran, dihydrothiopyran, dioxine, dihydrodioxine, oxazine, dihydrooxazine, thiazine, and dihydrothiazine, any of which is optionally substituted with one or more R⁷. In some embodiments, the heteroatom of the heterocycloalkenyl is not bound to the methylene carbon of Formula (IV).

In some embodiments, R² is a 5-membered heterocycloalkenyl. In some embodiments, R² is a 5-membered heterocycloalkenyl selected from pyrroline, pyrazoline, imidazoline, triazoline, dihydrofuran, dihydrothiophene, oxazoline, isooxazoline, thiazoline, isothiazoline, oxadiazoline, and thiadiazoline, any of which is optionally substituted with one or more R⁷. In some embodiments, the heteroatom of the heterocycloalkenyl is not bound to the methylene carbon of Formula (IV).

In some embodiments, R² is a 6-membered heterocycloalkyl. In some embodiments, R² is a 6-membered heterocycloalkyl selected from piperidine, piperazine, tetrahydrothiopyran, tetrahydropyran, dioxane, morpholine, and thiomorpholine, any of which is optionally substituted with one or more R⁷. In some embodiments, the heteroatom of the heterocycloalkyl is not bound to the methylene carbon of Formula (IV).

In some embodiments, R² is a 5-membered heterocycloalkyl. In some embodiments, R² is a 5-membered heterocycloalkyl selected from tetrahydrofuran, pyrrolidone, and tetrahydrothiophene, any of which is optionally substituted with one or more R⁷. In some embodiments, the heteroatom of the heterocycloalkyl is not bound to the methylene carbon of Formula (IV).

In some embodiments, R² is a 4-membered heterocycloalkyl. In some embodiments, R² is a 4-membered heterocycloalkyl selected from azetidine, oxetane, and thietane, any of which is optionally substituted with one or more R⁷. In some embodiments, the heteroatom of the heterocycloalkyl is not bound to the methylene carbon of Formula (IV).

In some embodiments, R² is a 6-6 fused ring system. In some embodiments, R² is a 6-6 fused ring system selected from naphthalene, quinoline, isoquinoline, pteridine, benzopyran, dihydroquinoline, dihydroisoquinoline, dihydronapthalene, and tetrahydronapthalene, any of which is optionally substituted with one or more R⁷.

In some embodiments, R² is a 5-6 fused ring system. In some embodiments, R² is a 5-6 fused ring system selected from benzimidazole, indole, azaindole, indazole, benzothiophene, benzofuran, benzoselenophene, benzimidazole, benzotriazole, benzothiazole, benzisothiazole, benzoxadiazole, benzoxazole, and benzothiadiazole, imidazo[4,5-b]-pyridine, imidazo[4,5-c]-pyridine, pyrazolo[3,4-b]pyridine, pyrazolo[3,4-c]pyridine, pyrazolo[4,3-b]pyridine, pyrazolo[4,3-b]pyridine, oxazolo[4,5-b]pyridine, oxazolo[4,5-c]pyridine, oxazolo[5,4-b]pyridine, oxazolo[5,4-c]pyridine, thiazolo[4,5-b]pyridine, thiazolo[4,5-c]pyridine, thiazolo[5,4-b]pyridine, thiazolo[5,4-c]pyridine, isooxazolo[4,5-b]pyridine, isooxazolo[4,5-c]pyridine, isooxazolo[3,4-b]pyridine, isooxazolo[3,4-c]pyridine, isooxazolo[4,3-b]pyridine, isooxazolo[4,3-c]pyridine, isooxazolo[5,4-b]pyridine, isooxazolo[5,4-c]pyridine, isothiazolo[4,5-b]pyridine, isothiazolo[4,5-c]pyridine, isothiazolo[5,4-b]pyridine, isothiazolo[5,4-c]pyridine, isothiazolo[3,4-b]pyridine, isothiazolo[3,4-c]pyridine, isothiazolo[4,3-b]pyridine, isothiazolo[4,3-c]pyridine and purine, any of which is optionally substituted with one or more R⁷.

In some embodiments, R² is a 5-5 fused ring system. In some embodiments, R² is a 5-5 fused ring system selected from pyrrolo[2,1-b]thiazole, pyrazolo[5,1-b]thiazole, imidazo[5,1-b]thiazole, imidazo[2,1-b]thiazole, thiazolo[3,2-b][1,2,4]triazole, thiazolo[3,2-c][1,2,4]triazole, imidazo[2,1-b]thiadiazole, imidazo[1,2-d][1,2,4]thiadiazole, thiazolo[3,2-d]tetrazole, thieno[3,2-b]thiophene, thieno[2,3-b]thiophene, thieno[3,4-b]thiophene, thieno[3,4-c]thiophene, any of which is optionally substituted with one or more R⁷.

In some embodiments, R² is a bridged ring system. In some embodiments, R² is a bridged ring system selected from norbornane, norbornene, bicyclo[1.1.0]butane, bicyclo[3.2.0]heptane, bicyclo[3.3.0]octane, bicyclo[4.3.0]nonane, bicyclo[1.1.1]pentane, bicyclo[2.2.1]hexane, bicyclo[3.3.1]nonane, bicyclo[2.2.2]octane, adamantane, cubane, housane, any of which is optionally substituted with one or more R⁷. In some embodiments, the bridged ring system can have 1, 2, 3, 4, or more degrees of unsaturation.

In some embodiments, each of R⁴ or R⁵ may independently be hydrogen, C₁₋₁₀ alkyl, C₂₋₁₀ alkenyl, or C₂₋₁₀ alkynyl, each of which is optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₁₋₆ cycloalkyl, and C₁₋₆ cycloalkenyl.

In some embodiments, each of R⁴ or R⁵ may independently be 3- to 10-membered heterocycle. In some embodiments, each of R⁴ or R⁵ may independently be 3- to 10-membered heterocycle selected from azacyclopropane, pyrrolidine, piperidine, azepane, azocane, azonane, morpholine, thiomorpholine, 1,4-piperazine, 1,4-oxazepane, and 1,4-diazepane.

In some embodiments, the compound of Formula (IV) is selected from:

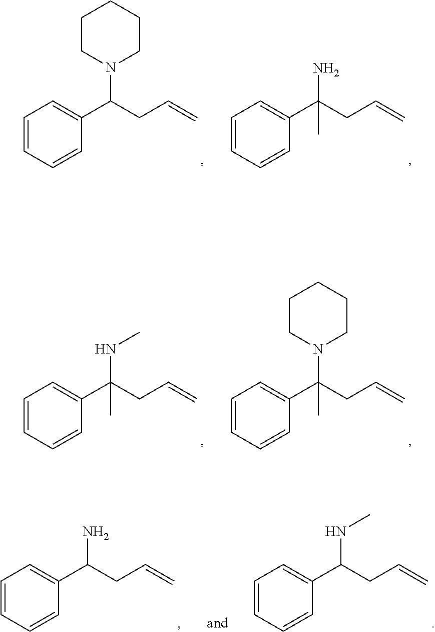

In another aspect, disclosed herein is a method for treating a physical, psychiatric, or neurological disorder comprising inhibiting an inotropic receptor by contacting the receptor with a pharmaceutical composition comprising a compound represented by Formula (V):

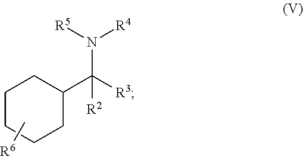

or an enantiomer, a mixture of enantiomers, or an isotopic variant thereof; or a pharmaceutically acceptable salt, solvate or hydrate thereof, wherein:
R² is selected from
   hydrogen;
   halogen;
   C₁₋₈ alkyl, C₂₋₈ alkenyl, and C₂₋₈ alkynyl, each of which is optionally substituted with one or more R⁷; and
   C₃₋₁₀ carbocycle and 3- to 10-membered heterocycle; wherein the C₃₋₁₀ carbocycle and 3-to 10-membered heterocycle are each optionally substituted with one or more R⁷;
R³ is selected from:
   C₁₋₆ alkyl, C₂₋₆ alkenyl, and C₂₋₆ alkynyl, each of which is optionally substituted with one or more R⁸; and
   C₃₋₁₀ carbocycle and 3- to 10-membered heterocycle; wherein the C₃₋₁₀ carbocycle and 3-to 10-membered heterocycle are each optionally substituted with one or more R¹;
R⁴, R⁵, R⁶, R⁷, and R⁸ are each independently selected from:
   hydrogen;
   C₁₋₁₀ alkyl, C₂₋₁₀ alkenyl, and C₂₋₁₀ alkynyl optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₁₋₆ cycloalkyl, and C₁₋₆ cycloalkenyl; and
   C₃₋₁₀ carbocycle or 3- to 10-membered heterocycle; wherein the C₃₋₁₀ carbocycle and 3- to 10-membered heterocycle are each optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), and C₁₋₈ alkyl.

In some embodiments, R² is hydrogen, C₁₋₈ alkyl, C₂₋₈ alkenyl, or C₂₋₈ alkynyl, each of which is optionally substituted with one or more R⁷.

In some embodiments, R² is C₃₋₇ carbocycle or 3- to 7-membered heterocycle; wherein the C₃₋₇ carbocycle and 3- to 7-membered heterocycle are each optionally substituted with one or more R⁷.

In some embodiments, R² is phenyl, optionally substituted with one or more R⁷.

In some embodiments, R² is a cycloalkyl. In some embodiments, R² is a cycloalkyl selected from cyclooctane, cycloheptane, cyclohexane, cyclopentane, cyclobutane, and cyclopropane, any of which is optionally substituted with one or more R⁷.

In some embodiments, R² is a cycloalkenyl. In some embodiments, R² is a cycloalkenyl selected from cyclooctene, cycloheptene, cyclohexene, cyclopentene, cyclobutene, and cyclopropane, any of which is optionally substituted with one or more R⁷. In some embodiments, the cycloalkenyl can have 1, 2, 3, 4, or more degrees of unsaturation.

In some embodiments, R² is a 6-membered heteroaryl. In some embodiments, R² is a 6-membered heteroaryl selected from pyridine, pyrazine, and pyrimidine, any of which is optionally substituted with one or more R⁷. In some embodiments, the heteroatom of the heteroaryl is not bound to the methylene carbon of Formula (V).

In some embodiments, R² is a 5-membered heteroaryl. In some embodiments, R² is a 5-membered heteroaryl selected from thiophene, furan, pyrrole, pyrazole, selenophene, imidazole, thiazole, isothiazole, oxatriazole, oxadiazole, oxazole, and thiadiazole, any of which is optionally substituted with one or more R⁷. In some embodiments, the heteroatom of the heteroaryl is not bound to the methylene carbon of Formula (V).

In some embodiments, R² is a 6-membered heterocycloalkenyl. In some embodiments, R² is a 6-membered heterocycloalkenyl selected from dihydropyridine, tetrahydropyridine, dihydropyridazine, tetrahydropyridazine, dihydropyrimidine, tetrahydropyrimidine, dihydropyrazine, tetrahydropyridazine, dihydropyrimidine, tetrahydropyrimidine, dihydropyrazine, tetrahydropyrazine, pyran, dihydropyran, thiopyran, dihydrothiopyran, dioxine, dihydrodioxine, oxazine, dihydrooxazine, thiazine, and dihydrothiazine, any of which is optionally substituted with one or more R⁷. In some embodiments, the heteroatom of the heterocycloalkenyl is not bound to the methylene carbon of Formula (V).

In some embodiments, R² is a 5-membered heterocycloalkenyl. In some embodiments, R² is a 5-membered heterocycloalkenyl selected from pyrroline, pyrazoline, imidazoline, triazoline, dihydrofuran, dihydrothiophene, oxazoline, isooxazoline, thiazoline, isothiazoline, oxadiazoline, and thiadiazoline, any of which is optionally substituted with one or more R⁷. In some embodiments, the heteroatom of the heterocycloalkenyl is not bound to the methylene carbon of Formula (V).

In some embodiments, R² is a 6-membered heterocycloalkyl. In some embodiments, R² is a 6-membered heterocycloalkyl selected from piperidine, piperazine, tetrahydrothiopyran, tetrahydropyran, dioxane, morpholine, and thiomorpholine, any of which is optionally substituted with one or more R⁷. In some embodiments, the heteroatom of the heterocycloalkyl is not bound to the methylene carbon of Formula (V).

In some embodiments, R² is a 5-membered heterocycloalkyl. In some embodiments, R² is a 5-membered heterocycloalkyl selected from tetrahydrofuran, pyrrolidone, and tetrahydrothiophene, any of which is optionally substituted with one or more R⁷. In some embodiments, the heteroatom of the heterocycloalkyl is not bound to the methylene carbon of Formula (V).

In some embodiments, R² is a 4-membered heterocycloalkyl. In some embodiments, R² is a 4-membered heterocycloalkyl selected from azetidine, oxetane, and thietane, any of which is optionally substituted with one or more R⁷. In some embodiments, the heteroatom of the heterocycloalkyl is not bound to the methylene carbon of Formula (V).

In some embodiments, R² is a 6-6 fused ring system. In some embodiments, R² is a 6-6 fused ring system selected from naphthalene, quinoline, isoquinoline, pteridine, benzopyran, dihydroquinoline, dihydroisoquinoline, dihydronapthalene, and tetrahydronapthalene, any of which is optionally substituted with one or more R⁷.

In some embodiments, R² is a 5-6 fused ring system. In some embodiments, R² is a 5-6 fused ring system selected from benzimidazole, indole, azaindole, indazole, benzothiophene, benzofuran, benzoselenophene, benzimidazole, benzotriazole, benzothiazole, benzisothiazole, benzoxadiazole, benzoxazole, and benzothiadiazole, imidazo[4,5-b]-pyridine, imidazo[4,5-c]-pyridine, pyrazolo[3,4-b]pyridine, pyrazolo[3,4-c]pyridine, pyrazolo[4,3-b]pyridine, pyrazolo[4,3-b]pyridine, oxazolo[4,5-b]pyridine, oxazolo[4,5-c]pyridine, oxazolo[5,4-b]pyridine, oxazolo[5,4-c]pyridine, thiazolo[4,5-b]pyridine, thiazolo[4,5-c]pyridine, thiazolo[5,4-b]pyridine, thiazolo[5,4-c]pyridine, isooxazolo[4,5-b]pyridine, isooxazolo[4,5-c]pyridine, isooxazolo[3,4-b]pyridine, isooxazolo[3,4-c]pyridine, isooxazolo[4,3-b]pyridine, isooxazolo[4,3-c]pyridine, isooxazolo[5,4-b]pyridine, isooxazolo[5,4-c]pyridine, isothiazolo[4,5-b]pyridine, isothiazolo[4,5-c]pyridine, isothiazolo[5,4-b]pyridine, isothiazolo[5,4-c]pyridine, isothiazolo[3,4-b]pyridine, isothiazolo[3,4-c]pyridine, isothiazolo[4,3-b]pyridine, isothiazolo[4,3-c]pyridine and purine, any of which is optionally substituted with one or more R⁷.

In some embodiments, R² is a 5-5 fused ring system. In some embodiments, R² is a 5-5 fused ring system selected from pyrrolo[2,1-b]thiazole, pyrazolo[5,1-b]thiazole, imidazo[5,1-b]thiazole, imidazo[2,1-b]thiazole, thiazolo[3,2-b][1,2,4]triazole, thiazolo[3,2-c][1,2,4]triazole, imidazo[2,1-b]thiadiazole, imidazo[1,2-d][1,2,4]thiadiazole, thiazolo[3,2-d]tetrazole, thieno[3,2-b]thiophene, thieno[2,3-b]thiophene, thieno[3,4-b]thiophene, thieno[3,4-c]thiophene, any of which is optionally substituted with one or more R⁷.

In some embodiments, R² is a bridged ring system. In some embodiments, R² is a bridged ring system selected from norbornane, bicyclo[1.1.0]butane, bicyclo[3.2.0]heptane, bicyclo[3.3.0]octane, bicyclo[4.3.0]nonane, bicyclo[1.1.1]pentane, bicyclo[2.2.1]hexane, bicyclo[3.3.1]nonane, bicyclo[2.2.2]octane, adamantane, cubane, housane, any of which is optionally substituted with one or more R⁷. In some embodiments, the bridged ring system can have 1, 2, 3, 4, or more degrees of unsaturation.

In some embodiments, R³ is hydrogen, C₁₋₆ alkyl, C₂₋₆ alkenyl, or C₂₋₆ alkynyl, each of which is optionally substituted with one or more R⁸.

In some embodiments, R³ is C₃₋₇ carbocycle or 3- to 7-membered heterocycle; wherein the C₃₋₇ carbocycle and 3- to 7-membered heterocycle are each optionally substituted with one or more R⁸.

In some embodiments, R³ is phenyl, optionally substituted with one or more R⁸.

In some embodiments, R³ is a cycloalkyl. In some embodiments, R³ is a cycloalkyl selected from cyclooctane, cycloheptane, cyclohexane, cyclopentane, cyclobutane, and cyclopropane, any of which is optionally substituted with one or more R⁸.

In some embodiments, R³ is a cycloalkenyl. In some embodiments, R³ is a cycloalkenyl selected from cyclooctene, cycloheptene, cyclohexene, cyclopentene, cyclobutene, and cyclopropane, any of which is optionally substituted with one or more R⁸. In some embodiments, the cycloalkenyl can have 1, 2, 3, 4, or more degrees of unsaturation.

In some embodiments, R³ is a 6-membered heteroaryl. In some embodiments, R³ is a 6-membered heteroaryl selected from pyridine, pyrazine, and pyrimidine, any of which is optionally substituted with one or more R⁸. In some embodiments, the heteroatom of the heteroaryl is not bound to the methylene carbon of Formula (V).

In some embodiments, R³ is a 5-membered heteroaryl. In some embodiments, R³ is a 5-membered heteroaryl selected from thiophene, furan, pyrrole, pyrazole, selenophene, imidazole, thiazole, isothiazole, oxatriazole, oxadiazole, oxazole, and thiadiazole, any of which is optionally substituted with one or more R⁸. In some embodiments, the heteroatom of the heteroaryl is not bound to the methylene carbon of Formula (V).

In some embodiments, R³ is a 6-membered heterocycloalkenyl. In some embodiments, R³ is a 6-membered heterocycloalkenyl selected from dihydropyridine, tetrahydropyridine, dihydropyridazine, tetrahydropyridazine, dihydropyrimidine, tetrahydropyrimidine, dihydropyrazine, tetrahydropyridazine, dihydropyrimidine, tetrahydropyrimidine, dihydropyrazine, tetrahydropyrazine, pyran, dihydropyran, thiopyran, dihydrothiopyran, dioxine, dihydrodioxine, oxazine, dihydrooxazine, thiazine, and dihydrothiazine, any of which is optionally substituted with one or more R⁸. In some embodiments, the heteroatom of the heterocycloalkenyl is not bound to the methylene carbon of Formula (V).

In some embodiments, R³ is a 5-membered heterocycloalkenyl. In some embodiments, R³ is a 5-membered heterocycloalkenyl selected from pyrroline, pyrazoline, imidazoline, triazoline, dihydrofuran, dihydrothiophene, oxazoline, isooxazoline, thiazoline, isothiazoline, oxadiazoline, and thiadiazoline, any of which is optionally substituted with one or more R⁸. In some embodiments, the heteroatom of the heterocycloalkenyl is not bound to the methylene carbon of Formula (V).

In some embodiments, R³ is a 6-membered heterocycloalkyl. In some embodiments, R³ is a 6-membered heterocycloalkyl selected from piperidine, piperazine, tetrahydrothiopyran, tetrahydropyran, dioxane, morpholine, and thiomorpholine, any of which is optionally substituted with one or more R⁸. In some embodiments, the heteroatom of the heterocycloalkyl is not bound to the methylene carbon of Formula (V).

In some embodiments, R³ is a 5-membered heterocycloalkyl. In some embodiments, R³ is a 5-membered heterocycloalkyl selected from tetrahydrofuran, pyrrolidone, and tetrahydrothiophene, any of which is optionally substituted with one or more R⁸. In some embodiments, the heteroatom of the heterocycloalkyl is not bound to the methylene carbon of Formula (V).

In some embodiments, R³ is a 4-membered heterocycloalkyl. In some embodiments, R³ is a 4-membered heterocycloalkyl selected from azetidine, oxetane, and thietane, any of which is optionally substituted with one or more R⁸. In some embodiments, the heteroatom of the heterocycloalkyl is not bound to the methylene carbon of Formula (V).

In some embodiments, R³ is a 6-6 fused ring system. In some embodiments, R³ is a 6-6 fused ring system selected from naphthalene, quinoline, isoquinoline, pteridine, benzopyran, dihydroquinoline, dihydroisoquinoline, dihydronapthalene, and tetrahydronapthalene, any of which is optionally substituted with one or more R⁸.

In some embodiments, R³ is a 5-6 fused ring system. In some embodiments, R³ is a 5-6 fused ring system selected from benzimidazole, indole, azaindole, indazole, benzothiophene, benzofuran, benzoselenophene, benzimidazole, benzotriazole, benzothiazole, benzisothiazole, benzoxadiazole, benzoxazole, and benzothiadiazole, imidazo[4,5-b]-pyridine, imidazo[4,5-c]-pyridine, pyrazolo[3,4-b]pyridine, pyrazolo[3,4-c]pyridine, pyrazolo[4,3-b]pyridine, pyrazolo[4,3-b]pyridine, oxazolo[4,5-b]pyridine, oxazolo[4,5-c]pyridine, oxazolo[5,4-b]pyridine, oxazolo[5,4-c]pyridine, thiazolo[4,5-b]pyridine, thiazolo[4,5-c]pyridine, thiazolo[5,4-b]pyridine, thiazolo[5,4-c]pyridine, isooxazolo[4,5-b]pyridine, isooxazolo[4,5-c]pyridine, isooxazolo[3,4-b]pyridine, isooxazolo[3,4-c]pyridine, isooxazolo[4,3-b]pyridine, isooxazolo[4,3-c]pyridine, isooxazolo[5,4-b]pyridine, isooxazolo[5,4-c]pyridine, isothiazolo[4,5-b]pyridine, isothiazolo[4,5-c]pyridine, isothiazolo[5,4-b]pyridine, isothiazolo[5,4-c]pyridine, isothiazolo[3,4-b]pyridine, isothiazolo[3,4-c]pyridine, isothiazolo[4,3-b]pyridine, isothiazolo[4,3-c]pyridine and purine, any of which is optionally substituted with one or more R⁸.

In some embodiments, R³ is a 5-5 fused ring system. In some embodiments, R³ is a 5-5 fused ring system selected from pyrrolo[2,1-b]thiazole, pyrazolo[5,1-b]thiazole, imidazo[5,1-b]thiazole, imidazo[2,1-b]thiazole, thiazolo[3,2-b][1,2,4]triazole, thiazolo[3,2-c][1,2,4]triazole, imidazo[2,1-b]thiadiazole, imidazo[1,2-d][1,2,4]thiadiazole, thiazolo[3,2-d]tetrazole, thieno[3,2-b]thiophene, thieno[2,3-b]thiophene, thieno[3,4-b]thiophene, thieno[3,4-c]thiophene, any of which is optionally substituted with one or more R⁸.

In some embodiments, R³ is a bridged ring system. In some embodiments, R³ is a bridged ring system selected from norbornane, norbornene, bicyclo[1.1.0]butane, bicyclo[3.2.0]heptane, bicyclo[3.3.0]octane, bicyclo[4.3.0]nonane, bicyclo[1.1.1]pentane, bicyclo[2.2.1]hexane, bicyclo[3.3.1]nonane, bicyclo[2.2.2]octane, adamantane, cubane, housane, any of which is optionally substituted with one or more R⁸. In some embodiments, the bridged ring system can have 1, 2, 3, 4, or more degrees of unsaturation.

In some embodiments, each of R⁴ or R⁵ may independently be hydrogen, C₁₋₁₀ alkyl, C₂₋₁₀ alkenyl, or C₂₋₁₀ alkynyl, each of which is optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₁₋₆ cycloalkyl, and C₁₋₆ cycloalkenyl.

In some embodiments, each of R⁴ or R⁵ may independently be 3- to 10-membered heterocycle. In some embodiments, each of R⁴ or R⁵ may independently be 3- to 10-membered heterocycle selected from azacyclopropane, pyrrolidine, piperidine, azepane, azocane, azonane, morpholine, thiomorpholine, and 1,4-piperazine, 1,4-oxazepane, 1,4-diazepane.

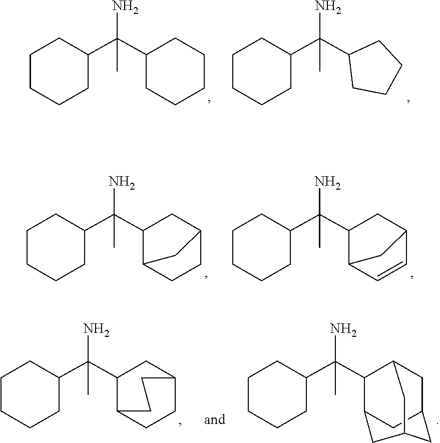

In some embodiments, compound of Formula (V) is selected from: NH₂ NH₂ NH₂ NH₂ NH₂ NH₂ and

In another aspect, disclosed herein is a method for treating a physical, psychiatric, or neurological disorder comprising inhibiting an inotropic receptor by contacting the receptor with a pharmaceutical composition comprising a compound represented by the structure of Formula (VI):

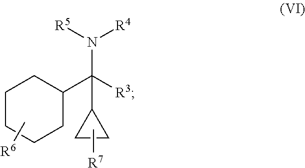

or an enantiomer, a mixture of enantiomers, or an isotopic variant thereof; or a pharmaceutically acceptable salt, solvate or hydrate thereof, wherein:
R³ is selected from:
   C₁₋₁₀ alkyl, C₂₋₁₀ alkenyl, and C₂₋₁₀ alkynyl, each of which is optionally substituted with one or more R⁸; and
   C₃₋₁₀ carbocycle and 3- to 10-membered heterocycle; wherein the C₃₋₁₀ carbocycle and 3-to 10-membered heterocycle are each optionally substituted with one or more R¹;
R⁴, R⁵, R⁶, R⁷, and R⁸ are each independently selected from:
   hydrogen;
   C₁₋₆ alkyl, C₂₋₆ alkenyl, and C₂₋₆ alkynyl optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₁₋₆ cycloalkyl, and C₁₋₆ cycloalkenyl; and
   C₃₋₁₀ carbocycle or 3- to 10-membered heterocycle; wherein the C₃₋₁₀ carbocycle and 3- to 10-membered heterocycle are each optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), and C₁₋₈ alkyl.

In some embodiments, R³ is hydrogen, C₁₋₆ alkyl, C₂₋₆ alkenyl, or C₂₋₆ alkynyl, each of which is optionally substituted with one or more R⁸.

In some embodiments, R³ is C₃₋₇ carbocycle or 3- to 7-membered heterocycle; wherein the C₃₋₇ carbocycle and 3- to 7-membered heterocycle are each optionally substituted with one or more R⁸.

In some embodiments, R³ is phenyl, optionally substituted with one or more R⁸.

In some embodiments, R³ is a cycloalkyl. In some embodiments, R³ is a cycloalkyl selected from cyclooctane, cycloheptane, cyclohexane, cyclopentane, cyclobutane, and cyclopropane, any of which is optionally substituted with one or more R⁸.

In some embodiments, R³ is a cycloalkenyl. In some embodiments, R³ is a cycloalkenyl selected from cyclooctene, cycloheptene, cyclohexene, cyclopentene, cyclobutene, and cyclopropane, any of which is optionally substituted with one or more R⁸. In some embodiments, the cycloalkenyl can have 1, 2, 3, 4, or more degrees of unsaturation.

In some embodiments, R³ is a 6-membered heteroaryl. In some embodiments, R³ is a 6-membered heteroaryl selected from pyridine, pyrazine, and pyrimidine, any of which is optionally substituted with one or more R⁸. In some embodiments, the heteroatom of the heteroaryl is not bound to the methylene carbon of Formula (VI).

In some embodiments, R³ is a 5-membered heteroaryl. In some embodiments, R³ is a 5-membered heteroaryl selected from thiophene, furan, pyrrole, pyrazole, selenophene, imidazole, thiazole, isothiazole, oxatriazole, oxadiazole, oxazole, and thiadiazole, any of which is optionally substituted with one or more R⁸. In some embodiments, the heteroatom of the heteroaryl is not bound to the methylene carbon of Formula (VI).

In some embodiments, R³ is a 6-membered heterocycloalkenyl. In some embodiments, R³ is a 6-membered heterocycloalkenyl selected from dihydropyridine, tetrahydropyridine, dihydropyridazine, tetrahydropyridazine, dihydropyrimidine, tetrahydropyrimidine, dihydropyrazine, tetrahydropyridazine, dihydropyrimidine, tetrahydropyrimidine, dihydropyrazine, tetrahydropyrazine, pyran, dihydropyran, thiopyran, dihydrothiopyran, dioxine, dihydrodioxine, oxazine, dihydrooxazine, thiazine, and dihydrothiazine, any of which is optionally substituted with one or more R⁸. In some embodiments, the heteroatom of the heterocycloalkenyl is not bound to the methylene carbon of Formula (VI).

In some embodiments, R³ is a 5-membered heterocycloalkenyl. In some embodiments, R³ is a 5-membered heterocycloalkenyl selected from pyrroline, pyrazoline, imidazoline, triazoline, dihydrofuran, dihydrothiophene, oxazoline, isooxazoline, thiazoline, isothiazoline, oxadiazoline, and thiadiazoline, any of which is optionally substituted with one or more R⁸. In some embodiments, the heteroatom of the heterocycloalkenyl is not bound to the methylene carbon of Formula (VI).

In some embodiments, R³ is a 6-membered heterocycloalkyl. In some embodiments, R³ is a 6-membered heterocycloalkyl selected from piperidine, piperazine, tetrahydrothiopyran, tetrahydropyran, dioxane, morpholine, and thiomorpholine, any of which is optionally substituted with one or more R⁸. In some embodiments, the heteroatom of the heterocycloalkyl is not bound to the methylene carbon of Formula (VI).

In some embodiments, R³ is a 5-membered heterocycloalkyl. In some embodiments, R³ is a 5-membered heterocycloalkyl selected from tetrahydrofuran, pyrrolidone, and tetrahydrothiophene, any of which is optionally substituted with one or more R⁸. In some embodiments, the heteroatom of the heterocycloalkyl is not bound to the methylene carbon of Formula (VI).

In some embodiments, R³ is a 4-membered heterocycloalkyl. In some embodiments, R³ is a 4-membered heterocycloalkyl selected from azetidine, oxetane, and thietane, any of which is optionally substituted with one or more R⁸. In some embodiments, the heteroatom of the heterocycloalkyl is not bound to the methylene carbon of Formula (VI).

In some embodiments, R³ is a 6-6 fused ring system. In some embodiments, R³ is a 6-6 fused ring system selected from naphthalene, quinoline, isoquinoline, pteridine, benzopyran, dihydroquinoline, dihydroisoquinoline, dihydronapthalene, and tetrahydronapthalene, any of which is optionally substituted with one or more R⁸.

In some embodiments, R³ is a 5-6 fused ring system. In some embodiments, R³ is a 5-6 fused ring system selected from benzimidazole, indole, azaindole, indazole, benzothiophene, benzofuran, benzoselenophene, benzimidazole, benzotriazole, benzothiazole, benzisothiazole, benzoxadiazole, benzoxazole, and benzothiadiazole, imidazo[4,5-b]-pyridine, imidazo[4,5-c]-pyridine, pyrazolo[3,4-b]pyridine, pyrazolo[3,4-c]pyridine, pyrazolo[4,3-b]pyridine, pyrazolo[4,3-b]pyridine, oxazolo[4,5-b]pyridine, oxazolo[4,5-c]pyridine, oxazolo[5,4-b]pyridine, oxazolo[5,4-c]pyridine, thiazolo[4,5-b]pyridine, thiazolo[4,5-c]pyridine, thiazolo[5,4-b]pyridine, thiazolo[5,4-c]pyridine, isooxazolo[4,5-b]pyridine, isooxazolo[4,5-c]pyridine, isooxazolo[3,4-b]pyridine, isooxazolo[3,4-c]pyridine, isooxazolo[4,3-b]pyridine, isooxazolo[4,3-c]pyridine, isooxazolo[5,4-b]pyridine, isooxazolo[5,4-c]pyridine, isothiazolo[4,5-b]pyridine, isothiazolo[4,5-c]pyridine, isothiazolo[5,4-b]pyridine, isothiazolo[5,4-c]pyridine, isothiazolo[3,4-b]pyridine, isothiazolo[3,4-c]pyridine, isothiazolo[4,3-b]pyridine, isothiazolo[4,3-c]pyridine and purine, any of which is optionally substituted with one or more R⁸.

In some embodiments, R³ is a 5-5 fused ring system. In some embodiments, R³ is a 5-5 fused ring system selected from pyrrolo[2,1-b]thiazole, pyrazolo[5,1-b]thiazole, imidazo[5,1-b]thiazole, imidazo[2,1-b]thiazole, thiazolo[3,2-b][1,2,4]triazole, thiazolo[3,2-c][1,2,4]triazole, imidazo[2,1-b]thiadiazole, imidazo[1,2-d][1,2,4]thiadiazole, thiazolo[3,2-d]tetrazole, thieno[3,2-b]thiophene, thieno[2,3-b]thiophene, thieno[3,4-b]thiophene, thieno[3,4-c]thiophene, any of which is optionally substituted with one or more R⁸.

In some embodiments, R³ is a bridged ring system. In some embodiments, R³ is a bridged ring system selected from norbornane, norbornene, bicyclo[1.1.0]butane, bicyclo[3.2.0]heptane, bicyclo[3.3.0]octane, bicyclo[4.3.0]nonane, bicyclo[1.1.1]pentane, bicyclo[2.2.1]hexane, bicyclo[3.3.1]nonane, bicyclo[2.2.2]octane, adamantane, cubane, housane, any of which is optionally substituted with one or more R⁸. In some embodiments, the bridged ring system can have 1, 2, 3, 4, or more degrees of unsaturation.

In some embodiments, each of R⁴ or R⁵ may independently be hydrogen, C₁₋₁₀ alkyl, C₂₋₁₀ alkenyl, or C₂₋₁₀ alkynyl, each of which is optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₁₋₆ cycloalkyl, and C₁₋₆ cycloalkenyl.

In some embodiments, each of R⁴ or R⁵ may independently be 3- to 10-membered heterocycle. In some embodiments, each of R⁴ or R⁵ may independently be 3- to 10-membered heterocycle selected from azacyclopropane, pyrrolidine, piperidine, azepane, azocane, azonane, morpholine, thiomorpholine, 1,4-piperazine, 1,4-oxazepane, and 1,4-diazepane.

In some embodiments, compound of Formula (VI) is selected from:

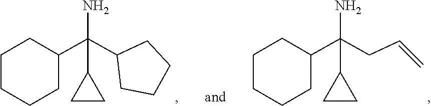 [SMILES: NC(C1CCCCC1)(C1CCCC1)C1CC1 ; C=CCC(N)(C1CCCCC1)C1CC1]

In some embodiments, provided herein is a method of treating a physical, psychiatric, or neurological disorder comprises administering a composition comprising a compound of Table 1. In some embodiments, provided herein is a method of treating a physical, psychiatric, or neurological disorder comprises administering a composition comprising a compound of Table 1a.

In some aspects, the method of treating a physical, psychiatric, or neurological disorder comprises reducing an activity of a N-methyl-D-aspartate receptor (NMDAR). An inhibitor compound or salt of Formula (I), (II), (III), (IV), (V), or (VI) may inhibit activity of NMDAR by about 5% to about 90% relative to pre-treatment value or an untreated control NMDAR. In some instances, an inhibitor or antagonist compound of Formula (I), (II), (III), (IV), (V), or (VI) may inhibit activity of NMDAR by about 5% to about 90% relative to ketamine, phencyclidine (PCP), or other inhibitor of NMDAR. An inhibitor may inhibit activity of NMDAR by at least about 5% relative to pre-treatment value or an untreated control NMDAR, such as without any agonist or antagonist bound. An inhibitor may inhibit activity of NMDAR by at most about 90% relative to pre-treatment value or an untreated control NMDAR, such as without any agonist or antagonist bound. An inhibitor may inhibit activity of NMDAR by about 5% to about 10%, about 5% to about 20%, about 5% to about 30%, about 5% to about 40%, about 5% to about 50%, about 5% to about 60%, about 5% to about 70%, about 5% to about 80%, about 5% to about 90%, about 10% to about 20%, about 10% to about 30%, about 10% to about 40%, about 10% to about 50%, about 10% to about 60%, about 10% to about 70%, about 10% to about 80%, about 10% to about 90%, about 20% to about 30%, about 20% to about 40%, about 20% to about 50%, about 20% to about 60%, about 20% to about 70%, about 20% to about 80%, about 20% to about 90%, about 30% to about 40%, about 30% to about 50%, about 30% to about 60%, about 30% to about 70%, about 30% to about 80%, about 30% to about 90%, about 40% to about 50%, about 40% to about 60%, about 40% to about 70%, about 40% to about 80%, about 40% to about 90%, about 50% to about 60%, about 50% to about 70%, about 50% to about 80%, about 50% to about 90%, about 60% to about 70%, about 60% to about 80%, about 60% to about 90%, about 70% to about 80%, about 70% to about 90%, or about 80% to about 90% relative to pre-treatment capacity or an untreated control NMDAR. An inhibitor may inhibit activity of NMDAR by about 5%, about 10%, about 20%, about 30%, about 40%, about 50%, about 60%, about 70%, about 80%, or about 90% relative to pre-treatment capacity or an untreated control NMDAR.

In some embodiments, the compounds of Formula (I), (II), (III), (IV), (V), or (VI) show PCP site NMDAR binding affinities (e.g., measured as a dissociation constant Kd or Ki) in the range of 0.1-100,000. In some instances, the inhibitors may fully inhibit NMDARs. In some embodiments, the inhibitors may inhibit 100% activity of NMDARs. In some embodiments, the compound may inhibit activity NMDAR such as by competitively binding to NMDAR, such as displacing MK-801 or TCP. In some embodiments, the compounds of Formula (I), (II), (III), (IV), (V), or (VI) may displace a ligand bound to NMDAR. In some embodiments, the compound binds to the PCP site within the NMDAR, thus acting as a channel blocker. The binding activity of the compound may be determined by a radioligand binding assay. In some embodiments, the binding activity of the compound may be determined by a competitive radioligand binding assay. In some instances, the measured activity of the compound may be determined by a competitive radioligand binding assay relative to ³H-MK-801 or ³H-TCP. In some embodiments, the compound binds to NMDAR with a binding affinity (e.g., Kᵢ). In some embodiments, the compound binds with a Kᵢ less than 100 μM. In some embodiments, the compound binds with a Kᵢ of about 0.1 nM to about 1×10⁶ nM, about 0.2 nM to about 9×10⁵ nM, about 0.3 nM to about 8×10⁵ nM, about 0.4 nM to about 7×10⁵ nM, about 0.5 nM to about 6×10⁵ nM, about 0.6 nM to about 5×10⁵ nM, about 0.7 nM to about 4×10⁵ nM, about 0.8 nM to about 3×10⁵ nM, about 0.9 nM to about 2×10⁵ nM, about 1 nM to about 1×10⁵ nM, about 5 nM to about 5×10⁴ nM, about 10 nM to about 1×10³ nM, about 50 nM to about 5×10³ nM, about 100 nM to about 2×10³ nM, or about 500 nM to about 1×10³ nM. In some embodiments, the compound may bind to NMDARs with a Kᵢ of about 5×10⁴ nM, about 2×10⁴ nM, about 1×10⁴ nM, 5×10³ nM, about 2×10³ nM, about 1×10³ nM, about 500 nM, about 200 nM, about 100 nM, about 50 nM, about 20 nM, or about 10 nM.

In some aspects, the method of treating a physical, psychiatric, or neurological disorder comprises reducing an activity of a dopamine transporter (DAT). An inhibitor compound or salt of Formula (I), (II), (III), (IV), (V), or (VI) may inhibit activity of DAT by about 5% to about 100% relative to pre-treatment value or an untreated control DAT. In some instances, an inhibitor or antagonist compound of Formula (I), (II), (III), (IV), (V), or (VI) may inhibit activity of DAT by about 5% to about 90% relative to desipramine, GBR 12935, cocaine, methylphenidate, bupropion 12935 or other inhibitor of DAT. An inhibitor may inhibit activity of DAT by at least about 5% relative to pre-treatment value or an untreated control DAT. An inhibitor may inhibit activity of DAT by at most about 90% relative to pre-treatment value or an untreated control DAT. An inhibitor may inhibit activity of DAT at a physiologically relevant concentration by about 5% to about 10%, about 5% to about 20%, about 5% to about 30%, about 5% to about 40%, about 5% to about 50%, about 5% to about 60%, about 5% to about 70%, about 5% to about 80%, about 5% to about 90%, about 10% to about 20%, about 10% to about 30%, about 10% to about 40%, about 10% to about 50%, about 10% to about 60%, about 10% to about 70%, about 10% to about 80%, about 10% to about 90%, about 10% to about 100%, about 20% to about 30%, about 20% to about 40%, about 20% to about 50%, about 20% to about 60%, about 20% to about 70%, about 20% to about 80%, about 20% to about 90%, about 30% to about 40%, about 30% to about 50%, about 30% to about 60%, about 30% to about 70%, about 30% to about 80%, about 30% to about 90%, about 40% to about 50%, about 40% to about 60%, about 40% to about 70%, about 40% to about 80%, about 40% to about 90%, about 50% to about 60%, about 50% to about 70%, about 50% to about 80%, about 50% to about 90%, about 50% to about 100%, about 60% to about 70%, about 60% to about 80%, about 60% to about 90%, about 60% to about 100%, about 70% to about 80%, about 70% to about 90%, or about 80% to about 90% relative to pre-treatment capacity or an untreated control DAT. In some embodiments, an inhibitor may inhibit DAT activity at physiologically relevant concentrations by about 60% to about 100%, about 70% to about 100%, about 80% to about 100%, about 60% to about 90%, about 70% to about 90%, or about 50% to about 100%. An inhibitor may inhibit activity of DAT by about 5%, about 10%, about 20%, about 30%, about 40%, about 50%, about 60%, about 70%, about 80%, or about 90% relative to pre-treatment capacity or an untreated control DAT. A physiologically relevant inhibitor of DAT may have a potency estimate, such as a receptor binding affinity (e.g., K_{d} or Kᵢ) of about 0.1 to about 100,000 nM. The binding affinity of the compounds disclosed herein may be determined by a radioligand binding assay. Alternatively, potency to inhibit substrate uptake at DAT (IC₅₀) can be measured from a transporter functional assay. In some embodiments, the binding affinity of the compounds disclosed herein may be determined by a competitive radioligand binding assay or a functional assay of DAT transport.

In some aspects, the method of treating a physical, psychiatric, or neurological disorder comprises binding to and reducing an activity of an ionotropic receptor, such as NMDAR, or a transporter such as DAT, or SERT. An inhibitor of Formula (I), (II), (III), (IV), (V), or (VI) may show selectivity for NMDARs relative to SERT and/or DAT by about 1-fold, 2-fold, 3-fold, 5-fold, 10-fold, about 20-fold, about 30-fold, about 50-fold, about 100-fold, about 200-fold, about 300-fold, about 500-fold, about 1,000-fold, about 2,000-fold, about 3,000-fold, 5,000-fold or about 10,000-fold. In some embodiments, the inhibitor reduces activity of the ionotropic receptor with selectivity for NMDAR relative to SERT and/or DAT by about 10-fold to about 1,000-fold, by about 20-fold to about 500-fold, by about 30-fold to about 300-fold, by about 50-fold to about 200-fold, by about 10-fold to about 500-fold, by about 20-fold to about 1,000-fold, by about 30-fold to about 2,000-fold, by about 50-fold to about 5,000-fold, by about 1,000-fold to about 5,000-fold, by about 1-fold to about 10-fold, by about 2-fold to about 20-fold, by about 3-fold to about 30-fold, or by about 5-fold to about 50-fold. The binding affinity of the compounds disclosed herein may be determined by a radioligand binding assay. In some embodiments, the binding affinity of the compounds disclosed herein may be determined by a competitive radioligand binding assays. In some instances, selectivity can be determined using functional assays of channel or transporter activity. In some instances, selectivity can be determined using a combination of radioligand binding and functional assays. In some instances, the inhibitor of Formula (I), (II), (III), (IV), (V), or (VI) selectively inhibits NMDARs with selectivity over other targets, such as the monoamine transporters DAT or SERT. In some instances, the inhibitor of Formula (I), (II), (III), (IV), (V), or (VI) shows polypharmacology for NMDAR and one or more additional receptors or transporters such as the monoamine transporters DAT, NET and SERT.

In some aspects, the method of treating a physical, psychiatric, or neurological disorder comprises reducing an activity of a serotonin receptor (SERT). An inhibitor compound or salt of Formula (I), (II), (III), (IV), (V), or (VI) may inhibit activity of SERT by about 5% to about 100% relative to pre-treatment value or an untreated control SERT. In some instances, an inhibitor or antagonist compound of Formula (I), (II), (III), (IV), (V), or (VI) may inhibit activity of SERT at a physiologically relevant concentration by about 5% to about 90% relative to untreated control. An inhibitor may inhibit activity of SERT at a physiologically relevant concentration by at least about 5% relative to pre-treatment value or an untreated control SERT, such as without any inhibitor bound. An inhibitor may inhibit activity of SERT at a physiologically relevant concentration by up to 100% relative to pre-treatment value or an untreated control SERT, such as without any inhibitor bound. An inhibitor may inhibit activity of SERT at a physiologically relevant concentration by about 5% to about 10%, about 5% to about 20%, about 5% to about 30%, about 5% to about 40%, about 5% to about 50%, about 5% to about 60%, about 5% to about 70%, about 5% to about 80%, about 5% to about 90%, about 10% to about 20%, about 10% to about 30%, about 10% to about 40%, about 10% to about 50%, about 10% to about 60%, about 10% to about 70%, about 10% to about 80%, about 10% to about 90%, about 20% to about 30%, about 20% to about 40%, about 20% to about 50%, about 20% to about 60%, about 20% to about 70%, about 20% to about 80%, about 20% to about 90%, about 30% to about 40%, about 30% to about 50%, about 30% to about 60%, about 30% to about 70%, about 30% to about 80%, about 30% to about 90%, about 40% to about 50%, about 40% to about 60%, about 40% to about 70%, about 40% to about 80%, about 40% to about 90%, about 50% to about 60%, about 50% to about 70%, about 50% to about 80%, about 50% to about 90%, about 60% to about 70%, about 60% to about 80%, about 60% to about 90%, about 70% to about 80%, about 70% to about 90%, or about 80% to about 90% relative to pre-treatment capacity or an untreated control SERT. An inhibitor may inhibit activity of SERT at a physiologically relevant concentration by about 5%, about 10%, about 20%, about 30%, about 40%, about 50%, about 60%, about 70%, about 80%, about 90%, or about 100% relative to pre-treatment capacity or an untreated control. A physiologically relevant inhibitor of SERT may have a potency estimate, such as a receptor binding affinity (e.g., K_{d} or Kᵢ) of about 0.1 to about 100,000 nM. The binding activity of the compounds disclosed herein may be determined by a radioligand binding assay. In some embodiments, the binding activity of the compounds disclosed herein may be determined by a competitive radioligand binding assay.

In some embodiments, the method of treating a physical, psychiatric, or neurological disorder comprises selectively binding to an NMDAR as compared to another ionotropic receptor. In some embodiments the other ionotropic receptor comprises SERT or DAT. In some embodiments, at a given physiologically relevant concentration, the compound selectively binds to NMDAR by about 10% to about 100%, by about 20% to about 100%, by about 30% to about 100%, by about 40% to about 100%, by about 50% to about 100%, by about 60% to about 100%, by about 70% to about 100%, by about 80% to about 100%, by about 90% to about 100%, by about 10% to about 90%, by about 20% to about 90%, by about 30% to about 90%, by about 40% to about 90%, by about 50% to about 90%, by about 60% to about 90%, by about 70% to about 90%, by about 80% to about 90%, by about 20% to about 80%, by about 30% to about 80%, by about 40% to about 80%, by about 50% to about 80%, by about 60% to about 80%, by about 70% to about 80%, by about 80% to about 80%, by about 20% to about 70%, by about 30% to about 70%, by about 40% to about 70%, by about 50% to about 70%, by about 60% to about 70%, by about 70% to about 70%, or by about 80% to about 70% as compared to binding of the compound to another ionotropic receptor. In some embodiments, binding of the compound to NMDAR may be about 10%, about 20%, about 30%, about 40%, about 50%, about 60%, about 70%, about 80%, about 90%, or about 100% greater than binding of the compound to another ionotropic receptor.

In some aspects, the method of the method of treating a physical, psychiatric, or neurological disorder comprises binding an compound of Formula (I), (II), (III), (IV), (V), or (VI) to NMDARs with a Kᵢ of about 0.1 nM to about 100,000 nM. In some embodiments, the inhibitor may bind to NMDARs with a Kᵢ of about 0.1 nM to about 1×10⁶ nM, about 0.2 nM to about 9×10⁵ nM, about 0.3 nM to about 8×10⁵ nM, about 0.4 nM to about 7×10⁵ nM, about 0.5 nM to about 6×10⁵ nM, about 0.6 nM to about 5×10⁵ nM, about 0.7 nM to about 4×10⁵ nM, about 0.8 nM to about 3×10⁵ nM, about 0.9 nM to about 2×10⁵ nM, about 1 nM to about 1×10⁵ nM, about 5 nM to about 5×10⁴ nM, about 10 nM to about 1×10³ nM, about 50 nM to about 5×10³ nM, about 100 nM to about 2×10³ nM, or about 500 nM to about 1×10³ nM. In some embodiments, the compound may bind to NMDARs with a Kᵢ of about 5×10⁴ nM, about 2×10⁴ nM, about 1×10⁴ nM, 5×10³ nM, about 2×10³ nM, about 1×10³ nM, about 500 nM, about 200 nM, about 100 nM, about 50 nM, about 20 nM, or about 10 nM. The binding affinity of the compounds disclosed herein may be determined by a radioligand binding assay. In some embodiments, the binding affinity of the compounds disclosed herein may be determined by a competitive radioligand binding assay.

### DEFINITIONS

Unless defined otherwise, all technical and scientific terms used herein have the same meaning as is commonly understood by one of skill in the art to which this disclosure belongs.

As used in the specification and claims, the singular form “a”, “an” and “the” includes plural references unless the context clearly dictates otherwise.

The term “C_{x-y}” or “Cₓ-C_{y}” when used in conjunction with a chemical moiety, such as alkyl, alkenyl, or alkynyl is meant to include groups that contain from x to y carbons in the chain.

For example, the term “C₁₋₆alkyl” refers to substituted or unsubstituted saturated hydrocarbon groups, including straight-chain alkyl and branched-chain alkyl groups that contain from 1 to 6 carbons. The term —C_{x-y}alkylene-refers to a substituted or unsubstituted alkylene chain with from x to y carbons in the alkylene chain. For example —C₁₋₆alkylene-may be selected from methylene, ethylene, propylene, butylene, pentylene, and hexylene, any one of which is optionally substituted.

The terms “C_{x-y} alkenyl” and “C_{x-y} alkynyl” refer to substituted or unsubstituted unsaturated aliphatic groups analogous in length and possible substitution to the alkyls described above, but that contain at least one double or triple bond, respectively. The term —C_{x-y}alkenylene-refers to a substituted or unsubstituted alkenylene chain with from x to y carbons in the alkenylene chain. For example, —C₂₋₆alkenylene-may be selected from ethenylene, propenylene, butenylene, pentenylene, and hexenylene, any one of which is optionally substituted. An alkenylene chain may have one double bond or more than one double bond in the alkenylene chain. The term —C_{x-y}alkynylene-refers to a substituted or unsubstituted alkynylene chain with from x to y carbons in the alkenylene chain. For example, —C₂₋₆alkynylene-may be selected from ethynylene, propynylene, butynylene, pentynylene, and hexynylene, any one of which is optionally substituted. An alkynylene chain may have one triple bond or more than one triple bond in the alkynylene chain.

“Alkylene” refers to a straight divalent hydrocarbon chain linking the rest of the molecule to a radical group, consisting solely of carbon and hydrogen, containing no unsaturation, and having from one to twelve carbon atoms, for example, methylene, ethylene, propylene, butylene, and the like. The alkylene chain is attached to the rest of the molecule through a single bond and to the radical group through a single bond. The points of attachment of the alkylene chain to the rest of the molecule and to the radical group are through the terminal carbons respectively. In other embodiments, an alkylene comprises one to five carbon atoms (i.e., C₁-C₅ alkylene). In other embodiments, an alkylene comprises one to four carbon atoms (i.e., C₁-C₄ alkylene). In other embodiments, an alkylene comprises one to three carbon atoms (i.e., C₁-C₃ alkylene). In other embodiments, an alkylene comprises one to two carbon atoms (i.e., C₁-C₂ alkylene). In other embodiments, an alkylene comprises one carbon atom (i.e., C₁ alkylene). In other embodiments, an alkylene comprises five to eight carbon atoms (i.e., C₅-C₈ alkylene). In other embodiments, an alkylene comprises two to five carbon atoms (i.e., C₂-C₅ alkylene). In other embodiments, an alkylene comprises three to five carbon atoms (i.e., C₃-C₅ alkylene).

Unless stated otherwise specifically in the specification, an alkylene chain is optionally substituted by one or more substituents such as those substituents described herein.

“Alkenylene” refers to a straight divalent hydrocarbon chain linking the rest of the molecule to a radical group, consisting solely of carbon and hydrogen, containing at least one carbon-carbon double bond, and having from two to twelve carbon atoms. The alkenylene chain is attached to the rest of the molecule through a single bond and to the radical group through a single bond. The points of attachment of the alkenylene chain to the rest of the molecule and to the radical group are through the terminal carbons, respectively. In other embodiments, an alkenylene comprises two to five carbon atoms (i.e., C₂-C₅ alkenylene). In other embodiments, an alkenylene comprises two to four carbon atoms (i.e., C₂-C₄ alkenylene). In other embodiments, an alkenylene comprises two to three carbon atoms (i.e., C₂-C₃ alkenylene). In other embodiments, an alkenylene comprises two carbon atoms (i.e., C₂ alkenylene). In other embodiments, an alkenylene comprises five to eight carbon atoms (i.e., C₅-C₈ alkenylene). In other embodiments, an alkenylene comprises three to five carbon atoms (i.e., C₃-C₅ alkenylene).

Unless stated otherwise specifically in the specification, an alkenylene chain is optionally substituted by one or more substituents such as those substituents described herein.

“Alkynylene” refers to a straight divalent hydrocarbon chain linking the rest of the molecule to a radical group, consisting solely of carbon and hydrogen, containing at least one carbon-carbon triple bond, and having from two to twelve carbon atoms. The alkynylene chain is attached to the rest of the molecule through a single bond and to the radical group through a single bond. The points of attachment of the alkynylene chain to the rest of the molecule and to the radical group are through the terminal carbons respectively. In other embodiments, an alkynylene comprises two to five carbon atoms (i.e., C₂-C₅ alkynylene). In other embodiments, an alkynylene comprises two to four carbon atoms (i.e., C₂-C₄ alkynylene). In other embodiments, an alkynylene comprises two to three carbon atoms (i.e., C₂-C₃ alkynylene). In other embodiments, an alkynylene comprises two carbon atoms (i.e., C₂ alkynylene). In other embodiments, an alkynylene comprises five to eight carbon atoms (i.e., C₅-C₈ alkynylene). In other embodiments, an alkynylene comprises three to five carbon atoms (i.e., C₃-C₅ alkynylene).

Unless stated otherwise specifically in the specification, an alkynylene chain is optionally substituted by one or more substituents such as those substituents described herein.

The term “carbocycle” as used herein refers to a saturated, unsaturated or aromatic ring in which each atom of the ring is carbon. Carbocycle includes 3- to 10-membered monocyclic rings, 5- to 12-membered bicyclic rings, spiro bicycles, and 5- to 12-membered bridged rings.

Each ring of a bicyclic carbocycle may be selected from saturated, unsaturated, and aromatic rings. In some embodiments, an aromatic ring, e.g., phenyl, may be fused to a saturated or unsaturated ring, e.g., cyclohexane, cyclopentane, or cyclohexene. A bicyclic carbocycle includes any combination of saturated, unsaturated and aromatic bicyclic rings, as valence permits. A bicyclic carbocycle further includes spiro bicylic rings such as spiropentane. A bicyclic carbocycle includes any combination of ring sizes such as 3-3 spiro ring systems, 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. In some embodiments, carbocycles include cyclopropyl, cyclobutyl, cyclopentyl, cyclohexyl, cyclohexenyl, norbornyl, adamantyl, phenyl, indanyl, naphthyl, and bicyclo[1.1.1]pentanyl.

The term “aryl” refers to an aromatic monocyclic or aromatic multicyclic hydrocarbon ring system. The aromatic monocyclic or aromatic multicyclic hydrocarbon ring system contains only hydrogen and carbon and from five to eighteen carbon atoms, where at least one of the rings in the ring system is aromatic, i.e., it contains a cyclic, delocalized (4n+2) 7π-electron system in accordance with the Hückel theory. The ring system from which aryl groups are derived include, but are not limited to, groups such as benzene, fluorene, indane, indene, tetralin and naphthalene. Unless stated otherwise specifically in the specification, the term “aryl” or the prefix “ar” (such as in “aralkyl”) is meant to include aryl radicals optionally substituted by one or more substituents such as those substituents described herein.

The term “cycloalkyl” refers to a saturated ring in which each atom of the ring is carbon. Cycloalkyl may include monocyclic and polycyclic rings such as 3- to 10-membered monocyclic rings, 5- to 12-membered bicyclic rings, spiro bicycles, and 5- to 12-membered bridged rings. In certain embodiments, a cycloalkyl comprises three to ten carbon atoms. In other embodiments, a cycloalkyl comprises five to seven carbon atoms. The cycloalkyl may be attached to the rest of the molecule by a single bond. Examples of monocyclic cycloalkyls include, e.g., cyclopropyl, cyclobutyl, cyclopentyl, cyclohexyl, cycloheptyl, and cyclooctyl. Polycyclic cycloalkyl radicals include, for example, adamantyl, spiropentane, norbornyl (i.e., bicyclo[2.2.1]heptanyl), decalinyl, 7,7 dimethyl bicyclo[2.2.1]heptanyl, bicyclo[1.1.1]pentanyl, and the like. Unless otherwise stated specifically in the specification, the term “cycloalkyl” is meant to include cycloalkyl radicals that are optionally substituted by one or more substituents such as those substituents described herein.

The term “cycloalkenyl” refers to a saturated ring in which each atom of the ring is carbon and there is at least one double bond between two ring carbons. Cycloalkenyl may include monocyclic and polycyclic rings such as 3- to 10-membered monocyclic rings, 6- to 12-membered bicyclic rings, and 5- to 12-membered bridged rings. In other embodiments, a cycloalkenyl comprises five to seven carbon atoms. The cycloalkenyl may be attached to the rest of the molecule by a single bond. Examples of monocyclic cycloalkenyls include, e.g., cyclopentenyl, cyclohexenyl, cycloheptenyl, and cyclooctenyl. Unless otherwise stated specifically in the specification, the term “cycloalkenyl” is meant to include cycloalkenyl radicals that are optionally substituted by one or more substituents such as those substituents described herein.

The term “halo” or, alternatively, “halogen” or “halide,” means fluoro, chloro, bromo or iodo. In some embodiments, halo is fluoro, chloro, or bromo.

The term “haloalkyl” refers to an alkyl radical, as defined above, that is substituted by one or more halo radicals, for example, trifluoromethyl, dichloromethyl, bromomethyl, 2,2,2-trifluoroethyl, 1-chloromethyl-2-fluoroethyl, and the like. In some embodiments, the alkyl part of the haloalkyl radical is optionally further substituted as described herein.

The term “heterocycle” as used herein refers to a saturated, unsaturated or aromatic ring comprising one or more heteroatoms. In some embodiments, heteroatoms include N, O, Si, P, B, S, and Se atoms. Heterocycles include 3- to 10-membered monocyclic rings, 6- to 12-membered bicyclic rings, and 5- to 12-membered bridged rings. A bicyclic heterocycle includes any combination of saturated, unsaturated and aromatic bicyclic rings, as valence permits. In some embodiments, an aromatic ring, e.g., pyridyl, may be fused to a saturated or unsaturated ring, e.g., cyclohexane, cyclopentane, morpholine, piperidine or cyclohexene. A bicyclic heterocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. A bicyclic heterocycle further includes spiro bicylic rings such as 2-oxa-6-azaspiro[3.3]heptane. The term “heterocycle” is meant to encompass “heteroaryl,” “heterocycloalkyl,” and “heterocycloalkenyl.”

The term “heteroaryl” refers to a radical derived from a 5 to 18 membered aromatic ring radical that comprises two to seventeen carbon atoms and from one to six heteroatoms selected from nitrogen, oxygen, sulfur and selenium. As used herein, the heteroaryl radical is a monocyclic, bicyclic, tricyclic or tetracyclic ring system, wherein at least one of the rings in the ring system is aromatic, i.e., it contains a cyclic, delocalized (4n+2) 71-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzoxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, selenophenyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl).

The term “heterocycloalkyl” refers to a saturated ring with carbon atoms and at least one heteroatom. In some embodiments, heteroatoms include N, O, Si, P, B, S, and Se atoms.

Heterocycloalkyl may include monocyclic and polycyclic rings such as 3- to 10-membered monocyclic rings, 6- to 12-membered bicyclic rings, spiro bicycles, and 5- to 12-membered bridged rings. The heteroatoms in the heterocycloalkyl radical are optionally oxidized. One or more nitrogen atoms, if present, are optionally quaternized. The heterocycloalkyl is attached to the rest of the molecule through any atom of the heterocycloalkyl, valence permitting, such as any carbon or nitrogen atoms of the heterocycloalkyl. Examples of heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 2-oxa-6-azaspiro[3.3]heptane, and 1,1-dioxo-thiomorpholinyl. Unless stated otherwise specifically in the specification, the term “heterocycloalkyl” is meant to include heterocycloalkyl radicals as defined above that are optionally substituted by one or more substituents such as those substituents described herein.

The term “heterocycloalkenyl” refers to an unsaturated ring with carbon atoms and at least one heteroatom and there is at least one double bond between two ring carbons. Heterocycloalkenyl does not include heteroaryl rings. In some embodiments, heteroatoms include N, O, Si, P, B, S, and Se atoms. Heterocycloalkenyl may include monocyclic and polycyclic rings such as 3- to 10-membered monocyclic rings, 6- to 12-membered bicyclic rings, and 5- to 12-membered bridged rings. In other embodiments, a heterocycloalkenyl comprises five to seven ring atoms. The heterocycloalkenyl may be attached to the rest of the molecule by a single bond. Examples of monocyclic cycloalkenyls include, e.g., pyrroline (dihydropyrrole), pyrazoline (dihydropyrazole), imidazoline (dihydroimidazole), triazoline (dihydrotriazole), dihydrofuran, dihydrothiophene, oxazoline (dihydrooxazole), isoxazoline (dihydroisoxazole), thiazoline (dihydrothiazole), isothiazoline (dihydroisothiazole), oxadiazoline (dihydrooxadiazole), thiadiazoline (dihydrothiadiazole), dihydropyridine, tetrahydropyridine, dihydropyridazine, tetrahydropyridazine, dihydropyrimidine, tetrahydropyrimidine, dihydropyrazine, tetrahydropyrazine, pyran, dihydropyran, thiopyran, dihydrothiopyran, dioxine, dihydrodioxine, oxazine, dihydrooxazine, thiazine, and dihydrothiazine. Unless otherwise stated specifically in the specification, the term “heterocycloalkenyl” is meant to include heterocycloalkenyl radicals that are optionally substituted by one or more substituents such as those substituents described herein.

The term “alkoxy” or “alkoxyl” refers to a radical bonded through an oxygen atom of the formula —O-alkyl, where alkyl is an alkyl chain as defined above.

The term “substituted” refers to moieties having substituents replacing a hydrogen on one or more carbons or substitutable heteroatoms, e.g., an NH or NH₂ of a compound. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. In some embodiments, substitution may cause a compound to have a formal charge.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl, any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

Double bonds to oxygen atoms, such as oxo groups, are represented herein as both “═O” and “(0)”. Double bonds to nitrogen atoms are represented as both “═NR” and “(NR)”. Double bonds to sulfur atoms are represented as both “═S” and “(S)”.

The phrases “parenteral administration” and “administered parenterally” as used herein means modes of administration other than enteral and topical administration, usually by injection, and includes, without limitation, intravenous, intramuscular, intra-arterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

### Further Forms of Compounds

In one aspect, compounds described herein are in the form of pharmaceutically acceptable salts. As well, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate, cyclodextrins; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; (21) preservatives, (22) antioxidants, and (23) osmolality modifying substances, and (24) other non-toxic compatible substances employed in pharmaceutical formulations.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, hydroiodic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, ion exchange resins, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

It should be understood that a reference to a pharmaceutically acceptable salt includes the solvent addition forms. In some embodiments, solvates contain either stoichiometric or non-stoichiometric amounts of a solvent, and are formed during the process of crystallization with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

The methods and formulations described herein include the use of N-oxides (if appropriate), or pharmaceutically acceptable salts of compounds having the structure disclosed herein, as well as active metabolites of these compounds having the same type of activity.

In some embodiments, sites on the organic radicals (e.g. alkyl groups, aromatic rings) of compounds disclosed herein are susceptible to various metabolic reactions. Incorporation of appropriate substituents on the organic radicals will reduce, minimize, or eliminate this metabolic pathway. In specific embodiments, the appropriate substituent to decrease or eliminate the susceptibility of the aromatic ring to metabolic reactions is, by way of example only, a halogen, deuterium, an alkyl group, a haloalkyl group, or a deuteroalkyl group.

In another embodiment, the compounds described herein are labeled isotopically (e.g. with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine chlorine, iodine, phosphorus, such as, for example, ²H, ³H, ¹³C, ¹⁴C, ¹⁵N, ¹⁸O, ¹⁷O, ³⁵S, ¹⁸F, ³⁶Cl, ¹²³I, ¹²⁴I, ¹²⁵I, ¹³¹I, ³²P and ³³P. In one aspect, isotopically-labeled compounds described herein, for example those into which radioactive isotopes such as ³H and 14C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements.

In some embodiments, the compounds disclosed herein possess one or more stereocenters and each stereocenter exists independently in either the R or S configuration. In some embodiments, the compound disclosed herein exists in the R configuration. In some embodiments, the compound disclosed herein exists in the S configuration. The compounds presented herein include all diastereomeric, individual enantiomers, atropisomers, and epimeric forms as well as the appropriate mixtures thereof. The compounds and methods provided herein include all cis, trans, syn, anti, entgegen (E), and zusammen (Z) isomers as well as the appropriate mixtures thereof.

Individual stereoisomers are obtained, if desired, by methods such as, stereoselective synthesis and/or the separation of stereoisomers by chiral chromatographic columns or the separation of diastereomers by either non-chiral or chiral chromatographic columns or crystallization and recrystallization in a proper solvent or a mixture of solvents. In certain embodiments, compounds disclosed herein are prepared as their individual stereoisomers by reacting a racemic mixture of the compound with an optically active resolving agent to form a pair of diastereoisomeric compounds/salts, separating the diastereomers and recovering the optically pure individual enantiomers. In some embodiments, resolution of individual enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley And Sons, Inc., 1981. In some embodiments, stereoisomers are obtained by stereoselective synthesis.

In some embodiments, compounds described herein are prepared as prodrugs. A “prodrug” refers to an agent that is converted into the parent drug in vivo. Prodrugs are often useful because, in some situations, they are easier to administer than the parent drug. They are, for instance, bioavailable by oral administration whereas the parent is not. Further or alternatively, the prodrug also has improved solubility in pharmaceutical compositions over the parent drug. In some embodiments, the design of a prodrug increases the effective water solubility. An example, without limitation, of a prodrug is a compound described herein, which is administered as an ester (the “prodrug”) but then is metabolically hydrolyzed to provide the active entity. A further example of a prodrug is a short peptide (polyaminoacid) bonded to an acid group where the peptide is metabolized to reveal the active moiety. In certain embodiments, upon in vivo administration, a prodrug is chemically converted to the biologically, pharmaceutically, or therapeutically active form of the compound. In certain embodiments, a prodrug is enzymatically metabolized by one or more steps or processes to the biologically, pharmaceutically, or therapeutically active form of the compound.

Prodrugs of the compounds described herein include, but are not limited to, esters, ethers, carbonates, thiocarbonates, N-acyl derivatives, N-acyloxyalkyl derivatives, N-alkyloxyacyl derivatives, quaternary derivatives of tertiary amines, N-Mannich bases, Schiff bases, amino acid conjugates, phosphate esters, and sulfonate esters. See for example Design of Prodrugs, Bundgaard, A. Ed., Elseview, 1985 and Method in Enzymology, Widder, K. et al., Ed.; Academic, 1985, vol. 42, p. 309-396; Bundgaard, H. “Design and Application of Prodrugs” in A Textbook of Drug Design and Development, Krosgaard-Larsen and H. Bundgaard, Ed., 1991, Chapter 5, p. 113-191; and Bundgaard, H., Advanced Drug Delivery Review, 1992, 8, 1-38, each of which is incorporated herein by reference. In some embodiments, a hydroxyl group in the compounds disclosed herein is used to form a prodrug, wherein the hydroxyl group is incorporated into an acyloxyalkyl ester, alkoxycarbonyloxyalkyl ester, alkyl ester, aryl ester, phosphate ester, sugar ester, ether, and the like. In some embodiments, a hydroxyl group in the compounds disclosed herein is a prodrug wherein the hydroxyl is then metabolized in vivo to provide a carboxylic acid group. In some embodiments, a carboxyl group is used to provide an ester or amide (i.e. the prodrug), which is then metabolized in vivo to provide a carboxylic acid group. In some embodiments, compounds described herein are prepared as alkyl ester prodrugs.

### Examples

Chemicals and Reagents—All reagents for used for synthesis were >90% in purity and purchased from the following commercial suppliers: Sigma-Aldrich (St. Louis, MO), Fisher Scientific (Waltham, MA), 1PlusChem (San Diego, CA), Oakwood Chemical (Estill, SC), AK Scientific (Union City, CA), Combi-Blocks (San Diego, CA), Enamine (Kyiv, Ukraine), Accela (San Diego, CA), Pharmco (Brookfield, CT), and Alfa Aesar (Ward Hill, MA). All reagents were used without further purification. Dry solvents were dried over 3 Å molecular sieves (Alfa Aesar, Ward Hill, MA) under argon atmosphere for at a minimum of 3 days prior to use. The following reagents were used for HPLC: ammonium formate (LiChropurTM, ≥99.0%, Sigma-Aldrich, St. Louis, MO), formic acid (≥98%, Sigma-Aldrich, St. Louis, MO), water (HPLC grade, Sigma-Aldrich, St. Louis, MO), acetonitrile (HPLC grade, Sigma-Aldrich, St. Louis, MO).

Nuclear Magnetic Resonance Spectroscopy—¹H and ¹³C NMR spectra data were obtained using a Bruker Avance III with PA BBO 400S1 BBF-H-D-05 Z plus probe (Bruker Corporation, Billerica, MA, USA). Samples were prepared at a concentration of −20 mg/mL in DMSO-d₆ (Sigma-Aldrich, St. Louis, MO) or CDCl₃ (Sigma-Aldrich, St. Louis, MO). ¹H spectra were collected at 400 MHz, ¹³C (APT or CPD experiments) at 100.6 MHz, and ¹⁹F (decoupled) at 376.5 MHz. Chemical shifts (δ) are reported in parts per million (ppm) and spectra were standardized to either residual solvent signals (d6-DMSO ¹H: 2.5 ppm, ¹³C: 39.52 ppm, CDCl₃: ¹H 7.26 ppm, ¹³C: 77.2 ppm) or tetramethylsilane (TMS). ¹⁹F reference CFCl₃ was set to 0.00 ppm.

pH Measurements—pH readings were obtained using an Orion 3 star (Thermo Scientific, USA) pH meter coupled with either a Thermo pH electrode (9142BN) or an Orion 8103BNUWP Ross Ultra Semi-micro pH probe (Thermo Scientific, USA) containing 3M KCl ROSS Orion filling solution (Thermos Scientific, USA).

High Performance Liquid Chromatography—HPLC spectra were obtained on an Agilent 1260 Infinity system that includes a 1260 quaternary pump VL, a 1260 ALS autosampler, a 1260 Thermostatted Column Compartment, and a 1200 DAD Multiple Wavelength Detector (Agilent Technologies, Santa Clara, CA, USA). The detection wavelength was set at 220 nm for purity. Separation for compounds was achieved using a Zorbax Eclipse Plus-C18 analytical column (5 μm, 4.6×150 mm) from Agilent (Agilent Technologies, Santa Clara, CA, USA). Mobile phase A consisted of 10 mM aqueous ammonium formate buffer titrated to pH 4.5 (with 10 mM formic acid) and mobile phase B consisted of acetonitrile. Compounds were prepared as 2 mg/mL solutions in 70:30 A:B or 50:50 A:B. The injection volume of samples was 10 μL, flow rate was 1.0 mL/min, and the column temperature was set at 25 ° C Run time was 10 minutes with a mobile phase ratio (isocratic) of 70% A and 30% B or 50% A and 50% B. All samples were run in duplicate with a wash of the injector (70:30 A:B or 50:50 A:B) between runs. Chromatograms were analyzed using the Agilent ChemStation Software (Agilent Technologies, Santa Clara, CA, USA).

Atmospheric Solids Analysis Probe Mass Spectroscopy—Low resolution mass spectra were obtained using a Advion Expressions CMS Spectrometer equipped with a quadrupole mass analyzer. Samples were ionized via Atmospheric Solids Analysis (ASAP) source using an Atmospheric Pressure Chemical Ionization (APCI) attachment. Data was processed and analyzed in Advion Data Express software. The following parameters were used for measurement: Capillary Temperature=100 ° C, Capillary Voltage=50 V, Source Gas Temperature=130 ° C, and APCI corona discharge=5 μA.

Biotage AutomatedFlash Chromatography—Automated flash chromatography was performed using Isolera™ One Flash Chromatography Systems from Biotage (Uppsala, Sweden). Separations were performed using SiO₂ (Sigma-Aldrich, St. Louis, MO) as a stationary phase and the following organic solvents as mobile phases: hexanes (Sigma-Aldrich, St. Louis, MO) and ethyl acetate (EtOAc) (Sigma-Aldrich, St. Louis, MO). In some cases, triethylamine was used as an additive. SiO₂ was manually loaded into either SNAP KP Sil 50 g or SNAP KP Sil 100 g cartridges and equilibrated with the selected mobile phase prior to loading the sample. Flow rate of solvent was 100 mL/min. UV absorbance was monitored (254 and 280 nm) and the absorbance threshold for fraction collection was set to either 10 or 40 mAu. Fractions (14 mL) were collected in a 16×100 mm rack. Chromatograms were analyzed in the internal Spektra software (Biotage, Uppsala, Sweden).

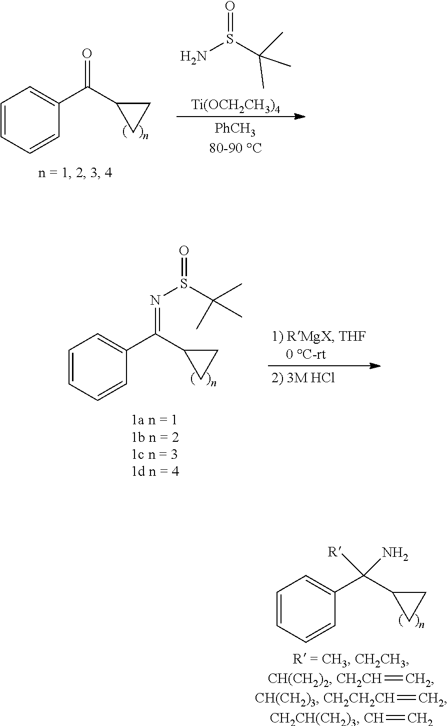

### Example 1: Synthesis of Compounds

### Synthesis of N-(cyclopropyl(phenyl)methylene)-2-methylpropane-2-sulfinamide

(1a) To a heated dried 3-neck round-bottom flask containing a Teflon coated magnetic stir bar was added racemic tert-butyl sulfinamide (5.04 g, 41.5 mmol) and dry (3 Å molecular sieves) toluene (50 mL) under argon atmosphere. To the solution was added in a continuous portion Ti(OEt)₄ (5.8 mL, 6.31 g, 27.7 mmol) via syringe and the round-bottom was fitted with a reflux condenser. The solution was heated with a heating mantle to maintain a temperature between 80-90 ° C and then cyclopropyl phenyl ketone (1.9 mL, 2.01 g, 13.7 mmol) in dry (3 molecular sieves) toluene (10 mL) was added dropwise over −1 hour (h) via an addition funnel. The reaction was allowed to stir between 80-90 ° C for 1 h and then cooled to room temperature (rt) overnight. The reaction was quenched by the addition of a saturated NaHCO₃ solution (~20 mL), diluted with EtOAc (~100 mL), and the resulting thick white suspension filtered through a pad of celite. The filter cake was washed with EtOAc (3×75 mL) and the combined filtrate and washes was transferred to a separatory funnel. The organic phase was washed with brine (2×50 mL), dried over anhydrous Na₂SO₄, and concentrated under vacuum to a crude yellow oil. The product was purified via flash chromatography (SiO₂, 8:2 hexanes:EtOAc to 6:4 hexanes:EtOAc) to afford 1a (2.52 g, 73.9%) as a viscous, yellow oil.

### Synthesis of N-(cyclopentyl(phenyl)methylene)-2-methylpropane-2-sulfinamide (1c)

To a heated dried 3-neck round-bottom flask containing a Teflon coated magnetic stir bar was added racemic tert-butyl sulfinamide (15.0 g, 124 mmol) and dry (3 Å molecular sieves) toluene (150 mL) under argon atmosphere. To the solution was added Ti(OEt)₄ (24 mL, 26.1 g, 114 mmol) via syringe in a single portion and the round-bottom was fitted with a reflux condenser. The solution was heated with a heating mantle to maintain a temperature between 80-90 ° C and then cyclopentyl phenyl ketone (9.8 mL, 10.2 g, 58.2 mmol) was added over 5 min via syringe. The reaction was allowed to stir between 80-90 ° C for 4 h and then cooled to rt overnight. The reaction was quenched with brine (~20 mL), diluted with EtOAc (~100 mL), and the resulting thick white suspension filtered through a pad of celite. The filter cake was washed with EtOAc (3×50 mL) and the combined washes and filtrate were transferred to a separatory funnel. The organic phase was washed with brine (2×50 mL), dried over anhydrous Na₂SO₄, and concentrated under vacuum to a crude yellow oil. The product was purified via flash chromatography (SiO₂, 8:2 hexanes:EtOAc to 7:3 hexanes:EtOAc) to afford 1c (9.0 g, 55%) as a viscous, yellow oil.

### Synthesis of N-(cyclohexyl(phenyl)methylene)-2-methylpropane-2-sulfinamide (1d)

To a heated dried 3-neck round-bottom flask containing a Teflon coated magnetic stir bar was added racemic tert-butyl sulfinamide (3.90 g, 32.1 mmol) and dry (3 Å molecular sieves) toluene (30 mL) under argon atmosphere. To the solution was added Ti(OEt)₄ (4.5 mL, 4.89 g, 21.4 mmol) in a single portion via syringe and the round-bottom was fitted with a reflux condenser. The solution was heated with a heating mantle to maintain a temperature between 80-90 ° C and then cyclohexyl phenyl ketone (2.02 g, 10.7 mmol) in dry (3 Å molecular sieves) toluene (20 mL) was added over 30 minutes (min) via an addition funnel. The reaction was allowed to stir between 80-90 ° C for 2.5 h, cooled to rt, and quenched with brine (~20 mL). The mixture was diluted with EtOAc (~100 mL) and the resulting thick white suspension filtered through a pad of celite. The filter cake was washed with EtOAc (3×50 mL) and the combined washes and filtrate were transferred to a separatory funnel. The organic phase was washed with brine (2×50 mL), dried over anhydrous Na₂SO₄, and concentrated under vacuum to a crude yellow oil. The product was purified via flash chromatography (SiO₂, 8:2 hexanes:EtOAc) to afford id (2.51 g, 81%) as a viscous, yellow oil that solidified upon storage in a −20 ° C freezer.

1-cyclopropyl-1-phenylethan-1-amine (Compound 2). To a heated dried round-bottom flask containing a Teflon coated magnetic stir bar was added a solution of 1a (1.0 g, 4.01 mmol) in dry (3 Å molecular sieves) THE (15 mL) under argon atmosphere. The solution was cooled to 0 ° C and a solution of methylmagnesium bromide (1.4 M in THF:toluene, 8.6 mL, 12.0 mmol) was added dropwise over 15 min. The reaction was allowed to warm to rt, stirred for ~5 h, and carefully quenched with 3M HCl (15 mL). The biphasic mixture was stirred vigorously for 1 h at rt and then diluted with diH₂O (deionized water, 15 mL) and EtOAc (~100 mL). The mixture was extracted and the organic layer was extracted further with 1M HCl (3×100 mL). The combined aqueous layers were basified with NH₄OH and extracted with EtOAc (3×75 mL). The pooled organic extractions were washed with diH₂O (20 mL) and brine (20 mL), dried over anhydrous Na₂SO₄, and concentrated under vacuum to afford a colorless transparent oil. The oil was directly converted to the HCl salt by dissolving in EtOH (20 mL) and titrating with concentrated HCl until pH<2 as determined using a pH meter. The mixture was concentrated under a warm stream of air with repeat evaporation of EtOH additions (3×~5 mL) to remove excess HCl and water. The resulting solids were washed with Et₂O (3×~5 mL) and dried to afford 1-cyclopropyl-1-phenylethan-1-amine (2) hydrochloride (200 mg, 30.9% yield) as a white powder. The solid was crystalized 3× by dissolving in a minimum volume of EtOH and layering with Et₂O and storing at 0 ° C to give a white crystalline solid. ¹H NMR HCl salt (400 MHz, DMSO-d₆) δ ppm=8.78 (s, NH₃⁺), 7.59 (dm, J=7.9 Hz, 2H), 7.41 (tm, J=7.5 Hz, 2H), 7.34 (tm, J=7.2 Hz, 1H), 1.46 (s, 3H), 1.36 (tt, J=8.4, 5.5 Hz, 1H), 0.59-0.37 (m, 4H). ¹³C NMR HCl salt (100.6 MHz, DMSO-d₆) δ ppm=141.43 (1 C), 128.32 (2 C), 127.79 (1 C), 125.68 (2 C), 58.56 (1 C), 22.27 (1 C), 20.26 (1 C), 2.06 (1 C), 1.52 (1 C). Melting point (mp): 195.9-197.8 ° C HPLC Purity (70:30 Formate buffer:ACN, 220 nm): 96.6%. ASAP MS: 146.1 (30%, [M+2]—NH₃), 145.1 (100%, [M+1]—NH₃). HRMS: m/z calcd for C₁₁H₁₅N+H [M+H]⁺, 162.1277; found 162.1279, Δppm=1.23; m/z calcd for CnH₁₃ [M−NH₃]⁺, 145.1012; found 145.1013, Δppm=0.69.

1-cyclopentyl-1-phenylethan-1-amine (Compound 3). To a heated, dried round-bottom flask containing a Teflon coated stir bar was added a solution of 1c (3.0 g, 10.8 mmol) in dry (3 Å molecular sieves) THF (50 mL). The solution was cooled to 0 ° C and a solution of methylmagnesium bromide (1.4 M in THF:toluene, 23 mL, 33.2 mmol) was added via syringe over 1 min. The reaction was allowed to warm naturally to rt overnight and then quenched with 3M HCl (50 mL). The biphasic mixture was stirred vigorously for 2 h at rt and then diluted with diH₂O (50 mL) and EtOAc (~150 mL). The aqueous layer was collected and the organic layer was extracted further with 1M HCl (3×100 mL). The combined aqueous layers were basified with NH₄0H and extracted with EtOAc (3×100 mL). The organic layer was washed with diH₂O (50 mL) and brine (50 mL), dried over Na₂SO₄, and concentrated under vacuum to afford a light yellow oil. The oil was directly converted to the HCl salt by dissolving in EtOH (40 mL) and adding concentrated HCl (485 μL). The mixture was concentrated under a warm stream of air with EtOH additions (3×~5 mL) to remove excess HCl. The resulting solid was washed with Et₂O (3×~10 mL) and dried to afford 1-cyclopentyl-1-phenylethan-1-amine (3) hydrochloride (1.10 g, 67.9% yield) as a light yellow powdery solid. The solid was crystalized 3× by dissolving in a minimum volume of EtOH and layering with Et₂O to give a white crystalline solid. ¹H NMR HCl salt (400 MHz, DMSO-d₆) δ ppm=8.69 (s, NH₃⁺), 7.54 (dm, J=8.1 Hz, 2H), 7.42 (tm, J=7.3 Hz, 2H), 7.34 (tm, J=7.2 Hz, 1H), 2.47-2.35 (m, 1H), 1.62 (s, 3H, overlap with 1.60-1.53), 1.60-1.53 (m, 1H, overlap with 1.62), 1.52-1.30 (m, 6H), 1.27-1.13 (m, 1H). ¹³C NMR HCl salt (100.6 MHz, DMSO-d₆) δ ppm=141.54 (1 C), 128.26 (2 C), 127.58 (1 C), 125.69 (2 C), 60.41 (1 C), 49.28 (1 C), 26.91 (1 C), 26.80 (1 C), 24.84 (1 C), 24.65 (1 C), 21.28 (1 C). mp: 245.3-245.7 ° C HPLC Purity (70:30 Formate buffer:ACN, 220 nm): 97.7%. ASAP MS: 191.4 (5%, [M+2]), 190.0 (10%, [M+1]), 174.2 (15%, [M+2]—NH₃) 173.2 (100%, [M+1]—NH₃). HRMS: m/z calcd for C₁₃H₁₉N+H [M+H]⁺, 190.1590; found 190.1590, Δppm=0.00; m/z calcd for C₁₃H₁₇ [M−NH₃]⁺, 173.1325; found 173.1326, Δppm=0.58.

1-cyclopentyl-1-phenylpropan-1-amine (Compound 4). To a heated, dried round-bottom flask containing a Teflon coated magnetic stir bar was added a solution of 1c (720 mg, 2.59 mmol) in dry (3 Å molecular sieves) THE (15 mL) under argon atmosphere. The solution was cooled to 0 ° C using an ice water bath and a solution of ethylmagnesium bromide (3 M in diethyl ether, 2.5 mL, 7.5 mmol) was added via syringe over 1 min. The reaction was allowed to warm naturally to rt overnight and then quenched with 3M HCl (15 mL). The biphasic mixture was stirred vigorously for 1.5 h at rt and then diluted with deionized water (diH₂O) (15 mL) and EtOAc (~100 mL). The aqueous layer was collected and the organic layer was extracted further with 1M HCl (3×100 mL). The combined aqueous extractions were basified with NH₄OH and extracted with EtOAc (3×100 mL). The organic layer was washed with diH₂O (20 mL) and brine (20 mL), dried over anhydrous Na₂SO₄, and concentrated under vacuum to afford a colorless transparent oil. The oil was directly converted to the HCl salt by dissolving in EtOH (20 mL) and adding concentrated HCl (189 μL). The mixture was concentrated under a warm stream of air with evaporation of additional EtOH additions (3×~5 mL) to remove excess HCl and water. The resulting solids were washed with Et₂O (2×~10 mL) and dried to afford 1-cyclopentyl-1-phenylpropan-1-amine (4) hydrochloride (430 mg, 69.2% yield) as a white solid. The solid was crystalized 3× by dissolving in a minimum volume of EtOH and layering with Et₂O then storing at 0 ° C to give a white crystalline solid. ¹H NMR HCl salt (400 MHz, DMSO-d₆) δ ppm=8.59 (s, NH₃⁺), 7.47-7.36 (m, 4H), 7.31 (tm, J=6.9 Hz, 1H), 2.45-2.30 (m, 1H), 2.19 (sextet, 1H), 2.00 (sextet, 1H), 1.61-1.50 (m, 1H), 1.50-1.28 (m, 7H), 0.74 (t, J=7.3 Hz, 3H). ¹³C NMR HCl salt (100.6 MHz, DMSO-d₆) δ ppm=139.11 (1 C), 128.26 (2 C), 127.32 (1 C), 126.13 (2 C), 64.34 (1 C), 49.24 (1 C), 28.20 (1 C), 26.55 (1 C), 26.29 (1 C), 24.71 (1 C), 24.48 (1 C), 7.80 (1 C). mp: >260 ° C HPLC Purity (70:30 Formate buffer:ACN, 220 nm): 99.3%. ASAP MS: 205.4 (10%, [M+2]), 204.3 (50%, [M+1]), 188.2 (20%, [M+2]—NH₃) 187.2 (100%, [M+1]—NH₃). HRMS: m/z calcd for C₁₄H₂₁N+H [M+H]⁺, 204.1747; found 204.1747, Δppm=0.00; m/z calcd for C₁₃H₁₇ [M−NH₃]⁺, 187.1481; found 187.1483, Δppm=1.07.

Cyclopentyl(cyclopropyl)(phenyl)methanamine (Compound 5). To a heated, dried round-bottom flask containing a Teflon coated stir bar was added crushed Mg turnings (260 mg, 10.7 mmol), dry (3 Å molecular sieves) THE (15 mL), and a catalytic amount of iodine crystals under argon atmosphere. Bromocyclopropane (800 μL, 1.21 g, 10.0 mmol) was added via syringe and the mixture was gently heated until initiation. The resulting dark solution then returned to rt and stirred for 1 hr. The reaction was cooled to 0 ° C and a solution of 1c (1.0 g, 3.60 mmol) in dry (3 Å molecular sieves) THE (5 mL) was added via syringe over 5 min. The mixture was allowed to warm to rt overnight and then carefully quenched with 3M HCl (30 mL). The biphasic mixture was vigorously stirred for 3 h at rt and then diluted with diH₂O (60 mL) and EtOAc (~100 mL). The aqueous layer was collected and the organic layer was extracted further with 1M HCl (2×100 mL). The combined aqueous layers were basified with NH₄OH and extracted with EtOAc (3×100 mL). The organic layer was washed with diH₂O (20 mL) and brine (20 mL), dried over Na₂SO₄, and concentrated under vacuum to afford a colorless transparent oil. The oil was directly converted to the HCl salt by dissolving in EtOH (30 mL) and titrating with concentrated HCl until pH<2 as determined by pH probe. The mixture was concentrated under a warm stream of air with evaporations of repeated EtOH additions (3×~5 mL) to remove excess HCl and water. The resulting solid was washed with Et₂O (3×~10 mL) and dried to afford cyclopentyl(cyclopropyl)(phenyl)methanamine (Compound 5) hydrochloride (600 mg, 66.0% yield) as a white powder. The solid was crystalized 3× by dissolving in a minimum volume of EtOH and layering with Et₂O followed by storing at 0 ° C ¹H NMR HCl salt (400 MHz, DMSO-d₆) δ ppm=8.39 (s, NH₃⁺), 7.65 (dm, J=8.1 Hz, 2H), 7.39 (tm, J=7.7 Hz, 2H), 7.31 (tm, J=7.3 Hz, 1H), 2.72-2.58 (m, 1H), 1.61-1.37 (m, 9H), 0.78-0.68 (m, 1H), 0.58 (tt, J=8.8, 5.6 Hz, 1H), 0.48 (tt, J=9.1, 5.3 Hz, 1H), 0.43-0.33 (m, 1H). ¹³C NMR HCl salt (100.6 MHz, DMSO-d₆) δ ppm=140.29 (1 C), 128.02 (2 C), 127.52 (1 C), 126.45 (2 C), 62.91 (1 C), 48.67 (1 C), 26.97 (1 C), 26.63 (1 C), 24.79 (1 C), 24.77 (1 C), 16.91 (1 C), 2.43 (1 C), 0.27 (1 C). mp: >260 ° C HPLC Purity (70:30 Formate buffer:ACN, 220 nm): 100%. ASAP MS: 217.2 (5%, [M+2]), 216.3 (30%, [M+1]), 200.2 (20%, [M+2]—NH₃) 199.2 (100%, [M+1]—NH₃). HRMS: m/z calcd for C₁₅H₂₁N+H [M+H]⁺, 216.1747; found 216.1748, Δppm=0.46; m/z calcd for C₁₅H₁₉ [M−NH₃]⁺, 199.1481; found 199.1483, Δppm=1.00.

1-cyclopentyl-1-phenylbut-3-en-1-amine (Compound 6). To a heated, dried round-bottom flask containing a Teflon coated stir bar was added a solution of 1c (1.0 g, 3.60 mmol) in dry (3 Å molecular sieves) THF (20 mL) under argon atmosphere. The solution was cooled to 0 ° C and a solution of allylmagnesium chloride (2.0 M in THF:toluene, 5.4 mL, 10.8 mmol) was added dropwise via addition funnel over 20 min. The reaction was allowed to warm to rt over 3 h and then quenched with 3M HCl (20 mL). The biphasic mixture was stirred vigorously for 2 h at rt and then diluted with diH₂O (20 mL) and EtOAc (~100 mL). The aqueous layer was collected and the organic layer was extracted further with 1M HCl (3×100 mL). The combined aqueous layers were basified with NH₄OH and extracted with EtOAc (3×75 mL). The organic layer was washed with diH₂O (20 mL) and brine (20 mL), dried over anhydrous Na₂SO₄, and concentrated under vacuum to afford a colorless transparent oil. The oil was directly converted to the HCl salt by dissolving in EtOH (20 mL) and adding concentrated HCl (259 μL). The mixture was concentrated under a warm stream of air with evaporations of repeated EtOH additions (3×~5 mL) to remove excess HCl and water. The resulting solid was washed with Et₂O (3×~10 mL) and dried to afford 1-cyclopentyl-1-phenylbut-3-en-1-amine (Compound 6) hydrochloride (510 mg, 56.0% yield) as a white powdery solid. The solid was crystalized 3 times by dissolving in a minimum volume of EtOH and layering with Et₂O followed by storing at 0 ° C ¹H NMR HCl salt (400 MHz, DMSO-d₆) δ ppm=8.63 (s, NH₃⁺), 7.47 (dm, J=8.2 Hz, 2H), 7.40 (tm, J=7.5 Hz, 2H), 7.31 (tm, J=7.2 Hz, 1H), 5.66-5.51 (m, 1H), 5.22 (dd, J=17.1, 1.3 Hz, 1H), 5.07 (dd, J=10.4, 1.8 Hz, 1H), 3.01 (dd, J=15.0, 7.0 Hz, 1H), 2.75 (dd, J=15.0, 7.0 Hz, 1H), 2.46-2.32 (m, 1H), 1.64-1.52 (m, 1H), 1.52-1.28 (m, 7H). ¹³C NMR HCl salt (100.6 MHz, DMSO-d₆) δ ppm=138.92 (1 C), 131.69 (1 C), 128.17 (2 C), 127.46 (1 C), 126.31 (2 C), 120.32 (1 C), 63.48 (1 C), 48.90 (1 C), 39.86 (1 C, overlap with DMSO-d₆), 26.54 (1 C), 26.30 (1 C), 24.65 (1 C), 24.46 (1 C). mp: >260 ° C HPLC Purity (70:30 Formate buffer:ACN, 220 nm): 100%. ASAP MS: 217.4 (5%, [M+2]), 216.3 (40%, [M+1]), 200.2 (20%, [M+2]—NH₃), 199.2 (90%, [M+1]—NH₃). HRMS: m/z calcd for C₁₅H₂₁N+H [M+H]⁺, 216.1747; found 216.1748, Δppm=0.46; m/z calcd for C₁₅H₁₉ [M−NH₃]⁺, 199.1481; found 199.1482, Δppm=0.50.

Cyclobutyl(cyclopentyl)(phenyl)methanamine (Compound 7). To a heated, dried pear-shaped flask containing a Teflon coated stir bar was added crushed Mg turnings (460 mg, 18.9 mmol), dry (3 Å molecular sieves) THE (10 mL), and a catalytic amount of iodine crystals under argon atmosphere. Bromocyclobutane (1.5 mL, 2.15 g, 15.9 mmol) was added via syringe in one portion and the mixture immediately became warm. The mixture was cooled briefly (~2 min) on an ice bath and then allowed to return to rt for 4 h. In a separate round-bottom flask, a solution of 1c (1.52 g, 5.47 mmol) in dry (3 Å molecular sieves) THE (20 mL) was stirred at 0 ° C and the flask was fitted with an addition funnel. The freshly prepared Grignard reagent was transferred to the addition funnel via syringe and added dropwise over 10 min. The reaction was allowed to naturally warm to rt overnight and then carefully quenched with 3M HCl (30 mL). The biphasic mixture was vigorously stirred for 3 h at rt and immediately basified with KOH solution (pH>12). The aqueous phase was extracted with EtOAc (3×100 mL) and the combined organic layers were washed with diH₂O (20 mL) and brine (20 mL). The organic layer was then dried over anhydrous Na₂SO₄ and concentrated under vacuum to afford a colorless transparent oil. The crude product was purified by flash chromatography with a stepwise gradient (SiO₂, 9:1 hexanes:EtOAc to 8:2 hexanes:EtOAc to 7:3 hexanes:EtOAc) to afford cyclobutyl(cyclopentyl)(phenyl)methanamine (7) (300 mg, 24%) as a colorless transparent oil.

The purified freebase was converted to the hydrochloride salt by dissolving in absolute EtOH (20 mL) and adding concentrated HCl (119 μL). The mixture was concentrated under a warm stream of air with evaporation of repeated EtOH additions (3×~5 mL) to remove excess HCl and water. The resulting solids were washed with Et₂O (3×~5 mL) and dried to afford a white powdery solid. The salt was crystallized by dissolving in a minimum volume of absolute EtOH and layering with Et₂O followed by storing at 0 ° C to give a white crystalline powder. ¹H NMR HCl salt (400 MHz, DMSO-d₆) δ ppm=8.49 (s, NH₃⁺), 7.50 (dm, J=8.2 Hz, 2H), 7.39 (tm, J=7.6 Hz, 2H), 7.30 (tm, J=7.2 Hz, 1H), 3.25-3.09 (m, 1H), 2.47-2.32 (m, 1H), 2.18 (pentet, 1H), 1.98-1.81 (m, 2H), 1.78-1.59 (m, 2H), 1.59-1.30 (m, 8H), 1.29-1.09 (m, 1H). ¹³C NMR HCl salt (100.6 MHz, DMSO-d₆) δ ppm=138.17 (1 C), 128.12 (2 C), 127.35 (1 C), 126.25 (2 C), 65.03 (1 C), 46.95 (1 C), 41.73 (1 C), 26.85 (1 C), 26.13 (1 C), 24.79 (1 C), 24.10 (1 C), 23.60 (1 C), 23.13 (1 C), 16.57 (1 C). mp: >260 ° C HPLC Purity: (70:30 Formate buffer:ACN, 200 nm): 100%. ASAP MS: 231.4 (10%, [M+2]), 230.3 (60%, [M+1]), 214.2 (20%, [M+2]—NH₃) 213.3 (100%, [M+1]—NH₃). HRMS: m/z calcd for C₁₆H₂₃N+H [M+H]⁺, 230.1903; found 230.1904, Δppm=0.43; m/z calcd for C₁₆H₂₁ [M−NH₃]⁺, 213.1638; found 213.1639, Δppm=0.47. 1-cyclopentyl-1-phenylpent-4-en-1-amine (Compound 8). To a heated, dried pear-shaped flask containing a Teflon coated stir bar was added crushed Mg turnings (310 mg, 12.8 mmol), dry (3 Å molecular sieves) THF (10 mL), and a catalytic amount of iodine crystals under argon atmosphere. Bromomethylcyclopropane (1.0 mL, 1.39 g, 10.3 mmol) was added via syringe in one portion and stirred vigorously. As the reaction became warm, it was cooled on a cool water bath and then allowed to stir for 2 h. In a separate round-bottom flask, a solution of 1c (1.0 g, 3.60 mmol) in dry (3 Å molecular sieves) THE (10 mL) was stirred at 0 ° C The freshly prepared Grignard reagent was added to the imine solution via syringe over 10 min and stirred for 1 h at 0 ° C The reaction was then allowed to warm to rt for 2 h and carefully quenched with 3M HCl (30 mL). The biphasic mixture was vigorously stirred for 2 h at rt and then diluted with diH₂O (30 mL) and EtOAc (~100 mL). The aqueous phase was collected and the organic layer was extracted further with 1M HCl (3×100 mL). The combined aqueous layers were basified with NH₄OH and extracted with EtOAc (3×75 mL). The organic layer was washed with diH₂O (20 mL) and brine (20 mL), dried over anhydrous Na₂SO₄, and concentrated under vacuum to afford a yellow oil. The crude product was purified by flash chromatography (SiO₂, 8:2 hexanes:EtOAc) to afford 1-cyclopentyl-1-phenylpent-4-en-1-amine (8) (320 mg, 38.8%) as a colorless transparent oil. The purified freebase was converted to the hydrochloride salt by dissolving in absolute EtOH (20 mL) and adding a slight excess of concentrated HCl (128 μL). The mixture was concentrated under a warm stream of air with evaporation of repeat EtOH additions (3×~5 mL) to remove excess HCl and water. The resulting solids were washed with hexanes (3×~10 mL) and dried to afford a white powdery solid. ¹H NMR HCl salt (DMSO-d₆, 400 MHz) δ ppm=8.67 (s, NH₃⁺), 7.45 (dm, J=7.9 Hz, 2H overlap with 7.41), 7.41 (tm, J=7.5 Hz, 2H, overlap with 7.45), 7.32 (tm, J=7.0 Hz, 1H), 5.85-5.68 (m, 1H), 4.90 (d, J=18.9 Hz, 1H), 4.94 (d, J=11.3 Hz, 1H), 2.47-2.33 (m, 1H), 2.29-2.16 (m, 1H), 2.12-1.97 (m, 2H), 1.76-1.63 (m, 1H), 1.63-1.52 (m, 1H), 1.52-1.28 (m, 7H). ¹³C NMR HCl salt (DMSO-d₆, 100.6 MHz) δ ppm=139.22 (1 C), 137.55 (1 C), 128.34 (2 C), 127.42 (1 C), 125.90 (2 C), 115.11 (1 C), 63.73 (1 C), 49.40 (1 C), 34.65 (1 C), 27.22 (1 C), 26.51 (1 C), 26.25 (1 C), 24.72 (1 C), 24.48 (1 C). mp: >260 ° C HPLC Purity: (50:50 Formate buffer:ACN, 220 nm): 99.1%. ASAP MS: 231.6 (10%, [M+1]), 230.2 (40%, [M+2]), 214.3 (20%, [M+2]—NH₃], 213.2 (100%, [M+1]—NH₃]. HRMS: m/z calcd for C₁₆H₂₃N+H [M+H]⁺, 230.1904; found 230.1903, Δppm=−0.43; m/z calcd for C₁₆H₂₁ [M−NH₃]⁺, 213.1638; found 213.1639, Δppm=0.46.

2-cyclobutyl-1-cyclopentyl-1-phenylethan-1-amine (Compound 9). To a heated, dried pear-shaped flask containing a Teflon coated stir bar was added crushed Mg turnings (310 mg, 12.8 mmol), dry (3 Å molecular sieves) THE (10 mL), and a catalytic amount of iodine crystals under argon atmosphere. Bromomethylcyclobutane (1.2 mL, 1.59 g, 10.7 mmol) was added via syringe in one portion and stirred vigorously. As the reaction became warm, it was cooled on a cool water bath and then allowed to stir for 2 h. In a separate round-bottom flask, a solution of 1c (1.0 g, 3.60 mmol) in dry (3 Å molecular sieves) THE (10 mL) was stirred at 0 ° C The freshly prepared Grignard reagent was added to the imine solution via syringe over ~1 min at 0 ° C and allowed to naturally return to rt overnight. The reaction was then carefully quenched with 3M HCl (30 mL) and the biphasic mixture was vigorously stirred for 3 h at rt. The mixture was diluted with diH₂O (30 mL) and EtOAc and then extracted. The organic layer was extracted further with 1M HCl (3×100 mL) and the combined aqueous layers were basified with NH₄OH. The aqueous layer was extracted with EtOAc (3×75 mL), washed with diH₂O (20 mL) and brine (20 mL), and dried over anhydrous Na₂SO₄. The organic layer was concentrated under vacuum to afford a yellow oil. The crude product was purified by flash chromatography (SiO₂, 9:1 hexanes:EtOAc to 7:3 hexanes:EtOAc) to afford 2-cyclobutyl-1-cyclopentyl-1-phenylethan-1-amine (9) (200 mg, 22.8%) as a colorless transparent oil. The purified freebase was converted to the hydrochloride salt by dissolving in absolute EtOH (20 mL) and adding a slight excess of concentrated HCl (75 μL). The mixture was concentrated under a warm stream of air with evaporation of repeat EtOH additions (3×~5 mL) to remove excess HCl and water. The resulting solid was washed with hexanes (3×~10 mL) and dried to afford a shiny white solid. ¹H NMR HCl salt (DMSO-d₆, 400 MHz) δ ppm=8.49 (s, NH₃⁺), 7.47 (dm, J=8.0 Hz, 2H), 7.37 (tm, J=7.6 Hz, 2H), 7.29 (tm, J=7.2 Hz, 1H), 2.48-2.41 (m, 1H, overlap with DMSO-d₆), 2.34-2.21 (m, 1H, overlap with 2.20), 2.20 (dd, J=14.0, 7.3 Hz, 1H, overlap with 2.34-2.21), 2.06 (dd, J=14.4, 5.4 Hz, 1H), 1.94-1.79 (m, 1H), 1.67-1.51 (m, 4H), 1.51-1.23 (m, 9H). ¹³C NMR HCl salt (DMSO-d₆, 100.6 MHz) δ ppm=139.55 (1 C), 127.97 (2 C), 127.25 (1 C), 126.07 (2 C), 63.97 (1 C), 48.20 (1 C), 43.59 (1 C), 30.65 (1 C), 29.31 (1 C), 29.07 (1 C), 26.52 (1 C), 26.16 (1 C), 24.86 (1 C), 24.52 (1 C), 18.53 (1 C). mp: >260 ° C HPLC Purity: (50:50 Formate buffer:ACN, 220 nm): 100%. ASAP MS: 245.2 (5%, [M+2]), 244.2 (20%, [M+1]), 228.0 (10%, [M+2]—NH₃), 227.2 (50%, [M+1]—NH₃). HRMS: m/z calcd for C₁₇H₂₅N+H [M+H]⁺, 244.2060; found 244.2059, Δppm=−0.41; m/z calcd for C₁₆H₂₃ [M−NH₃]⁺, 227.1794; found 227.1794, Δppm=0.00.

1-cyclopentyl-1-phenylprop-2-en-1-amine (Compound 10). To a heated, dried round-bottom flask containing a Teflon coated stir bar was added a solution of 1c (1.01 g, 3.64 mmol) in dry (3 Å molecular sieves) THF (30 mL) under argon atmosphere. The solution was cooled to 0 ° C and a solution of vinylmagnesium bromide (1.0 M in THF, 11 mL, 11 mmol) was added dropwise via syringe over 5 min. The reaction was allowed to warm to rt overnight and then quenched with 3M HCl (30 mL). The biphasic mixture was stirred vigorously for 3 h at rt and then diluted with diH₂O (30 mL) and EtOAc (~100 mL). The aqueous layer was collected and the organic layer was extracted further with 1M HCl (3×100 mL). The combined aqueous layers were basified with NH₄0H and extracted with EtOAc (3×100 mL). The organic layer was washed with diH₂O (20 mL) and brine (20 mL), dried over anhydrous Na₂SO₄, and concentrated under vacuum to afford a yellow transparent oil. The crude product was purified by flash chromatography with a stepwise gradient (SiO₂, 8:2 hexanes:EtOAc to 7:3 hexanes:EtOAc) to afford 1-cyclopentyl-1-phenylprop-2-en-1-amine (10) (200 mg, 27.3%) as a colorless transparent oil. The purified freebase was converted to the hydrochloride salt by dissolving in absolute EtOH (15 mL) and adding a slight excess of concentrated HCl (91 μL). The mixture was concentrated under a warm stream of air with evaporation of repeat EtOH additions (3×~5 mL) to remove excess HCl and water. The resulting solid was washed with hexanes (3×~10 mL) and dried to afford a white powdery solid. ¹H NMR HCl salt (400 MHz, DMSO-d₆) δ ppm=8.88 (s, NH₃⁺), 7.58 (dm, J=7.7 Hz, 2H), 7.45-7.37 (m, 2H), 7.37-7.30 (m, 1H), 6.36-6.23 (m, 1H), 5.52 (dm, J=12.0 Hz, 1H), 5.46 (d, J=17.7 Hz, 1H), 2.85-2.71 (m, 1H), 1.69-1.57 (m, 1H), 1.57-1.20 (m, 7H). ¹³C NMR HCl salt (100.6 MHz, DMSO-d₆) δ ppm=140.25 (1 C), 135.85 (1 C), 128.37 (2 C), 128.00 (1 C), 126.66 (2 C), 117.39 (1 C), 64.30 (1 C), 47.12 (1 C), 27.25 (1 C), 27.04 (1 C), 25.40 (1 C), 25.18 (1 C). mp: >260 ° C HPLC Purity: (70:30 Formate buffer:ACN, 200 nm): 100%. ASAP MS: 203.3 (5%, [M+2]), 202.3 (25%, [M+1]), 186.3 (20%, [M+2]—NH₃), 185.3 (100%, [M+1]—NH₃). HRMS: m/z calcd for C₁₄H₁₉N+H [M+H]⁺, 202.1590; found 202.1591, Δppm=0.49; m/z calcd for C₁₄H₁₇N [M−NH₃]⁺, 185.1325; found 185.1326, Δppm=0.54.

1-cyclohexyl-1-phenylethan-1-amine (Compound 11). To a heated, dried round-bottom flask containing a Teflon coated stir bar was added a solution of 1d (2.0 g, 6.89 mmol) in dry (3 Å molecular sieves) THF (50 mL). The solution was cooled to 0 ° C and a solution of methylmagnesium bromide (1.4 M in THF:toluene, 14 mL, 19.6 mmol) was added via syringe over 1 min. The reaction was allowed to warm naturally to rt overnight and then quenched with 3M HCl (30 mL). The biphasic mixture was stirred vigorously for 1 h at rt and then diluted with diH₂O (40 mL) and EtOAc (~100 mL). The aqueous layer was collected and the organic layer was extracted further with 1M HCl (3×100 mL). The combined aqueous layers were basified with NH₄0H and extracted with EtOAc (3×100 mL). The organic layer was washed with diH₂O (20 mL) and brine (20 mL), dried over anhydrous Na₂SO₄, and concentrated under vacuum to afford a colorless transparent oil. The oil was directly converted to the HCl salt by dissolving in EtOH (40 mL) and adding concentrated HCl (575 μL). The mixture was concentrated under a warm stream of air with EtOH additions (3×~5 mL) to remove excess HCl. The resulting solid was washed with Et₂O (3×~10 mL) and dried to afford 1-cyclohexyl-1-phenylethan-1-amine (11) (870 mg, 52.6% yield) as a white powdery solid. The solid was crystalized 3× by dissolving in a minimum volume of EtOH and layering with Et₂O to give a white crystalline solid. ¹H NMR HCl salt (400 MHz, DMSO-d₆) δ ppm=8.66 (s, NH₃⁺), 7.54-7.48 (dm, J=8.1 Hz, 2H), 7.44-7.38 (tm, J=7.4 Hz, 2H), 7.37-7.31 (tm, J=7.3 Hz, 1H), 1.89-1.76 (m, 2H), 1.76-1.67 (dm, J=12.5 Hz, 1H), 1.65-1.51 (m, 2H, overlap with 1.58), 1.58 (s, 3H, overlap with 1.65-1.51), 1.32-1.23 (dm, J=12.3 Hz, 1H), 1.22-1.07 (m, 1H), 1.07-0.88 (m, 3H), 0.88-0.74 (m, 1H). ¹³C NMR HCl salt (100.6 MHz, DMSO-d₆) δ ppm=141.41 (1 C), 128.27 (2 C), 127.63 (1 C), 125.88 (2 C), 61.34 (1 C), 46.73 (1 C), 26.75 (1 C), 26.68 (1 C), 25.75 (2 C), 25.57 (1 C), 20.15 (1 C). mp: 246.8-249.4 ° C HPLC Purity (70:30 Formate buffer:ACN, 220 nm): 97.4%. ASAP MS: 205.3 (10%, [M+2]), 204.3 (60%, [M+1]), 188.2 (15%, [M+2]—NH₃), 187.2 (100%, [M+1]—NH₃). HRMS: m/z calcd for C₁₄H₂₁N+H [M+H]⁺, 204.1747; found 204.1748, Δppm=0.49; m/z calcd for C₁₄H₁₉ [M−NH₃]⁺, 187.1481; found 187.1482, Δppm=0.53.

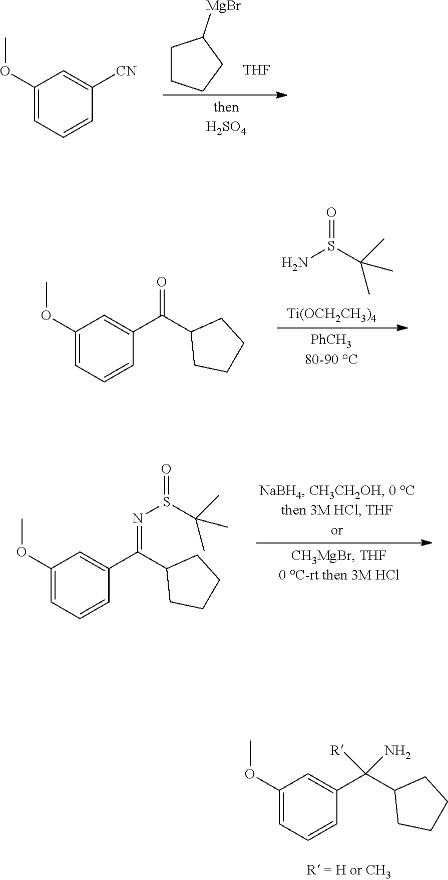

Cyclopentyl(3-methoxyphenyl)methanone. To a heated, dried 3-neck round-bottom flask containing a Teflon coated stir bar was added Mg⁰ turnings (10.9 g, 448 mmol) and dry (3 Å molecular sieves) THE (200 mL) at rt under argon atmosphere. Cyclopentyl bromide (16.1 mL, 22.4 g, 150 mmol) was added in one portion and vigorously stirred at rt until signs of reaction initiation (grey slurry and heat). The mixture was cooled on a water bath until it returned to rt and then stirred at rt for an additional 1.5 h. The mixture was then cooled to 0 ° C and fitted with an addition funnel. A solution of 3-methoxybenzonitrile (9.2 mL, 10.0 g, 75.1 mmol) in dry (3 Å molecular sieves) THE (50 mL) was then added dropwise over 30 min at 0 ° C and the mixture was allowed to warm to rt overnight. The reaction was slowly poured into a 5% H₂SO₄ solution (300 mL) with vigorous stirring in a 1 L Erlenmeyer flask at rt and allowed to stir at rt for 30 min. The mixture was extracted with EtOAc (3×100 mL) and the combined organics were washed with KOH solution (~1M, 100 mL), diH₂O (100 mL), and brine (100 mL). The organics were dried over anhydrous Na₂SO₄ and concentrated under vacuum to a crude dark amber oil. The product was purified via flash chromatography (SiO₂, 100% hexanes to 9:1 hexanes:EtOAc) to afford cyclopentyl(3-methoxyphenyl)methanone (4.80 g, 31.3%) as a yellow oil.

N-(cyclopentyl(3-methoxyphenyl)methylene)-2-methylpropane-2-sulfinamide. To a heated, dried 3-neck round-bottom flask containing a Teflon coated stir bar was added racemic tert-butyl sulfinamide (7.1 g, 58.5 mmol) and dry (3 Å molecular sieves) toluene (100 mL) under argon atmosphere. To the solution was added Ti(OEt)₄ (8.2 mL, 8.92 g, 39.1 mmol) via syringe and the round-bottom was fitted with a reflux condenser. The solution was heated with a heating mantle to maintain a temperature between 80-90 ° C and then a solution of cyclopentyl(3-methoxyphenyl)methanone (4.01 g, 19.6 mmol) in dry (3 Å molecular sieves) toluene (15 mL) was added over 45 min via syringe. The reaction was allowed to stir between 80-90 ° C for 2.5 h and then cooled to rt overnight. The reaction was quenched with 1:1 sat'd NaHCO₃:brine (~20 mL), diluted with EtOAc (~150 mL), and the resulting suspension filtered through a pad of celite.

The filter cake was washed with EtOAc (2×100 mL) and the filtrate was transferred to a separatory funnel. The organic phase was washed with brine (2×50 mL), dried over anhydrous Na₂SO₄, and concentrated under vacuum to a crude orange oil. The product was purified via flash chromatography (SiO₂, 9:1 hexanes:EtOAc to 2:1 hexanes:EtOAc) to afford N-(cyclopentyl(3-methoxyphenyl)methylene)-2-methylpropane-2-sulfinamide (3.20 g, 53.3%) as a viscous, yellow oil.

Cyclopentyl(3-methoxyphenyl)methanamine (Compound 12). To a heated, dried round-bottom flask containing a Teflon coated magnetic stir bar was added N-(cyclopentyl(3-methoxyphenyl)methylene)-2-methylpropane-2-sulfinamide (1.50 g, 4.88 mmol) and absolute EtOH (25 mL) under argon atmosphere. The solution was cooled to 0 ° C on an ice water bath and NaBH₄ (550 mg, 14.5 mmol) was added in one portion. The reaction was allowed to warm naturally to rt overnight and then quenched with KOH solution (~1 M, 200 mL). The mixture was extracted with EtOAc (3×100 mL) and the combined organic layers were washed with diH₂O (50 mL) and brine (50 mL). The organic layer was dried over anhydrous Na₂SO₄ and concentrated under vacuum to afford a yellow transparent oil. The intermediate was dissolved in THE (30 mL) and 3M HCl (30 mL) was added. The biphasic mixture was allowed to stir at rt for 4 h and then diluted with diH₂O (30 mL) and EtOAc (100 mL). The mixture was extracted and the organic layer was extracted further with 1M HCl (3×100 mL). The combined aqueous layers were basified with NH₄0H and extracted with EtOAc (3×100 mL). The organic layer was washed with diH₂O (20 mL) and brine (20 mL), dried over anhydrous Na₂SO₄, and concentrated under vacuum to afford a yellow cloudy oil. The oil was directly converted to the HCl salt by dissolving in EtOH (40 mL) and adding a slight excess of concentrated HCl (308 μL). The mixture was concentrated under a warm stream of air with evaporations of repeated EtOH additions (3×~5 mL) to remove excess HCl and water. The resulting solid was washed with Et₂O (3×~20 mL) and dried to afford cyclopentyl(3-methoxyphenyl)methanamine (12) hydrochloride (730 mg, 61.8% yield) as a white fluffy solid. The solid was crystalized 3× by dissolving in a minimum volume of EtOH and layering with Et₂O followed by storing at 0 ° C to give a white fluffy solid. ¹H NMR HCl salt (400 MHz, DMSO-d₆) δ ppm=8.65 (s, NH₃⁺), 7.30 (t, J=7.9 Hz, 1H), 7.22-7.17 (m, 1H), 7.09 (d, J=7.6 Hz, 1H), 6.91 (dd, J=8.3, 2.2 Hz, 1H), 3.99-3.88 (1H), 3.76 (s, 3H), 2.43-2.28 (m, 1H), 1.95-1.84 (m, 1H), 1.70-1.57 (m, 1H), 1.57-1.33 (m, 4H), 1.27-1.15 (m, 1H), 1.13-0.99 (m, 1H). ¹³C NMR HCl salt (100.6 MHz, DMSO-d₆) δ ppm=159.36 (1 C), 140.01 (1 C), 129.60 (1 C), 119.78 (1 C), 113.70 (1 C), 113.24 (1 C), 59.08 (1 C), 55.22 (1 C), 44.46 (1 C), 29.84 (1 C), 29.46 (1 C), 24.79 (1 C), 24.29 (1 C). mp: >260 ° C HPLC Purity: (70:30 Formate buffer:ACN, 200 nm): 100%. ASAP MS: 207.3 (10%, [M+2]), 206.3 (35%, [M+1]), 190.3 (15%, [M+2]—NH₃), 189.2 (100%, [M+1]—NH₃). HRMS: m/z calcd for C₁₃H₁₉NO+H [M+H]⁺, 206.1539; found 206.1541, Δppm=0.97; C₁₃H₁₇0+H [M+H]⁺, 189.1274; found 189.1275, Δppm=0.53.

1-cyclopentyl-1-(3-methoxyphenyl)ethan-1-amine (Compound 13). To a heated, dried round-bottom flask containing a Teflon coated magnetic stir bar was added a solution of N-(cyclopentyl(3-methoxyphenyl)methylene)-2-methylpropane-2-sulfinamide (1.50 g, 4.88 mmol) in dry (3 Å molecular sieves) THE (25 mL) under argon atmosphere. The solution was cooled to 0 ° C and a solution of methylmagnesium bromide (1.4 M in THF:toluene, 10 mL, 14 mmol) was added via syringe over 5 min. The reaction was allowed to warm to rt overnight and then carefully quenched with 3M HCl (30 mL). The biphasic mixture was stirred vigorously for 5 h at rt and then diluted with diH₂O (30 mL) and EtOAc (~100 mL). The aqueous phase was collected and the organic layer was extracted further with 1M HCl (3×100 mL). The combined aqueous layers were basified with NH₄OH and extracted with EtOAc (3×100 mL). The organic layer was washed with diH₂O (20 mL) and brine (20 mL), dried over anhydrous Na₂SO₄, and concentrated under vacuum to afford an amber transparent oil. The product was purified via flash chromatography (SiO₂, 4:1 hexanes:EtOAc to 1:1 hexanes:EtOAc) to afford 1-cyclopentyl-1-(3-methoxyphenyl)ethan-1-amine (13) (470 mg, 43.9%) as a cloudy, colorless oil. The purified free base was converted to the hydrochloride salt by dissolving in absolute EtOH (20 mL) and adding a slight excess of concentrated HCl (196 μL). The mixture was concentrated under a warm stream of air with evaporation of repeated EtOH additions (3×~5 mL) to remove excess HCl and water. The resulting salt was a gummy yellow solid that was vigorously scratched in the presence of hexanes. The salt was cooled to −20 ° C and scratched with a metal spatula periodically over 3 days to afford a light yellow powdery solid. ¹H NMR HCl salt (400 MHz, DMSO-d₆) δ ppm=8.67 (s, NH₃⁺), 7.32 (t, J=8.0 Hz, 1H), 7.18-7.14 (m, 1H), 7.07 (dm, J=7.7 Hz, 1H), 6.90 (dd, J=8.2, 2.1 Hz, 1H), 3.78 (s, 3H), 2.46-2.34 (m, 1H), 1.62-1.53 (m, 1H, overlap with 1.60), 1.60 (s, 3H, overlap with 1.62-1.53), 1.53-1.30 (m, 6H), 1.30-1.15 (m, 1H). ¹³C NMR HCl salt (100.6 MHz, DMSO-d₆) δ ppm=159.20 (1 C), 143.23 (1 C), 129.34 (1 C), 117.87 (1 C), 112.76 (1 C), 112.06 (1 C), 60.42 (1 C), 55.30 (1 C), 49.20 (1 C), 26.91 (1 C), 26.80 (1 C), 24.87 (1 C), 24.68 (1 C), 21.26 (1 C). mp: 112.4-115.8 ° C HPLC Purity: (70:30 Formate buffer:ACN, 200 nm): 100%. ASAP MS: 221.3 (10%, [M+2]), 220.3 (50%, [M+1]), 204.3 (20%, [M+2]—NH₃), 203.3 (100%, [M+1]—NH₃).

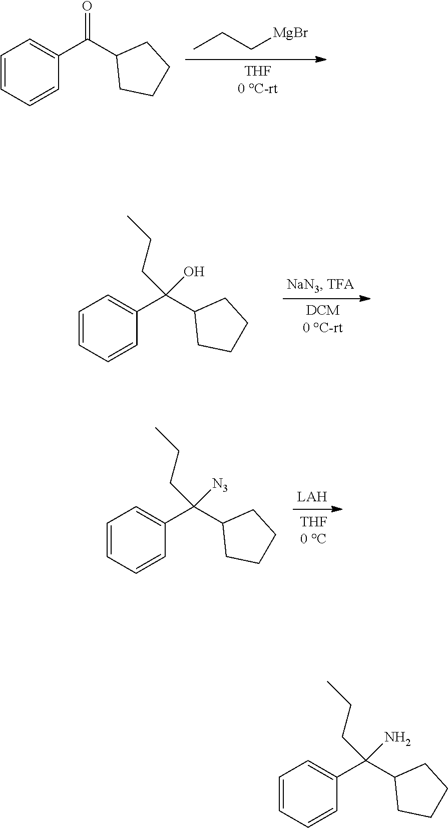

1-cyclopentyl-1-phenylbutan-1-ol. To a heated, dried round-bottom flask containing a Teflon coated magnetic stir bar was added a solution of cyclopentyl phenyl ketone (965 μL, 1.0 g, 5.73 mmol) in dry (3 Å molecular sieves) THE (15 mL) under argon atmosphere. The solution was cooled to 0 ° C and a solution of n-propylmagnesium bromide (2.0 M in diethyl ether, 8.6 mL, 17.2 mmol) was added via syringe over 10 min. The reaction was allowed to warm naturally to rt overnight and then slowly quenched with saturated NH₄Cl (15 mL) at 0 ° C The mixture was then diluted with diH₂O (~50 mL), transferred to a separatory funnel, and the aqueous was extracted with EtOAc (3×75 mL). The combined organic layers were washed with diH₂O (20 mL) and brine (20 mL), dried over anhydrous Na₂SO₄, and concentrated under vacuum to afford 1-cyclopentyl-1-phenylbutan-1-ol (1.18 g, 94.4%) as transparent colorless oil. The intermediate was used without further purification.

(1-azido-1-cyclopentylbutyl)benzene. A heated, dried round-bottom flask containing a Teflon coated magnetic stir bar was charged with dry (3 Å molecular sieves) DCM (20 mL) and NaN₃ (703 mg, 10.8 mmol) under argon atmosphere. The solution was cooled to 0 ° C and TFA (1.24 mL, 1.85 g, 16.2 mmol) was added slowly via micropipette. The flask was fitted with an addition funnel and a solution of 1-cyclopentyl-1-phenylbutan-1-ol (1.18 g, 5.40 mmol) in dry (3 Å molecular sieves) DCM (20 mL) was added dropwise. After addition, the reaction was allowed to naturally warm to rt overnight. The mixture was quenched with saturated NaHCO₃ until pH tested ~8 via pH strips and diluted with EtOAc (~100 mL). The mixture was transferred to a separatory funnel, the organic phase collected, and the aqueous layer further extracted with EtOAc (3×50 mL). The combined organics were washed with brine (20 mL), dried over anhydrous Na₂SO₄, and concentrated under vacuum to afford (1-azido-1-cyclopentylbutyl)benzene (1.19 g, ⁹0.8%) as a cloudy colorless oil. The intermediate was used without further purification.

1-cyclopentyl-1-phenylbutan-1-amine (Compound 14). A heated, dried round-bottom flask containing a Teflon coated magnetic stir bar was charged with dry (3 Å molecular sieves) THE (30 mL) and cooled to 0 ° C under argon atmosphere. With vigorous stirring, LAH (620 mg, 16.6 mmol) was added and the flask was fitted with an addition funnel. A solution of (1-azido-1-cyclopentylbutyl)benzene (1.19 g, 4.89 mmol) in dry (3 Å molecular sieves) THE (10 mL) was added dropwise over at 0 ° C The slurry was allowed to stir at 0 ° C and monitored by GC-MS for completion (1.5 h). Once complete, the mixture was quenched with ice cold THF:diH₂O (1:1 mixture, 50 mL) dropwise via addition funnel over 30 min. The mixture was diluted with EtOAc (100 mL), basified with KOH solution (1M, −4 mL), and filtered. The filter cake was washed with EtOAc (100 mL) and the filtrate was transferred to a separatory funnel. The organic layer was extracted with 1M HCl (4×100 mL) and the combined aqueous layers were basified with NH₄OH until a cloudy suspension formed. The aqueous layers were extracted with EtOAc (3×75 mL) and combined organics were washed with diH₂O (20 mL) and brine (20 mL). The organic layer was dried over anhydrous Na₂SO₄ and concentrated under vacuum to an oil. The oil was converted to the hydrochloride salt by dissolving in absolute EtOH (20 mL) and adding a slight excess of concentrated HCl (121 μL). The solvent was then evaporated under a warm stream of air with evaporation of repeated EtOH additions (3×~5 mL) to remove excess HCl and water. The resulting solids were washed with Et₂O (3×~10 mL) and dried to afford 1-cyclopentyl-1-phenylbutan-1-amine (14) hydrochloride (210 mg, 16.9%) as a white powdery solid. The solid was crystalized 3× by dissolving in a minimum volume of EtOH and layering with Et₂O followed by storing at 0 ° C to give a white crystalline powder. ¹H NMR HCl salt (DMSO-d₆, 400 MHz) δ ppm=8.57 (s, NH₃⁺), 7.43 (dm, J=8.1 Hz, 2H, overlap with 7.40), 7.40 (tm, J=7.5 Hz, 2H, overlap with 7.43), 7.31 (tm, J=6.9 Hz, 1H), 2.44-2.92 (m, 1H), 2.11 (ddd, J=15.0, 13.7, 3.9 Hz, 1H), 1.90 (ddd, J=14.8, 13.2, 4.5 Hz, 1H), 1.61-1.31 (m, 8H), 1.61-1.19 (m, 1H), 1.05-0.90 (m, 1H), 0.85 (t, J=7.2 Hz, 3H). ¹³C NMR HCl salt (DMSO-d₆, 100.6 MHz) δ ppm=139.55 (1 C), 128.27 (2 C), 127.32 (1 C), 125.92 (2 C), 63.94 (1 C), 49.47 (1 C), 37.51 (1 C), 26.50 (1 C), 26.29 (1 C), 24.72 (1 C), 24.51 (1 C), 16.21 (1 C), 14.03 (1 C). mp: >260 ° C HPLC Purity: (70:30 Formate buffer:ACN, 220 nm): 100%. ASAP MS: 219.2 (10%, [M+2]), 218.4 (30%, [M+1]), 202.2 (15%, [M+2]—NH₃), 201.3 (100%, [M+1]—NH₃).

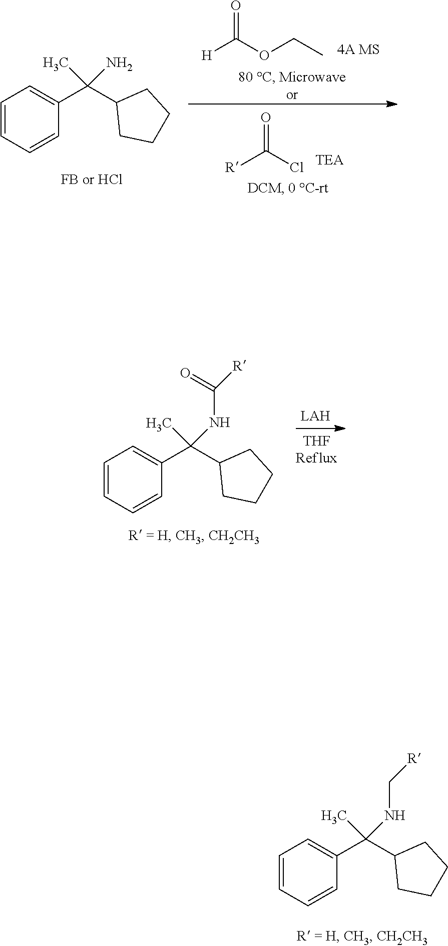

N-(1-cyclopentyl-1-phenylethyl)formamide. To a heated, dried microwave tube was added 1-cyclopentyl-1-phenylethan-1-amine (Compound 3) (1.50 g, 7.92 mmol), ethyl formate (15 mL), and 4 Å molecular sieves under argon atmosphere. The tube was placed in a CEM Discover SP microwave synthesizer and heated for a total of 10 h with the following parameters set as: Temperature=80 ° C, Max Pressure=300 PSI, Max microwave=200 W. The progress of the reaction was monitored by GC-MS. Once complete, the solution was diluted with EtOAc (150 mL), transferred to a separatory funnel, and washed with 0.5 M HCl (50 mL) and brine (50 mL). The organic layer was dried over anhydrous Na₂SO₄ and concentrated under vacuum to a brown oil to yield N-(1-cyclopentyl-1-phenylethyl)formamide (1.26 g, 73.0%). The intermediate was used without further purification.

1-cyclopentyl-N-methyl-1-phenylethan-1-amine (Compound 15). A heated, dried 3-neck round-bottom flask containing a Teflon coated magnetic stir bar was charged with dry (3× molecular sieves) THE (25 mL) and cooled to 0 ° C under argon atmosphere. With vigorous stirring, LAH (680 mg, 17.9 mmol) was added and a solution of N-(1-cyclopentyl-1-phenylethyl)formamide (1.26 g, 5.79 mmol) in dry (3 Å molecular sieves) THE (25 mL) was added via syringe over 5 min at 0 ° C The slurry was brought to reflux and monitored by GC-MS for completion (~2 h). Once complete, the mixture was cooled again to 0 ° C and quenched with ice cold THF:diH₂O (1:1 mixture, 50 mL) dropwise via addition funnel over 30 min. The mixture was diluted with EtOAc (100 mL), basified with KOH solution (1M, −4 mL), and filtered. The filter cake was washed with EtOAc (100 mL) and the filtrate was transferred to a separatory funnel. The organic layer was extracted with 1M HCl (4×100 mL) and the combined aqueous layers were basified with NH₄OH until a cloudy suspension formed. The aqueous layers were extracted with EtOAc (3×75 mL) and combined organics were washed with diH₂O (20 mL) and brine (20 mL). The organic layer was dried over anhydrous Na₂SO₄ and concentrated under vacuum to a yellow oil. The oil was converted to the hydrochloride salt by dissolving in absolute EtOH (20 mL) and adding concentrated HCl (338 μL). The mixture was concentrated under a warm stream of air with evaporations of repeated EtOH additions (3×~5 mL) to remove excess HCl and water. The resulting solid was washed with Et₂O (3×~10 mL) and dried to afford 1-cyclopentyl-N-methyl-1-phenylethan-1-amine (15) hydrochloride (870 mg, 63%) as a white powdery solid. The product was crystalized 3 times by dissolving in a minimum volume of EtOH and layering with Et₂O followed by storing at 0 ° C to give a white crystalline solid. ¹H NMR HCl salt (DMSO-d₆, 400 MHz) δ ppm=9.70 (s, 1NH⁺), 9.41 (s, 1NH⁺), 7.59 (dm, J=8.7 Hz, 2H), 7.44 (tm, J=7.6 Hz, 2H), 7.37 (tm, J=7.1 Hz, 1H), 2.67-2.54 (m, 1H), 2.17 (s, 3H), 1.87-1.75 (m, 1H), 1.61 (s, 3H), 1.57-1.44 (m, 3H), 1.44-1.29 (m, 2H), 1.22-1.11 (m, 1H), 1.11-0.99 (m, 1H). ¹³C NMR HCl salt (DMSO-d₆, 100.6 MHz) δ ppm=138.31 (1 C), 128.61 (2 C), 128.14 (1 C), 126.77 (2 C), 65.95 (1 C), 49.16 (1 C), 27.59 (1 C), 27.38 (1 C), 26.92 (1 C), 25.13 (1 C), 24.44 (1 C), 15.71 (1 C). mp: 130.0-134.5 ° C HPLC Purity: (70:30 Formate buffer:ACN, 220 nm): 100%. ASAP MS: 205.4 (<5%, [M+2]), 204.1 (10%, [M+1]), 174.2 (20%, [M+2]—NH₃), 173.2 (100%, [M+1]—NH₃).

N-(1-cyclopentyl-1-phenylethyl)acetamide. In a heated, dried round-bottom flask containing a Teflon coated stir bar was added dry (3 Å molecular sieves) DCM (15 mL), 1-cyclopentyl-1-phenylethan-1-amine (3) hydrochloride (800 mg, 4.23 mmol), and TEA (881 μL, 639 mg, 6.31 mmol) under argon atmosphere. The solution was stirred at 0 ° C and acetyl chloride (361 μL, 398 mg, 5.07 mmol) was added in one portion via micropipette. The mixture was allowed to stir at 0 ° C for 1.5 h and then diluted with EtOAc (75 mL). Saturated NaHCO₃ solution (50 mL) was carefully added, the solution was then transferred to a separatory funnel, and organic layer collected. The aqueous layer was further extracted with EtOAc (75 mL) and the combined organics were washed with diH₂O (20 mL) and brine (20 mL). The organics were dried over anhydrous Na₂SO₄ and concentrated under vacuum to afford a light yellow solid. The solid was suspended in hexanes, filtered, and washed with hexanes (3×~10 mL) to afford N-(1-cyclopentyl-1-phenylethyl)acetamide (770 mg, 79.0%) as a white solid. The intermediate was used without further purification.

1-cyclopentyl-N-ethyl-1-phenylethan-1-amine (Compound 16). A heated, dried round-bottom flask containing a Teflon coated magnetic stir bar was charged with dry (3 Å molecular sieves) THE (15 mL) and cooled to 0 ° C under argon atmosphere. With vigorous stirring, LAH (350 mg, 9.22 mmol) was added and a solution of N-(1-cyclopentyl-1-phenylethyl)acetamide (700 mg, 3.02 mmol) in dry (3 Å molecular sieves) THE (15 mL) was added dropwise via addition funnel over 45 min at 0 ° C The slurry was brought to reflux and monitored by GC-MS for completion (~10 h). Once complete, the mixture was cooled again to 0 ° C and quenched with ice cold THF:diH₂O (1:1 mixture, 10 mL) dropwise via addition funnel over 30 min. The mixture was diluted with EtOAc (75 mL), basified with KOH solution (1M, −4 mL), and filtered. The filter cake was washed with EtOAc (3×75 mL) and the filtrate was transferred to a separatory funnel. The organic layer was extracted with 1M HCl (3×100 mL) and the combined aqueous layers were basified with NH₄OH until a cloudy suspension formed. The aqueous layers were extracted with EtOAc (3×100 mL) and combined organics were washed with diH₂O (20 mL) and brine (20 mL). The organic layer was dried over anhydrous Na₂SO₄ and concentrated under vacuum to a colorless transparent oil. The oil was converted to the hydrochloride salt by dissolving in absolute EtOH (20 mL) and adding concentrated HCl (165 μL). The mixture was concentrated under a warm stream of air with evaporation of repeated EtOH additions (3×~5 mL) to remove excess HCl and water. The resulting solid was washed with Et₂O (3×~10 mL) and dried to afford 1-cyclopentyl-N-ethyl-1-phenylethan-1-amine (16) hydrochloride (400 mg, 52%) as a white powdery solid. The solid was crystalized 3× by dissolving in a minimum volume of EtOH and layering with Et₂O followed by storing at 0 ° C to give a white crystalline powder. ¹H NMR HCl salt (DMSO-d₆, 400 MHz) δ ppm=9.36 (s, 1NH⁺), 9.16 (s, 1NH⁺), 7.63 (dm, J=7.3 Hz, 2H), 7.44 (tm, J=7.5 Hz, 2H), 7.36 (tm, J=7.3 Hz, 1H), 2.85-2.66 (m, 2H), 2.37-2.22 (m, 1H), 1.95-1.80 (m, 1H), 1.63 (s, 3H), 1.60-1.28 (m, 5H), 1.20 (t, J=7.2 Hz, 3H), 1.11-0.94 (m, 2H). ¹³C NMR (DMSO-d₆, 100.6 MHz) δ ppm=139.13 (1 C), 128.67 (2 C), 128.14 (1 C), 126.55 (2 C), 66.31 (1 C), 48.83 (1 C), 37.84 (1 C), 27.78 (1 C), 26.98 (1 C), 25.34 (1 C), 24.33 (1 C), 15.22 (1 C), 11.49 (1 C). mp: 233.3-234.6 ° C HPLC Purity: (70:30 Formate buffer:ACN, 220 nm): 98.2%. ASAP MS: 219.5 (<5%, [M+2]), 218.1 (5%, [M+1]), 174.2 (20%, [M+2]—NH₃), 173.3 (100%, [M+1]—NH₃).

N-(1-cyclopentyl-1-phenylethyl)propionamide. In a heated, dried round-bottom flask containing a Teflon coated magnetic stir bar was added dry (3 Å molecular sieves) DCM (10 mL) under argon atmosphere, 1-cyclopentyl-1-phenylethan-1-amine (3) hydrochloride (600 mg, 2.65 mmol), and TEA (555 μL, 403 mg, 3.98 mmol). The solution was stirred at 0 ° C and propionyl chloride (280 μL, 297 mg, 3.21 mmol) was added in one portion via micropipette. The mixture was allowed to stir at 0 ° C for 2 h and then allowed to warm to rt overnight. The reaction was quenched with 5% NaHCO₃ (200 mL), diluted with DCM (100 mL), and transferred to a separatory funnel. The mixture was extracted and the aqueous layer was further extracted with DCM (2×100 mL). The combined organics were washed with 0.5 M HCl (20 mL) and brine (20 mL), dried over anhydrous Na₂SO₄, and concentrated under vacuum to afford a white solid. The solid was dried to afford N-(1-cyclopentyl-1-phenylethyl)propionamide (620 mg, 95.3%) as a white solid. The intermediate was used without further purification.

N-(1-cyclopentyl-1-phenylethyl)propan-1-amine (Compound 17). A heated, dried round-bottom flask containing a Teflon coated magnetic stir bar was charged with dry (3 Å molecular sieves) THE (15 mL) and cooled to 0 ° C under argon atmosphere. With vigorous stirring, LAH (480 mg, 12.6 mmol) was added and a solution of N-(1-cyclopentyl-1-phenylethyl)propionamide (600 mg, 2.45 mmol) in dry (3 Å molecular sieves) THF (15 mL) was added dropwise via syringe over 5 min at 0 ° C The slurry was brought to reflux and monitored by GC-MS for completion (8 h). Once complete, the mixture was cooled again to 0 ° C and quenched with ice cold THF:diH₂O (1:1 mixture, 20 mL) dropwise via addition funnel over 30 min. The mixture was diluted with EtOAc (100 mL), basified with KOH solution (1M, −4 mL), and filtered. The filter cake was washed with EtOAc (2×100 mL) and the filtrate was transferred to a separatory funnel. The organic layers were washed with a solution of KOH (100 mL, pH>12) and the aqueous layer was further extracted with EtOAc (2×100 mL). The combined organics were washed with diH₂O (20 mL) and brine (20 mL), dried over anhydrous Na₂SO₄, and concentrated under vacuum to a clear oil. The oil was further purified via acid/base extraction by dissolving the oil in EtOAc (100 mL) and extracting the organic layer with 1M HCl (3×100 mL). The combined aqueous layers were basified with NH₄OH until a cloudy suspension formed. The aqueous layers were extracted with EtOAc (3×75 mL) and combined organics were washed with diH₂O (20 mL) and brine (20 mL). The organic layer was dried over anhydrous Na₂SO₄ and concentrated under vacuum to a colorless transparent oil. The oil was converted to the hydrochloride salt by dissolving in absolute EtOH (20 mL) and adding concentrated HCl (71 μL). The mixture was concentrated under a warm stream of air with evaporations of repeated EtOH additions (3×~5 mL) to remove excess HCl and water. The resulting solid was washed with Et₂O (3×~10 mL) and dried to afford 1-cyclopentyl-N-(1-cyclopentyl-1-phenylethyl)propan-1-amine (17) (180 mg, 27.4%) as a white powdery solid. The solid was crystalized 3× by dissolving in a minimum volume of EtOH and layering with Et₂O followed by storing at 0 ° C to give a white powder. ¹H NMR HCl salt (400 MHz, DMSO-d₆) δ ppm=9.45-9.27 (m, 1NH⁺), 9.27-9.07 (m, 1NH⁺), 7.63 (dm, J=8.0 Hz, 2H), 7.44 (tm, J=7.5 Hz, 2H), 7.37 (tm, J=7.2 Hz, 1H), 2.82-2.70 (m, 1H, overlap with 2.71-2.60), 2.71-2.60 (m, 1H, overlap with 2.76), 2.23-2.07 (m, 1H), 1.96-1.81 (m, 1H), 1.81-1.66 (m, 1H, overlap with 1.64 and 1.67-1.29), 1.67-1.29 (m, 6H, overlap with 1.81-1.66 and 1.64), 1.64 (s, 3H, overlap with 1.81-1.66 and 1.60-1.29), 1.11-0.94 (m 2H), 0.74 (t, J=7.4 Hz, 3H). ¹³C NMR HCl salt (100.6 MHz, DMSO-d₆) δ ppm=139.09 (1 C), 128.67 (2 C), 128.17 (1 C), 126.57 (2 C), 66.49 (1 C), 48.69 (1 C), 44.31 (1 C), 27.83 (1 C), 27.01 (1 C), 25.32 (1 C), 24.32 (1 C), 19.38 (1 C), 15.23 (1 C), 11.14 (1 C). mp: 216.7-217.7 ° C HPLC Purity: (50:50 Formate buffer:ACN, 200 nm): 100%. ASAP MS: 221.3 (10%, [M+2]), 220.3 (50%, [M+1]), 204.3 (20%, [M+2]—NH₃), 203.3 (100%, [M+1]—NH₃).

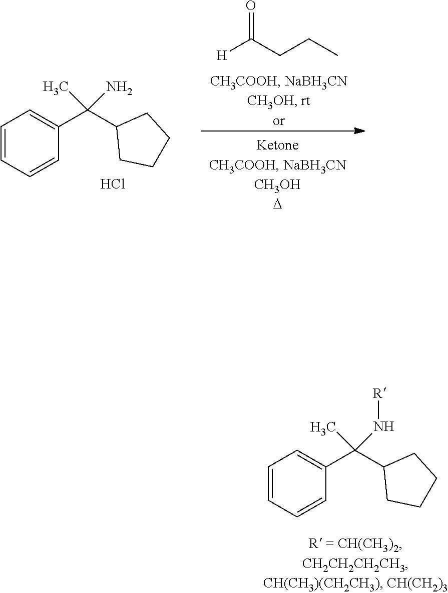

N-(1-cyclopentyl-1-phenylethyl)butan-1-amine (Compound 18). To a heated, dried round-bottom flask containing a Teflon coated magnetic stir bar was added 1-cyclopentyl-1-phenylethan-1-amine (Compound 3) hydrochloride (300 mg, 1.33 mmol), MeOH (30 mL), and glacial acetic acid (AcOH) (7.6 μL, 7.97 mg, 0.133 mmol) at rt under argon atmosphere. Solid NaBH₃CN (180 mg, 2.66 mmol) was added in one portion followed by butyraldehyde (360 μL, 288 mg, 3.99 mmol) and the reaction was allowed to stir at rt until complete by GC/MS (1 h). The mixture was quenched with KOH solution (~100 mL, pH>12) and diluted with EtOAc (75 mL). The mixture was extracted and the aqueous phase was extracted further with EtOAc (2×75 mL). The combined organic phases were washed with diH₂O (20 mL) and brine (20 mL), dried over anhydrous Na₂SO₄ and concentrated under vacuum to a colorless transparent oil. The oil was purified by flash chromatography (SiO₂, 80:20 hexanes:EtOAc) to afford N-(1-cyclopentyl-1-phenylethyl)butan-1-amine (18) (150 mg, 46.0%) as a transparent colorless oil. The oil was converted to the hydrochloride salt by dissolving in absolute EtOH (20 mL) and adding concentrated HCl (56 μL). The mixture was concentrated under a warm stream of air with evaporation of repeated EtOH additions (3×~5 mL) to remove excess HCl and water. The resulting solids were washed with hexanes (3×~10 mL) and dried to afford a white powdery solid. ¹H NMR HCl salt (400 MHz, DMSO-d₆) δ ppm=9.39-9.24 (m, 1NH⁺), 9.24-9.10 (m, 1NH⁺), 7.62 (dm, J=7.6 Hz, 2H), 7.44 (tm, J=7.3 Hz, 2H), 7.37 (tm, J=7.1 Hz, 1H), 2.84-2.63 (m, 2H), 2.27-2.12 (m, 1H), 1.96-1.80 (m, 1H), 1.74-1.45 (m, 5H, overlap with 1.64), 1.64 (s, 3H, overlap with 1.74-1.45), 1.45-1.29 (m, 2H), 1.20-1.08 (m, 2H), 1.08-0.96 (m, 2H), 0.76 (t, J=7.3 Hz, 3H). ¹³C NMR HCl salt (100.6 MHz, DMSO-d₆) δ ppm=139.10 (1C), 128.66 (2C), 128.17 (1C), 126.53 (2C), 66.53 (1C), 48.66 (1C), 42.41 (1C), 27.88 (1C), 27.83 (1C), 27.01 (1C), 25.33 (1C), 24.31 (1C), 19.46 (1C), 15.18 (1C), 13.38 (1C). mp: 80.8-85.0 ° C HPLC Purity: (50:50 Formate buffer:ACN, 200 nm): 100%. ASAP MS: 247.3 (20%, [M+2]), 246.4 (90%, [M+1]), 174.2 (15%, [M+2]—NH₃), 173.3 (100%, [M+1]—NH₃). HRMS: m/z calcd for C₁₇H₂₇N+H [M+H]⁺, 246.2216; found 246.2216, Δppm=0.00.

N-(1-cyclopentyl-1-phenylethyl)propan-2-amine (Compound 19).To a heated, dried round-bottom flask containing a teflon coated magnetic stir bar was added 1-cyclopentyl-1-phenylethan-1-amine (Compound 3) hydrochloride (200 mg, 0.88 mmol), MeOH (15 mL), and glacial AcOH (111 μL, 116 mg, 1.93 mmol) at rt under argon atmosphere. Solid NaBH₃CN (167 mg, 2.65 mmol) was added in one portion followed by acetone (20 mL, 15.6 g, 268 mmol) and the flask was fitted with a reflux condenser. The mixture was brought to reflux and monitored for completion by GC-MS (2 h). Once complete, the mixture was cooled to rt, diluted with EtOAc (100 mL) and the organic layer was extracted with 1M HCl (3×100 mL). The combined aqueous layers were basified with NH₄OH and extracted with EtOAc (3×100 mL) and combined organics were washed with diH₂O (20 mL) and brine (20 mL). The organic layer was dried over anhydrous Na₂SO₄ and concentrated under vacuum to a colorless transparent oil. The oil was converted to the hydrochloride salt by dissolving in absolute EtOH (20 mL) and adding concentrated HCl (79 μL). The mixture was concentrated under a warm stream of air with evaporations of repeated EtOH additions (3×~5 mL) to remove excess HCl and water. The resulting solid was washed with Et₂O (3×~10 mL) and dried to afford N-(1-cyclopentyl-1-phenylethyl)propan-2-amine (19) hydrochloride (400 mg, 52.0%) as a white powdery solid. The solid was crystalized 3× by dissolving in a minimum volume of EtOH and layering with Et₂O followed by storing at 0 ° C to give a white crystalline powder. ¹H NMR HCl salt (DMSO-d₆, 400 MHz) δ ppm=9.08 (d, J=11.9 Hz, 1NH⁺), 8.71 (bs, 1NH⁺), 7.69 (dm, J=8.2 Hz, 2H), 7.45 (tm, J=7.5 Hz, 2H), 7.38 (tm, J=7.2 Hz, 1H), 3.06-2.87 (m, 2H), 2.00-1.84 (m, 1H), 1.70 (s, 3H), 1.64-1.45 (m, 3H), 1.45-1.28 (m, 2H), 1.15 (d, J=6.5 Hz, 3H), 1.10 (d, J=6.6 Hz, 3H), 1.07-0.90 (m, 2H). ¹³C NMR HCl salt (DMSO-d₆, 100.6 MHz) δ ppm=138.95 (1 C), 128.55 (2 C), 128.31 (1 C), 126.86 (2 C), 67.08 (1 C), 48.79 (1 C), 48.52 (1 C), 27.84 (1 C), 26.98 (1 C), 25.47 (1 C), 24.34 (1 C), 21.33 (1 C), 20.77 (1 C), 16.67 (1 C). mp: 195.6-196.3 ° C HPLC Purity: (50:50 Formate buffer:ACN, 220 nm): 99.4%. ASAP MS: 233.5 (<5%, [M+2]), 232.3 (10%, [M+1]), 174.2 (20%, [M+2]—NH₃), 173.2 (100%, [M+1]—NH₃). HRMS: m/z calcd for C₁₆H₂₅N+H [M+H]⁺, 232.2060; found 232.2060, Δppm=0.00.

N-(1-cyclopentyl-1-phenylethyl)butan-2-amine (Compound 20). To a heated, dried round-bottom flask containing a Teflon coated magnetic stir bar was added 1-cyclopentyl-1-phenylethan-1-amine (Compound 3) hydrochloride (500 mg, 2.21 mmol), MeOH (10 mL), and glacial AcOH (253 μL, 265 mg, 4.41 mmol) at rt under argon atmosphere. Solid NaBH₃CN (460 mg, 7.32 mmol) was added in one portion followed by methyl ethyl ketone (10 mL, 8.05 g, 111 mmol) and the flask was fitted with a reflux condenser. The mixture was brought to reflux and monitored for completion by GC-MS (~6 h). Once complete, the mixture was cooled to rt and poured into a solution of KOH (200 mL). The reaction was transferred to a separatory funnel and extracted with EtOAc (3×100 mL). The combined organics were washed with diH₂O (20 mL) and brine (20 mL), dried over anhydrous Na₂SO₄, and concentrated under vacuum to a cloudy oil. The oil was purified using flash chromatography (SiO₂, 9:1 hexanes:EtOAc) to provide N-(1-cyclopentyl-1-phenylethyl)butan-2-amine (20) (290 mg, 53.5%) as a colorless transparent oil. The freebase was converted to the hydrochloride salt by dissolving in absolute EtOH (20 mL) and adding a slight excess of concentrated HCl (108 μL). The mixture was concentrated under a warm stream of air with evaporations of repeated EtOH additions (3×~5 mL) to remove excess HCl and water. The resulting salt was hygroscopic and required heating at −60 ° C to solidify. The solid was washed with Et₂O (3×10 mL) and lyophilized to produce a shiny white crystalline solid. This material was found to be a mixture of two diastereomers in an ~4:1 ratio. ¹H NMR HCl salt (DMSO-d₆, 400 MHz) δ ppm=9.04 (d, J=12.0 Hz, 0.8 NH⁺), 8.88 (d, J=12.0 Hz, 0.2NH⁺), 8.67 (d, J=12.0 Hz, 0.2NH⁺), 8.47 (d, J=12.0 Hz, 0.8NH⁺), 7.70 (dm, J=8.8 Hz, 2H), 7.45 (tm, J=7.6 Hz, 2H), 7.38 (tm, J=7.2 Hz, 1H), 3.04-2.90 (m, 1H), 2.84-2.65 (m, 1H), 1.97-1.84 (m, 1H), 1.69 (s, 3H), 1.66-1.28 (m, 7H), 1.18-1.04 (m, 2H), 1.03-0.92 (m, 1H, overlap with 1.00), 1.00 (d, J=6.6 Hz, 3H), 0.70 (t, J=7.5 Hz, 2.3H, overlap with 0.65), 0.65 (t, J=7.4 Hz, 0.7H, overlap with 0.70). ¹³C NMR HCl salt (DMSO-d₆, 100.6 MHz) δ ppm=138.68 (1 C, minor), 138.68 (1 C, major), 128.59 (2 C, minor), 128.49 (2 C, major), 128.43 (1 C, major), 128.41 (1 C, minor), 127.07 (2C, major), 126.87 (2 C, minor), 67.51 (1 C, minor), 67.31 (1C, major), 53.76 (1 C, major), 53.73 (1 C, minor), 48.29 (1 C, minor), 48.07 (1 C, major), 27.99 (apparent 2 C), 27.51 (1 C, minor), 27.33 (1 C, major), 27.04 (1 C, major), 26.98 (1 C, minor, 25.99 (1 C, minor), 25.47 (1 C, major), 24.39 (apparent 2C), 17.64 (1 C, major), 17.19 (1 C, minor), 16.75 (1 C, major), 16.62 (1 C, minor), 10.15 (1 C, major), 9.93 (1 C, minor). mp: 56.3-61.9 ° C HPLC Purity (50:50 Formate buffer:ACN, 220 nm) 99.3%. ASAP MS: 247.6 (<1%, [M+2]), 246.4 (10%, [M+1]), 174.2 (20%, [M+2]—NH₃), 173.2 (100%, [M+1]—NH₃). HRMS: m/z calcd for C₁₇H₂₇N+H [M+H]⁺, 246.2216; found 246.2218, Δppm=0.81; m/z calcd for C₁₃H₁₇ [M−NH₃]⁺, 173.1325; found 173.1327, Δppm=1.16.

N-(1-cyclopentyl-1-phenylethyl)cyclobutanamine (Compound 21). To a heated, dried round-bottom flask containing a Teflon coated magnetic stir bar was added 1-cyclopentyl-1-phenylethan-1-amine (3) hydrochloride (290 mg, 1.28 mmol), MeOH (10 mL), and glacial AcOH (152 μL, 159 mg, 2.65 mmol) at rt. Solid NaBH₃CN (250 mg, 3.97 mmol) was added in one portion followed by cyclobutanone (993 μL, 931 mg, 13.3 mmol) under argon atmosphere. The reaction was fitted with a reflux condenser and brought to −50 ° C on a heating mantle. The mixture was allowed to stir at −50 ° C until complete by GC/MS (5 h). The mixture was quenched with KOH solution (250 mL, pH>12) and diluted with EtOAc (75 mL). The mixture was extracted and the aqueous phase was extracted further with EtOAc (2×75 mL). The combined organic phases were washed with diH₂O (20 mL) and brine (20 mL), dried over anhydrous Na₂SO₄ and concentrated under vacuum to a colorless transparent oil. The oil was purified by flash chromatography (SiO₂, 80:20 Hexanes:EtOAc) to afford N-(1-cyclopentyl-1-phenylethyl)cyclobutanamine (21) (210 mg, 67.5%) as a transparent colorless oil. The oil was converted to the hydrochloride salt by dissolving in absolute EtOH (20 mL) and adding a slight excess of concentrated HCl (79 μL). The mixture was concentrated under a warm stream of air with evaporation of repeated EtOH additions (3×~5 mL) to remove excess HCl and water. The resulting solids were washed with hexanes (3×~10 mL) and dried to afford a white powdery solid. ¹H NMR HCl salt (400 MHz, DMSO-d₆) δ ppm=9.74 (d, J=10.5 Hz, 1NH⁺), 9.21 (d, J=10.5 Hz, 1NH⁺), 7.63 (dm, J=7.7 Hz, 2H), 7.42 (tm, J=7.5 Hz, 2H), 7.36 (tm, J=7.1 Hz, 1H), 3.38-3.26 (m, 1H, overlap with H₂O), 2.87-2.75 (m, 1H), 2.43 (m, 1H), 2.27-2.12 (m, 1H), 2.04-1.92 (m, 1H), 1.92-1.82 (m, 1H), 1.63-1.30 (m, 8H, overlap with 1.60), 1.60 (s, 3H, overlap with 1.63-1.30), 1.14-1.01 (m, 1H), 1.01-0.87 (m, 1H). ¹³C NMR HCl salt (100.6 MHz, DMSO-d₆) δ ppm=138.74 (1 C), 128.46 (2 C), 128.23 (1 C), 126.92 (2 C), 66.16 (1 C), 48.33 (1 C), 48.30 (1 C), 28.32 (1 C), 27.85 (1 C), 27.68 (1 C), 26.96 (1 C), 25.43 (1 C), 24.38 (1 C), 15.82 (1 C), 15.62 (1 C). mp: 195.6-197.5 ° C HPLC Purity: (50:50 Formate buffer:ACN, 200 nm): 97.2%. ASAP MS: 245.3 (20%, [M+2]), 244.4 (80%, [M+1]), 174.2 (20%, [M+2]—NH₃), 173.2 (100%, [M+1]—NH₃). HRMS: m/z calcd for C₁₇H₂₅N+H [M+H]⁺, 244.2060; found 244.2060, Δppm=0.00; m/z calcd for C₁₃H₁₇ [M−NH₃]⁺, 173.1325; found 173.1326, Δppm=0.58.

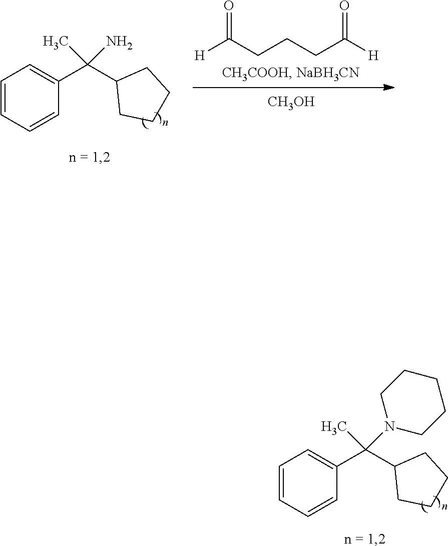

1-(1-cyclopentyl-1-phenylethyl)piperidine (Compound 22). To a heated, dried round-bottom flask containing a Teflon coated magnetic stir bar was added 1-cyclopentyl-1-phenylethan-1-amine (3) (2.01 g, 10.6 mmol), MeOH (40 mL), and glacial AcOH (725 μL, 760 mg, 12.7 mmol) at rt under argon atmosphere. Solid NaBH₃CN (2.0 g, 31.8 mmol) was added in one portion followed by glutaraldehyde (50 wt % in H₂O, d=1.106 g/mL, 5.8 mL, 3.2 g, 32.7 mmol). The mixture was stirred at rt overnight and poured into a solution of KOH (200 mL). The reaction Was transferred to a separatory funnel and extracted with EtOAc (3×150 mL). The combined organics were washed with diH₂O (20 mL) and brine (20 mL), dried over anhydrous Na₂SO₄, and concentrated under vacuum to an oil. The oil was purified using flash chromatography (SiO₂, 9:1 hexanes:EtOAc) to provide 1-(1-cyclopentyl-1-phenylethyl)piperidine (22) (1.41 g, 52.2%) as a chunky white solid. ¹H NMR freebase (CDCl₃, 400 MHz) δ ppm=7.50 (dm, J=7.8 Hz, 2H), 7.26 (tm, J=7.3 Hz, 2H, overlap with CDCl₃), 7.17 (tm, J=7.2 Hz, 1H), 2.51 (s, 2H), 2.34-2.27 (m, 2H, overlap with 2.27-2.19), 2.27-2.19 (m, 1H, overlap with 2.34-2.27), 1.61-1.38 (m, 9H), 1.37-1.23 (m, 3H, overlap with 1.32), 1.32 (s, 3H, overlap with 1.37-1.23), 1.23-1.06 (m, 2H), 0.95-0.81 (m, 1H). ¹³C NMR freebase (CDCl₃, 100.6 MHz) δ ppm=145.01 (1 C), 128.62 (2 C), 127.11 (2 C), 125.88 (1 C), 65.46 (1 C), 48.03 (2 C), 47.95 (1 C), 28.79 (1 C), 27.19 (2 C), 26.50 (1 C), 25.75 (1 C), 25.05 (1 C), 25.01 (1 C), 16.15 (1 C). mp: 35.1-37.0 ° C HPLC Purity: (50:50 Formate buffer:ACN, 220 nm): 100%. ASAP MS: 259.3 (20%, [M+2]), 258.3 (80%, [M+1]), 174.2 (20%, [M+2]—NH₃), 173.2 (100%, [M+1]—NH₃). HRMS: m/z calcd for C₁₈H₂₇N+H [M+H]⁺, 258.2216; found 258.2217, Δppm=0.39.

1-(1-cyclohexyl-1-phenylethyl)piperidine (Compound 23). To a heated, dried round-bottom flask containing a Teflon coated magnetic stir bar was added 1-cyclopentyl-1-phenylethan-1-amine (3) (820 mg, 4.03 mmol), MeOH (20 mL), and glacial AcOH (277 μL, 290 mg, 4.82 mmol) at rt under argon atmosphere. Solid NaBH₃CN (770 mg, 12.3 mmol) was added in one portion followed by glutaraldehyde (50 wt % in H₂O, d=1.106 g/mL, 1.5 mL, 829 mg, 8.28 mmol). The mixture was stirred at rt for 2 h and poured into a solution of HCl (1M, 100 mL). The mixture was diluted with EtOAc (100 mL) and transferred to a separatory funnel. The organic layer was further extracted with 1M HCl (2×100 mL) and the combined aqueous layers were basified with NH₄0H. The aqueous layer was extracted with EtOAc (2×150 mL) and the combined organics were washed with diH₂O (20 mL) and brine (20 mL). The organic layer was dried over anhydrous Na₂SO₄, concentrated under vacuum to an oil, and purified using flash chromatography (SiO₂, 75:25 hexanes:EtOAc) to provide 1-(1-cyclohexyl-1-phenylethyl)piperidine (23) (530 mg, 48.6%) as a white solid. The freebase was converted to the hydrochloride salt by dissolving in absolute EtOH (20 mL) and adding concentrated HCl (175 μL). The mixture was concentrated under a warm stream of air with evaporation of repeated EtOH additions (3×~5 mL) to remove excess HCl and water. The solids were washed with Et₂O (3×10 mL) and dried to afford a white crystalline solid. ¹H NMR HCl salt (DMSO-d₆, 400 MHz) δ ppm=9.48 (s, 1NH⁺), 7.67 (s, 2H), 7.51-7.40 (m, 3H), 3.49 (t, J=14.1 Hz, 2H), 2.79-2.65 (m, 1H), 2.56-2.45 (m, 1H, overlap with DMSO-d₆), 2.29 (q, J=13.1 Hz, 1H), 2.17-1.85 (m, 3H), 1.81 (s, 3H, overlap with 1.81-1.67), 1.81-1.67 (m, 2H), 1.67-1.52 (m, 4H), 1.46-1.30 (m, 1H), 1.21-1.14 (m, 2H), 1.14-1.01 (m, 3H), 1.00-0.86 (m, 1H). ¹³C NMR HCl salt (DMSO-d₆, 100.6 MHz) δ ppm=134.73 (1 C), 129.02 (2 C), 128.68 (3 C), 74.06 (1 C), 49.62 (1 C), 47.78 (1 C), 41.65 (1 C), 28.40 (1 C), 27.73 (1 C), 25.90 (2 C), 25.59 (1 C), 22.54 (1 C), 22.11 (1 C), 21.81 (1 C), 15.22 (1 C). mp: 186.2-187.0 ° C HPLC Purity: (50:50 Formate buffer:ACN, 220 nm): 98.5%. ASAP MS: 273.5 (20%, [M+2]), 272.4 (100%, [M+1]), 188.2 (20%, [M+2]—NH₃), 187.2 (80%, [M+1]—NH₃). HRMS: m/z calcd for C₁₉H₂₉N+H [M+H]⁺, 272.2373; found 272.2372, Δppm=−0.37.

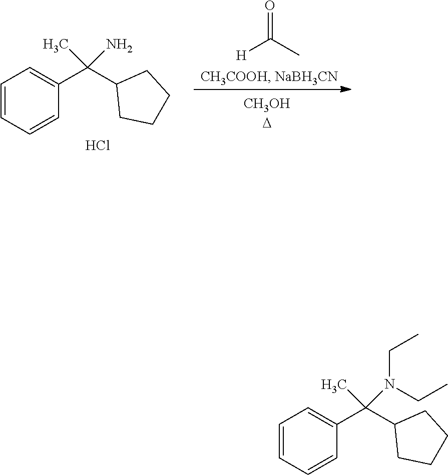

1-cyclopentyl-N,N-diethyl-1-phenylethan-1-amine (Compound 24). To a heated, dried round-bottom flask containing a Teflon coated magnetic stir bar was added 1-cyclopentyl-1-phenylethan-1-amine (Compound 3) hydrochloride (300 mg, 1.33 mmol), MeOH (20 mL), and glacial AcOH (7.6 μL, 7.97 mg, 0.13 mmol) at rt. Solid NaBH₃CN (91.7 mg, 1.46 mmol) was added in one portion followed by acetaldehyde (463 μL, 362 mg, 8.23 mmol) and the mixture was stirred at rt for 1 h. The reaction progress was followed by GC/MS and was exclusively mono-ethylated product. Additional portions of glacial AcOH (8 μL, 8.39 mg, 0.14 mmol), NaBH₃CN (91.7 mg, 1.46 mmol), and acetaldehyde (1000 μL, 784 mg, 17.8 mmol) were added and the reaction was brought to 50 ° C under a reflux condenser. The mixture was stirred at 50 ° C until complete by GC/MS (~6 h). The reaction was quenched by pouring into a KOH solution (~1M, 100 mL) which was then transferred to a separatory funnel. The aqueous phase was extracted with EtOAc (3×75 mL) and the combined organics washed with diH₂O (~20 mL) and brine (~20 mL). The organic phase was dried over anhydrous Na₂SO₄ and concentrated under vacuum to a crude red oil that was purified by flash chromatography (SiO₂, 8:2 hexanes:EtOAc to 1:1 hexanes:EtOAc) to afford 1-cyclopentyl-N,N-diethyl-1-phenylethan-1-amine (24) (130 mg, 39.9%) as a transparent yellow oil. 247.5 (10%, [M+2]), 246.4 (60%, [M+1]), 174.2 (20%, [M+2]—NH₃), 173.2 (100%, [M+1]—NH₃).

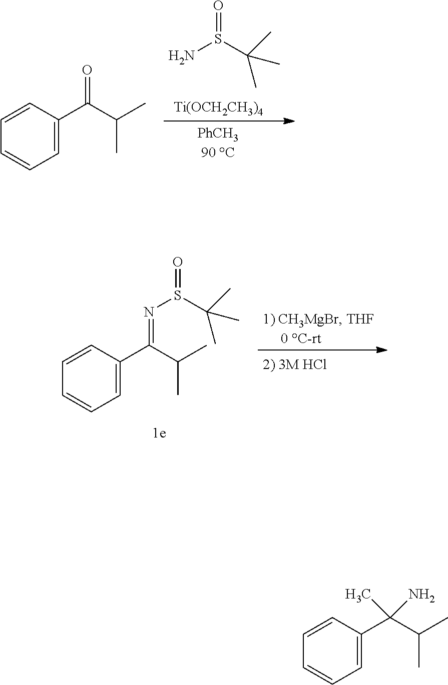

2-methyl-N-(2-methyl-1-phenylpropylidene)propane-2-sulfinamide (1e). Racemic t-butylsulfinamide (12.25 g, 101 mmol) was added to a heated, dried three-neck round bottom flask containing a Teflon coated magnetic stir bar containing dry (3 Å molecular sieves) toluene (150 mL) at rt under argon atmosphere. To this solution, Ti(OEt)₄ (14.1 mL, 15.4 g, 66.7 mmol) was added slowly in a single portion via syringe. The flask was fitted with a reflux condenser and heated to 90 ° C on a heating mantle. Once the mixture was refluxing, phenyl isopropyl ketone (5.0 g, 33.7 mmol) was dissolved in dry (3 Å molecular sieves) toluene (10 mL) and added dropwise over 20 minutes to the mixture via addition funnel. The mixture was allowed to stir at 90 ° C and monitored by TLC. After 4 h, the reaction appeared to be complete, and was cooled to rt and quenched with brine (20 mL). The resulting precipitate was filtered through a bed of celite and the filter cake was washed with EtOAc (50 mL). The organic layer was removed and then the aqueous layer was extracted with further EtOAc (3×75 mL). The pooled organic layers were washed with diH₂O (20 mL) and brine (20 mL). The organic layer was dried with anhydrous Na₂SO₄ and concentrated under vacuum to afford crude product as a viscous cloudy yellow oil. The oil was purified using flash chromatography (SiO₂, 4:1 hexanes:EtOAc) to provide 1e (4.51 g, 53.0%) as a yellow oil.

3-methyl-2-phenylbutan-2-amine (Compound 25). To a heated, dried round-bottom flask containing a Teflon coated magnetic stir bar was added a solution of 1e (2.0 g, 7.96 mmol) in dry (3 Å molecular sieves) THE (50 mL) at 0 ° C under argon atmosphere. To this mixture, methylmagnesium bromide solution (1.4M in THF/toluene, 17 mL, 23.9 mmol) was added dropwise over 30 minutes via syringe. The reaction was allowed to slowly return to rt and stirred for 20 h. The reaction was then determined to be complete by TLC. The reaction was carefully quenched with saturated ammonium chloride solution (100 mL) added dropwise for the first 50 mL. The mixture was transferred to a separatory funnel and extracted with EtOAc (3×75 mL). The combined organic layers were washed with DI-H₂O (20 mL) and brine (20 mL), dried over Na₂SO₄, and concentrated to give crude product as a yellow oil. The crude product was then stirred with dry (3× molecular sieves) THE (40 mL) in a round-bottom flask at rt. To this mixture, 3N HCl (40 mL) was added and the mixture allowed to stir for 2 h at rt. The mixture was then diluted with EtOAc (50 mL) and stirred vigorously. The mixture was basified with NH₄OH, transferred to a separatory funnel, and extracted. The aqueous layer was further extracted with EtOAc (3×75 mL) and the combined organic layers were washed with diH₂O (20 mL) and brine (20 mL). The organic layers were dried over anhydrous Na₂SO₄ and solvent was removed under vacuum to give the crude product as a faint yellow oil. The oil was directly converted to the HCl salt by dissolving in absolute EtOH (20 mL) and adding a slight excess of concentrated HCl (517 μL). Upon evaporation and evaporations of repeated addition of absolute EtOH (20 mL) and further evaporation, an off-white yellowish solid formed. The solids were washed with ether (3×~10 mL) and dried to give a sparkling white solid 3-methyl-2-phenylbutan-2-amine (25) (750 mg, 47.1% yield). The solids were crystalized by dissolving in absolute EtOH (20 mL) and titrated with diethyl ether until crystals were observed. After storage in a freezer for 16 h, a crop of fine white crystals had formed. The solids were collected by vacuum filtration and the solids were dried and recrystallized a further 2 times to yield a white glistening crystalline powder. ¹H NMR HCl salt (400 MHz, DMSO-d₆) δ ppm=8.66 (s, NH₃⁺), 7.54 (dm, J=7.6 Hz, 2H), 7.42 (tm, J=7.5 Hz, 2H), 7.35 (tm, J=7.1 Hz, 1H), 2.28-2.15 (m, 1H), 1.57 (s, 3H), 0.93 (d, J=6.9 Hz, 3H), 0.66 (d, J=6.9 Hz, 3H). ¹³C NMR HCl salt (100.6 MHz, DMSO-d₆) δ ppm=144.24 (1 C), 128.27 (2C), 127.68 (1 C), 125.87 (2 C), 61.52 (1 C), 36.64 (1 C), 19.54 (1 C), 17.14 (1 C), 17.07 (1 C). mp: 244.3-246.5 ° C HPLC Purity: (70:30 Formate buffer:ACN, 220 nm): 98.3%. ASAP MS: 165.2 (5%, [M+2]), 164.2 (30%, [M+1]), 148.2 (40%, [M+2]—NH₃), 147.1 (100%, [M+1]—NH₃). Δppm=0.61; m/z calcd for CnH₁₅ [M—NH₃], 147.1168; found 147.1169, Δppm=0.68.

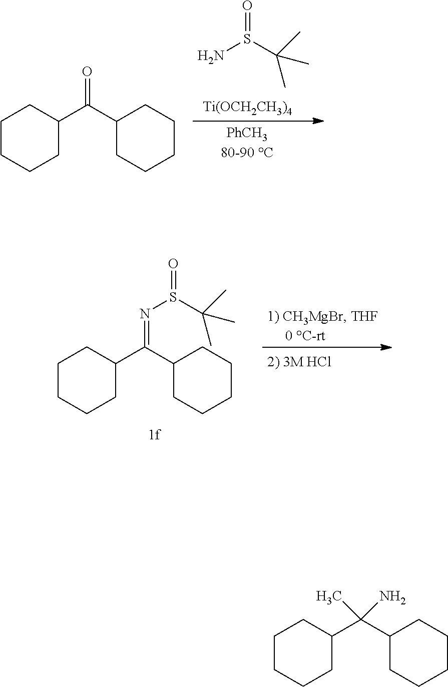

N-(dicyclohexylmethylene)-2-methylpropane-2-sulfinamide (if). To a heated, dried 3-neck round-bottom flask containing a Teflon coated magnetic stir bar was added racemic tert-butyl sulfinamide (4.9 g, 40.4 mmol) and dry (3 Å molecular sieves) toluene (50 mL) under argon atmosphere. To the solution was added Ti(OEt)₄ (6.8 mL, 7.39 g, 32.3 mmol) via syringe and the round-bottom was fitted with a reflux condenser. The solution was heated with a heating mantle to 90 ° C and then dicyclohexyl ketone (3.2 mL, 3.15 g, 16.2 mmol) was added over 5 min via syringe. The reaction was brought to reflux and allowed to stir for 6 h. The reaction was cooled to rt and quenched with brine (~20 mL), diluted with EtOAc (~100 mL), and filtered through a pad of celite. The filter cake was washed with EtOAc (3×100 mL) and the filtrate was transferred to a separatory funnel. The organic phase was washed with brine (2×50 mL), dried over anhydrous Na₂SO₄, and concentrated under vacuum to a crude orange oil. The product was purified via flash chromatography (SiO₂, 8:2 hexanes:EtOAc to 7:3 hexanes:EtOAc) to afford if (2.08 g, 43.2%) as a low-melting temperature yellow solid.

1,1-dicyclohexylethan-1-amine (Compound 26). To a heated. dried round-bottom flask containing a Teflon coated magnetic stir bar, was added a solution of if (1.0 g, 3.36 mmol) in dry (3 Å molecular sieves) THE (10 mL) under argon atmosphere. The solution was cooled to 0 ° C and a solution of methylmagnesium bromide (1.4 M in THF:toluene, 7.2 mL, 10.08 mmol) was added via syringe over 5 min. The reaction was allowed to warm naturally to rt overnight. After the overnight stir, an additional portion of methylmagnesium bromide (4.0 mL, 5.60 mmol) was added at rt and stirred for an additional 2 h. The reaction was carefully quenched with 3M HCl (20 mL). The biphasic mixture was stirred vigorously for 4 h at rt and then diluted with diH₂O (20 mL) and EtOAc (~100 mL). The mixture was extracted and the organic layer was extracted further with 1M HCl (3×100 mL). The combined aqueous layers were basified with NH₄OH and extracted with EtOAc (3×75 mL). The organic layer was washed with diH₂O (20 mL) and brine (20 mL), dried over anhydrous Na₂SO₄, and concentrated under vacuum to afford a colorless transparent oil. The oil was directly converted to the HCl salt by dissolving in EtOH (30 mL) and adding a slight excess of concentrated HCl (258 μL). The solution was concentrated under a warm stream of air with evaporation of repeat EtOH additions (3×~5 mL) to remove excess HCl and water. The resulting solids were washed with Et₂O (3×~10 mL) and dried to afford 1,1-dicyclohexylethan-1-amine (26) hydrochloride (560 mg, 68.0% yield) as a shiny white solid. The solid was crystalized 3× by dissolving in a minimum volume of EtOH and layering with Et₂O followed by storing at 0 ° C to give a shiny white crystalline solid. ¹H NMR HCl salt (400 MHz, DMSO-d₆) δ ppm=7.76 (s, NH₃⁺), 1.74 (d, J=10.8 Hz, 6H), 1.61 (q, J=12.0 Hz, 6H), 1.27-0.96 (m, 1OH, overlap with 1.05), 1.05 (s, 3H, overlap with 1.27-0.96). ¹³C NMR HCl salt (100.6 MHz, DMSO-d₆) δ ppm=60.56 (1 C), 41.98 (2 C), 26.24 (2 C), 26.18 (2 C), 26.09 (4 C), 25.77 (2 C), 17.81 (1 C). mp: 240.2-241.4 ° C ASAP MS: 211.4 (15%, [M+2]), 210.4 (85%, [M+1]), 194.3 (20%, [M+2]—NH₃), 193.3 (100%, [M+1]—NH₃). HRMS: m/z calcd for C₁₄H₂₇N+H [M+H]⁺, 210.2216; found 210.2218, Δppm=0.95. HRMS: m/z calcd for C₁₄H₂₇N+H [M+H]⁺, 210.2216; found 210.2218, Δppm=0.95.

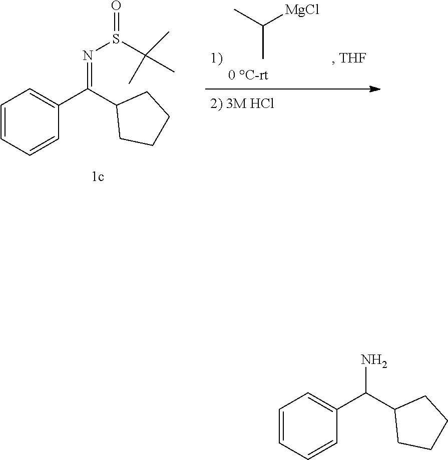

Cyclopentyl(phenyl)methanamine (Compound 27). To a heated, dried round-bottom flask containing a Teflon coated magnetic stir bar was added 1c (1.0 g, 3.60 mmol) and dry (3 Å molecular sieves) THF (20 mL) under argon atmosphere. The solution was cooled to 0 ° C and isopropyl magnesium chloride (2.0 M in THF, 5.4 mL, 10.8 mmol) was added via syringe over 5 min. The reaction was allowed to warm naturally to rt overnight and then quenched with 3M HCl (20 mL). The biphasic mixture was vigorously stirred at rt for 3 h and then diluted with diH₂O (20 mL) and EtOAc (~100 mL). The mixture was extracted and the organic layer was extracted further with 1M HCl (3×100 mL). The combined aqueous layers were basified with NH₄OH and extracted with EtOAc (3×75 mL). The organic layer was washed with diH₂O (20 mL) and brine (20 mL), dried over anhydrous Na₂SO₄, and concentrated under vacuum to a colorless transparent oil. The oil was directly converted to the HCl salt by dissolving in EtOH (40 mL) and adding a slight excess of concentrated HCl (236 μL). The mixture was concentrated under a warm stream of air with evaporation of repeat EtOH additions (3×~5 mL) to remove excess HCl and water. The resulting solid was washed with Et₂O (3×~10 mL) and dried to afford cyclopentyl(phenyl)methanamine (27) hydrochloride (500 mg, 65.6% yield) as a fluffy white solid. The solid was crystalized 3× by dissolving in a minimum volume of EtOH and layering with Et₂O followed by storing at 0 ° C to give a fluffy white crystalline solid. ¹H NMR HCl salt (400 MHz, DMSO-d₆) δ ppm=8.61 (s, NH₃⁺), 7.51 (dm, J=8.0 Hz, 2H), 7.40 (tm, J=7.1 Hz, 2H), 7.35 (tm, J=7.1 Hz, 1H), 3.96 (d, J=10.0 Hz, 1H), 2.44-2.29 (m, 1H), 1.96-1.84 (m, 1H), 1.70-1.58 (m, 1H), 1.58-1.31 (m, 4H), 1.26-1.13 (m, 1H), 1.05 (dq, J=12.6, 8.6 Hz, 1H). ¹³C NMR HCl salt (100.6 MHz, DMSO-d₆) δ ppm=138.45 (1 C), 128.56 (2 C), 128.32 (1 C), 127.57 (2 C), 59.10 (1 C), 44.50 (1 C), 29.82 (1 C), 29.47 (1 C), 24.78 (1 C), 24.28 (1 C). mp: >260 ° C HPLC Purity (70:30 Formate buffer:ACN, 220 nm): 98.9%. ASAP MS: 177.1 (40%, [M+2]), 176.2 (30%, [M+1]), 160.1 (20%, [M+2]—NH₃), 159.1 (100%, [M+1]—NH₃). HRMS: m/z calcd for C₁₂H₁₇N+H [M+H]⁺, 176.1434; found 176.1436, Δppm=1.14; C₁₂H₁₅+H [M+H]⁺, 159.1168; found 159.1171, Δppm=1.88.

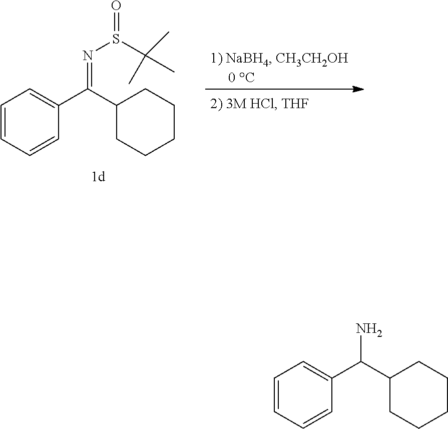

Cyclohexyl(phenyl)methanamine (Compound 28). To a heated, dried round-bottom flask containing a Teflon coated magnetic stir bar was added 1d (810 mg, 2.78 mmol) and dry (3 Å molecular sieves) THE (10 mL) under argon atmosphere. The solution was cooled to 0 ° C and NaBH₄ (310 mg, 8.19 mmol) was added in 2 portions over 5 min. The reaction was stirred for 1 h at 0 ° C and then slowly quenched with 3M HCl (10 mL). The mixture was vigorously stirred at 0 ° C for 1 h, diluted with diH₂O (10 mL), and basified with NH₄OH. The mixture was extracted with EtOAc (2×75 mL) and the combined organic layers were washed with diH₂O (20 mL) and brine (20 mL). The organic layer was dried over anhydrous Na₂SO₄ and concentrated under vacuum to afford an oil. The oil was directly converted to the HCl salt by dissolving in EtOH (40 mL) and adding concentrated HCl (460 μL). The mixture was concentrated under a warm stream of air with evaporation of repeat EtOH additions (3×~5 mL) to remove excess HCl and water. The resulting solid was washed with Et₂O (3×~10 mL) and dried to afford cyclohexyl(phenyl)methanamine (28) hydrochloride (410 mg, 65.3% yield) as a white solid. The solid was crystalized 3× by dissolving in a minimum volume of EtOH and layering with Et₂O followed by storing at 0 ° C to give a white crystalline solid. ¹H NMR HCl salt (400 MHz, DMSO-d₆) δ ppm=8.62 (s, NH₃⁺), 7.46 (dm, J=7.8 Hz, 2H), 7.41 (tm, J=6.9 Hz, 2H), 7.35 (tm, J=7.1 Hz, 1H), 3.93 (d, J=8.5 Hz, 1H), 1.96 (apparent d, J=12.5 Hz, 1H), 1.89-1.77 (m, 1H), 1.73 (dm, J=12.5 Hz, 1H), 1.64-1.49 (m, 2H), 1.28 (apparent d, J=12.5 Hz, 1H), 1.23-1.11 (m, 1H), 1.11-0.89 (m, 3H), 0.83-0.71 (m, 1H). ¹³C NMR HCl salt (100.6 MHz, DMSO-d₆) δ ppm=137.34 (1 C), 128.45 (2 C), 128.25 (1 C), 127.76 (2 C), 59.37 (1 C), 41.20 (1 C), 29.18 (1 C), 28.29 (1 C), 25.51 (1 C), 25.13 (1 C), 25.07 (1 C). mp: >260 ° C HPLC Purity (70:30 Formate buffer:ACN, 220 nm): 100%. ASAP MS: 191.3 (5%, [M+2]), 190.2 (25%, [M+1]), 174.3 (10%, [M+2]—NH₃), 173.2 (100%, [M+1]—NH₃). HRMS: m/z calcd for C₁₃H₁₉N+H [M+H]⁺, 190.1590; found 190.1589, Δppm=−0.53; C₁₃H₁₇+H [M+H]⁺, 173.1325; found 173.1325, Δppm=0.00.

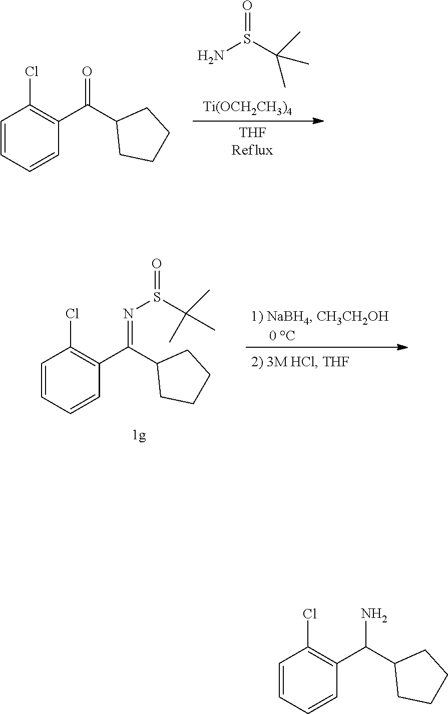

N-((2-chlorophenyl)(cyclopentyl)methylene)-2-methylpropane-2-sulfinamide (1 g). To a heated, dried round-bottom flask containing Teflon coated magnetic stir bar was added racemic tert-butyl sulfinamide (10.8 g, 89.1 mmol), 2-chlorophenyl cyclopentyl ketone (8.0 mL, 9.28 g, 44.5 mmol), and dry (3 Å molecular sieves) THE (150 mL) under argon atmosphere. To the solution was added Ti(Oet)₄ (18.6 mL, 20.2 g, 88.7 mmol) via syringe and the round-bottom was fitted with a reflux condenser. The solution was heated to reflux for 24 h, cooled back to rt, and quenched with saturated NaHCO₃ (15 mL). The mixture was diluted with EtOAc (~100 mL) and the resulting thick white suspension filtered through a pad of celite. The filter cake was washed with EtOAc (3×100 mL) and the filtrate was transferred to a separatory funnel. The organic phase was washed with brine (2×50 mL), dried over anhydrous Na₂SO₄, and concentrated under vacuum to a crude yellow oil. The product was purified via flash chromatography (SiO₂, 8:2 Hexanes:EtOAc) to afford 1 g (4.4 g, 31.7%) as a yellow oil.

To a heated, dried round-bottom flask containing a Teflon coated magnetic stir bar was added 1 g (1.0 g, 3.20 mmol) and absolute EtOH (15 mL) under argon atmosphere. The solution was cooled to 0 ° C and NaBH₄ (380 mg, 10.0 mmol) was added in one portion. The reaction was allowed to warm naturally to rt overnight and then quenched with KOH solution (~1 M, 300 mL). The mixture was extracted with EtOAc (2×100 mL) and the combined organic layers were washed with diH₂O (20 mL) and brine (20 mL). The organic layer was dried over Na₂SO₄ and concentrated on a rotary evaporator to afford a colorless transparent oil. The intermediate was dissolved in THE (20 mL) and 3M HCl (16 mL) was added. The biphasic mixture was allowed to stir overnight and then diluted with diH₂O (20 mL) and EtOAc (100 mL). The mixture was extracted and the organic layer was extracted further with 1M HCl (3×100 mL). The combined aqueous layers were basified with NH₄OH and extracted with EtOAc (3×75 mL). The organic layer was washed with diH₂O (20 mL) and brine (20 mL), dried over Na₂SO₄, and concentrated on a rotary evaporator to afford a colorless transparent oil. The oil was directly converted to the HCl salt by dissolving in EtOH (30 mL) and adding concentrated HCl (257 μL). The mixture was concentrated under a warm stream of air with evaporations of repeated EtOH additions (3×~5 mL) to remove excess HCl and water. The resulting solid was washed with Et₂O (3×~10 mL) and dried to afford (2-chlorophenyl)(cyclopentyl)methanamine (29) hydrochloride (480 mg, 61.0% yield) as a white powdery solid. The solid was crystalized 3× by dissolving in a minimum volume of EtOH and layering with Et₂O followed by storing at 0 ° C to give a white crystalline solid. ¹H NMR HCl salt (400 MHz, DMSO-d₆) δ ppm=8.80 (s, NH₃⁺), 7.87 (dd, J=7.6, 1.6 Hz, 1H), 7.51 (dd, J=7.9, 1.3 Hz, 1H), 7.45 (ddd, J=8.1, 7.6, 1.3 Hz, 1H), 7.39 (ddd, J=8.0, 7.6, 1.6 Hz, 1H), 4.41 (d, J=9.8 Hz, 1H), 2.54-2.39 (m, 1H, overlap with DMSO-d₆), 1.98-1.85 (m, 1H), 1.73-1.61 (m, 1H), 1.61-1.48 (m, 3H), 1.48-1.35 (m, 1H), 1.33-1.21 (m, 1H), 1.06-1.00 (m, 1H). ¹³C NMR HCl salt (100.6 MHz, DMSO-d₆) δ ppm=135.92 (1 C), 132.49 (1 C), 130.01 (1 C), 129.50 (1 C), 128.74 (1 C), 127.94 (1 C), 54.54 (1 C), 44.60 (1 C), 29.68 (1 C), 28.82 (1 C), 24.65 (1 C), 24.30 (1 C). mp: 247.8-248.2 ° C HPLC Purity (70:30 Formate buffer:ACN, 220 nm): 99.7%. 212.2 (40%, [M+1]³⁷Cl), 211.2 (20%, [M+2]³⁵Cl), 210.2 (80%, [M+1]³⁵Cl), 195.2 (30%, [M+2]—NH₃ ³⁷Cl), 194.2 (20%, [M+2]—NH₃ ³⁵Cl), 193.2 (100%, [M+1]—NH_{3 35}Cl). HRMS: m/z calcd for C₁₆H₂₅₃₅Cl N+H [M+H]⁺, 210.1044; found 210.1045, Δppm=0.48; m/z calcd for C₁₆H₂₅₃₇Cl N+H [M+H]⁺, 212.1015; found 212.1013, Δppm=−0.94; m/z calcd for C₁₂H₁₄³⁵Cl [M−NH₃]⁺, 193.0779; found 193.0780, Δppm=0.52; C₁₂H₁₄³⁷Cl [M−NH₃]⁺, 195.0749; found 195.0750, Δppm=0.51.

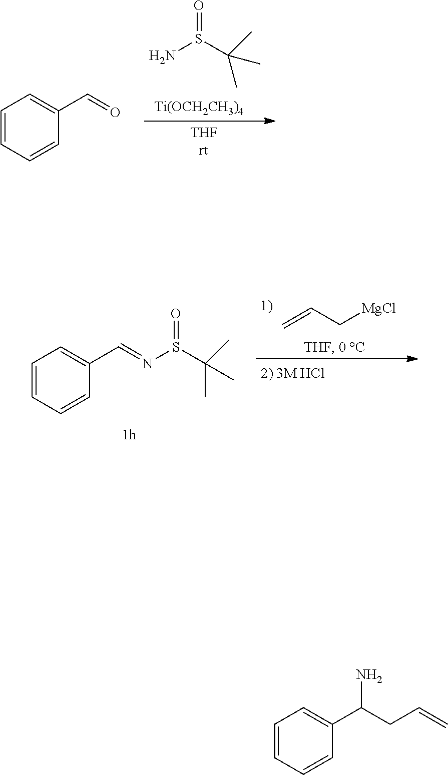

N-benzylidene-2-methylpropane-2-sulfinamide (1h). To a heated, dried round-bottom flask containing a Teflon coated magnetic stir bar was added racemic tert-butyl sulfinamide (1.15 g, 9.48 mmol), benzaldehyde (960 μL, 1.0 g, 9.44 mmol), and dry (3 Å molecular sieves) THF (20 mL) under argon atmosphere. To the solution was added Ti(Oet)₄ (4.0 mL, 4.35 g, 19.1 mmol) via syringe and the solution was stirred at rt overnight. The mixture was quenched with saturated NaHCO₃ (15 mL) and brine (15 mL), diluted with EtOAc (~100 mL) and filtered. The filter cake was washed with EtOAc (3×100 mL) and the filtrate was transferred to a separatory funnel. The organic phase was washed with brine (2×50 mL), dried over anhydrous Na₂SO₄, and concentrated under vacuum to a yellow oil that solidified upon storage at −20 ° C N-benzylidene-2-methylpropane-2-sulfinamide (1h) (1.75 g, 88.8%) was used without further purification.

1-phenylbut-3-en-1-amine (Compound 30). To a heated, dried round-bottom flask containing a Teflon coated magnetic stir bar was added a solution of (1h) (1.56 g, 7.45 mmol) in dry (3 Å molecular sieves) THE (30 mL) under argon atmosphere. The solution was cooled to 0 ° C and a solution of allylmagnesium chloride (2.0 M in THF, 8.6 mL, 12.0 mmol) was added via syringe in one portion. The reaction was allowed to warm naturally to rt overnight and then carefully quenched with 3M HCl (30 mL). The biphasic mixture was stirred vigorously for 2 h at rt and then diluted with diH₂O (30 mL) and EtOAc (~100 mL). The mixture was extracted and the organic layer was extracted further with 1M HCl (3×100 mL). The combined aqueous layers were basified with NH₄0H and extracted with EtOAc (3×75 mL). The organic layer was washed with diH₂O (20 mL) and brine (20 mL), dried over Na₂SO₄, and concentrated under vacuum to afford an oil. The oil was directly converted to the HCl salt by dissolving in EtOH (30 mL) and adding a slight excess of concentrated HCl (516 μL). The mixture was concentrated under a warm stream of air with evaporations of repeated EtOH additions (3×~5 mL) to remove excess HCl and water. The resulting solids were washed with Et₂O (3×~10 mL) and dried to afford 1-phenylbut-3-en-1-amine (30) hydrochloride (200 mg, 14.7% yield) as a white powder. The solid was crystalized 3× by dissolving in a minimum volume of EtOH and layering with Et₂O followed by storing at 0 ° C to give a white crystalline powder. ¹H NMR HCl salt (400 MHz, DMSO-d₆) δ ppm=8.69 (s, NH₃⁺), 7.51 (dm, J=8.0 Hz, 2H), 7.41 (tm, J=6.9 Hz, 2H), 7.35 (tm, J=7.1 Hz, 1H), 5.67-5.52 (m, 1H), 5.03 (dm, J=17.5 Hz, 1H), 5.00 (dm, J=11.7 Hz, 1H), 4.27 (dd, J=8.9, 5.8 Hz, 1H), 2.83-2.72 (m, 1H), 2.67-2.55 (m, 1H). ¹³C NMR HCl salt (100.6 MHz, DMSO-d₆) δ ppm=137.42 (1 C), 132.79 (1 C), 128.60 (2 C), 128.49 (1 C), 127.60 (2 C), 118.87 (1 C), 53.94 (1 C), 38.54 (1 C). mp: 231.9-233.2 ° C HPLC Purity (70:30 Formate buffer:ACN, 220 nm): 100%.ASAP MS: 149.1 (5%, [M+2]), 148.2 (30%, [M+1]), 132.2 (20%, [M+2]—NH₃), 131.1 (100%, [M+1]—NH₃). HRMS: m/z calcd for C₁₀H₁₃N+H [M+H]⁺, 148.1121; found 148.1122, Δppm=0.68; m/z calcd for C₁₀H₁₁ [M−NH₃]⁺, 131.0855; found 131.0858, Δppm=2.29.

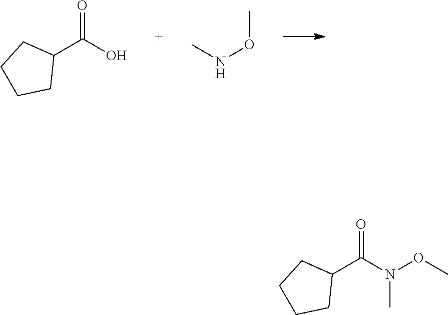

General Procedure A. Synthesis of N-methoxy-N-methylcyclopentanecarboxamide. To a dry single neck round-bottom flask containing Teflon coated magnetic stir bar was added DCM (500 mL), cyclopentanecarboxylic acid (9.6 mL, 10.1 g, 88.4 mmol), N,O-dimethylhydroxylamine hydrochloride (10.3 g, 105 mmol), 4-methylmorpholine (4.8 mL, 4.42 g, 43.6 mmol), EDAC HCl (33.6 g, 175 mmol), and HOBt hydrate (27.1 g, 17.7 g active HOBt, 130 mmol) at rt. The mixture was stirred at rt for 72 h and then slowly quenched with sat'd NaHCO₃ (200 mL). The biphasic mixture was stirred vigorously at rt for 5 min and then transferred to a separatory funnel. The organic phase was collected and the aqueous phase was extracted with DCM (2×100 mL). The combined organic phases were washed with 1M HCl (100 mL), dH₂O (100 mL), and brine (100 mL). The organic phase was dried over Na₂SO₄ and evaporated to afford N-methoxy-N-methylcyclopentanecarboxamide (13.0 g, 93.5%) a yellow oil. This intermediate was used without further purification.

Synthesis of N-methoxy-N-methylbicyclo[2.2.1]heptane-2-carboxamide. Prepared as described in General Procedure A except with DCM (100 mL), norbane-2-carboxylic acid (3.03 g, 21.6 mmol), N,O-dimethylhydroxylamine hydrochloride (2.50 g, 25.6 mmol), 4-methylmorpholine (1.2 mL, 1.1 g, 10.9 mmol), EDAC HCl (8.29 g, 43.2 mmol), and HOBt hydrate (5.45 g, 4.36 g active HOBt, 32.3 mmol) for 24 hr to afford N-methoxy-N-methylbicyclo[2.2.1]heptane-2-carboxamide (3.78 g, 95.6%) as a clear yellow oil. This intermediate was used without further purification.

Synthesis of N-methoxy-N-methylthiophene-2-carboxamide. Prepared as described in General Procedure A except with DCM (100 mL), thiophene-2-carboxylic acid (2.0 g, 15.6 mmol), N,O-dimethylhydroxylamine hydrochloride (1.90 g, 19.4 mmol), 4-methylmorpholine (857 μL, 788 mg, 7.79 mmol), EDAC HCl (6.0 g, 31.2 mmol), and HOBt hydrate (3.1 g, 2.67 g active HOBt, 19.7 mmol) for 24 hr. The reaction was also quenched with dH₂O (200 mL), which formed a white precipitate. The aqueous phase was extracted with DCM (2×100 mL). An emulsion formed in the aqueous phase from the solid which clarified upon basifying with sat'd NaHCO₃ allowing for the collection of the remaining organic phase. The combined organic phases were washed with sat'd NaHCO₃ (50 mL) and brine (50 mL). The product was further purified via flash chromatography (SiO₂, 1:1 hexanes:EtOAc) to afford N-methoxy-N-methylthiophene-2-carboxamide (2.36 g, 88.4%) as a clear, light yellow oil.

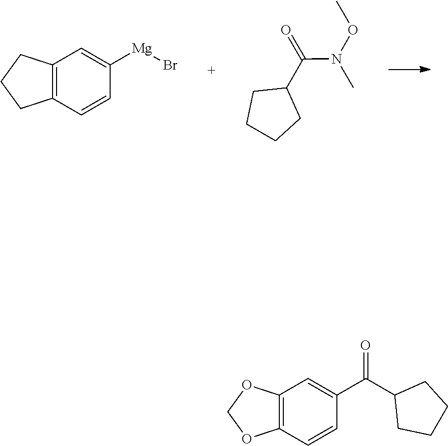

General Procedure B: Synthesis of benzo[d][1,3]dioxol-5-yl(cyclopentyl)methanone. In a dry pear-shaped flask containing a Teflon coated magnetic stir bar, crushed Mg turnings (1.1 g, 45.2 mmol) were added to dry (3 Å molecular sieves) THF (50 mL) followed by a catalytic I₂ crystal. The mixture was stirred vigorously at rt and 1-bromo-3,4-(methylenedioxyl)benzene (4.4 mL, 7.34 g, 36.5 mmol) was added via syringe in small portions over 10 min. The solution became clear and internal temperature increase to ~40° C At this time, the reaction was cooled to 0° C on an ice bath and allowed to stir for 1 h. In a separate dry round-bottom flask containing a stir bar, a solution of N-methoxy-N-methylcyclopentanecarboxamide (2.9 g, 18.4 mmol) in dry (3 Å molecular sieves) THF (50 mL) was added and the mixture was cooled to 0° C The freshly prepared Grignard reagent was added to the solution of amide via syringe at 0 ° C over −2 min. The reaction was stirred for 1 h at 0 ° C, quenched with sat'd NH₄Cl (100 mL), and stirred for 5 min at 0° C The mixture was transferred to a separatory funnel and the aqueous phase was extracted with EtOAc (3×100 mL). The combined organics were washed with brine (50 mL), dried over Na₂SO₄, and concentrated to an amber oil. The crude oil was purified via flash chromatography (SiO₂, 9:1 hexanes:EtOAc to 7:3 hexanes:EtOAc) to afford benzo[d][1,3]dioxol-5-yl(cyclopentyl)methanone (2.50 g, 62.3%) as a cloudy, yellow oil.

Synthesis of cyclopentyl(furan-2-yl)methanone. Prepared as described in General Procedure B except the nucleophile was prepared as follows: furan (2.8 mL, 2.62 g, 38.5 mmol) was added to a pear-shaped flask containing dry (3 Å molecular sieves) THF (25 mL) and the mixture was cooled to −78 ° C n-Butyl lithium solution (2.5 M in hexanes, 15 mL, 37.5 mmol) was added via syringe over 5 min and allowed to stir at −78 ° C for 30 min. The furan nucleophile was added as described in General Procedure B to a solution of N-methoxy-N-methylcyclopentanecarboxamide (3.0 g, 19.1 mmol) in dry (3 Å molecular sieves) THE (50 mL) at 0 ° C and allowed to stir overnight. After workup, cyclopentyl(furan-2-yl)methanone (3.0 g, 95.8%) was isolated as a dark red oil that was used without further purification.

Synthesis of cyclopentyl(4-fluorophenyl)methanone. Prepared as described in General Procedure B except the Grignard reagent was formed directly in a 3-neck round-bottom flask with 1-bromo-4-fluoro-benzene (4.2 mL, 6.69 g, 38.2 mmol), crushed Mg turnings (1.2 g, 49.3 mmol), catalytic iodine, and dry (3 Å molecular sieves) THE (75 mL). A solution of N-methoxy-N-methylcyclopentanecarboxamide (3.0 g, 19.1 mmol) in dry (3 Å molecular sieves) THE (25 mL) was added directly to the Grignard reagent at 0 ° C, over 5 min via syringe and allowed to naturally warm to rt overnight. After workup, cyclopentyl(4-fluorophenyl)methanone (3.39 g, 92%) was isolated as a clear, amber oil that was used without further purification.

Synthesis of cyclopentyl(4-methoxyphenyl)methanone. Prepared as described in General Procedure B except the Grignard reagent was formed directly in a 3-neck round-bottom flask with 4-bromoanisole (4.8 mL, 7.17 g, 38.3 mmol), crushed Mg turnings (1.25 g, 51.4 mmol), catalytic iodine, and dry (3× molecular sieves) THF (50 mL). A solution of N-methoxy-N-methylcyclopentanecarboxamide (3.0 g, 19.1 mmol) in dry (3× molecular sieves) THE (30 mL) was added directly to the Grignard reagent at 0 ° C, over 5 min via syringe and allowed to stir for 1 h. After workup, cyclopentyl(4-methoxyphenyl)methanone was isolated as a mixture with anisole as a clear, amber oil (6.59 g, >100%) that was used without further purification. Quantitative yield of cyclopentyl(4-methoxyphenyl)methanone (3.89 g) was assumed for the subsequent reaction.

Synthesis of cyclopentyl(3-(trifluoromethyl)phenyl)methanone. Prepared as described in General Procedure B except the Grignard reagent was formed directly in a 3-neck round-bottom flask with 3-bromobenzotrifluoride (5.3 mL, 8.54 g, 37.9 mmol), crushed Mg turnings (1.2 g, 49.4 mmol), catalytic iodine, and dry (3 Å molecular sieves) THE (75 mL). A solution of N-methoxy-N-methylcyclopentanecarboxamide (3.01 g, 19.1 mmol) in dry (3 Å molecular sieves) THE (25 mL) was added directly to the Grignard reagent at 0 ° C, over 5 min via addition funnel and allowed to stir for 3.5 h. After workup, cyclopentyl(3-(trifluoromethyl)phenyl)methanone was isolated as a mixture with trifluorotoluene as a clear, amber oil (5.43 g, >100%) that was used without further purification. Quantitative yield of cyclopentyl(3-(trifluoromethyl)phenyl)methanone (4.62 g) was assumed for the subsequent reaction.

Synthesis of bicyclo[2.2.1]heptan-2-yl)(phenyl)methanone. Prepared as described in General Procedure B except the Grignard reagent was formed directly in a 3-neck round-bottom flask with bromobenzene (4.4 mL, 6.50 g, 41.8 mmol), crushed Mg turnings (1.3 g, 53.5 mmol), catalytic iodine, and dry (3 Å molecular sieves) THF (75 mL). A solution of N-methoxy-N-methylbicyclo[2.2.1]heptane-2-carboxamide (3.78 g, 20.6 mmol) in dry (3 Å molecular sieves) THE (25 mL) was added directly to the Grignard reagent at 0 ° C, over 5 min via addition funnel and allowed to stir for 2 h. After workup, bicyclo[2.2.1]heptan-2-yl)(phenyl)methanone (3.96 g, 96.1%) was isolated as a clear, amber oil that was used without further purification.

Synthesis of benzo[b]thiophen-2-yl(cyclopentyl)methanone. Prepared as described in General Procedure B except the Grignard reagent was formed directly in a 3-neck round-bottom flask with 2-bromobenzothiophene (2.58 g, 12.1 mmol), crushed Mg turnings (530 mg, 21.8 mmol), catalytic iodine, and dry (3 Å molecular sieves) THF (30 mL). A solution of N-methoxy-N-methylcyclopentanecarboxamide (1.5 g, 9.54 mmol) in dry (3 Å molecular sieves) THE (20 mL) was added directly to the Grignard reagent at 0 ° C, over 1 min via syringe and allowed to naturally warm to rt overnight. After workup, the crude product was purified via flash chromatography (SiO₂, 100% hexanes to 9:1 hexanes:EtOAc) to afford benzo[b]thiophen-2-yl(cyclopentyl)methanone (1.84 g, 84%) as a clear, orange oil that solidified upon storage in a fridge.

Synthesis of cyclopentyl(selenophen-2-yl)methanone. Prepared as described in General Procedure B except the nucleophile was prepared as follows: selenophene (2.8 mL, 2.62 g, 38.5 mmol) was added to a 3-neck round-bottom flask containing dry (3× molecular sieves) THE (50 mL) and the mixture was cooled to −78 ° C n-Butyl lithium solution (2.5 M in hexanes, 9.6 mL, 24 mmol) was added via syringe over 10 min and allowed to stir at −78 ° C for 50 min. A solution of N-methoxy-N-methylcyclopentanecarboxamide (3.0 g, 19.1 mmol) in dry (3× molecular sieves) THE (25 mL) was added via syringe at 0 ° C over 5 min and allowed to stir for 1 2.5 h. After workup, cyclopentyl(selenophen-2-yl)methanone (4.37 g, quant) was isolated as a burnt orange oil that was used without further purification.

Synthesis of cyclopentyl(thiophen-2-yl)methanone. In a dry, 3-neck round-bottom flask containing a Teflon coated magnetic stir bar was added a solution of N-methoxy-N-methylthiophene-2-carboxamide (2.0 g, 11.7 mmol) in dry (3 Å molecular sieves) THF (50 mL). The mixture was cooled to 0 ° C and cyclopentylmagnesium bromide (2.0 M in Et₂O, 15 mL, 30 mmol) was added via syringe over 20 min. The reaction was stirred at 0 ° C for 1 h and then warmed to rt for 3 h. Upon completion, the reaction was quenched with sat'd NH₄Cl (50 mL) at rt and allowed to stir overnight. The mixture was transferred to a separatory funnel, diluted with dH₂O (100 mL), and the aqueous phase was extracted with EtOAc (3×100 mL). The combined organic phases were washed with sat'd NaHCO₃ (50 mL), dH₂O (50 mL), and brine (50 mL). The organic phase was dried over Na₂SO₄ and concentrated to a yellow oil that was purified via flash chromatography (SiO₂, 9:1 hexanes:EtOAc) to afford cyclopentyl(thiophen-2-yl)methanone (2.0 g, 95%) as a clear yellow oil.

General Procedure C: Synthesis of N-(benzo[d][1,3]dioxol-5-yl(cyclopentyl)methylene)-2-methylpropane-2-sulfinamide. In a dry 3-neck round-bottom flask containing a Teflon coated magnetic stir bar was added dry (3 Å molecular sieves) toluene (50 mL), benzo[d][1,3]dioxol-5-yl(cyclopentyl)methanone (2.5 g, 11.4 mmol), racemic tert-butyl sulfinamide (2.1 g, 17.3 mmol), and Ti(OEt)₄ (4.8 mL, 5.22 g, 22.9 mmol). With constant argon flushing, the flask was fitted with a reflux condenser and heated with a heating mantle to maintain a temperature of 80-90 ° C (determined by IR heat gun). This temperature was maintained for 2 h (reaction times ranged from 1-8 h) and then the reaction was cooled to rt to stir overnight. The mixture was quenched with brine (20-50 mL), diluted with EtOAc (75-150 mL), and allowed to stir at rt for 5 min. The precipitate was removed by filtration through celite and the filter cake was washed with EtOAc (2×100 mL). The filtrate was transferred to a separatory funnel and the aqueous layer was drained. The organic phase was washed with brine (2×50 mL), dried over Na₂SO₄, and concentrated to a yellow oil. The crude product was purified via flash chromatography (SiO₂, 9:1 hexanes:EtOAc to 7:3 hexanes:EtOAc) to afford N-(benzo[d][1,3]dioxol-5-yl(cyclopentyl)methylene)-2-methylpropane-2-sulfinamide (2.51 g, 68.6%) as a clear, bright yellow oil.

Synthesis of N-(cyclobutyl(phenyl)methylene)-2-methylpropane-2-sulfinamide. Prepared as described in General Procedure C except using cyclobutyl phenyl ketone (3.8 mL, 4.0 g, 24.9 mmol), racemic tert-butyl sulfinamide (4.53 g, 37.4 mmol), Ti(OEt)₄ (10.4 mL, 11.3 g, 49.5 mmol) and dry (3 Å molecular sieves) toluene (100 mL). The product was purified via flash chromatography (SiO₂, 9:1 hexanes:EtOAc to 7:3 hexanes:EtOAc) to afford N-(cyclobutyl(phenyl)methylene)-2-methylpropane-2-sulfinamide (4.4 g, 67%) as a clear, bright yellow oil.

Synthesis of N-(cyclopentyl(thiophen-2-yl)methylene)-2-methylpropane-2-sulfinamide. Prepared as described in General Procedure C except with cyclopentyl(thiophen-2-yl)methanone (2.24 g, 12.4 mmol), racemic tert-butyl sulfinamide (3.16 g, 26.1 mmol), Ti(OEt)₄ (5.2 mL, 5.66 g, 24.8 mmol) and dry (3 Å molecular sieves) toluene (75 mL). The product was purified via flash chromatography (SiO₂, 9:1 hexanes:EtOAc to 8:2 hexanes:EtOAc) to afford N-(cyclopentyl(thiophen-2-yl)methylene)-2-methylpropane-2-sulfinamide (2.1 g, 59.8%) as a dark, amber oil.

Synthesis of N-(cyclopentyl(m-tolyl)methylene)-2-methylpropane-2-sulfinamide. Prepared as described in General Procedure C except using cyclopentyl(3-methylphenyl)methanone (4.46 g, 23.7 mmol), racemic tert-butyl sulfinamide (6.0 g, 49.5 mmol), Ti(OEt)₄ (10 mL, 10.9 g, 47.8 mmol) and dry (3 Å molecular sieves) toluene (100 mL). The product was purified via flash chromatography (SiO₂, 8:2 hexanes:EtOAc) to afford N-(cyclopentyl(m-tolyl)methylene)-2-methylpropane-2-sulfinamide (3.5 g, 50.7%) as a clear, yellow oil.

Synthesis of N-(cyclopentyl(4-fluorophenyl)methylene)-2-methylpropane-2-sulfinamide. Prepared as described in General Procedure C except using cyclopentyl(4-fluorophenyl)methanone (3.39 g, 17.6 mmol), racemic tert-butyl sulfinamide (3.21 g, 26.4 mmol), Ti(OEt)₄ (7.4 mL, 8.05 g, 35.2 mmol) and dry (3 Å molecular sieves) toluene (100 mL). The product was purified via flash chromatography (SiO₂, 8:2 hexanes:EtOAc to 7:3 hexanes:EtOAc) to afford N-(cyclopentyl(4-fluorophenyl)methylene)-2-methylpropane-2-sulfinamide (1.61 g, 31%) as a clear, amber oil.

Synthesis of N-(cyclopentyl(4-methoxyphenyl)methylene)-2-methylpropane-2-sulfinamide. Prepared as described in General Procedure C except using cyclopentyl(4-methoxyphenyl)methanone (6.59 g crude mixture, 3.90 g ketone, 19.1 mmol), racemic tert-butyl sulfinamide (3.52 g, 29.0 mmol), Ti(OEt)₄ (8.0 mL, 8.70 g, 38.1 mmol) and dry (3 Å molecular sieves) toluene (75 mL). The product was purified via flash chromatography (SiO₂, 8:2 hexanes:EtOAc to 7:3 hexanes:EtOAc) to afford N-(cyclopentyl(4-methoxyphenyl)methylene)-2-methylpropane-2-sulfinamide (3.07 g, 52.3%) as a clear, yellow oil.

Synthesis of N-(cyclopentyl(3-(trifluoromethyl)phenyl)methylene)-2-methylpropane-2-sulfinamide. Prepared as described in General Procedure C except using cyclopentyl(3-(trifluoromethyl)phenyl)methanone (5.43 g crude mix, 4.62 g ketone, 19.1 mmol), racemic tert-butyl sulfinamide (3.46 g, 28.5 mmol), Ti(OEt)₄ (8.0 mL, 8.70 g, 38.1 mmol) and dry (3 Å molecular sieves) toluene (75 mL). The mixture was stirred at 80-90 ° C for 4 h and then quenched. The product was purified via flash chromatography (SiO₂, 8:2 hexanes:EtOAc) to afford N-(cyclopentyl(3-(trifluoromethyl)phenyl)methylene)-2-methylpropane-2-sulfinamide (3.0 g, 45.5%) as a clear, yellow oil.

Synthesis of bicyclo[2.2.1]heptan-2-yl)(phenyl)methylene)-2-methylpropane-2-sulfinamide. Prepared as described in General Procedure C except using bicyclo[2.2.1]heptan-2-yl)(phenyl)methanone (3.96 g, 19.8 mmol),), racemic tert-butyl sulfinamide (3.62 g, 29.7 mmol), Ti(OEt)₄ (8.0 mL, 8.70 g, 38.1 mmol) and dry (3 Å molecular sieves) toluene (100 mL). The product was purified via flash chromatography (SiO₂, 9:1 hexanes:EtOAc to 8:2 hexanes:EtOAc) to afford bicyclo[2.2.1]heptan-2-yl)(phenyl)methylene)-2-methylpropane-2-sulfinamide (2.70 g, 45%) as a clear, orange oil.

Synthesis of N-(benzo[b]thiophen-2-yl(cyclopentyl)methylene)-2-methylpropane-2-sulfinamide. Prepared as described in General Procedure C except using benzo[b]thiophen-2-yl(cyclopentyl)methanone (1.84 g, 7.99 mmol), racemic tert-butyl sulfinamide (1.52 g, 12.5 mmol), Ti(OEt)₄ (3.6 mL, 3.91 g, 17.1 mmol) and dry (3 Å molecular sieves) toluene (75 mL). The product was purified via flash chromatography (SiO₂, 9:1 hexanes:EtOAc to 8:2 hexanes:EtOAc) to afford N-(benzo[b]thiophen-2-yl(cyclopentyl)methylene)-2-methylpropane-2-sulfinamide (2.08 g, 78.2%) as a bright yellow oil.

Synthesis of N-(cyclopentyl(selenophen-2-yl)methylene)-2-methylpropane-2-sulfinamide. Prepared as described in General Procedure C except using cyclopentyl(selenophen-2-yl)methanone (4.37 g, 19.2 mmol), racemic tert-butyl sulfinamide (3.50 g, 28.9 mmol), Ti(OEt)₄ (8.0 mL, 8.70 g, 38.1 mmol) and dry (3 molecular sieves) toluene (100 mL). The reaction was also quenched with sat'd NaHCO₃. The product was purified via flash chromatography (SiO₂, 8:2 hexanes:EtOAc) to afford N-(cyclopentyl(selenophen-2-yl)methylene)-2-methylpropane-2-sulfinamide (3.1 g, 48.9%) as a dark red oil.

Synthesis of 1-cyclopentyl-1-(m-tolyl)ethan-1-amine (Compound 36). Prepared as described for Compound 3 except using N-(cyclopentyl(m-tolyl)methylene)-2-methylpropane-2-sulfinamide (2.0 g, 6.86 mmol), methylmagnesium bromide (1.4 M in THF:toluene, 15 mL, 21 mmol), and dry (3 Å molecular sieves) THE (50 mL). The crude product was purified via flash chromatography (SiO₂, 8:2 hexanes:EtOAc to 7:3 hexanes:EtOAc) to afford 1-cyclopentyl-1-(m-tolyl)ethan-1-amine (790 mg, 56.6% yield) as a clear, yellow oil. The purified freebase was converted to the fumarate salt by dissolving in EtOH (10 mL) and adding a solution of fumaric acid (445.9 mg) in EtOH (~25 mL). The solution was evaporated to afford a solid that was crystallized 2× by dissolving in boiling EtOH (~20-30 mL) and layering with Et₂O to afford 1-cyclopentyl-1-(m-tolyl)ethan-1-amine hemifumarate as a white crystalline solid. ¹H NMR hemifumarate salt (400 MHz, DMSO-d₆) δ ppm=7.34 (s, 1H), 7.28 (apparent d, J=8.1 Hz, 1H), 7.20 (t, J=7.6 Hz, 1H), 7.04 (apparent d, J=7.3 Hz, 1H), 6.40 (s. 1H equiv, fumarate), 2.35-2.21 (m, 1H, overlap with 2.30), 2.30 (s, 3H, overlap with 2.35-2.21), 1.52-1.32 (m, 6H, overlap with 1.45), 1.45 (s, 3H, overlap with 1.52-1.32), 1.32-1.18 (m, 2H). ¹³C NMR hemifumarate salt (100.6 MHz, DMSO-d₆) δ ppm=168.26 (fumarate), 145.97 (1C), 136.81 (1C), 135.50 (fumarate), 127.76 (1C), 127.05 (1C), 126.26 (1C), 122.73 (1C), 57.95 (1C), 50.25 (1C), 26.86 (1C), 26.77 (1C), 25.28 (1C), 25.08 (2C), 21.29 (1C). HPLC Purity (70:30 Formate buffer:ACN, 220 nm): 99.9%. HRMS: m/z calcd for C₁₄H₂₁N+H [M+H]⁺, 204.1747; found 204.1744, Δppm=−1.46; m/z calcd for C₁₄H₁₉ [M−NH₃]⁺, 187.1481; found 187.1480, Δppm=−0.53.

Synthesis of 1-cyclopentyl-1-(thiophen-2-yl)ethan-1-amine (Compound 45). Prepared as described for Compound 3 except using N-(cyclopentyl(thiophen-2-yl)methylene)-2-methylpropane-2-sulfinamide (2.0 g, 7.06 mmol), methylmagnesium bromide (1.4 M in THF:toluene, 15 mL, 21 mmol), and dry (3 Å molecular sieves) THF (50 mL). The crude freebase was purified via flash chromatography (SiO₂, 8:2 hexanes:EtOAc to 7:3 hexanes:EtOAc) to afford 1-cyclopentyl-1-(thiophen-2-yl)ethan-1-amine (720 mg, 52.2% yield) as a clear, colorless oil. The HCl salt was prepared by in EtOH (30 mL) and by adding concentrated HCl (337 μL) to afford 1-cyclopentyl-1-(thiophen-2-yl)ethan-1-amine hydrochloride as an off-white crystalline solid. ¹H NMR HCl salt (400 MHz, DMSO-d₆) δ ppm=8.78 (bs, 3H), 7.55 (dd, J=5.1, 1.1 Hz, 1H), 7.34 (dd, J=3.7, 1.2 Hz, 1H), 7.07 (dd, J=5.1, 3.6 Hz, 1H), 2.57-2.47 (m, 1H), 1.73-1.66 (m, 1H, overlap with 1.65), 1.65 (s, 3H, overlap with 1.73-1.66), 1.56-1.36 (m, 6H), 1.35-1.21 (m, 1H). ¹³C NMR HCl salt (100.6 MHz, DMSO-d₆) δ ppm=144.83 (1C), 127.02 (1C). 125.82 (1C), 125.71 (1C), 58.76 (1C), 49.36 (1C), 27.20 (1C), 27.00 (1C), 25.14 (1C), 24.98 (1C), 23.08 (1C). mp: 202.1-202.5 ° C HPLC Purity (70:30 Formate buffer:ACN, 220 nm): 99.3%. HRMS: m/z calcd for C₁₁H₁₇NS+H [M+H]⁺, 196.1154; found 196.1156, Δppm=−1.02; m/z calcd for C₁₁H₁₅S [M−NH₃]⁺, 179.0889; found 179.0890, Δppm=0.56.

Synthesis of 1-(benzo[d][1,3]dioxol-5-yl)-1-cyclopentylethan-1-amine) (Compound 46). Prepared as described for compound 3 except using N-(benzo[d][1,3]dioxol-5-yl(cyclopentyl)methylene)-2-methylpropane-2-sulfinamide (1.5 g, 4.67 mmol), methylmagnesium bromide (1.4 M in THF:toluene, 10 mL, 14 mmol), and dry (3 Å molecular sieves) THE (20 mL). The crude freebase was converted to the fumarate salt by dissolving in MeOH (50 mL) and adding solid fumaric acid (413 mg) with stirring. Once dissolved, the solution was evaporated to afford a solid that was crystallized 2× by dissolving in a minimum amount of hot MeOH and layering with Et₂O to afford 1-(benzo[d][1,3]dioxol-5-yl)-1-cyclopentylethan-1-amine fumarate (stoichiometric salt) (1.47 g, 90.1% yield) as a white, chunky crystalline solid. ¹H NMR fumarate salt (400 MHz, DMSO-d₆) δ ppm=8.93 (bs, NH₃), 7.15 (d, J=1.5 Hz, 1H), 6.95 (dd, J=8.3 Hz, 1H), 6.88 (d, J=8.2 Hz, 1H), 6.45 (s, 2H equivalent, fumarate), 6.01-5.99 (m, 2H), 2.39-2.25 (m, 1H), 1.56-1.13 (m, 8H, overlap with 1.51), 1.51 (s, 3H, overlap with 1.56-1.13). ¹³C NMR fumarate salt (100.6 MHz, DMSO-d₆) δ ppm=167.79 (fumarate), 147.25 (1C), 146.08 (1C), 137.43 (1C), 135.13 (fumarate), 118.99 (1C), 107.58 (1C), 106.63 (1C), 101.03 (1C), 59.24 (1C), 49.71 (1C), 26.88 (1C), 26.76 (1C), 25.00 (1C), 24.81 (1C), 22.69 (1C). mp: 226.9-227.4 ° C HPLC Purity (70:30 Formate buffer:ACN, 220 nm): 97.4%. HRMS: m/z calcd for C₁₄H₁₉NO₂+H [M+H]⁺, 234.1489; found 234.1487, Δppm=−0.85; m/z calcd for C₁₄H₁₇O₂[M−NH₃]⁺, 217.1223; found 217.1224, Δppm=0.46.

Synthesis of 1-cyclobutyl-1-phenylethan-1-amine (Compound 47). Prepared as described for Compound 3 except using N-(cyclobutyl(phenyl)methylene)-2-methylpropane-2-sulfinamide (1.5 g, 5.69 mmol), methylmagnesium bromide (1.4 M in THF:toluene, 12 mL, 16.8 mmol), and dry (3 Å molecular sieves) THE (25 mL). The HCl salt was prepared by in MeOH (20 mL) and by adding concentrated HCl (141 μL) to afford 1-cyclobutyl-1-phenylethan-1-amine hydrochloride (180 mg, 15.0% yield) as a white crystalline solid, which was only crystalized 1× by dissolving in a minimum amount of MeOH and layering with Et₂O. ¹H NMR HCl salt (400 MHz, DMSO-d₆) δ ppm=8.69 (bs, NH₃), 7.53-7.48 (dm, J=7.8 Hz, 2H), 7.44-7.37 (tm, J=7.3 Hz, 2H), 7.36-7.30 (tm, J=7.2 Hz, 1H), 2.81-2.74 (m, 1H), 2.02-1.92 (m, 1H), 1.92-1.81 (m, 1H), 1.79-1.62 (m, 3H), 1.59 (s, 3H), 1.56-1.47 (m, 1H). ¹³C NMR HCl salt (100.6 MHz, DMSO-d₆) δ ppm=140.45 (1C), 128.32 (2C), 127.63 (1C), 125.53 (2C), 59.51 (1C), 44.06 (1C), 22.79 (1C), 22.70 (1C), 21.22 (1C), 16.02 (1C). mp: 253.5-254.9 ° C HPLC Purity (70:30 Formate buffer:ACN, 220 nm): 100%. HRMS: m/z calcd for C₁₂H₁₇N+H [M+H]⁺, 176.1434; found 176.1436, Δppm=−1.14; m/z calcd for C₁₂H₁₅ [M−NH₃]⁺, 159.1168; found 159.1171, Δppm=1.88.

Synthesis of 1-cyclopentyl-1-(4-fluorophenyl)ethan-1-amine (Compound 48). Prepared as described for Compound 3 except using N-(cyclopentyl(4-fluorophenyl)methylene)-2-methylpropane-2-sulfinamide (1.0 g, 3.38 mmol), methylmagnesium bromide (1.4 M in THF:toluene, 7.2 mL, 10.1 mmol), and dry (3 Å molecular sieves) THE (20 mL). The acetate salt was prepared by in MeOH (30 mL) and by adding glacial acetic acid (183 μL) to afford 1-cyclopentyl-1-(4-fluorophenyl)ethan-1-amine acetate (440 mg, 48.7% yield) as a white crystalline solid that was crystallized 1× from a minimum amount of hot MeOH and Et₂O. ¹H NMR acetate salt (400 MHz, DMSO-d₆) δ ppm=7.55-7.47 (m, 2H), 7.11-7.03 (tm, J=8.4 Hz, 2H), 4.85 (bs, 3H), 2.27-2.11 (m, 1H), 1.87 (s, 3H, acetate), 1.54-1.35 (m, 6H), 1.33 (s, 3H), 1.26-1.06 (m, 2H). ¹³C NMR acetate salt (100.6 MHz, DMSO-d₆) δ ppm=172.24 (acetate), [161.60, 159.20 (d, J=241 Hz, 1C)], [145.63, 145.60 (d, J=2.8 Hz, 1C)], [127.58, 127.50 (d, J=7.9 Hz, 2C)], [114.12, 113.92 (d, J=20.6 Hz, 2C)], 56.17 (1C), 51.07 (1C), 28.70 (1C), 26.91 (1C), 26.65 (1C), 25.40 (1C), 25.25 (1C), 21.52 (acetate). mp: 118.3-118.6 ° C HPLC Purity (70:30 Formate buffer:ACN, 220 nm): 97.9%. HRMS: m/z calcd for C₁₃H₁₈FN+H [M+H]⁺, 208.1496; found 208.1498, Δppm=0.96; m/z calcd for C₁₃H₁₆F [M−NH₃]⁺, 191.1231; found 191.1232, Δppm=0.52.

Synthesis of 1-cyclopentyl-1-(4-methoxyphenyl)ethan-1-amine (Compound 49). Prepared as described for Compound 3 except using N-(cyclopentyl(4-methoxyphenyl)methylene)-2-methylpropane-2-sulfinamidewil (1.50 g, 4.89 mmol), methylmagnesium bromide (1.4 M in THF:toluene, 10.2 mL, 14.3 mmol), and dry (3 Å molecular sieves) THE (25 mL) and was quenched after 1.5 h. The acetate salt was prepared by in MeOH (40 mL) and by adding glacial acetic acid (238 μL). The solvent was removed under a warm stream of air with MeOH additions (3×~5 mL) resulting in an oily solid. The mixture was layered with Et₂O (~100 mL) and allowed to crystalize at −20 ° C to produce needle-like crystals. The crystals were collected by vacuum filtration, washed with Et₂O (3×25 mL), and dried to afford 1-cyclopentyl-1-(4-methoxyphenyl)ethan-1-amine acetate (700 mg, 51.4% yield) as white needle-like crystals. ¹H NMR acetate salt (400 MHz, DMSO-d₆) δ ppm=7.39 (dm, J=8.8 Hz, 2H), 6.84 (dm, J=8.8 Hz, 2H), 5.82+5.73 (2×bs, 3H, NH₃), 3.72 (s, 3H), 2.26-2.13 (m, 1H), 1.84 (s, 3H, acetate), 1.53-1.17 (m, 8H, overlap with 1.33), 1.33 (d, J=2.1 Hz, 3H, overlap with 1.53-1.17). ¹³C NMR acetate salt (100.6 MHz, DMSO-d₆) δ ppm=172.40 (acetate), 157.25 (1C), 140.75 (1C), 140.57 (1C), 126.67 (2C), 112.89 (2C), 56.24 (1C), 56.16 (1C), 54.90 (1C), 51.01 (1C), 50.97 (1C), 28.16 (1C), 27.98 (1C), 26.97 (1C), 25.35 (1C), 25.33 (1C), 21.93 (1C, acetate), 21.84 (1C, acetate). ¹³C NMR Freebase (run because of conformational effects observed in acetate spectra) (100.6 MHz, CDCl₃) δ ppm=158.15 (1C), 142.14 (1C), 127.05 (2C), 113.61 (2C), 56.38 (1C), 55.68 (1C), 52.46 (1C), 29.94 (1C), 27.99 (1C), 27.87 (1C), 26.32 (1C), 26.16 (1C). mp: 115.5-115.7 ° C HPLC Purity (70:30 Formate buffer:ACN, 220 nm): 99.4%. HRMS: m/z calcd for C₁₄H₂₁NO+H [M+H]⁺, 220.1696; found 220.1699, Δppm=1.36; m/z calcd for C₁₄H₁₉0 [M−NH₃]⁺, 203.1430; found 203.1433, Δppm=1.47.

Synthesis of 1-cyclopentyl-1-(3-(trifluoromethyl)phenyl)ethan-1-amine (Compound 50). Prepared as described for Compound 3 except using N-(cyclopentyl(3-(trifluoromethyl)phenyl)methylene)-2-methylpropane-2-sulfinamide (1.5 g, 4.37 mmol), methylmagnesium bromide (1.4 M in THF:toluene, 9.4 mL, 13.2 mmol), and dry (3 Å molecular sieves) THE (25 mL). The acetate salt was prepared by in MeOH (20 mL) and by adding glacial acetic acid (125 μL) and evaporating with MeOH washes (3×~5 mL). The resulting oil was suspended in hexanes (~40 mL) and allowed to evaporate. When solid formed, the mixture was cooled to −20 ° C overnight. The remaining hexanes were decanted and the solid was washed with hexanes (3×5 mL) to afford 1-cyclopentyl-1-(3-(trifluoromethyl)phenyl)ethan-1-amine acetate (340 mg, 24.6% yield) as a white crystalline solid that was crystallized 1× by dissolving in a minimum amount of hot EtOAc and layering with hexanes. ¹H NMR acetate salt (400 MHz, DMSO-d₆) δ ppm=7.87 (s, 1H), 7.81-7.75 (m, 1H), 7.55-7.47 (m, 2H), 5.31 (bs, 3H), 2.31-2.17 (m, 1H), 1.89 (s, 3H, acetate), 1.59-1.38 (m, 6H), 1.36 (s, 3H), 1.19-1.08 (m, 2H). ¹⁹F NMR acetate Salt (376.5 MHz, DMSO-d₆) δ ppm=60.55 ppm. mp: 82.3-83.2 ° C HPLC Purity (70:30 Formate buffer:ACN, 220 nm): 97.8%. HRMS: m/z calcd for C₁₄H₁₈F3N+H [M+H]⁺, 258.1464; found 258.1465, Δppm=0.39; m/z calcd for C₁₄H₁₆F₃[M−NH₃]⁺, 241.1199; found 241.1198, Δppm=−0.41.

Synthesis of 1-(benzo[b]thiophen-2-yl)-1-cyclopentylethan-1-amine (Compound 51). Prepared as described for Compound 3 except using N-(benzo[b]thiophen-2-yl(cyclopentyl)methylene)-2-methylpropane-2-sulfinamide (1.52 g, 4.56 mmol), methylmagnesium bromide (1.4 M in THF:toluene, 9.6 mL, 13.4 mmol), and dry (3 Å molecular sieves) THE (30 mL). The crude product was purified via flash chromatography (SiO₂, 8:2 hexanes:EtOAc to 7:3 hexanes:EtOAc) to afford 1-(benzo[b]thiophen-2-yl)-1-cyclopentylethan-1-amine (730 mg, 65.7% yield) as a clear, yellow oil. The fumarate salt was prepared from the purified freebase (560 mg) by in EtOH (20 mL) and by adding solid fumaric acid (265 mg) with stirring. Once dissolved, the solution was evaporated and the resulting solid was washed with Et₂O (3×10 mL) to afford 1-(benzo[b]thiophen-2-yl)-1-cyclopentylethan-1-amine fumarate (stoichiometric salt) as a white, chunky solid that was crystalized 1× from hot EtOH (20 mL) and Et₂O (100 mL). ¹H NMR fumarate salt (400 MHz, DMSO-d₆) δ ppm=8.44 (bs, NH₃), 7.90 (d, J=7.7 Hz, 1H), 7.77 (dd, J=7.0, 1.2 Hz, 1H), 7.39 (s, 1H), 7.34 (td, J=7.3, 1.1 Hz, 1H, overlap with 7.30), 7.30 (td, J=7.3, 1.1 Hz, 1H, overlap with 7.34), 6.53 (s, 2H equivalents, fumarate), 2.47-2.36 (m, 1H), 1.62-1.53 (m, 2H, overlap with 1.56), 1.56 (s, 3H, overlap with 1.62-1.53), 1.53-1.35 (m, 6H). ¹³C NMR fumarate salt (100.6 MHz, DMSO-d₆) δ ppm=167.07 (fumarate), 152.22 (1C), 139.35 (1C), 138.50 (1C), 134.68 (fumarate), 124.29 (1C), 124.03 (1C), 123.33 (1C), 122.18 (1C), 120.28 (1C), 57.49 (1C), 50.41 (1C), 27.01 (1C), 26.96 (1C), 26.54 (1C), 25.36 (1C), 25.30 (1C). mp: 177.1-177.7 ° C HPLC Purity (70:30 Formate buffer:ACN, 220 nm): 99.7%. HRMS: m/z calcd for C₁₅H₁₉NS+H [M+H]⁺, 246.1311; found 246.1312, Δppm=0.41; m/z calcd for C₁₅H₁₇S [M−NH₃]⁺, 229.1045; found 229.1047, Δppm=0.87.

Synthesis of 1-cyclopentyl-1-phenylpentan-1-amine (Compound 52). Prepared as described for Compound 3 except using N-(cyclopentyl(phenyl)methylene)-2-methylpropane-2-sulfinamide (1c) (1.01 g, 3.64 mmol), butylmagnesium chloride (2.0 M in THF, 5.4 mL, 10.8 mmol), and dry (3 Å molecular sieves) THE (30 mL). The crude freebase was purified via flash chromatography (SiO₂, 9:1 hexanes:EtOAc to 7:3 hexanes:EtOAc) to afford 1-cyclopentyl-1-phenylpentan-1-amine (580 mg, 65.0% yield) as a colorless transparent oil. The purified freebase was converted to the hydrochloride salt by dissolving in MeOH (~30 mL) and adding a concentrated HCl (218 μL). The resulting solid was washed with Et₂O (3×20 mL) and dried to afford 1-cyclopentyl-1-phenylpentan-1-amine hydrochloride a white powdery solid. ¹H NMR HCl salt (400 MHz, DMSO-d₆) δ ppm=8.59 (bs, NH₃), 7.46-7.42 (dm, J=7.8 Hz, 2H, overlap with 7.42-7.37), 7.42-7.37 (tm, J=7.6 Hz, 2H, overlap with 7.46-7.42), 7.34-7.27 (tm, J=6.9 Hz, 1H), 2.44-2.31 (m, 1H), 2.20-2.06 (m, 1H), 2.00-1.88 (m, 1H), 1.60-1.51 (m, 1H), 1.50-1.31 (m, 7H), 1.30-1.17 (m, 3H), 1.00-0.87 (m, 1H), 0.81 (t, J=7.0 Hz, 3H). ¹³C NMR HCl salt (100.6 MHz, DMSO-d₆) δ ppm=139.54 (1C), 128.23 (2C), 127.27 (1C), 125.93 (2C), 63.90 (1C), 49.41 (1C), 35.13 (1C), 26.50 (1C), 26.27 (1C), 24.92 (1C), 24.69 (1C), 24.47 (1C), 22.30 (1C), 13.79 (1C). mp: >260 ° C HPLC Purity: (70:30 Formate buffer:ACN, 200 nm): 98.0%. HRMS: m/z calcd for C₁₆H₂₅N+H [M+H]⁺, 232.2060; found 232.2061, Δppm=0.43; m/z calcd for C₁₆H₂₃ [M−NH₃]⁺, 215.1794; found 215.1796, Δppm=0.93.

Synthesis of 1-cyclopentyl-1-phenylprop-2-yn-1-amine (Compound 53). Prepared as described for Compound 3 except using N-(cyclopentyl(phenyl)methylene)-2-methylpropane-2-sulfinamide (1c) (1.0 g, 3.60 mmol), ethynylmagnesium bromide (0.5 M in THF, 19 mL, 9.5 mmol), and dry (3 Å molecular sieves) THE (20 mL). The crude freebase was purified via flash chromatography (SiO₂, 9:1 hexanes:EtOAc to 8:2 hexanes:EtOAc) to afford 1-cyclopentyl-1-phenylprop-2-yn-1-amine (210 mg, 29.3 yield) as a colorless transparent oil. The purified freebase was converted to the hydrochloride salt by dissolving in MeOH (~20 mL) and adding a concentrated HCl (96.8 μL). The resulting solid was washed with Et₂O (3×~5 mL) and dried to afford 1-cyclopentyl-1-phenylprop-2-yn-1-amine hydrochloride a white powdery solid. ¹H NMR HCl salt (400 MHz, DMSO-d₆) δ ppm=9.30 (bs, NH₃), 7.84-7.79 (dm, J=7.7 Hz, 2H), 7.49-7.43 (tm, J=7.3 Hz, 2H), 7.43-7.38 (tm, J=7.2 Hz, 1H), 4.14 (s, 1H), 2.79-2.64 (m, 1H), 1.97-1.86 (m, 1H), 1.81-1.61 (m, 2H), 1.61-1.46 (m, 2H), 1.46-1.33 (m, 1H), 1.25-1.02 (m, 2H). ¹³C NMR HCl salt (100.6 MHz, DMSO-d₆) δ ppm=138.22 (1C), 128.76 (1C), 128.59 (2C), 126.24 (2C), 81.39 (1C), 78.85 (1C), 60.79 (1C), 49.23 (1C), 28.32 (1C), 28.08 (1C), 25.36 (1C), 24.76 (1C). 253.0-253.9 ° C HPLC Purity: (70:30 Formate buffer:ACN, 200 nm): 96.5%. HRMS: m/z calcd for C₁₄H₁₇N+H [M+H]⁺, 200.1434; found 200.1434, Δppm=0.00; m/z calcd for C₁₄H₁₅ [M−NH₃]⁺, 183.1168; found 183.1170, Δppm=1.09.

Synthesis of 1-cyclopentyl-1-(selenophen-2-yl)ethan-1-amine (Compound 54). Prepared as described for Compound 3 except using N-(cyclopentyl(selenophen-2-yl)methylene)-2-methylpropane-2-sulfinamide (1.51 g, 4.57 mmol), methylmagnesium bromide (1.4 M in THF:toluene, 9.8 mL, 13.7 mmol), and dry (3 Å molecular sieves) THE (25 mL) and was quenched after 2.5 h. The fumarate salt was prepared by in MeOH (30 mL) and by adding solid fumaric acid (321 mg) with stirring. Once dissolved, the solvent was removed under vacuum and the resulting reddish/orange solid was washed with Et₂O (3×20 mL) to afford 1-cyclopentyl-1-(selenophen-2-yl)ethan-1-amine hemifumarate (880 mg, 64.14% yield). The solid was crystalized 1× from hot MeOH (~15 mL) and Et₂O (80 mL) to afford a white, crystalline solid. ¹H NMR hemifumarate salt (400 MHz, DMSO-d₆) δ ppm=7.98 (dd, J=5.5, 1.2 Hz, 1H), 7.21-7.15 (m, 2H), 6.47 (s, 1H equiv, fumarate), 2.31 (m, 1H), 1.59-1.49 (m, 2H, overlap with 1.49-1.44), 1.49-1.44 (m, 7H, overlap with 1.59-1.49, 1.44-1.30), 1.44-1.30 (m, 2H, overlap with 1.49-1.44). ¹³C NMR Hemifumarate salt (100.6 MHz, DMSO-d6) δ ppm=167.66 (fumarate), 160.52 (1C), 135.07 (fumarate), 129.79 (1C), 129.12 (1C), 124.97 (1C), 58.24 (1C), 50.98 (1C), 28.25 (1C), 27.14 (1C), 27.10 (1C), 25.39 (1C), 25.35 (1C). mp: 216.4-217.6 ° C MS ASAP, MW: 243.1, m/z: 243.1 M+1 peaks: (3%), 244.1 (8%), 245.1 (4%), 246.1 (5%), M-NH₃ peaks: 229.15 (40%), 228.17 (30%), 227.21 (100%), 225.17 (80%), 224.15 (40%), 223.14 (45%).

Synthesis of cyclopentyl(m-tolyl)methanamine (Compound 37). Prepared as described for Compound 29 except using N-(cyclopentyl(m-tolyl)methylene)-2-methylpropane-2-sulfinamide (1.50 g, 5.15 mmol), NaBH₄ (580 mg, 15.3 mmol), and absolute EtOH (25 mL). The resulting freebase was directly converted to the HCl salt by dissolving in EtOH (20 mL) and adding concentrated HCl (431 μL) to afford cyclopentyl(m-tolyl)methanamine hydrochloride (910 mg, 78.4% yield) as a white fluffy solid. ¹H NMR HCl salt (400 MHz, DMSO-d₆) δ ppm=8.60 (bs, NH₃⁺), 7.35-7.24 (m, 3H), 7.18-7.14 (dm, J=7.1 Hz, 1H), 3.90 (d, J=10.0 Hz, 1H), 2.43-2.32 (m, 1H), 2.31 (s, 3H), 1.95-1.83 (m, 1H), 1.70-1.57 (m, 1H), 1.57-1.32 (m, 4H), 1.26-1.15 (m, 1H), 1.11-0.96 (m, 1H). ¹³C NMR HCl salt (100.6 MHz, DMSO-d₆) δ ppm=138.39 (1C), 137.65 (1C), 128.88 (1C), 128.45 (1C), 128.13 (1C), 124.62 (1C), 59.10 (1C), 44.43 (1C), 29.82 (1C), 29.48 (1C), 24.77 (1C), 24.26 (1C), 21.02 (1C). mp: >260 ° C HPLC Purity: (70:30 Formate buffer:ACN, 200 nm): 98.4%. HRMS: m/z calcd for C₁₃H₁₉N+H [M+H]⁺, 190.1590; found 190.1590, Δppm=0.0; C₁₃H₁₇+H [M+H]⁺, 173.1325; found 173.1326, Δppm=0.58.

Synthesis of benzo[d][1,3]dioxol-5-yl(cyclopentyl)methanamine (Compound 34). Prepared as described for Compound 29 except using N-(benzo[d][1,3]dioxol-5-yl(cyclopentyl)methylene)-2-methylpropane-2-sulfinamide (1.0 g, 3.11 mmol), NaBH₄ (360 mg, 9.51 mmol), and absolute EtOH (15 mL). The resulting freebase was directly converted to the HCl salt by dissolving in EtOH (30 mL) and adding concentrated HCl (313 μL) to afford benzo[d][1,3]dioxol-5-yl(cyclopentyl)methanamine hydrochloride (780 mg, 98.1% yield) as a white, crystalline solid. The solid was crystalized 3× by dissolving in MeOH and layering with Et₂O. ¹H NMR HCl salt (400 MHz, DMSO-d₆) δ ppm 8.54 (bs, NH₃⁺), 7.17 (d, J=1.4 Hz, 1H), 6.90 (dd, J=8.0, 1.4 Hz, 1H), 6.91 (d, J=8.0 Hz, 1H), 6.02 (s, 2H), 3.89 (d, J=10.1 Hz, 1H), 2.41-2.26 (m, 1H), 1.93-1.82 (m, 1H), 1.70-1.58 (m, 1H), 1.58-1.47 (m, 2H), 1.47-1.34 (m, 2H), 1.30-1.18 (m, 1H), 1.10-0.97 (m, 1H). ¹³C NMR HCl salt (100.6 MHz, DMSO-d₆) δ ppm 147.33 (1C), 147.04 (1C), 132.25 (1C), 121.42 (1C), 108.11 (1C), 107.69 (1C), 101.14 (1C), 58.92 (1C), 44.43 (1C), 29.82 (1C), 29.50 (1C), 24.82 (1C), 24.27 (1C). mp: 254.4-254.8 ° C HPLC Purity (70:30 Formate buffer:ACN, 220 nm): 100%. HRMS: m/z calcd for C₁₃H₁₇NO₂+H [M+H]⁺, 220.1332; found 220.1321, Δppm=−4.99; C₁₃H₁₅O₂[M+H]⁺, 203.1067; found 203.1059, Δppm=−3.94.

Synthesis of cyclopentyl(furan-2-yl)methanamine (Compound 40). Prepared as described for Compound 29 except using N-(cyclopentyl(furan-2-yl)methylene)-2-methylpropane-2-sulfinamide (740 mg, 2.77 mmol), NaBH₄ (320 mg, 8.46 mmol), and absolute EtOH (15 mL). The resulting freebase was directly converted to the HCl salt by dissolving in MeOH (15 mL) and adding concentrated HCl (227 μL) to afford cyclopentyl(furan-2-yl)methanamine hydrochloride (410 mg, 73.5% yield) as a white, crystalline solid. The solid was crystalized 3× by dissolving in MeOH and layering with Et₂O. ¹H NMR HCl salt (400 MHz, DMSO-d₆) δ ppm=8.66 (bs, NH₃⁺), 7.70 (dd, J=1.9, 0.8 Hz, 1H), 6.55 (dd, J=3.3, 0.7 Hz, 1H), 6.47 (dd, J=3.3, 1.8 Hz, 1H), 4.18 (d, J=8.0 Hz, 1H), 2.48-2.36 (m, 1H), 1.89-1.77 (m, 1H), 1.64-1.32 (m, 6H), 1.24-1.10 (m, 1H). ¹³C NMR HCl salt (100.6 MHz, DMSO-d₆) δ ppm=150.61 (1C), 143.07 (1C), 110.61 (1C), 109.08 (1C), 51.77 (1C), 42.09 (1C), 29.31 (1C), 28.77 (1C), 24.81 (1C), 24.47 (1C). mp: 229.1-229.7 ° C HPLC Purity (70:30 Formate buffer:ACN, 220 nm): 100%. HRMS: m/z calcd for C₁₀H₁₅NO+H [M+H]⁺, 166.1226; found 166.1228, Δppm=1.20; C₁₀H₁₃0 [M+H]⁺, 149.0961; found 149.0963, Δppm=1.34.

Synthesis of cyclobutyl(phenyl)methanamine (Compound 33). Prepared as described for Compound 29 except using N-(cyclobutyl(phenyl)methylene)-2-methylpropane-2-sulfinamide (1.0 g, 3.79 mmol), NaBH₄ (430 mg, 11.4 mmol), and absolute EtOH (20 mL). The resulting freebase was directly converted to the HCl salt by dissolving in EtOH (30 mL) and adding concentrated HCl (284 μL) to afford cyclobutyl(phenyl)methanamine hydrochloride (520 mg, 69.4% yield) as a white, crystalline solid. The solid was crystalized 3× by dissolving in EtOH and layering with Et₂O. ¹H NMR HCl salt (400 MHz, DMSO-d₆) δ ppm=8.58 (bs, NH₃), 7.51-7.47 (dm, J=7.9 Hz, 2H), 7.42-7.37 (tm, J=7.3 Hz, 2H), 7.36-7.31 (tm, J=7.2 Hz, 1H), 4.15 (d, J=9.9 Hz, 1H), 2.87-2.70 (m, 1H), 2.13-1.94 (m, 2H), 1.85-1.54 (m, 4H). ¹³C NMR HCl salt (100.6 MHz, DMSO-d₆) δ ppm=136.84 (1C), 128.53 (2C), 128.34 (1C), 127.50 (2C), 59.01 (1C), 38.81 (1C), 25.56 (1C), 24.29 (1C), 16.83 (1C). mp: >260 ° C HPLC Purity (70:30 Formate buffer:ACN, 220 nm): 97.8%. HRMS: m/z calcd for C₁₁H₁₅N+H [M+H]⁺, 162.1277; found 162.1279, Δppm=1.23; C₁₁H₁₃ [M+H]⁺, 145.1012; found 145.1014, Δppm=1.38.

Synthesis of cyclopentyl(4-methoxyphenyl)methanamine (Compound 41). Prepared as described for Compound 29 except using N-(cyclopentyl(4-methoxyphenyl)methylene)-2-methylpropane-2-sulfinamide (1.34 g, 4.36 mmol), NaBH₄ (490 mg, 12.9 mmol), and MeOH (20 mL). The resulting freebase was directly converted to the HCl salt by dissolving in MeOH (25 mL) and adding concentrated HCl (344 μL) to afford cyclopentyl(4-methoxyphenyl)methanamine hydrochloride (830 mg, 79.0% yield) as a white, fluffy solid. The solid was crystalized 3× by dissolving in EtOH and layering with Et₂O. ¹H NMR HCl salt (400 MHz, CDCl₃) δ ppm=8.69 (bs, NH₃), 7.31 (d, J=8.6 Hz, 2H), 6.82 (d, J=8.6 Hz, 2H), 3.85-3.76 (m, 1H, overlap with 3.78), 3.78 (s, 3H, overlap with 3.85-3.76), 2.51-2.33 (m, 1H), 1.99-1.86 (m, 1H), 1.73-1.59 (m, 1H), 1.59-1.30 (m, 5H), 1.06-0.92 (m, 1H). ¹³C NMR HCl salt (100.6 MHz, CDCl₃) δ ppm=159.79 (1C), 129.11 (2C), 128.90 (1C), 114.23 (2C), 61.03 (1C), 55.24 (1C), 44.78 (1C), 31.05 (1C), 30.36 (1C), 25.67 (1C), 24.98 (1C). mp: 235.3-235.7 ° C HPLC Purity (70:30 Formate buffer:ACN, 220 nm): 100%. HRMS: m/z calcd for C₁₃H₁₉NO+H [M+H]⁺, 206.1539; found 206.1540, Δppm=0.49; C₁₃H₁₇O [M+H]⁺, 189.1274; found 189.1276, Δppm=1.06.

Synthesis of cyclopentyl(4-fluorophenyl)methanamine (Compound 44). Prepared as described for Compound 29 except with N-(cyclopentyl(4-fluorophenyl)methylene)-2-methylpropane-2-sulfinamide (0.610 g, 2.06 mmol), NaBH₄ (235 mg, 6.19 mmol), and MeOH (20 mL). The resulting freebase was directly converted to the HCl salt by dissolving in MeOH (25 mL) and adding concentrated HCl (344 μL) to afford cyclopentyl(4-fluorophenyl)methanamine hydrochloride (360 mg, 90.5% yield) as a fluffy white crystalline solid. The solids were crystalized (3X) by dissolving in boiling MeOH and layering with Et₂O followed by storage in a −20 ° C freezer. ¹H NMR HCl salt (400 MHz, DMSO-d₆) δ ppm=8.66 (bs, NH₃), 7.62-7.55 (m, 2H), 7.27-7.20 (tm, J=8.8 Hz, 2H), 4.01 (d, J=10.1 Hz, 1H), 2.45-2.29 (m, 1H), 1.96-1.82 (m, 1H), 1.69-1.58 (m, 1H), 1.58-1.33 (m, 4H), 1.26-1.13 (m, 1H), 1.09-0.95 (m, 1H). ¹³C NMR HCl salt (100.6 MHz, DMSO-d₆) δ ppm=[163.08, 160.66 (d, J=243 Hz, 1C)], [134.79, 134.76 (d, J=3.0 Hz, 1C)], [129.84, 129.76 (d, J=8.0 Hz, 2C)], [115.43, 115.22 (d, J=21.1 Hz, 2C)], 58.30 (1C), 44.45 (1C), 29.81 (1C), 29.45 (1C), 24.79 (1C), 24.28 (1C). ¹⁹F NMR (376 MHz, DMSO-d₆) δ ppm=−113.42. mp: >260 ° C HPLC Purity (70:30 Formate buffer:ACN, 220 nm): HRMS: m/z calcd for C₁₂H₁₆FN+H [M+H]⁺, 193.1340; found 193.1341, Δppm=0.52; C₁₂H₁₄F [M+H]⁺, 177.1074; found 177.1076, Δppm=1.13.

Synthesis of cyclopentyl(3-(trifluoromethyl)phenyl)methanamine (Compound 43). Prepared as described for Compound 29 except using N-(cyclopentyl(3-(trifluoromethyl)phenyl)methylene)-2-methylpropane-2-sulfinamide (1.36 g, 3.94 mmol), NaBH₄ (470 mg, 12.4 mmol), and MeOH (20 mL). The resulting freebase was directly converted to the HCl salt by dissolving in MeOH (40 mL) and adding concentrated HCl (248 μL) and the crude salt was crystallized by dissolving in hot EtOAc (35 mL) and layering with hexanes (150 mL).

The solid was filtered and washed with hexanes (3×20 mL) to afford cyclopentyl(3-(trifluoromethyl)phenyl)methanamine hydrochloride (800 mg, 74.7% yield) as a white, crystalline solid. ¹H NMR HCl salt (400 MHz, CDCl₃) δ ppm=8.77 (bs, NH₃), 7.96 (s, 1H), 7.87 (apparent d, J=7.6 Hz, 1H), 7.73 (apparent d, J=7.8 Hz, 1H), 7.65 (apparent t, J=7.7 Hz, 1H), 4.17 (d, J=10.0 Hz, 1H), 2.47-2.32 (m, 1H), 1.99-1.86 (m, 1H), 1.71-1.58 (m, 1H), 1.58-1.34 (m, 4H), 1.24-1.12 (m, 1H), 1.12-0.99 (m, 1H). ¹³C NMR HCl salt (100.6 MHz, DMSO-d₆) δ ppm=139.85 (1C), 131.86 (1C), 129.67 (1C), [129.71, 129.38, 129.06, 128.75 (q, J=32.0 Hz, 1C)], [128.14, 125.43, 122.72, 120.02 (q, J=273 Hz, 1C)], [125.11, 125.07, 125.04, 125.00 (q, J=3.8 Hz, 1C), [124.52, 124.48, 124.45, 124.41 (q, J=3.8 Hz, 1C)], 58.36 (1C), 44.33 (1C), 29.82 (1C), 29.33 (1C), 24.76 (1C), 24.26 (1C). mp: >260 ° C HPLC Purity (70:30 Formate buffer:ACN, 220 nm): 100%. HRMS: m/z calcd for C₁₃H₁₆F₃N+H [M+H]⁺, 244.1308; found 244.1302, Δppm=−2.46; C₁₃H₁₄F₃[M+H]⁺, 227.1042; found 227.1037, Δppm=−2.20.

Synthesis of benzo[b]thiophen-2-yl(cyclopentyl)methanamine (Compound 35). Prepared as described for Compound 29 except using N-(benzo[b]thiophen-2-yl(cyclopentyl)methylene)-2-methylpropane-2-sulfinamide (560 mg, 1.68 mmol), NaBH₄ (200 mg, 5.29 mmol), and MeOH (20 mL). The resulting freebase was directly converted to the HCl salt by dissolving in MeOH (30 mL) and adding concentrated HCl (130 μL) to afford benzo[b]thiophen-2-yl(cyclopentyl)methanamine hydrochloride (320 mg, 71.3% yield) as a white, crystalline solid. The solid was crystallized 1× by dissolving in MeOH and layering with Et₂O. ¹H NMR HCl salt (400 MHz, DMSO-d₆) δ ppm=8.87 (bs, NH₃), 8.04-7.99 (m, 1H), 7.91-7.86 (m, 1H), 7.63 (s, 1H), 7.45-7.38 (m, 2H), 4.50 (d, J=9.8 Hz, 1H), 2.57-2.51 (m, 1H, overlap with DMSO), 2.00-1.90 (m, 1H), 1.73-1.65 (m, 1H), 1.62-1.45 (m, 5H), 1.31-1.18 (m, 1H). ¹³C NMR HCl salt (100.6 MHz, DMSO-d6) δ ppm=141.36 (1C), 138.97 (1C), 138.71 (1C), 124.83 (1C), 124.67 (1C), 124.14 (1C), 123.83 (1C), 122.58 (1C), 54.68 (1C), 44.63 (1C), 29.78 (1C), 29.48 (1C), 24.98 (1C), 24.49 (1C). mp: 251.7-251.9 ° C HPLC Purity (70:30 Formate buffer:ACN, 220 nm): 98.9%. MS ASAP, MW: 231.4, m/z: 232.3 (22%, M+1), 233.3 (5%, M+2), 216.3 (20, M-NH₃), 215.2 (100, M-NH₃).

Synthesis of bicyclo[2.2.1]heptan-2-yl)(phenyl)methanamine (Compound 42). Prepared as described for Compound 29 except using bicyclo[2.2.1]heptan-2-yl)(phenyl)methylene)-2-methylpropane-2-sulfinamide (1.05 g, 3.46 mmol), NaBH₄ (390 mg, 10.3 mmol), and MeOH (15 mL). The resulting freebase was directly converted to the HCl salt by dissolving in MeOH (30 mL) and adding concentrated HCl (210 μL) to afford bicyclo[2.2.1]heptan-2-yl)(phenyl)methanamine hydrochloride (510 mg, 62.0% yield) as a white, crystalline solid and as an approximate 80:20 mixture of diastereomers as determined by HPLC. The solid was crystalized 1× by dissolving in MeOH and layering with Et₂O. mp: >260 ° C HPLC Purity (70:30 Formate buffer:ACN, 220 nm): 3.47 min (20%); 3.64 min (79.9%). HRMS: m/z calcd for C₁₄H₁₉N+H [M+H]⁺, 202.1590; found 202.1592, Δppm=0.99; C₁₄H₁₇+H [M+H]⁺, 185.1325; found 185.1327, Δppm=1.08.

Synthesis of cyclopentyl(selenophen-2-yl)methanamine (Compound 38). Prepared as described for Compound 29 except using N-(cyclopentyl(selenophen-2-yl)methylene)-2-methylpropane-2-sulfinamide(1.50 g, 4.54 mmol), NaBH₄ (520 mg, 13.7 mmol), and MeOH (25 mL). The resulting freebase was directly converted to the HCl salt by dissolving in MeOH (50 mL) and adding concentrated HCl (386 μL) to afford cyclopentyl(selenophen-2-yl)methanamine hydrochloride (1.01 g, 84.1% yield) as a yellow/orange solid. The solid was crystalized 2× by dissolving in MeOH and layering with Et₂O to afford an off-white, crystalline solid. ¹H NMR HCl salt (400 MHz, DMSO-d₆) δ ppm=8.66 (bs, NH₃), 8.20 (dd, J=5.6, 1.3 Hz, 1H), 7.42 (d, J=3.7 Hz, 1H), 7.23 (ddd, J=5.7, 3.7, 1.2 Hz, 1H), 4.38 (d, J=9.74 Hz, 1H), 2.44-2.30 (m, 1H), 1.93-1.82 (m, 1H), 1.69-1.58 (m, 1H, overlap with 1.59-1.37), 1.59-1.37 (m, 5H, overlap with 1.69-1.58), 1.21-1.09 (m, 1H). ¹³C NMR HCl salt (100.6 MHz, DMSO-d6) δ ppm=147.51 (1C), 132.91 (1C), 129.92 (1C), 128.69 (1C), 56.34 (1C), 45.75 (1C), 29.80 (1C), 29.69 (1C), 25.03 (1C), 24.43 (1C). mp: >260 ° C HPLC Purity (70:30 Formate buffer:ACN, 220 nm): 98.8%. MS ASAP, MW (M+1 peaks): 228.2, m/z: 228.2 (23%), 230.2 (3%), M-NH₃ peaks: 215.2 (30%), 213.15 (100%), 211.1 (60%), 210.1 (30%), 209.18 (30%).

Synthesis of 1-cyclopentyl-N-(cyclopropylmethyl)-1-phenylethan-1-amine. Prepared as described for Compound 18 except using 1-cyclopentyl-1-phenylethan-1-amine (3) (300 mg, 1.33 mmol), MeOH (10 mL), glacial AcOH (7.6 μL, 7.97 mg, 0.13 mmol), NaBH₃CN (180 mg, 2.86 mmol), and cyclopropane carboxaldehyde (149 μL, 139.7 mg, 1.99 mmol). The crude product was purified via flash chromatography (SiO₂, 9:1 hexanes:EtOAc to 8:2 hexanes:EtOAc) to afford 1-cyclopentyl-N-(cyclopropylmethyl)-1-phenylethan-1-amine (150 mg, 46.4% yield) as a cloudy oil. The purified freebase was converted to the hydrochloride salt by dissolving in MeOH (25 mL) and titrating with concentrated HCl until the pH<2. The resulting solid was washed with hexanes (3×10 mL) and dried to afford 1-cyclopentyl-N-(cyclopropylmethyl)-1-phenylethan-1-amine hydrochloride as a white, crystalline solid. ¹H NMR freebase (CDCl₃, 400 MHz) δ ppm 9.47 (bs, NH⁺), 9.26 (bs, NH⁺), 7.60 (d, J=7.8 Hz, 1H), 7.46-7.39 (tm, J=7.4 Hz, 1H), 7.38-7.32 (tm, J=7.2 Hz, 1H), 2.85-2.67 (m, 2H), 2.09-1.97 (m, 1H), 1.97-1.85 (m, 1H), 1.62 (s, 3H), 1.60-1.46 (m, 3H), 1.46-1.29 (m, 2H), 1.16-0.93 (m, 3H), 0.54-0.43 (m, 2H), 0.27-0.16 (m, 1H), 0.12-0.02 (m, 1H). ¹³C NMR freebase (CDCl₃, 100.6 MHz) δ ppm 139.12 (1C), 128.62 (2C), 128.14 (1C), 126.45 (2C), 66.39 (1C), 48.55 (1C), 47.75 (1C), 27.84 (1C), 27.10 (1C), 25.32 (1C), 24.25 (1C), 15.14 (1C), 7.57 (1C), 4.72 (1C), 4.57 (1C). mp: 220.7-221.2 ° C HPLC Purity: (50:50 Formate buffer:ACN, 220 nm): 97.9%. HRMS: m/z calcd for C₁₇H₂₅N+H [M+H]⁺, 244.2060; found 244.2061, Δppm=0.41.

### Alternative Synthetic Routes

Substituted methanamines may be prepared by condensation between an appropriately substituted ketone or aldehyde and an amine (ammonia, primary amine, or secondary amine or protected variants) to form an intermediate imine or iminium compound; which may or may not be isolated the imine/iminium compound would next be subjected to nucleophilic attack by hydride or an appropriate carbanion or organometallic reagent. The resulting product could be further modified if necessary. Other sources of nitrogen could be used, for example to form sulfonylimines or N-phosphonyl imines. Substituted methanamines may be prepared via nucleophilic reaction of a ketone with a carbanion or organometallic reagent to form an intermediate secondary or tertiary alcohol. The alcohol may be converted to an alkyl halide or sulfonate and reacted with an amine (ammonia, appropriately substituted primary amine, or appropriately substituted secondary amine) via a nucleophilic substitution reaction. The resulting product could be further modified if necessary.

### Example 2: NMDA Receptor Radio-Ligand Binding Studies

Rat forebrains from 7-8 week old Sprague-Dawley rats (BioChemed, USA) were homogenized in a buffer containing 10 mM HEPES and 1 mM EDTA (pH 7.4) using a mechanical homogenizer (Janke-Kunkel Ultra-Turrax T25). A 10 mL volume was used per brain for all steps involving suspension of the brain homogenate tissue. The brain homogenate was then centrifuged (15 minutes at 20,000 RPM and 4 ° C). The supernatant was carefully poured off and the pellet was resuspended in fresh 10 mM HEPES and 1 mM EDTA containing buffer. This process was repeated 5 times. Following the 5^{th} centrifugation, the buffer was replaced with a buffer containing 10 mM HEPES (pH 7.4). The homogenate was then incubated in a water bath at 37 ° C in the dark (covered with foil). Centrifugation was then repeated 3 additional times and after resuspending in the HEPES buffer, the final homogenate was aliquoted and stored at −80 ° C until use. Protein concentration of the homogenate was determined by utilizing the Bradford method using Coomassie protein assay reagent (Sigma, USA) with Bovine Serum albumin (Sigma, USA) as a standard.

At the time of the experiment, the homogenate was diluted to a final concentration of 133 g/ml in 10 mM HEPES buffer, 1 mM glutamate, and 0.1 mM glycine (pH 7.4) and combined with 1 nM (+)-[3H]-MK-801 (Perkin Elmer NET972250UC), unlabeled competitor compounds at varying concentrations, or 30 uM MK-801 to determine non-specific binding, in a total volume of 1 mL in a 96 well plate. The protein homogenate is always added to the wells last. The plates were covered with aluminum foil, and placed on a mechanical shaker (250 RPM) for 2 h at ambient temperature (−25 ° C). After 2 h, the homogenate was collected by vacuum filtration using a Unifilter-96 Cell Harvester (Perkin Elmer) over presoaked UniFilter-96 GF/C P Microplates (Perkin Elmer) and filters were washed with room temperature 10 mM HEPES buffer (pH 7.4) (3×1 mL). The filter plates were dried overnight in a fume hood. The next day, the bound tritium was measured via liquid scintillation counting with 30 L MicroScint-O (Perkin Elmer) added per well, using a MicroBeta2 Plate Reader with 6-detectors scintillation counter (Perkin Elmer) at 55% efficiency. IC₅₀ value estimates were determined in GraphPad Prism 9.3.1 using non-linear regression (single site fit) with log-concentration plotted against percent specific binding. Kᵢ values were calculated using the equation of Cheng and Prusoff. The K_{d} for MK-801 (1.67 nM), was determined via homologous binding experiments. Experiments were performed in duplicate. N=1-4 per compound. Results are presented in Table 2.

TABLE 2
NMDAR Binding Affinities determined using [³H]-MK-801 competition studies in
rat forebrain. (N = 1-4). (% inhibition of specific [³H]-MK-801 binding at 10,000 nM).

| Compound | Structure | pKᵢ (mean ± SEM) |
|---|---|---|
| Ketamine | 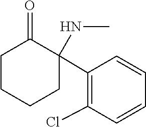 [SMILES: CNC1(c2ccccc2Cl)CCCCC1=O] | 6.50 ± 0.107 |
| 2 | 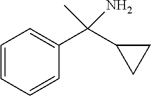 | 6.19 ± 0.162 |
| 3 | 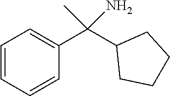 | 7.11 ± 0.041 |
| 4 | 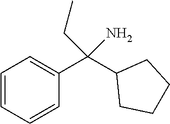 | 6.64 ± 0.101 |
| 5 | 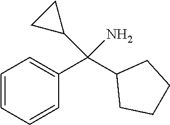 [SMILES: NC(c1ccccc1)(C1CCCC1)C1CC1] | 6.23 ± 0.046 |
| 6 | 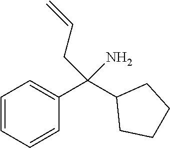 [SMILES: C=CCC(N)(c1ccccc1)C1CCCC1] | 5.89 ± 0.072 |
| 7 | 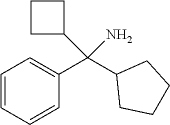 [SMILES: NC(c1ccccc1)(C1CCCC1)C1CCC1] | 5.63 ± 0.029 |
| 8 | 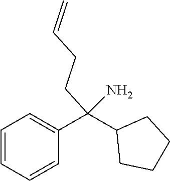 [SMILES: C=CCCC(N)(c1ccccc1)C1CCCC1] | 6.24 ± 0.444 |
| 9 | 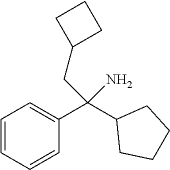 [SMILES: NC(CC1CCC1)(c1ccccc1)C1CCCC1] | 5.80 ± 0.095 |
| 10 | 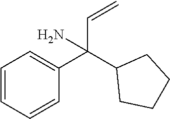 | 6.05 |
| 11 | 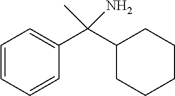 | 6.09 ± 0.120 |
| 13 | 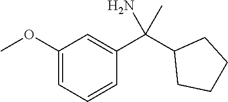 [SMILES: COc1cccc(C(C)(N)C2CCCC2)c1] | 6.82 ± 0.039 |
| 14 | 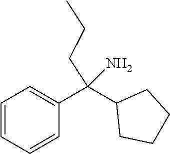 [SMILES: CCCC(N)(c1ccccc1)C1CCCC1] | 5.94 ± 0.166 |
| 15 | 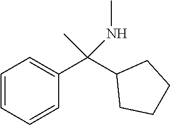 | 6.56 ± 0.064 |
| 16 | 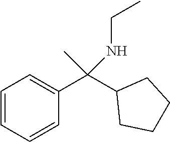 [SMILES: CCNC(C)(c1ccccc1)C1CCCC1] | 6.93 ± 0.073 |
| 19 | 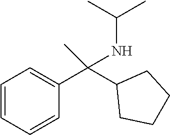 [SMILES: CC(C)NC(C)(c1ccccc1)C1CCCC1] | 7.34 ± 0.041 |
| 20 | 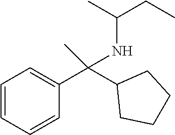 [SMILES: CCC(C)NC(C)(c1ccccc1)C1CCCC1] | 7.35 ± 0.075 |
| 21 | 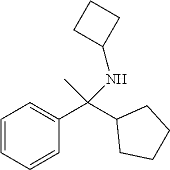 [SMILES: CC(NC1CCC1)(c1ccccc1)C1CCCC1] | 8.06 ± 0.021 |
| 22 | 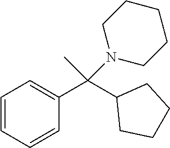 [SMILES: CC(c1ccccc1)(C1CCCC1)N1CCCCC1] | 6.08 ± .0633 |
| 23 | 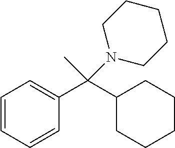 [SMILES: CC(c1ccccc1)(C1CCCCC1)N1CCCCC1] | 5.52 ± .118 |
| 25 | 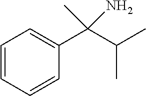 | 5.86 ± 0.044 |
| 26 | 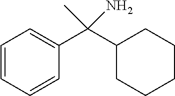 | <5.00 (18.4% inhibition) |
| 27 | 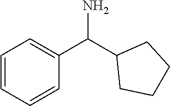 [SMILES: NC(c1ccccc1)C1CCCC1] | <5.00 (32.4% inhibition) |
| 28 | 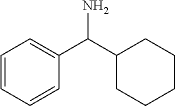 [SMILES: NC(c1ccccc1)C1CCCCC1] | 4.97 ± 0.054 |
| 29 | 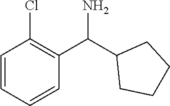 [SMILES: NC(c1ccccc1Cl)C1CCCC1] | 5.47 ± 0.030 |
| 30 | 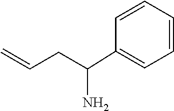 | <5.00 (43.9% inhibition) |
| 31 | 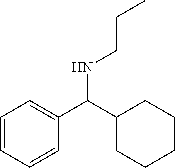 [SMILES: CCCNC(c1ccccc1)C1CCCCC1] | 5.76 |
| 32 | 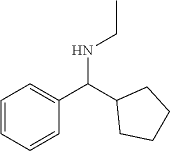 | 5.85 |
| 33 | 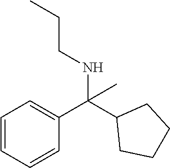 [SMILES: CCCNC(C)(c1ccccc1)C1CCCC1] | 7.43 ± 0.081 |
| 34 | 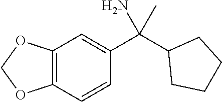 [SMILES: CC(N)(c1ccc2c(c1)OCO2)C1CCCC1] | 7.34 ± 0.002 |
| 45 | 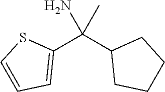 | 0.761 ± 0.030 |
| 47 | 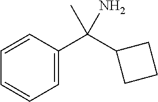 | 6.75 ± 0.016 |
| 48 | 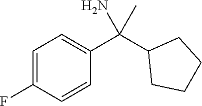 | 6.96 ± 0.019 |
| 49 | 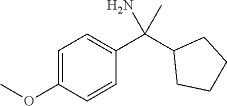 [SMILES: COc1ccc(C(C)(N)C2CCCC2)cc1] | 6.49 ± 0.0039 |
| 50 | 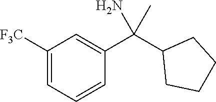 [SMILES: CC(N)(c1cccc(C(F)(F)F)c1)C1CCCC1] | 5.86 ± 0.11 |
| 51 | 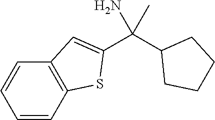 [SMILES: CC(N)(c1cc2ccccc2s1)C1CCCC1] | <5.00 (34.6% inhibition) |
| 52 | 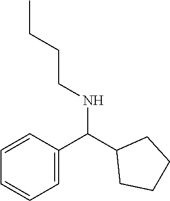 [SMILES: CCCCNC(c1ccccc1)C1CCCC1] | 5.81 ± 0.046 |
| 53 | 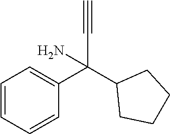 | <5.00 (45.0% inhibition) |
| 54 | 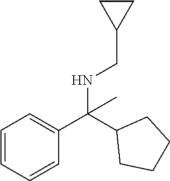 [SMILES: CC(NCC1CC1)(c1ccccc1)C1CCCC1] | 7.90 ± 0.040 |

### SERT Radio-Ligand Binding Studies

Valiscreen Serotonin Transporter (SERT) (Human) Cell Line (HEK293 host cells, Product No: RBHSTM-K) grown in DMEM/F12 media with 10% FBS, 0.1 mg/ml Geneticin, 1% GlutaMAX, and 1% Penicillin-Streptomycin added were used to prepare SERT membrane fractions. Cells were grown in 150 mm dishes and harvested between 70-90% confluency, passages 5-20. Cells were detached using a buffer containing 1 mM HEPES and 2 mM EDTA (pH 7.4). After detachment, the cells were homogenized using a handheld homogenizer, and the lysate was centrifuged (30 minutes at 30,000×G) at 4 ° C The resulting pellet was resuspended in storage buffer containing 20 mM HEPES, 10 mM MgCl₂, 0.1 mM EDTA, and 10% sucrose at pH 7.4. Protein concentration was determined via the Bradford method using Coomassie protein assay reagent (Sigma, USA) with Bovine Serum albumin (Sigma, USA) as standard.

Buffer containing 10 mM HEPES, 120 mM NaCl, and 5 mM KCl (pH 7.4) was added to SERT membranes to dilute to a final protein concentration of 5 g/mL per well. Additionally, 0.5 nM [3H]-Citalopram (Perkin Elmer NET1039250UC) was added, along with unlabeled competitors at various concentrations or 30 M citalopram for nonspecific binding controls, bringing the total volume to 1 ml in a 96 well plate. SERT membranes were always added last to each well. The plates were covered from light with aluminum foil and incubated at ambient temperature (−25 ° C) and placed on a mechanical shaker (250RPM) for 2 h. After 2 h, SERT protein was captured via vacuum filtration using a Unifilter-96 Cell Harvester (Perkin Elmer) over presoaked UniFilter-96 GF/C P Microplates (Perkin Elmer) and filters were washed with room temperature 10 mM HEPES, 120 mM NaCl, and 5 mM KCl buffer (pH 7.4) (3×1 mL). The filter plates were dried overnight in a fume hood. The next day, the SERT bound tritium was measured via liquid scintillation counting with MicroScint-O (Perkin Elmer), using a MicroBeta2 Plate Reader with 6-detectors scintillation counter (Perkin Elmer) at 55% efficiency. IC₅₀ value estimates were determined in GraphPad Prism 9.3.1 using non-linear regression (single site fit) with log-concentration plotted against percent specific binding. Compounds were initially screened at 10,000 nM and IC50 experiments were then conducted on those showing>5000 inhibition. Compounds that did not showing>50% inhibition were screened once more to confirm (n=2). For each experiments compounds were run in duplicate. N=1-3 repeats per compound. Results are presented in Table 3.

TABLE 3
SERT pKi Values Determined using [³H]-citalopram Competition Studies with
hSERT Expressing HEK293 Cell Membranes. <5 refers to pIC₅₀. (N = 1).

| Compound | Structure | pKᵢ |
|---|---|---|
| Escitalopram | 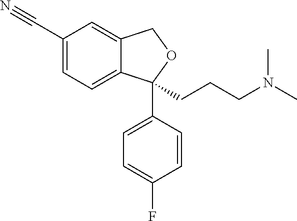 [SMILES: CN(C)CCC[C@@]1(c2ccc(F)cc2)OCc2cc(C#N)ccc21] | 9.12 |
| Desipramine | 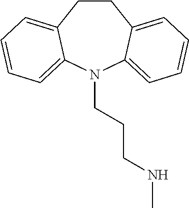 [SMILES: CNCCCN1c2ccccc2CCc2ccccc21] | 7.57 |
| 2 | 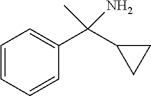 | <5.00 |
| 3 | 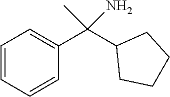 | <5.00 |
| 4 | 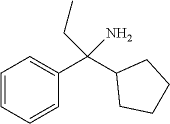 | <5.00 |
| 5 | 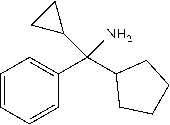 [SMILES: NC(c1ccccc1)(C1CCCC1)C1CC1] | <5.00 |
| 6 | 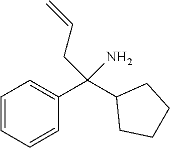 [SMILES: C=CCC(N)(c1ccccc1)C1CCCC1] | 5.50 |
| 9 | 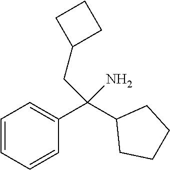 [SMILES: NC(CC1CCC1)(c1ccccc1)C1CCCC1] | <5.00 |
| 11 | 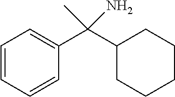 | <5.00 |
| 15 | 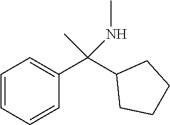 | 6.30 |
| 16 | 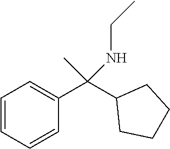 [SMILES: CCNC(C)(c1ccccc1)C1CCCC1] | 5.95 |
| 19 | 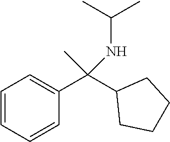 [SMILES: CC(C)NC(C)(c1ccccc1)C1CCCC1] | 6.82 |
| 20 | 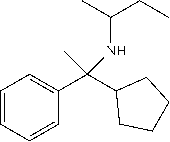 [SMILES: CCC(C)NC(C)(c1ccccc1)C1CCCC1] | 5.61 |
| 22 | 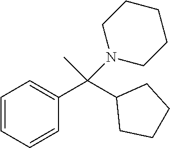 [SMILES: CC(c1ccccc1)(C1CCCC1)N1CCCCC1] | 6.32 |
| 23 | 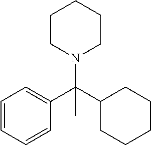 [SMILES: CC(c1ccccc1)(C1CCCCC1)N1CCCCC1] | 5.58 |
| 29 | 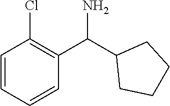 [SMILES: NC(c1ccccc1Cl)C1CCCC1] | <5.00 |

### Example 3: Human Dopamine Reuptake Transporter (DAT) Inhibition Functional Studies

CHO cells stably expressing human Dopamine Transporter Protein (Valiscreen® Perkin Elmer RBHDATM-K) were cultured in 150 mm Tissue Culture dishes (Cell Treat 229651) at 37 ° C and 5% CO₂. Ham's F-12 Nutrient Mix (Gibco, USA) was supplemented with 10% dialyzed FBS (Gibco, USA), 100 g/ml Geneticin (Gibco, USA), and 100 units/ml Penicillin and Streptomycin (Gibco, USA) as the culture media. Cells were maintained on a consistent splitting and assay schedule and utilized until passage 30 (no change in control experiments were observed up to this point).

Cells were detached from their growth dishes using Trypsin-EDTA (0.25%) (Gibco, USA) at 70-80% confluency. The cells were counted and diluted in culture media to a density of 300,000 cells/mL. 200 l of this mixture was added to each well of a clear bottom, black-walled 96 well plate (Coming 3603, USA). Plates were incubated in the laminar flow hood for one hour prior to being placed in the cell culture incubator in order to encourage even cell distribution across the entire well surface.

The next day, Krebs-Ringer HEPES (KRH) Buffer pH 7.4 was prepared (10 mM HEPES, 130 mM NaCl, 1.3 mM KCl, 2.2 mM CaCl₂, 1.2 mM MgSO₄, 1.2 mM KH₂PO₄) and titrated with 6N NaOH. This buffer was stored in a 4 ° C refrigerator for up to 4 days (after which it was discarded). Additionally, a solution containing KRH buffer and 330 M Trypan Blue (Sigma T6146-25G), a solution of 20 mM ASP+(MedChem Express, NJ, USA) dissolved in DMSO, were both prepared. The 20 mM DMSO stock of ASP+ was diluted to a working concentration of 40 M in KRH buffer. DMSO solutions of 40 mM GBR-12935 dihydrochloride (MedChem Express, NJ, USA) and 40 mM test compounds were also prepared. DMSO solutions of test compounds were then diluted to a range of 400 M-40 μM in KRH Buffer with 1% additional DMSO added to provide consistent DMSO concentration in all samples. Compounds were screened for inhibition of ASP+ specific DAT uptake at 10,000 nM. The final DMSO concentration in each well was 0.3%. A 400 M solution of GBR-12935 served as a non-specific fluorescence control to represent the amount of fluorescence present when substrate uptake via DAT is maximally inhibited. KRH buffer with DMSO “vehicle control” served as the total fluorescence control to represent the maximum amount of substrate uptake without DAT transporter inhibition.

At the time of assay, cell media was carefully removed from the wells via pipette aspiration. 100 μL of KRH buffer was then added to all wells. Both controls, non-specific and total fluorescence stock solutions were then added to the plate (50 L of either 400 M GBR-12935 or KRH DMSO buffer respectively). Additionally, GBR-12935 and test compounds were added to the plate (50 L for each sample). The 10,000 nM concentration of test compound was run in triplicate wells. Next, 50 L ASP+(final concentration of 10 μM) was added to each well initiating the experiment. Cells were placed in the cell culture incubator for one hour, at which point 20 L of 330 M Trypan Blue was added to each well to quench extracellular fluorescence. The cells were then placed in the microplate reader (BioTek Synergy Neo 2), using Gen5 Ver 3.11 software. The reader was preheated to 37 ° C and following a brief orbital shake, intracellular fluorescence was measured using a fluorescence intensity reading. Intracellular ASP+ was excited at 430 nm and emission read at 510 nm via a bottom read using the extended gain settings and high lamp energy setting. Results are presented in Table 4.

TABLE 4
hDAT Inhibition Screeening Assay

| | | DAT ASP+ Uptake |
|---|---|---|
| | | % Inhibition at |
| Compound No. | Structure | 10,000 nM |
| 21 | 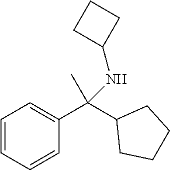 [SMILES: CC(NC1CCC1)(c1ccccc1)C1CCCC1] | 87.3% |
| 33 | 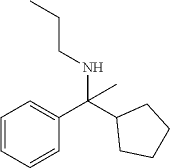 [SMILES: CCCNC(C)(c1ccccc1)C1CCCC1] | 16.3% |
| 20 | 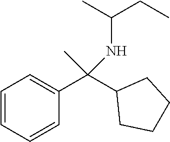 [SMILES: CCC(C)NC(C)(c1ccccc1)C1CCCC1] | 56.7% |
| 19 | 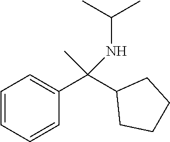 [SMILES: CC(C)NC(C)(c1ccccc1)C1CCCC1] | 39.2% |
| 3 | 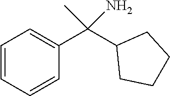 | 16.8% |
| 16 | 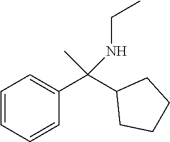 [SMILES: CCNC(C)(c1ccccc1)C1CCCC1] | 16.0% |
| 4 | 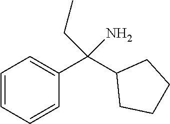 | 24.3% |
| 15 | 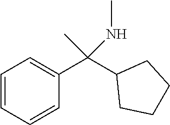 | 18.2% |
| 8 | 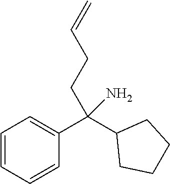 [SMILES: C=CCCC(N)(c1ccccc1)C1CCCC1] | 3.1% |
| 5 | 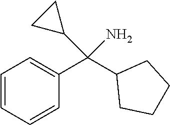 [SMILES: NC(c1ccccc1)(C1CCCC1)C1CC1] | 1.3% |
| 11 | 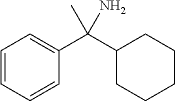 | 11.7% |
| 6 | 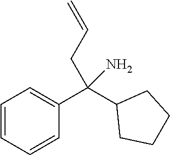 [SMILES: C=CCC(N)(c1ccccc1)C1CCCC1] | 23.1% |
| 25 | 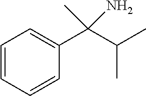 | 37.6% |
| 9 | 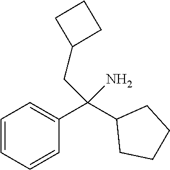 [SMILES: NC(CC1CCC1)(c1ccccc1)C1CCCC1] | −4.0% |
| 7 | 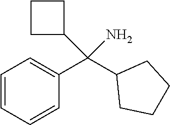 [SMILES: NC(c1ccccc1)(C1CCCC1)C1CCC1] | 24.1% |
| 23 | 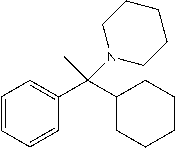 [SMILES: CC(c1ccccc1)(C1CCCCC1)N1CCCCC1] | 71.6% |
| 29 | 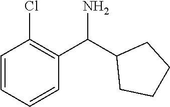 [SMILES: NC(c1ccccc1Cl)C1CCCC1] | 10.3% |
| 28 | 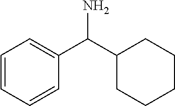 [SMILES: NC(c1ccccc1)C1CCCCC1] | 17.9% |

While preferred embodiments of the present disclosure have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein may be employed in practicing the disclosure. It is therefore contemplated that the disclosure shall also cover any such alternatives, modifications, variations or equivalents. It is intended that the following claims define the scope of the disclosure and that methods and structures within the scope of these claims and their equivalents be covered thereby.

What is claimed is:

1. A composition comprising a compound of Formula (I): 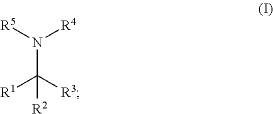
or an enantiomer, a mixture of enantiomers, or an isotopic variant thereof; or a pharmaceutically acceptable salt, solvate or hydrate thereof, wherein:
R¹ is C₃₋₁₀ carbocycle or 3- to 10-membered heterocycle; wherein the C₃₋₁₀ carbocycle and 3- to 10-membered heterocycle are each optionally substituted with one or more R⁶
R² is selected from
hydrogen;
halogen;
C₁₋₈ alkyl, C₂₋₈ alkenyl, and C₂₋₈ alkynyl, each of which is optionally substituted with one or more R⁷; and
C₃₋₁₀ carbocycle and 3- to 10-membered heterocycle; wherein the C₃₋₁₀ carbocycle and 3- to 10-membered heterocycle are each optionally substituted with one or more R⁷;
R³ is selected from:
C₁₋₆ alkyl, C₂₋₆ alkenyl, and C₂₋₆ alkynyl, each of which is optionally substituted with one or more R⁸; and
C₃₋₁₀ carbocycle and 3- to 10-membered heterocycle; wherein the C₃₋₁₀ carbocycle and 3- to 10-membered heterocycle are each optionally substituted with one or more R⁸;
R⁴, R⁵, R⁶, R⁷, and R⁸ are each independently selected from:
hydrogen;
C₁₋₁₀ alkyl, C₂₋₁₀ alkenyl, and C₂₋₁₀ alkynyl optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₁₋₆ cycloalkyl, and C₁₋₆ cycloalkenyl; and
C₃₋₁₀ carbocycle or 3- to 10-membered heterocycle; wherein the C₃₋₁₀ carbocycle and 3- to 10-membered heterocycle are each optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), and C₁₋₈ alkyl;
with the proviso that the compound of Formula I is not

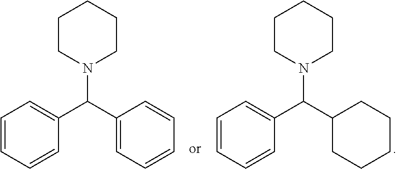

2. A pharmaceutical composition comprising a compound of Formula (I): 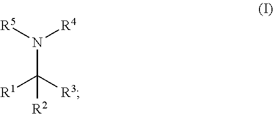
or an enantiomer, a mixture of enantiomers, or an isotopic variant thereof; or a pharmaceutically acceptable salt, solvate or hydrate thereof, wherein:
R¹ is C₃₋₁₀ carbocycle or 3- to 10-membered heterocycle; wherein the C₃₋₁₀ carbocycle and 3- to 10-membered heterocycle are each optionally substituted with one or more R⁶
R² is selected from
hydrogen;
halogen;
C₁₋₈ alkyl, C₂₋₈ alkenyl, and C₂₋₈ alkynyl, each of which is optionally substituted with one or more R⁷; and
C₃₋₁₀ carbocycle and 3- to 10-membered heterocycle; wherein the C₃₋₁₀ carbocycle and 3- to 10-membered heterocycle are each optionally substituted with one or more R⁷;
R³ is selected from: C₁₋₆ alkyl, C₂₋₆ alkenyl, and C₂₋₆ alkynyl, each of which is optionally substituted with one or more R⁸; and C₃₋₁₀ carbocycle and 3- to 10-membered heterocycle; wherein the C₃₋₁₀ carbocycle and 3- to 10-membered heterocycle are each optionally substituted with one or more R¹;
R⁴, R⁵, R⁶, R⁷, and R⁸ are each independently selected from: hydrogen; C₁₋₁₀ alkyl, C₂₋₁₀ alkenyl, C₂₋₁₀ alkynyl, and C₃₋₅ cycloalkyl, wherein C₁₋₁₀ alkyl, C₂₋₁₀ alkenyl, and C₂₋₁₀ alkynyl are optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₁₋₆ cycloalkyl, and C₁₋₆ cycloalkenyl; and C₃₋₁₀ carbocycle or 3- to 10-membered heterocycle; wherein the C₃₋₁₀ carbocycle and 3- to 10-membered heterocycle are each optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), and C₁₋₈ alkyl; or
R⁴ and R⁵ are combined with the nitrogen to which it is attached to form a 6-membered heterocycle;
with the proviso that the compound of Formula I is not

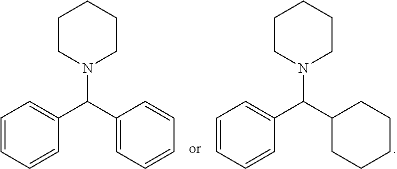

3. The compound of claim 2, or an enantiomer, a mixture of enantiomers, or an isotopic variant thereof; or a pharmaceutically acceptable salt, solvate or hydrate thereof, wherein:
R¹ is selected from phenyl and 5- to 10-membered heteroaryl; wherein the phenyl and 5-to 10-membered heteroaryl are each optionally substituted with one or more R⁶
R² is selected from hydrogen, C₁₋₈ alkyl, C₂₋₈ alkenyl, C₂₋₈ alkynyl, and C₃₋₅ carbocycle, each of which is optionally substituted with one or more R⁷;
R³ is C₃₋₆ carbocycle, wherein the C₃₋₆ carbocycle is optionally substituted with one or more R⁸
R⁴ and R⁵ are each independently selected from: hydrogen, C₁₋₁₀ alkyl, and C₃₋₅ cycloalkyl, wherein the C₁₋₁₀ alkyl is optionally substituted with C₃₋₅ cycloalkyl; or R⁴ and R⁵ are combined with the nitrogen to which it is attached to form a 6-membered heterocycle;
R⁶ is C₁₋₁₀ alkyl, wherein the C₁₋₁₀ alkyl is optionally substituted with halogen;
R⁷ is C₃₋₅ cycloalkyl; and
R⁸ is C₁₋₁₀ alkyl.

4. The compound of claim 3, or an enantiomer, a mixture of enantiomers, or an isotopic variant thereof; or a pharmaceutically acceptable salt, solvate or hydrate thereof, wherein:
R¹ is selected from phenyl, imidazolyl, thiophenyl, selenophenyl, and benzothiophenyl; wherein the phenyl, imidazolyl, thiophenyl, selenophenyl, and benzothiophenyl are each optionally substituted with one or more R⁶
R² is selected from hydrogen, methyl, ethyl, propyl, butyl, ethenyl, propenyl, butenyl, ethynyl, propynyl, butynyl, cyclopropyl, cyclobutyl, and cyclopentyl, each of which is optionally substituted with one or more R⁷;
R³ is cyclobutyl, cyclopentyl, or cyclohexyl;
R⁴ and R⁵ are each independently selected from: hydrogen, methyl, ethyl, n-propyl, isopropyl, n-butyl, sec-butyl, iso-butyl, tert-butyl, methyl-cyclopropyl, methyl-cyclobutyl, methyl-cyclopentyl, cyclopropyl, cyclobutyl, or cyclopentyl; or R⁴ and R⁵ are combined with the nitrogen to which it is attached to form piperidinyl;
R⁶ is methyl or CF₃; and
R⁷ is cyclopropyl or cyclobutyl.

5. The compound of claim 4, or an enantiomer, a mixture of enantiomers, or an isotopic variant thereof; or a pharmaceutically acceptable salt, solvate or hydrate thereof, wherein:
R¹ is selected from phenyl and selenophenyl; wherein the phenyl and selenophenyl are each optionally substituted with one or more R⁶
R² is selected from hydrogen, methyl, butyl, ethenyl, propenyl, butenyl, ethynyl, cyclopropyl, and cyclobutyl, each of which is optionally substituted with one or more R⁷;
R³ is cyclopentyl;
R⁴ and R⁵ are each independently selected from: hydrogen, methyl, ethyl, isopropyl, sec-butyl, methyl-cyclopropyl, cyclobutyl; or R⁴ and R⁵ are combined with the nitrogen to which it is attached to form piperidinyl;
R⁶ is CF₃; and
R⁷ is cyclopropyl or cyclobutyl.

6. The composition of claim 1, wherein R¹ is C₃₋₇ carbocycle or 3- to 7-membered heterocycle; wherein the C₃₋₇ carbocycle and 3- to 7-membered heterocycle are each optionally substituted with one or more R⁶.

7. The composition of claim 1 or 2, wherein R¹ is phenyl, optionally substituted with one or more R⁶.

8. The composition of claim 1 or 2, wherein R¹ is a cycloalkyl selected from cyclooctane, cycloheptane, cyclohexane, cyclopentane, cyclobutane, and cyclopropane, any of which is optionally substituted with one or more R⁶.

9. The composition of claim 1 or 2, wherein R¹ is a 6-membered heteroaryl selected from pyridine, pyrazine, and pyrimidine, any of which is optionally substituted with one or more R⁶.

10. The composition of claim 1 or 2, wherein R¹ is a 5-membered heteroaryl selected from thiophene, furan, pyrrole, pyrazole, selenophene, imidazole, thiazole, isothiazole, oxatriazole, oxadiazole, oxazole, and thiadiazole, any of which is optionally substituted with one or more R⁶.

11. The composition of claim 1 or 2, wherein R¹ is a 6-membered heterocycloalkenyl selected from dihydropyridine, tetrahydropyridine, dihydropyridazine, tetrahydropyridazine, dihydropyrimidine, tetrahydropyrimidine, dihydropyrazine, tetrahydropyridazine, dihydropyrimidine, tetrahydropyrimidine, dihydropyrazine, tetrahydropyrazine, pyran, dihydropyran, thiopyran, dihydrothiopyran, dioxine, dihydrodioxine, oxazine, dihydrooxazine, thiazine, and dihydrothiazine, any of which is optionally substituted with one or more R⁶.

12. The composition of claim 1 or 2, wherein R¹ is a 5-membered heterocycloalkenyl selected from pyrroline, pyrazoline, imidazoline, triazoline, dihydrofuran, dihydrothiophene, oxazoline, isooxazoline, thiazoline, isothiazoline, oxadiazoline, and thiadiazoline, any of which is optionally substituted with one or more R⁶.

13. The composition of claim 1 or 2, wherein R¹ is a 6-membered heterocycloalkyl selected from piperidine, piperazine, tetrahydrothiopyran, tetrahydropyran, dioxane, morpholine, and thiomorpholine, any of which is optionally substituted with one or more R⁶.

14. The composition of claim 1 or 2, wherein R¹ is a 5-membered heterocycloalkyl selected from tetrahydrofuran, pyrrolidone, and tetrahydrothiophene, any of which is optionally substituted with one or more R⁶.

15. The composition of claim 1 or 2, wherein R¹ is a 4-membered heterocycloalkyl selected from azetidine, oxetane, and thietane, any of which is optionally substituted with one or more R⁶.

16. The composition of claim 1, wherein R¹ is a 6-6 fused ring system selected from naphthalene, quinoline, isoquinoline, pteridine, benzopyran, dihydroquinoline, dihydroisoquinoline, dihydronapthalene, and tetrahydronapthalene, any of which is optionally substituted with one or more R⁶.

17. The composition of claim 1, wherein R¹ is a 5-6 fused ring system selected from benzimidazole, indole, azaindole, indazole, benzothiophene, benzofuran, benzoselenophene, benzimidazole, benzotriazole, benzothiazole, benzisothiazole, benzoxadiazole, benzoxazole, and benzothiadiazole, imidazo[4,5-b]-pyridine, imidazo[4,5-c]-pyridine, pyrazolo[3,4-b]pyridine, pyrazolo[3,4-c]pyridine, pyrazolo[4,3-b]pyridine, pyrazolo[4,3-b]pyridine, oxazolo[4,5-b]pyridine, oxazolo[4,5-c]pyridine, oxazolo[5,4-b]pyridine, oxazolo[5,4-c]pyridine, thiazolo[4,5-b]pyridine, thiazolo[4,5-c]pyridine, thiazolo[5,4-b]pyridine, thiazolo[5,4-c]pyridine, isooxazolo[4,5-b]pyridine, isooxazolo[4,5-c]pyridine, isooxazolo[3,4-b]pyridine, isooxazolo[3,4-c]pyridine, isooxazolo[4,3-b]pyridine, isooxazolo[4,3-c]pyridine, isooxazolo[5,4-b]pyridine, isooxazolo[5,4-c]pyridine, isothiazolo[4,5-b]pyridine, isothiazolo[4,5-c]pyridine, isothiazolo[5,4-b]pyridine, isothiazolo[5,4-c]pyridine, isothiazolo[3,4-b]pyridine, isothiazolo[3,4-c]pyridine, isothiazolo[4,3-b]pyridine, isothiazolo[4,3-c]pyridine and purine, any of which is optionally substituted with one or more R⁶.

18. The composition of claim 1, wherein R¹ is a 5-5 fused ring system selected from pyrrolo[2,1-b]thiazole, pyrazolo[5,1-b]thiazole, imidazo[5,1-b]thiazole, imidazo[2,1-b]thiazole, thiazolo[3,2-b][1,2,4]triazole, thiazolo[3,2-c][1,2,4]triazole, imidazo[2,1-b]thiadiazole, imidazo[1,2-d][1,2,4]thiadiazole, thiazolo[3,2-d]tetrazole, thieno[3,2-b]thiophene, thieno[2,3-b]thiophene, thieno[3,4-b]thiophene, thieno[3,4-c]thiophene, any of which is optionally substituted with one or more R⁶.

19. The composition of claim 1, wherein R¹ is a bridged ring system selected from norbornane, norbornene, bicyclo[1.1.0]butane, bicyclo[3.2.0]heptane, bicyclo[3.3.0]octane, bicyclo[4.3.0]nonane, bicyclo[1.1.1]pentane, bicyclo[2.2.1]hexane, bicyclo[3.3.1]nonane, bicyclo[2.2.2]octane, adamantane, cubane, housane, any of which is optionally substituted with one or more R⁶.

20. The composition of any of claims 1 to 19, wherein R³ is C₃₋₇ carbocycle or 3- to 7-membered heterocycle; wherein the C₃₋₇ carbocycle and 3- to 7-membered heterocycle are each optionally substituted with one or more R⁸.

21. The composition of any of claims 1 to 20, wherein R³ is phenyl, optionally substituted with one or more R⁸.

22. The composition of any of claims 1 to 20, wherein R³ is a cycloalkyl selected from cyclooctane, cycloheptane, cyclohexane, cyclopentane, cyclobutane, and cyclopropane, any of which is optionally substituted with one or more R⁸.

23. The composition of any of claims 1 to 20, wherein R³ is a 6-membered heteroaryl selected from pyridine, pyrazine, and pyrimidine, any of which is optionally substituted with one or more R⁸.

24. The composition of any of claims 1 to 20, wherein R³ is a 5-membered heteroaryl selected from thiophene, furan, pyrrole, pyrazole, selenophene, imidazole, thiazole, isothiazole, oxatriazole, oxadiazole, oxazole, and thiadiazole, any of which is optionally substituted with one or more R⁸.

25. The composition of any of claims 1 to 20, wherein R³ is a 6-membered heterocycloalkenyl selected from dihydropyridine, tetrahydropyridine, dihydropyridazine, tetrahydropyridazine, dihydropyrimidine, tetrahydropyrimidine, dihydropyrazine, tetrahydropyridazine, dihydropyrimidine, tetrahydropyrimidine, dihydropyrazine, tetrahydropyrazine, pyran, dihydropyran, thiopyran, dihydrothiopyran, dioxine, dihydrodioxine, oxazine, dihydrooxazine, thiazine, and dihydrothiazine, any of which is optionally substituted with one or more R⁸.

26. The composition of any of claims 1 to 20, wherein R³ is a 5-membered heterocycloalkenyl selected from pyrroline, pyrazoline, imidazoline, triazoline, dihydrofuran, dihydrothiophene, oxazoline, isooxazoline, thiazoline, isothiazoline, oxadiazoline, and thiadiazoline, any of which is optionally substituted with one or more R⁸.

27. The composition of any of claims 1 to 20, wherein R³ is a 6-membered heterocycloalkyl selected from piperidine, piperazine, tetrahydrothiopyran, tetrahydropyran, dioxane, morpholine, and thiomorpholine, any of which is optionally substituted with one or more R⁸.

28. The composition of any of claims 1 to 20, wherein R³ is a 5-membered heterocycloalkyl selected from tetrahydrofuran, pyrrolidone, and tetrahydrothiophene, any of which is optionally substituted with one or more R⁸.

29. The composition of any of claims 1 to 20, wherein R³ is a 4-membered heterocycloalkyl selected from azetidine, oxetane, and thietane, any of which is optionally substituted with one or more R⁸.

30. The composition of any of claims 1 to 19, wherein R³ is a 6-6 fused ring system selected from naphthalene, quinoline, isoquinoline, pteridine, benzopyran, dihydroquinoline, dihydroisoquinoline, dihydronapthalene, and tetrahydronapthalene, any of which is optionally substituted with one or more R⁸.

31. The composition of any of claims 1 to 19, wherein R³ is a 5-6 fused ring system selected from benzimidazole, indole, azaindole, indazole, benzothiophene, benzofuran, benzoselenophene, benzimidazole, benzotriazole, benzothiazole, benzisothiazole, benzoxadiazole, benzoxazole, and benzothiadiazole, imidazo[4,5-b]-pyridine, imidazo[4,5-c]-pyridine, pyrazolo[3,4-b]pyridine, pyrazolo[3,4-c]pyridine, pyrazolo[4,3-b]pyridine, pyrazolo[4,3-b]pyridine, oxazolo[4,5-b]pyridine, oxazolo[4,5-c]pyridine, oxazolo[5,4-b]pyridine, oxazolo[5,4-c]pyridine, thiazolo[4,5-b]pyridine, thiazolo[4,5-c]pyridine, thiazolo[5,4-b]pyridine, thiazolo[5,4-c]pyridine, isooxazolo[4,5-b]pyridine, isooxazolo[4,5-c]pyridine, isooxazolo[3,4-b]pyridine, isooxazolo[3,4-c]pyridine, isooxazolo[4,3-b]pyridine, isooxazolo[4,3-c]pyridine, isooxazolo[5,4-b]pyridine, isooxazolo[5,4-c]pyridine, isothiazolo[4,5-b]pyridine, isothiazolo[4,5-c]pyridine, isothiazolo[5,4-b]pyridine, isothiazolo[5,4-c]pyridine, isothiazolo[3,4-b]pyridine, isothiazolo[3,4-c]pyridine, isothiazolo[4,3-b]pyridine, isothiazolo[4,3-c]pyridine and purine, any of which is optionally substituted with one or more R⁸.

32. The composition of any of claims 1 to 19, wherein R³ is a 5-5 fused ring system selected from pyrrolo[2,1-b]thiazole, pyrazolo[5,1-b]thiazole, imidazo[5,1-b]thiazole, imidazo[2,1-b]thiazole, thiazolo[3,2-b][1,2,4]triazole, thiazolo[3,2-c][1,2,4]triazole, imidazo[2,1-b]thiadiazole, imidazo[1,2-d][1,2,4]thiadiazole, thiazolo[3,2-d]tetrazole, thieno[3,2-b]thiophene, thieno[2,3-b]thiophene, thieno[3,4-b]thiophene, thieno[3,4-c]thiophene, any of which is optionally substituted with one or more R⁸.

33. The composition of any of claims 1 to 19, wherein R³ is a bridged ring system selected from norbornane, norbornene, bicyclo[1.1.0]butane, bicyclo[3.2.0]heptane, bicyclo[3.3.0]octane, bicyclo[4.3.0]nonane, bicyclo[1.1.1]pentane, bicyclo[2.2.1]hexane, bicyclo[3.3.1]nonane, bicyclo[2.2.2]octane, adamantane, cubane, housane, any of which is optionally substituted with one or more R⁸.

34. The composition of any of claims 1 to 33, wherein R² is C₃₋₇ carbocycle or 3- to 7-membered heterocycle; wherein the C₃₋₇ carbocycle and 3- to 7-membered heterocycle are each optionally substituted with one or more R⁷.

35. The composition of any of claims 1 to 34, wherein R² is phenyl, optionally substituted with one or more R⁷.

36. The composition of any of claims 1 to 34, wherein R² is a cycloalkyl selected from cyclooctane, cycloheptane, cyclohexane, cyclopentane, cyclobutane, and cyclopropane, any of which is optionally substituted with one or more R⁷.

37. The composition of any of claims 1 to 34, wherein R² is a 6-membered heteroaryl selected from pyridine, pyrazine, and pyrimidine, any of which is optionally substituted with one or more R⁷.

38. The composition of any of claims 1 to 34, wherein R² is a 5-membered heteroaryl selected from thiophene, furan, pyrrole, pyrazole, selenophene, imidazole, thiazole, isothiazole, oxatriazole, oxadiazole, oxazole, and thiadiazole, any of which is optionally substituted with one or more R⁷.

39. The composition of any of claims 1 to 34, wherein R² is a 6-membered heterocycloalkenyl selected from dihydropyridine, tetrahydropyridine, dihydropyridazine, tetrahydropyridazine, dihydropyrimidine, tetrahydropyrimidine, dihydropyrazine, tetrahydropyridazine, dihydropyrimidine, tetrahydropyrimidine, dihydropyrazine, tetrahydropyrazine, pyran, dihydropyran, thiopyran, dihydrothiopyran, dioxine, dihydrodioxine, oxazine, dihydrooxazine, thiazine, and dihydrothiazine, any of which is optionally substituted with one or more R⁷.

40. The composition of claims 1 to 34, wherein R² is a 5-membered heterocycloalkenyl selected from pyrroline, pyrazoline, imidazoline, triazoline, dihydrofuran, dihydrothiophene, oxazoline, isooxazoline, thiazoline, isothiazoline, oxadiazoline, and thiadiazoline, any of which is optionally substituted with one or more R⁷.

41. The composition of any of claims 1 to 34, wherein R² is a 6-membered heterocycloalkyl selected from piperidine, piperazine, tetrahydrothiopyran, tetrahydropyran, dioxane, morpholine, and thiomorpholine, any of which is optionally substituted with one or more R⁷.

42. The composition of any of claims 1 to 34, wherein R² is a 5-membered heterocycloalkyl selected from tetrahydrofuran, pyrrolidone, and tetrahydrothiophene, any of which is optionally substituted with one or more R⁷.

43. The composition of any of claims 1 to 34, wherein R² is a 4-membered heterocycloalkyl selected from azetidine, oxetane, and thietane, any of which is optionally substituted with one or more R⁷.

44. The composition of any of claims 1 to 33, wherein R² is a 6-6 fused ring system selected from naphthalene, quinoline, isoquinoline, pteridine, benzopyran, dihydroquinoline, dihydroisoquinoline, dihydronapthalene, and tetrahydronapthalene, any of which is optionally substituted with one or more R⁷.

45. The composition of any of claims 1 to 33, wherein R² is a 5-6 fused ring system selected from benzimidazole, indole, azaindole, indazole, benzothiophene, benzofuran, benzoselenophene, benzimidazole, benzotriazole, benzothiazole, benzisothiazole, benzoxadiazole, benzoxazole, and benzothiadiazole, imidazo[4,5-b]-pyridine, imidazo[4,5-c]-pyridine, pyrazolo[3,4-b]pyridine, pyrazolo[3,4-c]pyridine, pyrazolo[4,3-b]pyridine, pyrazolo[4,3-b]pyridine, oxazolo[4,5-b]pyridine, oxazolo[4,5-c]pyridine, oxazolo[5,4-b]pyridine, oxazolo[5,4-c]pyridine, thiazolo[4,5-b]pyridine, thiazolo[4,5-c]pyridine, thiazolo[5,4-b]pyridine, thiazolo[5,4-c]pyridine, isooxazolo[4,5-b]pyridine, isooxazolo[4,5-c]pyridine, isooxazolo[3,4-b]pyridine, isooxazolo[3,4-c]pyridine, isooxazolo[4,3-b]pyridine, isooxazolo[4,3-c]pyridine, isooxazolo[5,4-b]pyridine, isooxazolo[5,4-c]pyridine, isothiazolo[4,5-b]pyridine, isothiazolo[4,5-c]pyridine, isothiazolo[5,4-b]pyridine, isothiazolo[5,4-c]pyridine, isothiazolo[3,4-b]pyridine, isothiazolo[3,4-c]pyridine, isothiazolo[4,3-b]pyridine, isothiazolo[4,3-c]pyridine and purine, any of which is optionally substituted with one or more R⁷.

46. The composition of any of claims 1 to 33, wherein R² is a 5-5 fused ring system selected from pyrrolo[2,1-b]thiazole, pyrazolo[5,1-b]thiazole, imidazo[5,1-b]thiazole, imidazo[2,1-b]thiazole, thiazolo[3,2-b][1,2,4]triazole, thiazolo[3,2-c][1,2,4]triazole, imidazo[2,1-b]thiadiazole, imidazo[1,2-d][1,2,4]thiadiazole, thiazolo[3,2-d]tetrazole, thieno[3,2-b]thiophene, thieno[2,3-b]thiophene, thieno[3,4-b]thiophene, thieno[3,4-c]thiophene, any of which is optionally substituted with one or more R⁷.

47. The composition of any of claims 1 to 33, wherein R² is a bridged ring system selected from norbornane, norbornene, bicyclo[1.1.0]butane, bicyclo[3.2.0]heptane, bicyclo[3.3.0]octane, bicyclo[4.3.0]nonane, bicyclo[1.1.1]pentane, bicyclo[2.2.1]hexane, bicyclo[3.3.1]nonane, bicyclo[2.2.2]octane, adamantane, cubane, housane, any of which is optionally substituted with one or more R⁷.

48. The composition of claims 1 to 47, wherein each of R⁴ or R⁵ is independently hydrogen or selected from azacyclopropane, azetidine, pyrrolidine, piperidine, azepane, azocane, azonane, morpholine, thiomorpholine, 1,4-piperazine, 1,4-oxazepane, 1,4-diazepane, tropane, azanorbornane, and 2-azaadamantane.

49. The composition of claim 1, wherein the compound has the structure: 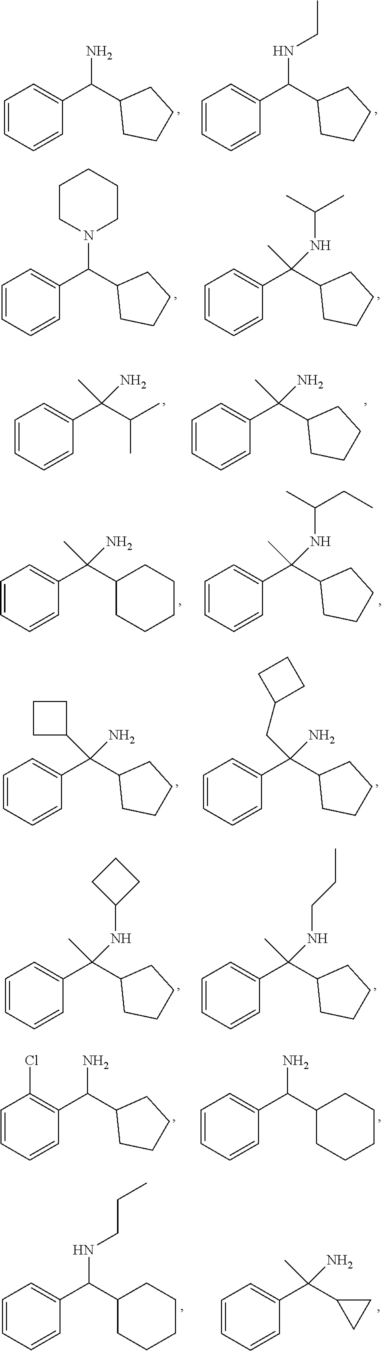 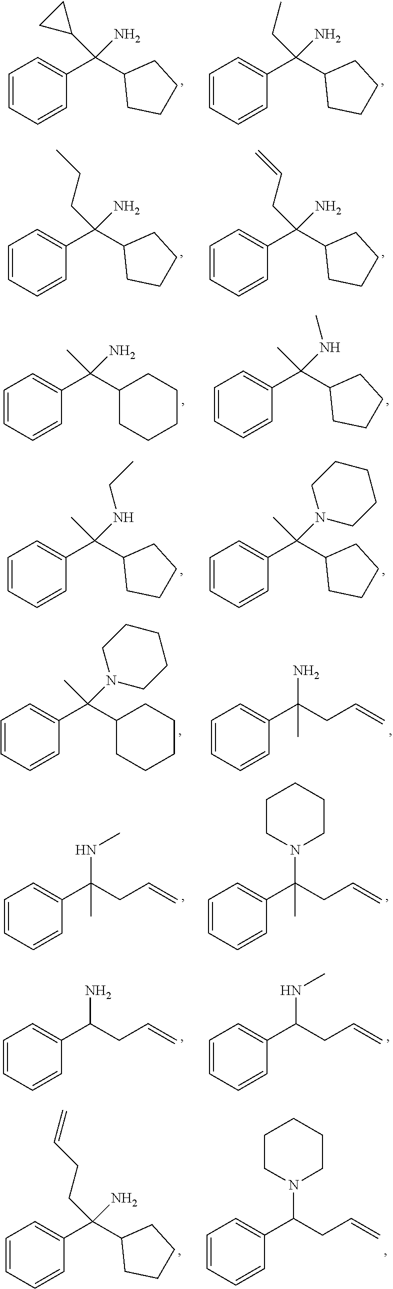 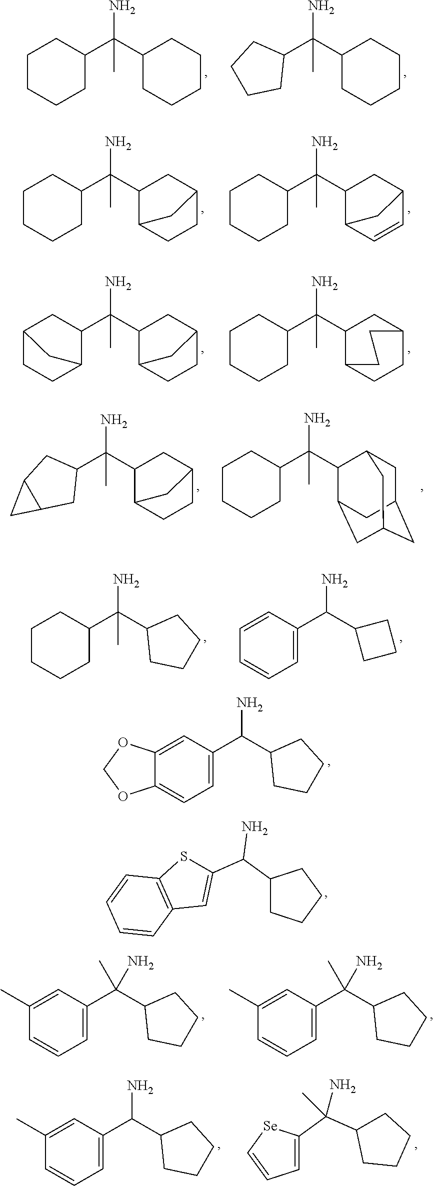 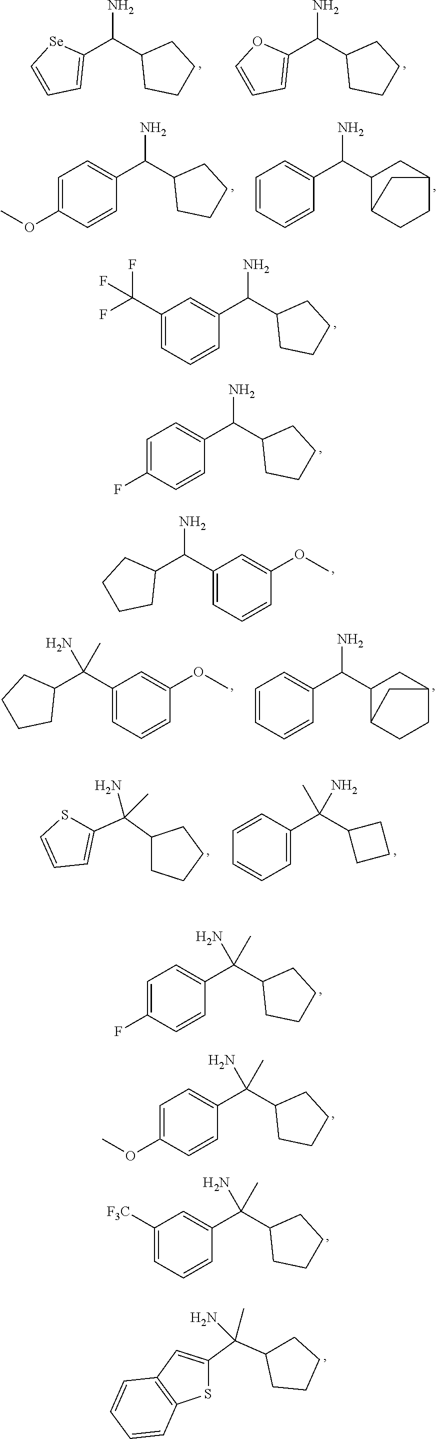 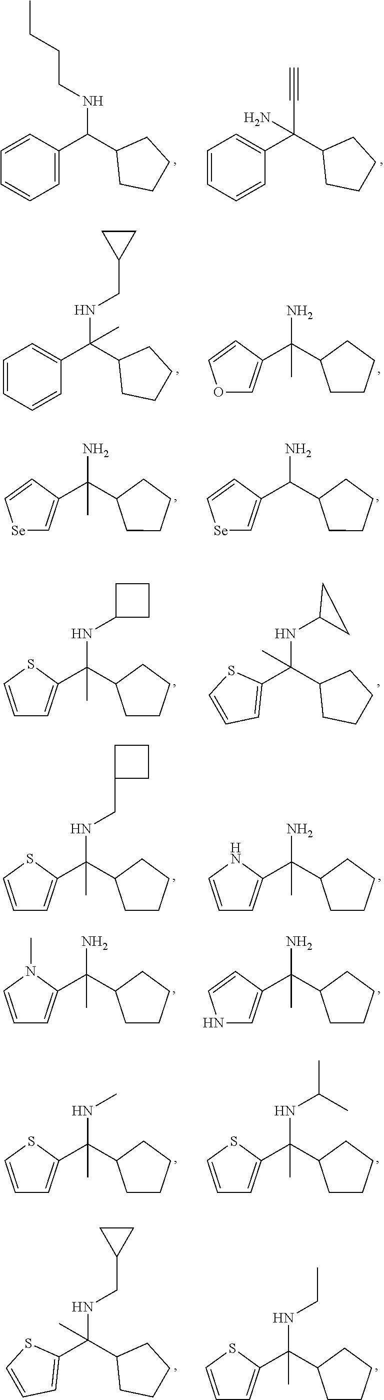 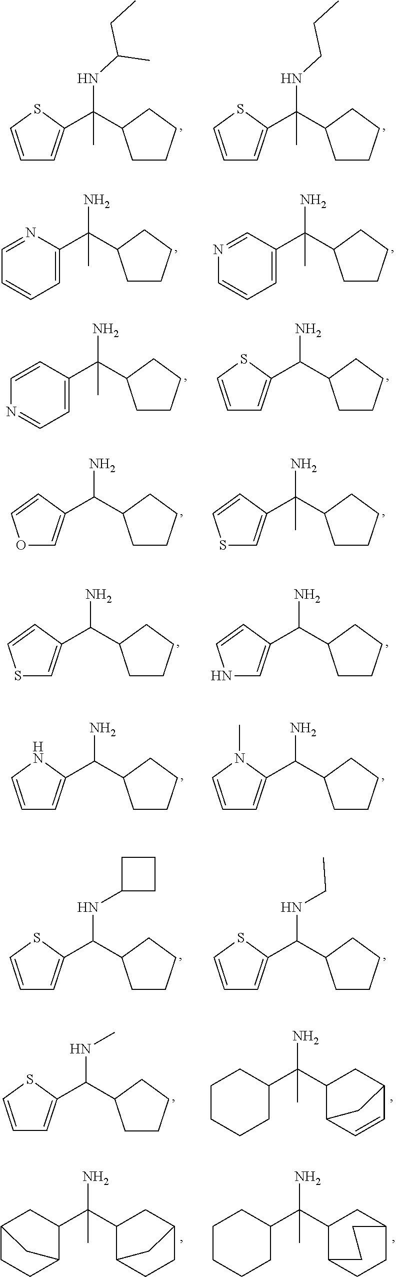 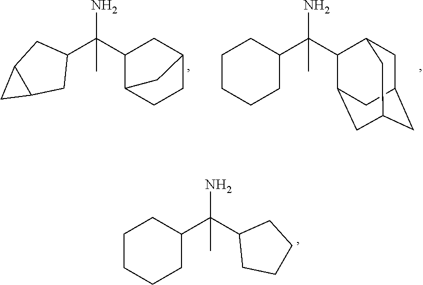
or a pharmaceutically acceptable salt of the compound.

50. A method for treating a physical, psychiatric, or neurological disorder comprising administering to a subject in need thereof the pharmaceutical composition of any one of claims 1 to 49.

51. A composition comprising a compound of Formula (II): 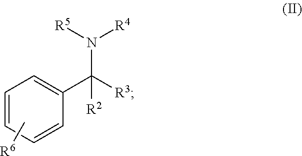
or an enantiomer, a mixture of enantiomers, or an isotopic variant thereof; or a pharmaceutically acceptable salt, solvate or hydrate thereof, wherein:
R² is selected from
hydrogen;
halogen;
C₁₋₈ alkyl, C₂₋₈ alkenyl, and C₂₋₈ alkynyl, each of which is optionally substituted with one or more R⁷; and
C₃₋₁₀ carbocycle and 3- to 10-membered heterocycle; wherein the C₃₋₁₀ carbocycle and 3- to 10-membered heterocycle are each optionally substituted with one or more R⁷;
R³ is selected from:
C₁₋₆ alkyl, C₂₋₆ alkenyl, and C₂₋₆ alkynyl, each of which is optionally substituted with one or more R⁸; and
C₃₋₁₀ carbocycle and 3- to 10-membered heterocycle; wherein the C₃₋₁₀ carbocycle and 3- to 10-membered heterocycle are each optionally substituted with one or more R⁸;
R⁴, R⁵, R⁶, R⁷, and R⁸ are each independently selected from:
hydrogen;
C₁₋₁₀ alkyl, C₂₋₁₀ alkenyl, and C₂₋₁₀ alkynyl optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₁₋₆ cycloalkyl, and C₁₋₆ cycloalkenyl; and
C₃₋₁₀ carbocycle or 3- to 10-membered heterocycle; wherein the C₃₋₁₀ carbocycle and 3- to 10-membered heterocycle are each optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), and C₁₋₈ alkyl;
with the proviso that the compound of Formula II is not

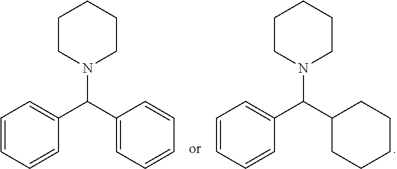

52. The composition of claim 51, wherein R³ is C₃₋₇ carbocycle or 3- to 7-membered heterocycle; wherein the C₃₋₇ carbocycle and 3- to 7-membered heterocycle are each optionally substituted with one or more R⁸.

53. The composition of claim 51 or 52, wherein R³ is phenyl, optionally substituted with one or more R⁸.

54. The composition of claim 51 or 52, wherein R³ is a cycloalkyl selected from cyclooctane, cycloheptane, cyclohexane, cyclopentane, cyclobutane, and cyclopropane, any of which is optionally substituted with one or more R⁸.

55. The composition of claim 51 or 52, wherein R³ is a 6-membered heteroaryl selected from pyridine, pyrazine, and pyrimidine, any of which is optionally substituted with one or more R⁸.

56. The composition of claim 51 or 52, wherein R³ is a 5-membered heteroaryl selected from thiophene, furan, pyrrole, pyrazole, selenophene, imidazole, thiazole, isothiazole, oxatriazole, oxadiazole, oxazole, and thiadiazole, any of which is optionally substituted with one or more R⁸.

57. The composition of claim 51 or 52, wherein R³ is a 6-membered heterocycloalkenyl selected from dihydropyridine, tetrahydropyridine, dihydropyridazine, tetrahydropyridazine, dihydropyrimidine, tetrahydropyrimidine, dihydropyrazine, tetrahydropyridazine, dihydropyrimidine, tetrahydropyrimidine, dihydropyrazine, tetrahydropyrazine, pyran, dihydropyran, thiopyran, dihydrothiopyran, dioxine, dihydrodioxine, oxazine, dihydrooxazine, thiazine, and dihydrothiazine, any of which is optionally substituted with one or more R⁸.

58. The composition of claim 51 or 52, wherein R³ is a 5-membered heterocycloalkenyl selected from pyrroline, pyrazoline, imidazoline, triazoline, dihydrofuran, dihydrothiophene, oxazoline, isooxazoline, thiazoline, isothiazoline, oxadiazoline, and thiadiazoline, any of which is optionally substituted with one or more R⁸.

59. The composition of claim 51 or 52, wherein R³ is a 6-membered heterocycloalkyl selected from piperidine, piperazine, tetrahydrothiopyran, tetrahydropyran, dioxane, morpholine, and thiomorpholine, any of which is optionally substituted with one or more R⁸.

60. The composition of claim 51 or 52, wherein R³ is a 5-membered heterocycloalkyl selected from tetrahydrofuran, pyrrolidone, and tetrahydrothiophene, any of which is optionally substituted with one or more R⁸.

61. The composition of claim 51 or 52, wherein R³ is a 4-membered heterocycloalkyl selected from azetidine, oxetane, and thietane, any of which is optionally substituted with one or more R⁸.

62. The composition of claim 51, wherein R³ is a 6-6 fused ring system selected from naphthalene, quinoline, isoquinoline, pteridine, benzopyran, dihydroquinoline, dihydroisoquinoline, dihydronapthalene, and tetrahydronapthalene, any of which is optionally substituted with one or more R⁸.

63. The composition of claim 51, wherein R³ is a 5-6 fused ring system selected from benzimidazole, indole, azaindole, indazole, benzothiophene, benzofuran, benzoselenophene, benzimidazole, benzotriazole, benzothiazole, benzisothiazole, benzoxadiazole, benzoxazole, and benzothiadiazole, imidazo[4,5-b]-pyridine, imidazo[4,5-c]-pyridine, pyrazolo[3,4-b]pyridine, pyrazolo[3,4-c]pyridine, pyrazolo[4,3-b]pyridine, pyrazolo[4,3-b]pyridine, oxazolo[4,5-b]pyridine, oxazolo[4,5-c]pyridine, oxazolo[5,4-b]pyridine, oxazolo[5,4-c]pyridine, thiazolo[4,5-b]pyridine, thiazolo[4,5-c]pyridine, thiazolo[5,4-b]pyridine, thiazolo[5,4-c]pyridine, isooxazolo[4,5-b]pyridine, isooxazolo[4,5-c]pyridine, isooxazolo[3,4-b]pyridine, isooxazolo[3,4-c]pyridine, isooxazolo[4,3-b]pyridine, isooxazolo[4,3-c]pyridine, isooxazolo[5,4-b]pyridine, isooxazolo[5,4-c]pyridine, isothiazolo[4,5-b]pyridine, isothiazolo[4,5-c]pyridine, isothiazolo[5,4-b]pyridine, isothiazolo[5,4-c]pyridine, isothiazolo[3,4-b]pyridine, isothiazolo[3,4-c]pyridine, isothiazolo[4,3-b]pyridine, isothiazolo[4,3-c]pyridine and purine, any of which is optionally substituted with one or more R⁸.

64. The composition of claim 51, wherein R³ is a 5-5 fused ring system selected from pyrrolo[2,1-b]thiazole, pyrazolo[5,1-b]thiazole, imidazo[5,1-b]thiazole, imidazo[2,1-b]thiazole, thiazolo[3,2-b][1,2,4]triazole, thiazolo[3,2-c][1,2,4]triazole, imidazo[2,1-b]thiadiazole, imidazo[1,2-d][1,2,4]thiadiazole, thiazolo[3,2-d]tetrazole, thieno[3,2-b]thiophene, thieno[2,3-b]thiophene, thieno[3,4-b]thiophene, thieno[3,4-c]thiophene, any of which is optionally substituted with one or more R⁸.

65. The composition of claim 51, wherein R³ is a bridged ring system selected from norbornane, norbornene, bicyclo[1.1.0]butane, bicyclo[3.2.0]heptane, bicyclo[3.3.0]octane, bicyclo[4.3.0]nonane, bicyclo[1.1.1]pentane, bicyclo[2.2.1]hexane, bicyclo[3.3.1]nonane, bicyclo[2.2.2]octane, adamantane, cubane, housane, any of which is optionally substituted with one or more R⁸.

66. The composition of any of claims 51 to 65, wherein R² is C₃₋₇ carbocycle or 3- to 7-membered heterocycle; wherein the C₃₋₇ carbocycle and 3- to 7-membered heterocycle are each optionally substituted with one or more R⁷.

67. The composition of any of claims 51 to 66, wherein R² is phenyl, optionally substituted with one or more R⁷.

68. The composition of any of claims 51 to 66, wherein R² is a cycloalkyl selected from cyclooctane, cycloheptane, cyclohexane, cyclopentane, cyclobutane, and cyclopropane, any of which is optionally substituted with one or more R⁷.

69. The composition of any of claims 51 to 66, wherein R² is a 6-membered heteroaryl selected from pyridine, pyrazine, and pyrimidine, any of which is optionally substituted with one or more R⁷.

70. The composition of any of claims 51 to 66, wherein R² is a 5-membered heteroaryl selected from thiophene, furan, pyrrole, pyrazole, selenophene, imidazole, thiazole, isothiazole, oxatriazole, oxadiazole, oxazole, and thiadiazole, any of which is optionally substituted with one or more R⁷.

71. The composition of any of claims 51 to 66, wherein R² is a 6-membered heterocycloalkenyl selected from dihydropyridine, tetrahydropyridine, dihydropyridazine, tetrahydropyridazine, dihydropyrimidine, tetrahydropyrimidine, dihydropyrazine, tetrahydropyridazine, dihydropyrimidine, tetrahydropyrimidine, dihydropyrazine, tetrahydropyrazine, pyran, dihydropyran, thiopyran, dihydrothiopyran, dioxine, dihydrodioxine, oxazine, dihydrooxazine, thiazine, and dihydrothiazine, any of which is optionally substituted with one or more R⁷.

72. The composition of any of claims 51 to 66, wherein R² is a 5-membered heterocycloalkenyl selected from pyrroline, pyrazoline, imidazoline, triazoline, dihydrofuran, dihydrothiophene, oxazoline, isooxazoline, thiazoline, isothiazoline, oxadiazoline, and thiadiazoline, any of which is optionally substituted with one or more R⁷.

73. The composition of any of claims 51 to 66, wherein R² is a 6-membered heterocycloalkyl selected from piperidine, piperazine, tetrahydrothiopyran, tetrahydropyran, dioxane, morpholine, and thiomorpholine, any of which is optionally substituted with one or more R⁷.

74. The composition of any of claims 51 to 66, wherein R² is a 5-membered heterocycloalkyl selected from tetrahydrofuran, pyrrolidone, and tetrahydrothiophene, any of which is optionally substituted with one or more R⁷.

75. The composition of any of claims 51 to 66, wherein R² is a 4-membered heterocycloalkyl selected from azetidine, oxetane, and thietane, any of which is optionally substituted with one or more R⁷.

76. The composition of any of claims 51 to 65, wherein R² is a 6-6 fused ring system selected from naphthalene, quinoline, isoquinoline, pteridine, benzopyran, dihydroquinoline, dihydroisoquinoline, dihydronapthalene, and tetrahydronapthalene, any of which is optionally substituted with one or more R⁷.

77. The composition of any of claims 51 to 65, wherein R² is a 5-6 fused ring system selected from benzimidazole, indole, indazole, benzothiophene, benzofuran, benzoselenophene, benzimidazole, benzotriazole, and purine, any of which is optionally substituted with one or more R⁷.

78. The composition of any of claims 51 to 65, wherein R² is a 5-5 fused ring system selected from pyrrolo[2,1-b]thiazole, pyrazolo[5,1-b]thiazole, imidazo[5,1-b]thiazole, imidazo[2,1-b]thiazole, thiazolo[3,2-b][1,2,4]triazole, thiazolo[3,2-c][1,2,4]triazole, imidazo[2,1-b]thiadiazole, imidazo[1,2-d][1,2,4]thiadiazole, thiazolo[3,2-d]tetrazole, thieno[3,2-b]thiophene, thieno[2,3-b]thiophene, thieno[3,4-b]thiophene, thieno[3,4-c]thiophene, any of which is optionally substituted with one or more R⁷.

79. The composition of any of claims 51 to 65, wherein R² is a bridged ring system selected from norbornane, norbornene, bicyclo[1.1.0]butane, bicyclo[3.2.0]heptane, bicyclo[3.3.0]octane, bicyclo[4.3.0]nonane, bicyclo[1.1.1]pentane, bicyclo[2.2.1]hexane, bicyclo[3.3.1]nonane, bicyclo[2.2.2]octane, adamantane, cubane, housane, any of which is optionally substituted with one or more R⁷.

80. The composition of claims 51 to 79, wherein each of R⁴ or R⁵ is independently selected from azacyclopropane, pyrrolidine, piperidine, azepane, azocane, azonane, morpholine, thiomorpholine, 1,4-piperazine, 1,4-oxazepane, and 1,4-diazepane.

81. The composition of claim 47, wherein the compound of Formula (II) is selected from:

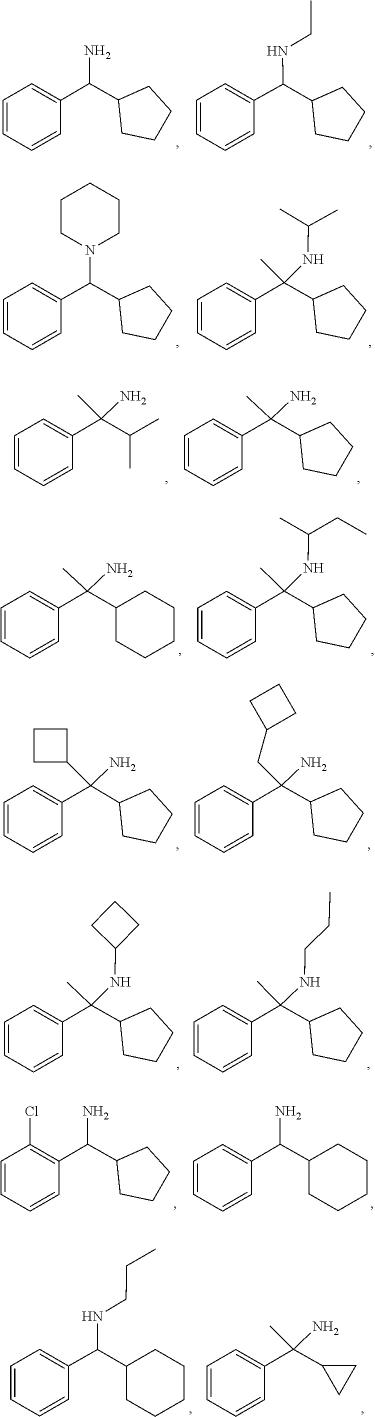

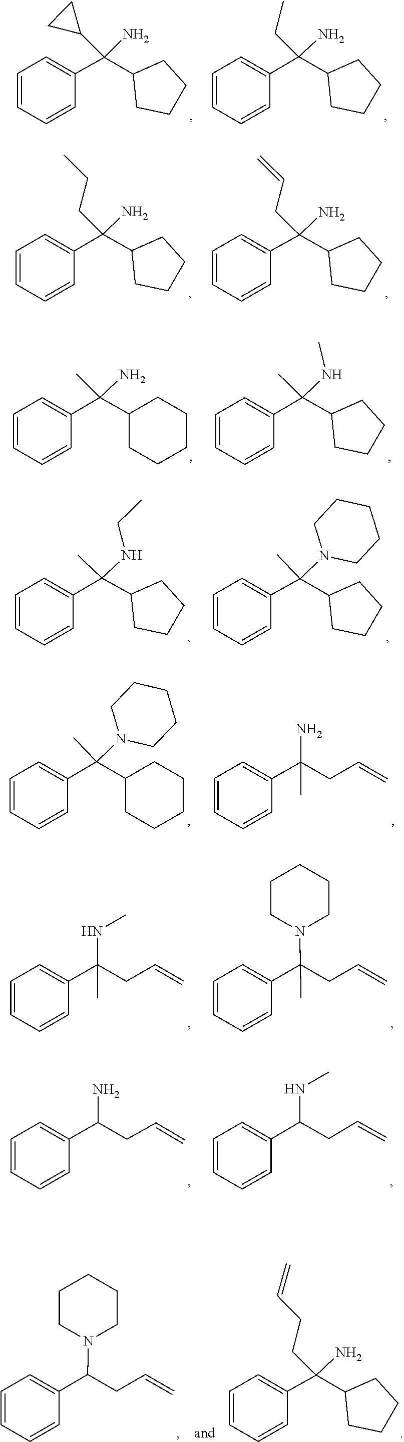

82. A method for treating a physical, psychiatric, or neurological disorder comprising administering to a subject in need thereof the pharmaceutical composition of any one of claims 51 to 81.

83. A composition comprising a compound of Formula (III): 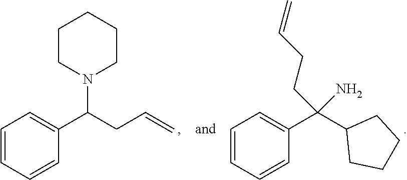 [SMILES: C=CCC(c1ccccc1)N1CCCCC1 ; C=CCCC(N)(c1ccccc1)C1CCCC1]
or an enantiomer, a mixture of enantiomers, or an isotopic variant thereof, or a pharmaceutically acceptable salt, solvate or hydrate thereof, wherein:
R² is selected from
hydrogen;
halogen;
C₁₋₈ alkyl, C₂₋₈ alkenyl, and C₂₋₈ alkynyl, each of which is optionally substituted with one or more R⁷; and
C₃₋₁₀ carbocycle and 3- to 10-membered heterocycle; wherein the C₃₋₁₀ carbocycle and 3- to 10-membered heterocycle are each optionally substituted with one or more R⁷;
R⁴, R⁵, R⁶, R⁷, and R⁸ are each independently selected from:
hydrogen;
C₁₋₁₀ alkyl, C₂₋₁₀ alkenyl, and C₂₋₁₀ alkynyl optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₁₋₆ cycloalkyl, and C₁₋₆ cycloalkenyl; and
C₃₋₁₀ carbocycle or 3- to 10-membered heterocycle; wherein the C₃₋₁₀ carbocycle and 3- to 10-membered heterocycle are each optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), and C₁₋₈ alkyl.

84. The composition of claim 83, wherein R² is C₃₋₇ carbocycle or 3- to 7-membered heterocycle; wherein the C₃₋₇ carbocycle and 3- to 7-membered heterocycle are each optionally substituted with one or more R⁷.

85. The composition of claim 83 or 84, wherein R² is phenyl, optionally substituted with one or more R⁷.

86. The composition of claim 83 or 84, wherein R² is a cycloalkyl selected from cyclooctane, cycloheptane, cyclohexane, cyclopentane, cyclobutane, and cyclopropane, any of which is optionally substituted with one or more R⁷.

87. The composition of claim 83 or 84, wherein R² is a 6-membered heteroaryl selected from pyridine, pyrazine, and pyrimidine, any of which is optionally substituted with one or more R⁷.

88. The composition of claim 83 or 84, wherein R² is a 5-membered heteroaryl selected from thiophene, furan, pyrrole, pyrazole, selenophene, imidazole, thiazole, isothiazole, oxatriazole, oxadiazole, oxazole, and thiadiazole, any of which is optionally substituted with one or more R⁷.

89. The composition of claim 83 or 84, wherein R² is a 6-membered heterocycloalkenyl selected from dihydropyridine, tetrahydropyridine, dihydropyridazine, tetrahydropyridazine, dihydropyrimidine, tetrahydropyrimidine, dihydropyrazine, tetrahydropyridazine, dihydropyrimidine, tetrahydropyrimidine, dihydropyrazine, tetrahydropyrazine, pyran, dihydropyran, thiopyran, dihydrothiopyran, dioxine, dihydrodioxine, oxazine, dihydrooxazine, thiazine, and dihydrothiazine, any of which is optionally substituted with one or more R⁷.

90. The composition of claim 83 or 84, wherein R² is a 5-membered heterocycloalkenyl selected from pyrroline, pyrazoline, imidazoline, triazoline, dihydrofuran, dihydrothiophene, oxazoline, isooxazoline, thiazoline, isothiazoline, oxadiazoline, and thiadiazoline, any of which is optionally substituted with one or more R⁷.

91. The composition of claim 83 or 84, wherein R² is a 6-membered heterocycloalkyl selected from piperidine, piperazine, tetrahydrothiopyran, tetrahydropyran, dioxane, morpholine, and thiomorpholine, any of which is optionally substituted with one or more R⁷.

92. The composition of claim 83 or 84, wherein R² is a 5-membered heterocycloalkyl selected from tetrahydrofuran, pyrrolidone, and tetrahydrothiophene, any of which is optionally substituted with one or more R⁷.

93. The composition of claim 83 or 84, wherein R² is a 4-membered heterocycloalkyl selected from azetidine, oxetane, and thietane, any of which is optionally substituted with one or more R⁷.

94. The composition of claim 83, wherein R² is a 6-6 fused ring system selected from naphthalene, quinoline, isoquinoline, pteridine, benzopyran, dihydroquinoline, dihydroisoquinoline, dihydronapthalene, and tetrahydronapthalene, any of which is optionally substituted with one or more R⁷.

95. The composition of claim 83, wherein R² is a 5-6 fused ring system selected from benzimidazole, indole, azaindole, indazole, benzothiophene, benzofuran, benzoselenophene, benzimidazole, benzotriazole, benzothiazole, benzisothiazole, benzoxadiazole, benzoxazole, and benzothiadiazole, imidazo[4,5-b]-pyridine, imidazo[4,5-c]-pyridine, pyrazolo[3,4-b]pyridine, pyrazolo[3,4-c]pyridine, pyrazolo[4,3-b]pyridine, pyrazolo[4,3-b]pyridine, oxazolo[4,5-b]pyridine, oxazolo[4,5-c]pyridine, oxazolo[5,4-b]pyridine, oxazolo[5,4-c]pyridine, thiazolo[4,5-b]pyridine, thiazolo[4,5-c]pyridine, thiazolo[5,4-b]pyridine, thiazolo[5,4-c]pyridine, isooxazolo[4,5-b]pyridine, isooxazolo[4,5-c]pyridine, isooxazolo[3,4-b]pyridine, isooxazolo[3,4-c]pyridine, isooxazolo[4,3-b]pyridine, isooxazolo[4,3-c]pyridine, isooxazolo[5,4-b]pyridine, isooxazolo[5,4-c]pyridine, isothiazolo[4,5-b]pyridine, isothiazolo[4,5-c]pyridine, isothiazolo[5,4-b]pyridine, isothiazolo[5,4-c]pyridine, isothiazolo[3,4-b]pyridine, isothiazolo[3,4-c]pyridine, isothiazolo[4,3-b]pyridine, isothiazolo[4,3-c]pyridine and purine, any of which is optionally substituted with one or more R⁷.

96. The composition of claim 79, wherein R² is a 5-5 fused ring system selected from pyrrolo[2,1-b]thiazole, pyrazolo[5,1-b]thiazole, imidazo[5,1-b]thiazole, imidazo[2,1-b]thiazole, thiazolo[3,2-b][1,2,4]triazole, thiazolo[3,2-c][1,2,4]triazole, imidazo[2,1-b]thiadiazole, imidazo[1,2-d][1,2,4]thiadiazole, thiazolo[3,2-d]tetrazole, thieno[3,2-b]thiophene, thieno[2,3-b]thiophene, thieno[3,4-b]thiophene, thieno[3,4-c]thiophene, any of which is optionally substituted with one or more R⁷.

97. The composition of claim 83, wherein R² is a bridged ring system selected from norbornane, norbornene, bicyclo[1.1.0]butane, bicyclo[3.2.0]heptane, bicyclo[3.3.0]octane, bicyclo[4.3.0]nonane, bicyclo[1.1.1]pentane, bicyclo[2.2.1]hexane, bicyclo[3.3.1]nonane, bicyclo[2.2.2]octane, adamantane, cubane, housane, any of which is optionally substituted with one or more R⁷.

98. The composition of claims 83 to 97, wherein each of R⁴ or R⁵ is independently hydrogen or a 3- to 10-membered heterocycle selected from azacyclopropane, pyrrolidine, piperidine, azepane, azocane, azonane, morpholine, thiomorpholine, 1,4-piperazine, 1,4-oxazepane, and 1,4-diazepane.

99. The composition of claim 83, wherein the compound of Formula (III) is selected from:

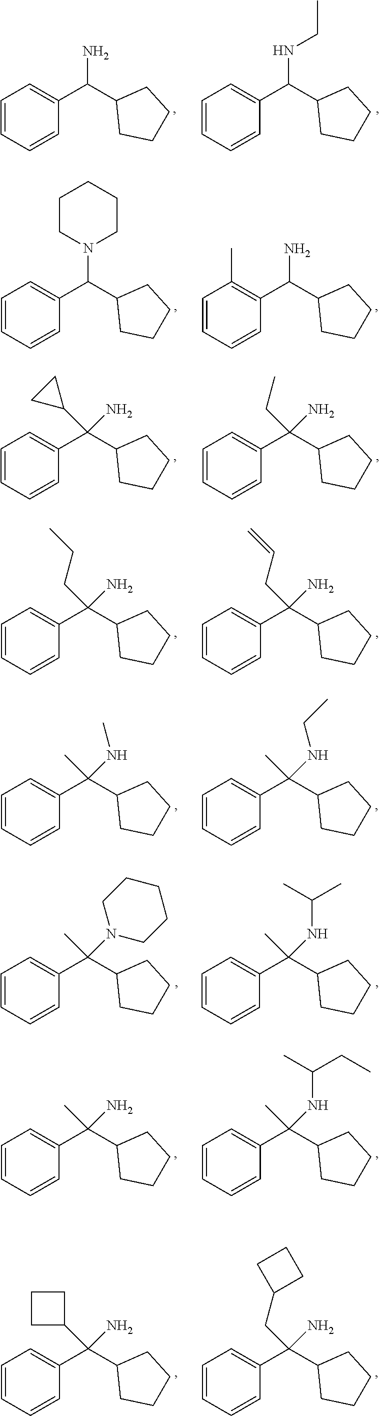

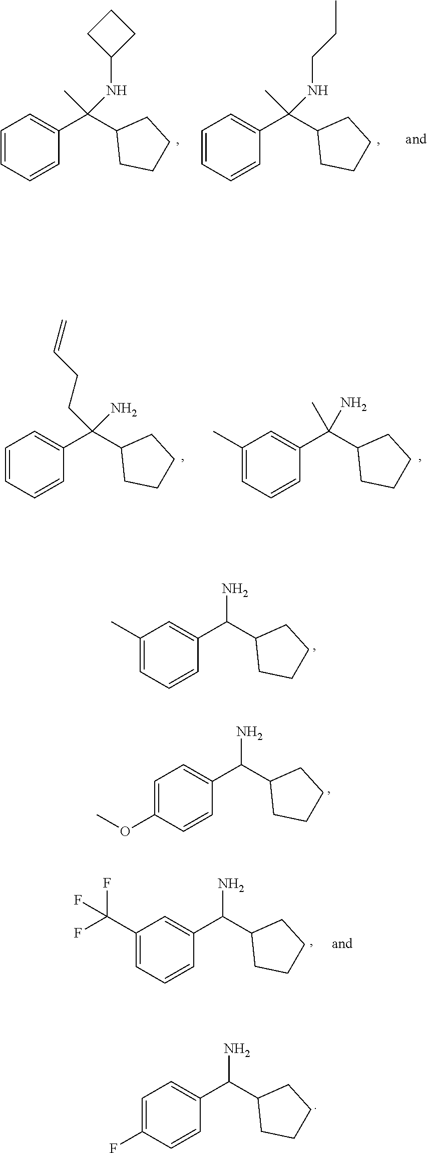

100. A method for treating a physical, psychiatric, or neurological disorder comprising administering to a subject in need thereof the pharmaceutical composition of any one of claims 83 to 99.

101. A composition comprising a compound of Formula (IV): 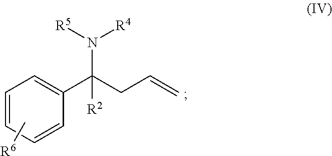
or an enantiomer, a mixture of enantiomers, or an isotopic variant thereof, or a pharmaceutically acceptable salt, solvate or hydrate thereof, wherein:
R² is selected from
hydrogen;
halogen;
C₁₋₈ alkyl, C₂₋₈ alkenyl, and C₂₋₈ alkynyl, each of which is optionally substituted with one or more R⁷; and
C₃₋₁₀ carbocycle and 3- to 10-membered heterocycle; wherein the C₃₋₁₀ carbocycle and 3- to 10-membered heterocycle are each optionally substituted with one or more R⁷;
R⁴, R⁵, R⁶, R⁷, and R⁸ are each independently selected from:
hydrogen;
C₁₋₁₀ alkyl, C₂₋₁₀ alkenyl, and C₂₋₁₀ alkynyl optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₁₋₆ cycloalkyl, and C₁₋₆ cycloalkenyl; and
C₃₋₁₀ carbocycle or 3- to 10-membered heterocycle; wherein the C₃₋₁₀ carbocycle and 3- to 10-membered heterocycle are each optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), and C₁₋₈ alkyl.

102. The composition of claim 101, wherein R² is C₃₋₇ carbocycle or 3- to 7-membered heterocycle; wherein the C₃₋₇ carbocycle and 3- to 7-membered heterocycle are each optionally substituted with one or more R⁷.

103. The composition of claim 101 or 102, wherein R² is phenyl, optionally substituted with one or more R⁷.

104. The composition of claim 101 or 102, wherein R² is a cycloalkyl selected from cyclooctane, cycloheptane, cyclohexane, cyclopentane, cyclobutane, and cyclopropane, any of which is optionally substituted with one or more R⁷.

105. The composition of claim 101 or 102, wherein R² is a 6-membered heteroaryl selected from pyridine, pyrazine, and pyrimidine, any of which is optionally substituted with one or more R⁷.

106. The composition of claim 101 or 102, wherein R² is a 5-membered heteroaryl selected from thiophene, furan, pyrrole, pyrazole, selenophene, imidazole, thiazole, isothiazole, oxatriazole, oxadiazole, oxazole, and thiadiazole, any of which is optionally substituted with one or more R⁷.

107. The composition of claim 101 or 102, wherein R² is a 6-membered heterocycloalkenyl selected from dihydropyridine, tetrahydropyridine, dihydropyridazine, tetrahydropyridazine, dihydropyrimidine, tetrahydropyrimidine, dihydropyrazine, tetrahydropyridazine, dihydropyrimidine, tetrahydropyrimidine, dihydropyrazine, tetrahydropyrazine, pyran, dihydropyran, thiopyran, dihydrothiopyran, dioxine, dihydrodioxine, oxazine, dihydrooxazine, thiazine, and dihydrothiazine, any of which is optionally substituted with one or more R⁷.

108. The composition of claim 101 or 102, wherein R² is a 5-membered heterocycloalkenyl selected from pyrroline, pyrazoline, imidazoline, triazoline, dihydrofuran, dihydrothiophene, oxazoline, isooxazoline, thiazoline, isothiazoline, oxadiazoline, and thiadiazoline, any of which is optionally substituted with one or more R⁷.

109. The composition of claim 101 or 102, wherein R² is a 6-membered heterocycloalkyl selected from piperidine, piperazine, tetrahydrothiopyran, tetrahydropyran, dioxane, morpholine, and thiomorpholine, any of which is optionally substituted with one or more R⁷.

110. The composition of claim 101 or 102, wherein R² is a 5-membered heterocycloalkyl selected from tetrahydrofuran, pyrrolidone, and tetrahydrothiophene, any of which is optionally substituted with one or more R⁷.

111. The composition of claim 101 or 102, wherein R² is a 4-membered heterocycloalkyl selected from azetidine, oxetane, and thietane, any of which is optionally substituted with one or more R⁷.

112. The composition of claim 101, wherein R² is a 6-6 fused ring system selected from naphthalene, quinoline, isoquinoline, pteridine, benzopyran, dihydroquinoline, dihydroisoquinoline, dihydronapthalene, and tetrahydronapthalene, any of which is optionally substituted with one or more R⁷.

113. The composition of claim 101, wherein R² is a 5-6 fused ring system selected from benzimidazole, indole, azaindole, indazole, benzothiophene, benzofuran, benzoselenophene, benzimidazole, benzotriazole, benzothiazole, benzisothiazole, benzoxadiazole, benzoxazole, and benzothiadiazole, imidazo[4,5-b]-pyridine, imidazo[4,5-c]-pyridine, pyrazolo[3,4-b]pyridine, pyrazolo[3,4-c]pyridine, pyrazolo[4,3-b]pyridine, pyrazolo[4,3-b]pyridine, oxazolo[4,5-b]pyridine, oxazolo[4,5-c]pyridine, oxazolo[5,4-b]pyridine, oxazolo[5,4-c]pyridine, thiazolo[4,5-b]pyridine, thiazolo[4,5-c]pyridine, thiazolo[5,4-b]pyridine, thiazolo[5,4-c]pyridine, isooxazolo[4,5-b]pyridine, isooxazolo[4,5-c]pyridine, isooxazolo[3,4-b]pyridine, isooxazolo[3,4-c]pyridine, isooxazolo[4,3-b]pyridine, isooxazolo[4,3-c]pyridine, isooxazolo[5,4-b]pyridine, isooxazolo[5,4-c]pyridine, isothiazolo[4,5-b]pyridine, isothiazolo[4,5-c]pyridine, isothiazolo[5,4-b]pyridine, isothiazolo[5,4-c]pyridine, isothiazolo[3,4-b]pyridine, isothiazolo[3,4-c]pyridine, isothiazolo[4,3-b]pyridine, isothiazolo[4,3-c]pyridine and purine, any of which is optionally substituted with one or more R⁷.

114. The composition of claim 101, wherein R² is a 5-5 fused ring system selected from pyrrolo[2,1-b]thiazole, pyrazolo[5,1-b]thiazole, imidazo[5,1-b]thiazole, imidazo[2,1-b]thiazole, thiazolo[3,2-b][1,2,4]triazole, thiazolo[3,2-c][1,2,4]triazole, imidazo[2,1-b]thiadiazole, imidazo[1,2-d][1,2,4]thiadiazole, thiazolo[3,2-d]tetrazole, thieno[3,2-b]thiophene, thieno[2,3-b]thiophene, thieno[3,4-b]thiophene, thieno[3,4-c]thiophene, any of which is optionally substituted with one or more R⁷.

115. The composition of claim 101, wherein R² is a bridged ring system selected from norbornane, norbornene, bicyclo[1.1.0]butane, bicyclo[3.2.0]heptane, bicyclo[3.3.0]octane, bicyclo[4.3.0]nonane, bicyclo[1.1.1]pentane, bicyclo[2.2.1]hexane, bicyclo[3.3.1]nonane, bicyclo[2.2.2]octane, adamantane, cubane, housane, any of which is optionally substituted with one or more R⁷.

116. The composition of claims 101 to 115, wherein each of R⁴ or R⁵ may independently be 3- to 10-membered heterocycle selected from azacyclopropane, pyrrolidine, piperidine, azepane, azocane, azonane, morpholine, thiomorpholine, 1,4-piperazine, 1,4-oxazepane, and 1,4-diazepane.

117. The composition of claim 101, wherein the compound of Formula (IV) is selected from:

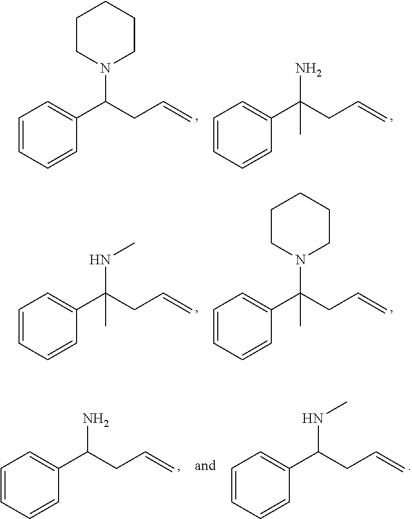

118. A method for treating a physical, psychiatric, or neurological disorder comprising administering to a subject in need thereof the pharmaceutical composition of any one of claims 101 to 117.

119. A pharmaceutical composition comprising a compound of Formula (V): 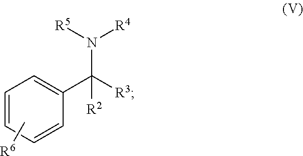
or an enantiomer, a mixture of enantiomers, or an isotopic variant thereof, or a pharmaceutically acceptable salt, solvate or hydrate thereof, wherein:
R² is selected from
hydrogen;
halogen;
C₁₋₈ alkyl, C₂₋₈ alkenyl, and C₂₋₈ alkynyl, each of which is optionally substituted with one or more R⁷; and
C₃₋₁₀ carbocycle and 3- to 10-membered heterocycle; wherein the C₃₋₁₀ carbocycle and 3- to 10-membered heterocycle are each optionally substituted with one or more R⁷;
R³ is selected from:
C₁₋₆ alkyl, C₂₋₆ alkenyl, and C₂₋₆ alkynyl, each of which is optionally substituted with one or more R⁸; and
C₃₋₁₀ carbocycle and 3- to 10-membered heterocycle; wherein the C₃₋₁₀ carbocycle and 3- to 10-membered heterocycle are each optionally substituted with one or more R⁸;
R⁴, R⁵, R⁶, R⁷, and R⁸ are each independently selected from:
hydrogen;
C₁₋₁₀ alkyl, C₂₋₁₀ alkenyl, and C₂₋₁₀ alkynyl optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), C₁₋₆ alkyl, C₁₋₆ cycloalkyl, and C₁₋₆ cycloalkenyl; and
C₃₋₁₀ carbocycle or 3- to 10-membered heterocycle; wherein the C₃₋₁₀ carbocycle and 3- to 10-membered heterocycle are each optionally substituted with one or more substituents independently selected from hydrogen, halogen, —CN, —OH, ═O, ═S, ═NH, —SH, —NO₂, —NH₂, —O—C₁₋₆ alkyl, —S—C₁₋₆ alkyl, —N(C₁₋₆ alkyl)₂, —NH(C₁₋₆ alkyl), and C₁₋₈ alkyl.

120. The composition of claim 119, wherein R³ is C₃₋₇ carbocycle or 3- to 7-membered heterocycle; wherein the C₃₋₇ carbocycle and 3- to 7-membered heterocycle are each optionally substituted with one or more R⁸.

121. The composition of claim 119 or 120, wherein R³ is phenyl, optionally substituted with one or more R⁸.

122. The composition of claim 119 or 120, wherein R³ is a cycloalkyl selected from cyclooctane, cycloheptane, cyclohexane, cyclopentane, cyclobutane, and cyclopropane, any of which is optionally substituted with one or more R⁸.

123. The composition of claim 119 or 120, wherein R³ is a 6-membered heteroaryl selected from pyridine, pyrazine, and pyrimidine, any of which is optionally substituted with one or more R⁸.

124. The composition of claim 119 or 120, wherein R³ is a 5-membered heteroaryl selected from thiophene, furan, pyrrole, pyrazole, selenophene, imidazole, thiazole, isothiazole, oxatriazole, oxadiazole, oxazole, and thiadiazole, any of which is optionally substituted with one or more R⁸.

125. The composition of claim 119 or 120, wherein R³ is a 6-membered heterocycloalkenyl selected from dihydropyridine, tetrahydropyridine, dihydropyridazine, tetrahydropyridazine, dihydropyrimidine, tetrahydropyrimidine, dihydropyrazine, tetrahydropyridazine, dihydropyrimidine, tetrahydropyrimidine, dihydropyrazine, tetrahydropyrazine, pyran, dihydropyran, thiopyran, dihydrothiopyran, dioxine, dihydrodioxine, oxazine, dihydrooxazine, thiazine, and dihydrothiazine, any of which is optionally substituted with one or more R⁸.

126. The composition of claim 119 or 120, wherein R³ is a 5-membered heterocycloalkenyl selected from pyrroline, pyrazoline, imidazoline, triazoline, dihydrofuran, dihydrothiophene, oxazoline, isooxazoline, thiazoline, isothiazoline, oxadiazoline, and thiadiazoline, any of which is optionally substituted with one or more R⁸.

127. The composition of claim 119 or 120, wherein R³ is a 6-membered heterocycloalkyl selected from piperidine, piperazine, tetrahydrothiopyran, tetrahydropyran, dioxane, morpholine, and thiomorpholine, any of which is optionally substituted with one or more R⁸.

128. The composition of claim 119 or 120, wherein R³ is a 5-membered heterocycloalkyl selected from tetrahydrofuran, pyrrolidone, and tetrahydrothiophene, any of which is optionally substituted with one or more R⁸.

129. The composition of claim 119 or 120 wherein R³ is a 4-membered heterocycloalkyl selected from azetidine, oxetane, and thietane, any of which is optionally substituted with one or more R⁸.

130. The composition of claim 119, wherein R³ is a 6-6 fused ring system selected from naphthalene, quinoline, isoquinoline, pteridine, benzopyran, dihydroquinoline, dihydroisoquinoline, dihydronapthalene, and tetrahydronapthalene, any of which is optionally substituted with one or more R⁸.

131. The composition of claim 119, wherein R³ is a 5-6 fused ring system selected from benzimidazole, indole, azaindole, indazole, benzothiophene, benzofuran, benzoselenophene, benzimidazole, benzotriazole, benzothiazole, benzisothiazole, benzoxadiazole, benzoxazole, and benzothiadiazole, imidazo[4,5-b]-pyridine, imidazo[4,5-c]-pyridine, pyrazolo[3,4-b]pyridine, pyrazolo[3,4-c]pyridine, pyrazolo[4,3-b]pyridine, pyrazolo[4,3-b]pyridine, oxazolo[4,5-b]pyridine, oxazolo[4,5-c]pyridine, oxazolo[5,4-b]pyridine, oxazolo[5,4-c]pyridine, thiazolo[4,5-b]pyridine, thiazolo[4,5-c]pyridine, thiazolo[5,4-b]pyridine, thiazolo[5,4-c]pyridine, isooxazolo[4,5-b]pyridine, isooxazolo[4,5-c]pyridine, isooxazolo[3,4-b]pyridine, isooxazolo[3,4-c]pyridine, isooxazolo[4,3-b]pyridine, isooxazolo[4,3-c]pyridine, isooxazolo[5,4-b]pyridine, isooxazolo[5,4-c]pyridine, isothiazolo[4,5-b]pyridine, isothiazolo[4,5-c]pyridine, isothiazolo[5,4-b]pyridine, isothiazolo[5,4-c]pyridine, isothiazolo[3,4-b]pyridine, isothiazolo[3,4-c]pyridine, isothiazolo[4,3-b]pyridine, isothiazolo[4,3-c]pyridine and purine, any of which is optionally substituted with one or more R⁸.

132. The composition of claim 119, wherein R³ is a 5-5 fused ring system selected from pyrrolo[2,1-b]thiazole, pyrazolo[5,1-b]thiazole, imidazo[5,1-b]thiazole, imidazo[2,1-b]thiazole, thiazolo[3,2-b][1,2,4]triazole, thiazolo[3,2-c][1,2,4]triazole, imidazo[2,1-b]thiadiazole, imidazo[1,2-d][1,2,4]thiadiazole, thiazolo[3,2-d]tetrazole, thieno[3,2-b]thiophene, thieno[2,3-b]thiophene, thieno[3,4-b]thiophene, thieno[3,4-c]thiophene, any of which is optionally substituted with one or more R⁸.

133. The composition of claim 119, wherein R³ is a bridged ring system selected from norbornane, norbornene, bicyclo[1.1.0]butane, bicyclo[3.2.0]heptane, bicyclo[3.3.0]octane, bicyclo[4.3.0]nonane, bicyclo[1.1.1]pentane, bicyclo[2.2.1]hexane, bicyclo[3.3.1]nonane, bicyclo[2.2.2]octane, adamantane, cubane, housane, any of which is optionally substituted with one or more R⁸.

134. The composition of any of claims 119 to 133, wherein R² is C₃₋₇ carbocycle or 3- to 7-membered heterocycle; wherein the C₃₋₇ carbocycle and 3- to 7-membered heterocycle are each optionally substituted with one or more R⁷.

135. The composition of any of claims 119 to 134, wherein R² is phenyl, optionally substituted with one or more R⁷.

136. The composition of any of claims 119 to 134, wherein R² is a cycloalkyl selected from cyclooctane, cycloheptane, cyclohexane, cyclopentane, cyclobutane, and cyclopropane, any of which is optionally substituted with one or more R⁷.

137. The composition of any of claims 119 to 134, wherein R² is a 6-membered heteroaryl selected from pyridine, pyrazine, and pyrimidine, any of which is optionally substituted with one or more R⁷.

138. The composition of any of claims 119 to 134, wherein R² is a 5-membered heteroaryl selected from thiophene, furan, pyrrole, pyrazole, selenophene, imidazole, thiazole, isothiazole, oxatriazole, oxadiazole, oxazole, and thiadiazole, any of which is optionally substituted with one or more R⁷.

139. The composition of any of claims 119 to 134, wherein R² is a 6-membered heterocycloalkenyl selected from dihydropyridine, tetrahydropyridine, dihydropyridazine, tetrahydropyridazine, dihydropyrimidine, tetrahydropyrimidine, dihydropyrazine, tetrahydropyridazine, dihydropyrimidine, tetrahydropyrimidine, dihydropyrazine, tetrahydropyrazine, pyran, dihydropyran, thiopyran, dihydrothiopyran, dioxine, dihydrodioxine, oxazine, dihydrooxazine, thiazine, and dihydrothiazine, any of which is optionally substituted with one or more R⁷.

140. The composition of any of claims 119 to 134, wherein R² is a 5-membered heterocycloalkenyl selected from pyrroline, pyrazoline, imidazoline, triazoline, dihydrofuran, dihydrothiophene, oxazoline, isooxazoline, thiazoline, isothiazoline, oxadiazoline, and thiadiazoline, any of which is optionally substituted with one or more R⁷.

141. The composition of any of claims 119 to 134, wherein R² is a 6-membered heterocycloalkyl selected from piperidine, piperazine, tetrahydrothiopyran, tetrahydropyran, dioxane, morpholine, and thiomorpholine, any of which is optionally substituted with one or more R⁷.

142. The composition of any of claims 119 to 134, wherein R² is a 5-membered heterocycloalkyl selected from tetrahydrofuran, pyrrolidone, and tetrahydrothiophene, any of which is optionally substituted with one or more R⁷.

143. The composition of any of claims 119 to 134, wherein R² is a 4-membered heterocycloalkyl selected from azetidine, oxetane, and thietane, any of which is optionally substituted with one or more R⁷.

144. The composition of any of claims 119 to 133, wherein R² is a 6-6 fused ring system selected from naphthalene, quinoline, isoquinoline, pteridine, benzopyran, dihydroquinoline, dihydroisoquinoline, dihydronapthalene, and tetrahydronapthalene, any of which is optionally substituted with one or more R⁷.

145. The composition of any of claims 119 to 133, wherein R² is a 5-6 fused ring system selected from benzimidazole, indole, azaindole, indazole, benzothiophene, benzofuran, benzoselenophene, benzimidazole, benzotriazole, benzothiazole, benzisothiazole, benzoxadiazole, benzoxazole, and benzothiadiazole, imidazo[4,5-b]-pyridine, imidazo[4,5-c]-pyridine, pyrazolo[3,4-b]pyridine, pyrazolo[3,4-c]pyridine, pyrazolo[4,3-b]pyridine, pyrazolo[4,3-b]pyridine, oxazolo[4,5-b]pyridine, oxazolo[4,5-c]pyridine, oxazolo[5,4-b]pyridine, oxazolo[5,4-c]pyridine, thiazolo[4,5-b]pyridine, thiazolo[4,5-c]pyridine, thiazolo[5,4-b]pyridine, thiazolo[5,4-c]pyridine, isooxazolo[4,5-b]pyridine, isooxazolo[4,5-c]pyridine, isooxazolo[3,4-b]pyridine, isooxazolo[3,4-c]pyridine, isooxazolo[4,3-b]pyridine, isooxazolo[4,3-c]pyridine, isooxazolo[5,4-b]pyridine, isooxazolo[5,4-c]pyridine, isothiazolo[4,5-b]pyridine, isothiazolo[4,5-c]pyridine, isothiazolo[5,4-b]pyridine, isothiazolo[5,4-c]pyridine, isothiazolo[3,4-b]pyridine, isothiazolo[3,4-c]pyridine, isothiazolo[4,3-b]pyridine, isothiazolo[4,3-c]pyridine and purine, any of which is optionally substituted with one or more R⁷.

146. The composition of any of claims 119 to 133, wherein R² is a 5-5 fused ring system selected from pyrrolo[2,1-b]thiazole, pyrazolo[5,1-b]thiazole, imidazo[5,1-b]thiazole, imidazo[2,1-b]thiazole, thiazolo[3,2-b][1,2,4]triazole, thiazolo[3,2-c][1,2,4]triazole, imidazo[2,1-b]thiadiazole, imidazo[1,2-d][1,2,4]thiadiazole, thiazolo[3,2-d]tetrazole, thieno[3,2-b]thiophene, thieno[2,3-b]thiophene, thieno[3,4-b]thiophene, thieno[3,4-c]thiophene, any of which is optionally substituted with one or more R⁷.

147. The composition of any of claims 119 to 133, wherein R² is a bridged ring system selected from norbornane, norbornene, bicyclo[1.1.0]butane, bicyclo[3.2.0]heptane, bicyclo[3.3.0]octane, bicyclo[4.3.0]nonane, bicyclo[1.1.1]pentane, bicyclo[2.2.1]hexane, bicyclo[3.3.1]nonane, bicyclo[2.2.2]octane, adamantane, cubane, and housane, any of which is optionally substituted with one or more R⁷.

148. The composition of claims 119 to 147, wherein each of R⁴ or R⁵ is independently selected from azacyclopropane, pyrrolidine, piperidine, azepane, azocane, azonane, morpholine, thiomorpholine, 1,4-piperazine, 1,4-oxazepane, and 1,4-diazepane.

149. The composition of claim 119, wherein the compound of Formula (V) is selected from:

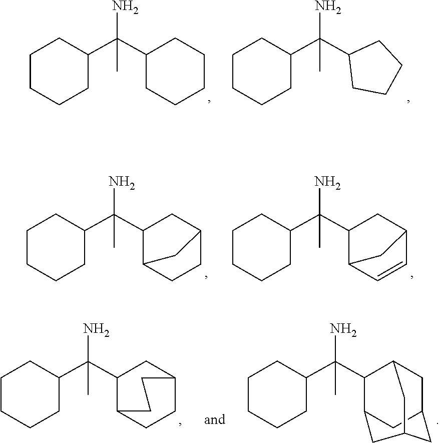

150. A method for treating a physical, psychiatric, or neurological disorder comprising administering to a subject in need thereof the pharmaceutical composition of any one of claims 119 to 149.

151. A compound selected from Table 1a.
